SEQUENCE LISTING (84 sequences)

SEQ ID NO: 1 - RNA, 12 nt, Artificial Sequence
  source: /note=“Description of Artificial Sequence Synthetic oligonucleotide”
uuuaaauuuaaa

SEQ ID NO: 2 - RNA, 85 nt, Homo sapiens
ccuuagcagagcuguggagugugacaaugguguuugugucuaaacuaucaaacgccauuaucacacuaaauagcuacugcuaggc

SEQ ID NO: 3 - RNA, 22 nt, Homo sapiens
uggagugugacaaugguguuug

SEQ ID NO: 4 - RNA, 22 nt, Homo sapiens
aacgccauuaucacacuaaaua

SEQ ID NO: 5 - RNA, 71 nt, Homo sapiens
gccaacccaguguucagacuaccuguucaggaggcucucaauguguacaguagucugcacauugguuaggc

SEQ ID NO: 6 - RNA, 23 nt, Homo sapiens
cccaguguucagacuaccuguuc

SEQ ID NO: 7 - RNA, 22 nt, Homo sapiens
acaguagucugcacauugguua

SEQ ID NO: 8 - RNA, 86 nt, Homo sapiens
ugccagucucuaggucccugagacccuuuaaccugugaggacauccagggucacaggugagguucuugggagccuggcgucuggcc

SEQ ID NO: 9 - RNA, 24 nt, Homo sapiens
ucccugagacccuuuaaccuguga

SEQ ID NO: 10 - RNA, 22 nt, Homo sapiens
acaggugagguucuugggagcc

SEQ ID NO: 11 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 12 - RNA, 22 nt, Homo sapiens
ucugaccuaugaauugacagcc

SEQ ID NO: 13 - RNA, 22 nt, Homo sapiens
cugccaauuccauaggucacag

SEQ ID NO: 14 - RNA, 83 nt, Homo sapiens
cggggugagguaguagguugugugguuucagggcagugauguugccccucggaagauaacuauacaaccuacugccuucccug

SEQ ID NO: 15 - RNA, 22 nt, Homo sapiens
ugagguaguagguugugugguu

SEQ ID NO: 16 - RNA, 22 nt, Homo sapiens
cuauacaaccuacugccuuccc

SEQ ID NO: 17 - RNA, 64 nt, Homo sapiens
ccccgcgacgagccccucgcacaaaccggaccugagcguuuuguucguucggcucgcgugaggc

SEQ ID NO: 18 - RNA, 23 nt, Homo sapiens
gcgacgagccccucgcacaaacc

SEQ ID NO: 19 - RNA, 22 nt, Homo sapiens
uuuguucguucggcucgcguga

SEQ ID NO: 20 - RNA, 85 nt, Homo sapiens
aggccucucucuccguguucacagcggaccuugauuuaaauguccauacaauuaaggcacgcggugaaugccaagaauggggcug

SEQ ID NO: 21 - RNA, 22 nt, Homo sapiens
cguguucacagcggaccuugau

SEQ ID NO: 22 - RNA, 22 nt, Homo sapiens
uaaggcacgcggugaaugccaa

SEQ ID NO: 23 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 24 - RNA, 22 nt, Homo sapiens
ggugcagugcugcaucucuggu

SEQ ID NO: 25 - RNA, 21 nt, Homo sapiens
ugagaugaagcacuguagcuc

SEQ ID NO: 26 - RNA, 87 nt, Homo sapiens
gacagugcagucacccauaaaguagaaagcacuacuaacagcacuggaggguguaguguuuccuacuuuauggaugaguguacugug

SEQ ID NO: 27 - RNA, 21 nt, Homo sapiens
cauaaaguagaaagcacuacu

SEQ ID NO: 28 - RNA, 23 nt, Homo sapiens
uguaguguuuccuacuuuaugga

SEQ ID NO: 29 - RNA, 110 nt, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 30 - RNA, 25 nt, Homo sapiens
agugguucuuaacaguucaacaguu

SEQ ID NO: 31 - RNA, 22 nt, Homo sapiens
gugaaauguuuaggaccacuag

SEQ ID NO: 32 - RNA, 80 nt, Homo sapiens
ugggaugagguaguagguuguauaguuuuagggucacacccaccacugggagauaacuauacaaucuacugucuuuccua

SEQ ID NO: 33 - RNA, 22 nt, Homo sapiens
ugagguaguagguuguauaguu

SEQ ID NO: 34 - RNA, 21 nt, Homo sapiens
cuauacaaucuacugucuuuc

SEQ ID NO: 35 - RNA, 71 nt, Homo sapiens
gcgacuguaaacauccucgacuggaagcugugaagccacagaugggcuuucagucggauguuugcagcugc

SEQ ID NO: 36 - RNA, 22 nt, Homo sapiens
uguaaacauccucgacuggaag

SEQ ID NO: 37 - RNA, 22 nt, Homo sapiens
cuuucagucggauguuugcagc

SEQ ID NO: 38 - RNA, 71 nt, Homo sapiens
ugggaaacauacuucuuuauaugcccauauggaccugcuaagcuauggaauguaaagaaguauguaucuca

SEQ ID NO: 39 - RNA, 22 nt, Homo sapiens
acauacuucuuuauaugcccau

SEQ ID NO: 40 - RNA, 22 nt, Homo sapiens
uggaauguaaagaaguauguau

SEQ ID NO: 41 - RNA, 85 nt, Homo sapiens
cgcuggcgacgggacauuauuacuuuugguacgcgcugugacacuucaaacucguaccgugaguaauaaugcgccguccacggca

SEQ ID NO: 42 - RNA, 21 nt, Homo sapiens
cauuauuacuuuugguacgcg

SEQ ID NO: 43 - RNA, 22 nt, Homo sapiens
ucguaccgugaguaauaaugcg

SEQ ID NO: 44 - RNA, 22 nt, Homo sapiens
caaacaccauugucacacucca

SEQ ID NO: 45 - RNA, 22 nt, Homo sapiens
uaaccaaugugcagacuacugu

SEQ ID NO: 46 - RNA, 24 nt, Homo sapiens
ucacagguuaaagggucucaggga

SEQ ID NO: 47 - RNA, 22 nt, Homo sapiens
ggcugucaauucauaggucaga

SEQ ID NO: 48 - RNA, 22 nt, Homo sapiens
aaccacacaaccuacuaccuca

SEQ ID NO: 49 - RNA, 22 nt, Homo sapiens
ucacgcgagccgaacgaacaaa

SEQ ID NO: 50 - RNA, 22 nt, Homo sapiens
uuggcauucaccgcgugccuua

SEQ ID NO: 51 - RNA, 21 nt, Homo sapiens
gagcuacagugcuucaucuca

SEQ ID NO: 52 - RNA, 23 nt, Homo sapiens
uccauaaaguaggaaacacuaca

SEQ ID NO: 53 - RNA, 22 nt, Homo sapiens
cuagugguccuaaacauuucac

SEQ ID NO: 54 - RNA, 22 nt, Homo sapiens
aacuauacaaccuacuaccuca

SEQ ID NO: 55 - RNA, 22 nt, Homo sapiens
cuuccagucgaggauguuuaca

SEQ ID NO: 56 - RNA, 22 nt, Homo sapiens
auacauacuucuuuacauucca

SEQ ID NO: 57 - RNA, 22 nt, Homo sapiens
cgcauuauuacucacgguacga

SEQ ID NO: 58 - RNA, 1705 nt, Homo sapiens
augugucaccagcaguuggucaucucuugguuuucccugguuuuucuggcaucuccccucguggccauaugggaacugaagaaagauguuuaugucguagaauuggauugguauccggaugccccuggagaaauggugguccucaccugugacaccccugaagaagaugguaucaccuggaccuuggaccagagcagugaggucuuaggcucuggcaaaacccugaccauccaagucaaagaguuuggagaugcuggccaguacaccugucacaaaggaggcgagguucuaagccauucgcuccugcugcuucacaaaaaggaagauggaauuugguccacugauauuuuaaaggaccagaaagaacccaaaaauaagaccuuucuaagaugcgaggccaagaauuauucuggacguuucaccugcugguggcugacgacaaucaguacugauuugacauucagugucaaaagcagcagaggcucuucugacccccaaggggugacgugcggagcugcuacacucucugcagagagagucagaggggacaacaaggaguaugaguacucaguggagugccaggaggacagugccugcccagcugcugaggagagucugcccauugaggucaugguggaugccguucacaagcucaaguaugaaaacuacaccagcagcuucuucaucagggacaucaucaaaccugacccacccaagaacuugcagcugaagccauuaaagaauucucggcagguggaggucagcugggaguacccugacaccuggaguacuccacauuccuacuucucccugacauucugcguucagguccagggcaagagcaagagagaaaagaaagauagagucuucacggacaagaccucagccacggucaucugccgcaaaaaugccagcauuagcgugcgggcccaggaccgcuacuauagcucaucuuggagcgaaugggcaucugugcccugcagugguggcgguggcggcggaucuagaaaccuccccguggccacuccagacccaggaauguucccaugccuucaccacucccaaaaccugcugagggccgucagcaacaugcuccagaaggccagacaaacucuagaauuuuacccuugcacuucugaagagauugaucaugaagauaucacaaaagauaaaaccagcacaguggaggccuguuuaccauuggaauuaaccaagaaugagaguugccuaaauuccagagagaccucuuucauaacuaaugggaguugccuggccuccagaaagaccucuuuuaugauggcccugugccuuaguaguauuuaugaagacuugaagauguaccagguggaguucaagaccaugaaugcaaagcuucugauggauccuaagaggcagaucuuucuagaucaaaacaugcuggcaguuauugaugagcugaugcaggcccugaauuucaacagugagacugugccacaaaaauccucccuugaagaaccggauuuuuauaaaacuaaaaucaagcucugcauacuucuucaugcuuucagaauucgggcagugacuauugauagagugaugagcuaucugaaugcuuccuaacaaacaccauugucacacuccauuuaaaauacauacuucuuuacauuccauuuaaacuagugguccuaaacauuucacuuuaaacuuccagucgaggauguuuaca

SEQ ID NO: 59 - RNA, 1599 nt, Homo sapiens
augugucaccagcaguuggucaucucuugguuuucccugguuuuucuggcaucuccccucguggccauaugggaacugaagaaagauguuuaugucguagaauuggauugguauccggaugccccuggagaaauggugguccucaccugugacaccccugaagaagaugguaucaccuggaccuuggaccagagcagugaggucuuaggcucuggcaaaacccugaccauccaagucaaagaguuuggagaugcuggccaguacaccugucacaaaggaggcgagguucuaagccauucgcuccugcugcuucacaaaaaggaagauggaauuugguccacugauauuuuaaaggaccagaaagaacccaaaaauaagaccuuucuaagaugcgaggccaagaauuauucuggacguuucaccugcugguggcugacgacaaucaguacugauuugacauucagugucaaaagcagcagaggcucuucugacccccaaggggugacgugcggagcugcuacacucucugcagagagagucagaggggacaacaaggaguaugaguacucaguggagugccaggaggacagugccugcccagcugcugaggagagucugcccauugaggucaugguggaugccguucacaagcucaaguaugaaaacuacaccagcagcuucuucaucagggacaucaucaaaccugacccacccaagaacuugcagcugaagccauuaaagaauucucggcagguggaggucagcugggaguacccugacaccuggaguacuccacauuccuacuucucccugacauucugcguucagguccagggcaagagcaagagagaaaagaaagauagagucuucacggacaagaccucagccacggucaucugccgcaaaaaugccagcauuagcgugcgggcccaggaccgcuacuauagcucaucuuggagcgaaugggcaucugugcccugcagugguggcgguggcggcggaucuagaaaccuccccguggccacuccagacccaggaauguucccaugccuucaccacucccaaaaccugcugagggccgucagcaacaugcuccagaaggccagacaaacucuagaauuuuacccuugcacuucugaagagauugaucaugaagauaucacaaaagauaaaaccagcacaguggaggccuguuuaccauuggaauuaaccaagaaugagaguugccuaaauuccagagagaccucuuucauaacuaaugggaguugccuggccuccagaaagaccucuuuuaugauggcccugugccuuaguaguauuuaugaagacuugaagauguaccagguggaguucaagaccaugaaugcaaagcuucugauggauccuaagaggcagaucuuucuagaucaaaacaugcuggcaguuauugaugagcugaugcaggcccugaauuucaacagugagacugugccacaaaaauccucccuugaagaaccggauuuuuauaaaacuaaaaucaagcucugcauacuucuucaugcuuucagaauucgggcagugacuauugauagagugaugagcuaucugaaugcuuccuaa

SEQ ID NO: 60 - RNA, 541 nt, Homo sapiens
auguggcugcagagccugcugcucuugggcacuguggccugcagcaucucugcacccgcccgcucgcccagccccagcacgcagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggagugacaaacaccauugucacacuccauuuaaaauacauacuucuuuacauuccauuuaaacuagugguccuaaacauuucacuuuaaacuuccagucgaggauguuuaca

SEQ ID NO: 61 - RNA, 435 nt, Homo sapiens
auguggcugcagagccugcugcucuugggcacuguggccugcagcaucucugcacccgcccgcucgcccagccccagcacgcagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggaguga

SEQ ID NO: 62 - RNA, 3822 nt, Artificial Sequence
  source: /note=“Description of Artificial Sequence Synthetic polynucleotide”
auguuuguuuuucuuguuuuauugccacuagucucuagucaguguguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacccgccggaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaa

SEQ ID NO: 63 - RNA, 3928 nt, Artificial Sequence
  source: /note=“Description of Artificial Sequence Synthetic polynucleotide”
auguuuguuuuucuuguuuuauugccacuagucucuagucaguguguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacccgccggaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaacaaacaccauugucacacuccauuuaaaauacauacuucuuuacauuccauuuaaacuagugguccuaaacauuucacuuuaaacuuccagucgaggauguuuaca

SEQ ID NO: 64 - RNA, 3899 nt, Artificial Sequence
  source: /note=“Description of Artificial Sequence Synthetic polynucleotide”
auguucguuuucuugguccugcuuccccuggugucuucacagugcgugaaucugaccaccagaacacagcugccuccagcauacaccaacagcuucaccagaggcguguauuauccugacaagguguuucgcuccagcgugcugcacagcacccaggaccuuuuucugccuuuuuucuccaacgugacaugguuccacgcaauccacgugagcggaaccaacggaacgaagagauucgacaacccugugcugcccuucaacgacggaguguacuucgccagcacagagaagagcaacaucauccggggcuggaucuucggaaccacccuggacagcaaaacccaaucucugcuuaucgugaacaacgcaaccaacguggugaucaaggugugugaauuccaauuuuguaacgacccauuccugggaguguacuaccauaagaacaacaagagcuggauggaaagcgaguuccggguguacagcagcgccaacaacugcaccuucgaguacgugagucagcccuuucugauggaccuggaaggcaagcagggaaauuucaagaaucugagagaguucguguucaaaaacaucgauggcuauuucaagaucuauagcaagcacaccccuaucaaccuggugagagaucugccccagggcuucagcgcccuggagccucugguagaccuaccuaucggcaucaacauaacgagauuucagacccuguuggcucuucauaggagcuaccugacccccggcgauucuagcagcggauggacagccggcgccgcugccuacuacguuggcuaccugcaaccucggacauuccugcugaaauacaaugagaacggcacuaucaccgaugccguggacugugcccuggauccucugagcgaaaccaagugcacccugaagagcuuuaccguggaaaagggcaucuaccagaccagcaauuuccgggugcagccuacagagagcaucgugagauuccccaacaucaccaaucuguguccuuucggcgagguguucaacgcuacaagauucgcaagcguguacgccuggaaucggaagcggaucaguaacuguguggccgauuauucggugcuguauaauucugccagcuuuagcaccuucaagugcuacggugugagcccuaccaaacucaacgaccugugcuucaccaacguguaugccgacucuuucgugauccggggcgacgaggugcggcagaucgcccccggacagacaggcaaaaucgccgacuacaacuacaagcugccugacgacuucacagggugcguuaucgccuggaacagcaacaaucuggauucaaaggugggcggaaacuacaacuaccuguacagacuguucagaaaguccaaccugaagcccuuugagagagacaucucuacagaaaucuaccaggccggcuccaccccaugcaacggcguggaaggcuucaacugcuacuucccccugcagagcuacggcuuucagccuaccaauggcgucgguuaccagccuuaccgcguggucguucuauccuucgagcugcugcacgccccugcuacagugugcggaccuaagaagagcacaaaccuggucaaaaacaagugugucaacuucaacuucaacggccugaccggcacaggcguacugacagaaagcaacaagaaguuccugccuuuccagcaguuuggcagagauaucgcugauaccacagacgccgugcgggauccucagacacuggaaauccuggacaucacacccugcagcuuuggcggcgugucugucaucaccccaggcaccaacacguccaaccaaguggccgugcuguaccaggacgucaacugcaccgaggugcccguugcuauccacgccgaucagcuaacuccuaccuggcggguuuauagcaccggaucuaacguguuccagaccagggccggaugucugaucggcgcugaacacguaaacaauagcuacgagugugauaucccuaucggagccggcaucugugccagcuaccagacccagacaaauucuccacggagagcuagaagcgucgccucucagagcaucaucgccuacaccaugagccugggcgccgaaaacucuguggccuacagcaacaacagcaucgccauucccaccaacuuuacaaucagcgugacaacagagauccugccugugagcaugaccaaaaccagcguggacugcaccauguacaucugcggcgacagcaccgaaugcucuaaccuucugcugcaauacggcagcuucugcacucagcugaacagagcccugaccggcaucgccguggagcaggauaagaacacccaggagguguucgcccaggugaaacaaaucuacaagacaccucccaucaaggacuucggcggauuuaacuucagccagauccugccugacccaucuaagccuagcaagcgguccuuuaucgaggacuugcuguucaacaaggugacccuggccgaugccggcuuuaucaagcaguacggcgacugccuuggcgacaucgccgccagagaccugaucugcgcccagaaguuuaacggccugacagugcugccuccucugcugaccgacgaaaugaucgcccaguauaccagcgcucugcuggcggguaccaucaccagcggcuggaccuucggcgccggcgcugcucugcagaucccuuucgccaugcagauggccuaccgguucaacggcauuggcgugacccagaacgugcuguacgagaaucagaagcugaucgccaaccaguucaacagcgcuaucggaaaaauccaggacucucugagcucuacugccucugcucuugggaaacugcaggacgugguuaaucagaacgcccaggcccucaauacccuggugaagcaacugucgagcaauuuuggcgccaucagcagcgugcugaaugacauucugucuagacuggacccuccagaagcugaggugcagauugacagacugaucacaggcagacugcagagccugcagaccuacgugacccaacaacugaucagagccgccgagauuagggccucugccaaucuggccgccacgaaaauguccgagugcguccugggccagucaaagagaguggauuucugcggcaagggauaccaccugaugagcuucccccagagcgcuccgcacggcgugguguuucugcacgugaccuacgugccagcccaggagaagaacuucaccacggccccugccaucugccacgacggcaaggcccacuuccccagagaaggaguguucgugagcaauggcacacacugguucgugacacaaagaaacuucuacgagccucagaucaucacaaccgauaacaccuucgugagcggcaauugcgacguggugaucggcauugugaacaacaccguguacgacccccugcagcccgagcuggauagcuucaaagaggaacuggauaaguacuucaagaaccacaccagcccugauguggaucugggcgacauuucuggcaucaacgccucugucgugaacauccagaaagagauagauagacugaacgagguugcaaagaaccugaacgaaagccugaucgacuugcaggagcucggcaaguacgagcaguacaucaaguggccuugguacauuuggcugggcuuuaucgccggacugaucgccaucgugauggucacaaucaugcugugcugcaugacaaguugcuguuccugccugaagggcugcuguagcuguggaagcugcuguaaauucgacgaagaugacagcgagccugugcugaagggcgugaagcugcacuacacaugacaaacaccauugucacacuccauuuaaaggcugucaauucauaggucaguuuaaacuuccagucgaggauguuuaca

SEQ ID NO: 65 - RNA, 3890 nt, Artificial Sequence
  source: /note=“Description of Artificial Sequence Synthetic polynucleotide”
auguucguguuccugguguuacugccccuggugucuucucagugcgucaacuucaccacaagaacacagcugccuccugccuauacaaacagcuuuacccggggaguguacuaccccgauaaaguguuccggagcucugugcugcacagcacacaggacuuguuccugccuuucuucagcaaugugacaugguuccacgccauccacgucuccggcacaaacggcaccaagagguucgccaauccugugcugccauucaaugauggcguguauuucgccagcacagagaagucuaacaucaucagaggcuggaucuucggcaccacccucgacucuaaaacccagagccugcugauagugaacaacgccacaaacguggugaucaaggucugugaauuccaguucugcaacgacccauuccugggcguguacuaccacaagaauaacaagagcuggauggaaucugaguucagaguguauucaucagccaacaacugcacauucgaguacgugucucagccauuccugauggaccuggaaggcaagcagggcaauuucaagaaucucagagaguucgucuucaagaacaucgacggcuacuuuaagaucuauagcaagcacaccccuaucaaccugguucggggccugccccagggcuuuagcgcccuggaaccucugguggaucugccaauuggcaucaacaucacccgguuucagacacugcacaucagcuaccugacaccuggcgacagcagcagcggcuggaccgccggcgccgccgccuacuacgucggcuaccugcagccccggaccuuccugcugaaguacaaugaaaauggcaccaucacagacgccguggauugcgcccuggacccucugucugaaacaaagugcacccugaaaagcuucaccguggaaaagggcauauaccagaccuccaacuuccgggugcagccuacagagucuaucgugagauuccccaacaucaccaaucuguguccuuuuggcgagguguucaacgccaccagauucgcaagcguguacgccuggaaccggaagaggaucagcaacugcguggcagauuacagcgugcucuacaacagcgccaguuucucuaccuuuaagugcuacggcgucagcccuacaaaacugaacgaucugugcuucaccaacguguacgccgauuccuuugugauacggggcgacgaaguuagacagaucgccccuggacagacaggaaauaucgccgacuacaacuauaagcugccugacgacuucaccggcugcgucaucgcuuggaacuccaacaaccuggauuccaaggugggcggaaacuacaacuaccuguacagacuguucagaaagagcaaccugaaaccuuucgagagggacaucagcacagagaucuaccaggccggcagcacccccuguaauggagucaaaggcuucaauugcuacuucccucugcagucuuacggcuuccagccaacauacggcgugggcuaccagcccuaccgggugguugugcuguccuucgagcugcugcaugccccagccacaguaugcgguccuaagaaaagcaccaaccuggugaagaacaaaugugugaacuuuaacuuuaacggccugaccggcaccggcgugcugaccgaauccaauaagaaguuccugccguuccagcaguuuggcagagauaucgccgacaccacagacgccgugagagacccccagacccuggaaauccuggacaucaccccuugcuccuuuggaggggugagcgugaucaccccgggcacaaacaccagcaaccagguggccgugcuguaccagggcgugaauuguaccgaggugccuguggcgauccacgccgaucagcugaccccuaccuggcggguguacagcaccggaucuaacguguuccaaacaagagccggcugucugaucggagcugaacacgugaacaacucuuacgagugugacauuccuaucggcgccggcaucugcgccucuuaucagacccagaccaacagccccagacgugccagaucuguggccucucagagcaucaucgccuacaccaugucucugggaguggaaaacuccguggcuuacagcaacaauucuaucgccauccccaccaacuuuacaaucagcgugaccaccgagauacugccuguguccaugacaaagaccagcguggacugcacuauguacaucugcggcgacagcacagaaugcagcaaccugcugcugcaguacggaagcuuuuguacucagcugaacagagcccugacuggcaucgcuguugagcaggauaagaauacucaggaggucuucgcucaagugaagcagaucuacaagaccccuccaaucaaggacuucggcggcuucaacuucagccaaauucugccugauccuagcaagcccagcaagcggagcuucaucgaggaccugcuguuuaacaaagugacacuugccgacgccggauucauuaagcaguauggcgacugccugggcgacaucgccgcgagagauuugaucugcgcccaaaaguucaacggccucaccgugcugccuccucuucugaccgacgagaugaucgcucaguacaccagcgcucuucuggccggcacaaucaccagcggcuggacauuuggcgcuggugccgcccuccagaucccuuucgccaugcagauggccuacagauucaacggcaucggcgucacccaaaacgugcucuaugagaaccagaaacuuaucgcuaaucaguucaacucugccaucggcaagauccaagauagccuguccuccaccgcuagcgcccugggaaagcuccaggacguggugaaucagaacgcccaagcccugaacacccuggugaaacagcugagcagcaacuucggcgcuaucagcuccguucugaacgacauucugucuagacuggacccuccugaggccgagguccagaucgauagacugaucacuggacgccugcaaucacugcaaacauacgugacccagcagcugauuagagccgccgagaucagagccucagcaaaucuggccgccacgaagaugagcgagugcgugcugggccagagcaagagagucgacuuuugcggcaaaggcuaccaccugaugagcuucccucagagcgccccacacggcgugguguuccugcaugugaccuacgugcccgcccaggaaaagaacuuuaccaccgccccugcuaucugucacgacggcaaggcccacuucccucgcgagggcguguucgucagcaacggcacccacugguucgugacacaacguaacuucuacgagccucagaucauaaccaccgauaacacauucgugagcggcaauugcgauguggugaucggaaucgugaacaacaccguguacgacccgcugcagcccgagcuggacagcuucaaagaggaacuggauaaguacuuuaagaaccacacuucuccagacguggaccugggcgauaucagcggaaucaacgcuuccguggugaacauccagaaggaaaucgacagacugaacgagguggcuaaaaaccugaaugagagccugaucgaccugcaggagcugggaaaauacgaacaguacaucaaguggccuugguacaucuggcugggcuuuaucgcuggccugaucgccaucgugauggugaccaucaugcugugcuguaugaccagcuguuguagcugccugaaggguugcuguuccugcggaagcugcugcaaguucgacgaggaugacagcgagcccgugcugaagggcguuaagcugcacuacaccugacaaacaccauugucacacuccauuuaaaggcugucaauucauaggucaguuuaaacuuccagucgaggauguuuaca

SEQ ID NO: 66 - RNA, 3922 nt, Artificial Sequence
  source: /note=“Description of Artificial Sequence Synthetic polynucleotide”
auguucgucuuccuggugcugcugccccuggugagcucucagugcgugaaucugagaacccggacacagcugccuccugccuacacaaacagcuuuacaagaggcgucuacuacccugacaagguguuccggucgagcgugcugcauucuacccaggauuuguuucuuccuuuuuucaguaacgugacaugguuccacgccauccacguguccggaaccaacggcaccaagagauucgacaacccugugcugccuuucaacgacggaguguauuucgccucuaccgagaagagcaacaucauucggggauggaucuucggaaccacccucgacagcaagacacagagccugcuaauagucaacaacgcuaccaacguggugauuaaggugugcgaguuccaauuuuguaacgauccuuuccugggaguuuauuaccauaagaacaauaaaagcuggauggaaagcgagguguacagcagcgcaaacaacugcacauucgaguaugugagccaaccuuuccugauggaccuggaaggcaagcaggggaacuucaagaaccugagggaauucguguuuaagaacaucgacggcuacuucaagaucuacagcaagcacacaccaauuaaccucguuagagaucugccacagggcuucagugcccuggaaccccugguggaucugcccaucggaaucaacaucaccagauuccagacccuccuggcccugcacagaagcuaucugaccccuggcgauucuagcucuggcuggacagcuggcgccgcugcuuacuacgugggcuaccugcagccuagaacauuccugcucaaguacaacgagaauggcacaaucaccgacgccguugacugcgcccuggauccuuugucugagacaaagugcacucugaagagcuucaccguggaaaagggcaucuaccagacaucuaacuucagagugcagccuacagagagcaucgugcgguuccccaacauaacaaaccuguguccauucggagaaguguuuaaugccaccagauucgcuagcguguacgccuggaaccggaagagaaucagcaacugcgucgccgacuacuccgugcuguacaauagcgccucuuucagcaccuuuaaguguuacggcgucucuccaacaaagcugaacgaccugugcuucacaaacguguacgccgacagcuucgugauccggggcgacgaagugcggcagauugcaccuggucagacugggaaaaucgcagauuacaacuacaagcugccagaugauuuuaccggcugugugaucgccuggaauagcaauaaccuggacagcaaagugggcggcaauuacaacuaccgguacagacuguuccggaagagcaaucugaagccuuuugagagagacaucuccacagagaucuaccaggccggcucuaagccuugcaacggcguggagggguuuaauugcuacuucccucugcagucuuacggguuccagcccaccaacggcgugggcuaucagccuuacagagugguggugcugucuuucgaacugcugcacgccccugcuaccgugugcgggccuaagaaguccaccaaccuugugaagaacaagugugugaacuucaacuucaauggccugaccggaaccggcguguugaccgaaucuaacaagaaauuccugccguuccaacaguucggcagagauauugccgacaccaccgaugccgugcgggacccccaaacccuggaaauccuggauaucaccccaugcagcuucggcggcgugucuguuaucacccccggcacaaacacgagcaaccaggucgccgugcucuaccagggcgugaacugcacagaagugcccguggcuauccacgccgaucagcugacacccacauggcggguguacagcacaggaucuaacguuuuccagacaagagcuggcugccuuauuggcgcugaacacgugaauaacagcuacgagugugacaucccaaucggcgccggcaucugcgccuccuaccagacccagaccaacagcagaaggagagcccggagcguggccagccagucuaucaucgccuacacaaugagccugggcgccgaaaacuccguggccuauagcaacaacuccaucgcuaucccuaccaacuucaccaucagcgugacaacggaaauucugccugugagcaugaccaagaccucuguggacuguacaauguacaucugcggcgacucuacagaaugcagcaaccugcugcugcaguacggcagcuuuugcacccagcuuaauagagcccugaccggaaucgccguggaacaggacaagaacacccaggaggucuucgcccaggugaaacagaucuacaagaccccuccuauuaaggacuucggcggauuuaacuucagccagauccugccugacccuagcaagcccagcaaaagaagcuucaucgaggaccuccuguucaacaaagugacccuggccgacgcuggcuuuaucaagcaguauggcgacugccugggcgacaucgcugcuagggaccugaucugugcccagaaguucaacggccugacagugcugccuccucugcugaccgaugaaaugaucgcccaguacacaagcgcccugcuggccggcaccaucaccagcggcuggaccuuuggagccggcgccgcccugcagauccccuuugccaugcagauggccuaucgguucaacggaaucggcgugacccaaaacguacuguacgagaaccagaagcugaucgccaaucaauuuaauagcgccaucgguaaaauccaggauagccugagcuccacugccagcgcccugggcaaacugcagaacguggugaaccagaacgcccaagcucugaacacccuggugaagcagcugucuuccaacuuuggugcuaucucuagcguccugaaugauauccugagcagacuggacccccccgaggccgaggugcagaucgauagacugaucaccggcagacugcaaucgcugcaaacuuacgugacccagcagcugaucagagccgccgagaucagagcuagcgccaaccuggccgccacuaagaugagcgagugcguucugggccagaguaagcggguggacuucuguggcaagggauaccaccugaugucuuuuccacagagcgccccucacggcgugguguuccugcacguuaccuacgugccagcccaggagaagaacuucaccacagccccugccaucugccacgacggcaaggcccacuucccuagagagggcguguucgucagcaacggcacccacugguucgugacgcaaagaaacuucuacgagccccagaucauuaccaccgacaauaccuucguauccggcaacugcgacguggugaucggcaucgugaacaacacaguguacgacccucugcagccugagcuggacucuuucaaggaagagcuggacaaguauuucaagaaccacaccagcccugauguggaccugggcgacaucagcggaaucaaugccucaguggugaacauccagaaagagaucgacagacugaacgaggucgccaagaaccugaaugagagccugaucgaccugcaggagcugggcaaguacgagcaauacaucaaguggccuugguacaucuggcugggcuucaucgccggccugauugccaucgugauggugaccaucaugcuguguugcaugaccaguugcuguaguugccugaaaggcugcuguucuugcggcagcugcugcaaauucgaugaggacgacuccgagcccgugcugaagggcgugaagcugcacuacaccugacaaacaccauugucacacuccauuuaaaauacauacuucuuuacauuccauuuaaacuagugguccuaaacauuucacuuuaaacuuccagucgaggauguuuaca

SEQ ID NO: 67 - RNA, 3887 nt, Artificial Sequence
  source: /note=“Description of Artificial Sequence Synthetic polynucleotide”
auguucguguuucugguccugcugccccugguguccucccagugcgugaaccugacgaccagaacacaacugccuccugccuacaccaacagcuuuacaagaggcgucuauuaccccgacaagguguuccggagcuccguccugcacucuacccaggaccuuuuccugccuuuuuucagcaacgugacaugguuccacgccaucagcgguaccaacggcaccaagcgcuucgacaacccugugcugccauuuaacgacggaguguauuucgccuccacagaaaagucgaacaucaucaggggcuggaucuucggcaccacacuggacagcaagacccagagccugcugaucgugaauaacgccacaaacguggucaucaaagucugcgaguuccaguucuguaaugaccccuuccugggcguucauaaaaacaacaaaagcuggauggaaagcgaguucagaguguauucuagcgccaauaacuguacauuugaguacgugucccagcccuuccugauggaccuggaaggcaagcaaggaaauuuuaagaaccugcgugaguuuguguucaagaacaucgaugguuauuucaaaaucuacagcaagcacacccccauuaaccuggucagagaccugccccagggcuucucugcccuggaaccucugguggaccugccgaucggaaucaacaucacacgguuccagacccugcuagcccugcauagaucuuaccugacccccggcgacagcucuuccggcuggacagccggcgccgcugcuuacuacgugggcuaccugcagccuagaaccuuccugcucaaguacaacgaaaauggcaccaucaccgacgccguggacugcgcccuggacccucuuagcgagacaaagugcacacugaagagcuucaccguggaaaagggcaucuaccagacaucgaacuucagagugcagccuaccgaguccaucgugagguuuccuaacaucaccaaccuguguccuuucggcgagguguucaacgccaccagauucgccagcguguacgccuggaauagaaagagaaucucuaacuguguggccgauuacagcgugcuguacaacucugccagcuuuagcaccuuuaaauguuacggcgugagcccuacaaagcugaacgaucugugcuucaccaauguguacgccgauucuuucgugauccggggcgaugaggugcggcagaucgccccaggccagacaggcaagaucgccgacuacaauuacaagcugccugaugacuucaccggcugcgugauugccuggaacagcaacaaucuggacagcaaggugggaggcaacuacaacuaccuguacagacucuuccggaagagcaaccugaaaccuuucgagagagauaucucaacugaaaucuaccaggccggcucaacacccugcaauggaguugagggcuucaauugcuacuucccccugcagucuuacggcuuucagccuacauacggcgugggcuaccagccuuaccgggugguuguguugagcuucgaacugcugcacgccccugcuaccgugugcgguccuaagaaaagcaccaaccuggugaagaacaagugcguaaacuucaacuucaacggccugacuggaacaggcguccugaccgaaagcaacaagaaguuccugccuuuucaacaauuuggcagagauauugaugauacaacagaugcugugcgggauccucagacacuggaaauccuggacaucacccccugcuccuucggcggagucagcgugauaaccccuggcacuaacaccagcaaucagguggccgugcucuaccagggcgucaacugcaccgaagucccuguugcuauccacgcugaccagcugacaccuaccuggagaguguauagcaccgguucuaacgucuuccagacccgcgccggcugucugaucggcgccgagcacgugaacaacagcuacgagugcgacauccccaucggcgcuggcaucugcgccucuuaucagacacagaccaacagccaccggagagcuagaagcguggccucucagucgaucauugccuacaccaugucccugggcgccgagaacucgguggccuacagcaacaauucuaucgccauccccaucaacuucaccaucagcgugacaaccgaaauucugccaguguccaugacgaagacauccguggacugcacaauguacaucugcggcgauagcacagaauguucuaaucugcugcuucaauauggaucuuucugcacccagcugaaccgggcccugacaggcaucgccguggaacaggacaaaaauacccaggagguguuugcccaggugaagcagaucuacaagaccccaccaaucaaggacuucggaggguuuaauuucagccagauccugcccgauccuagcaagccuuccaagcggaguuucaucgaggaccugcuguucaacaaagugacccuggcugaugccggcuucaucaagcaguacggcgacugccugggcgacaucgccgccagagaucugaucugcgcccagaaauuuaacgggcugaccgugcugccuccacugcugaccgacgagaugaucgcacaguacaccagcgcuuugcuggcgggcaccaucacgagcggcuggaccuucggggccggcgccgcccugcaaauuccuuucgccaugcagauggccuaccgguuuaacggcaucggcgugacacagaacgugcuauacgagaaccagaagcugauagcuaaucaguuuaacucugccaucggcaagauccaggacagccucuccagcaccgccagcgcccuggguaagcugcaggacguggugaaccagaacgcccaagcccugaacacccugguuaagcagcuguccagcaauuucggcgcuauuagcagcguucugaaugacauccuggccagacuggacccaccugaggccgaggugcagaucgauagacugaucacaggaagacugcagagccugcagaccuacgucacccaacaacucauccgggccgccgaaauccgggccagcgccaaccuugcagccaccaagaugagcgagugcgugcucggccagagcaaaagaguggacuuuugcggcaaaggcuaccaccugauguccuucccucagagcgccccacacggcgugguguuccugcacgugacauaugugcccgcacaggagaagaacuucacgacugcucccgccaucugccacgacggcaaggcccacuuccccagagaaggcguguucgugaguaacgggacccacugguucgugacccagagaaacuucuacgagccucagaucaucacaacccacaacacauucgugagcggaaacugcgauguggugaucggaaucgugaacaauaccguguacgacccucugcagccugagcuggacagcuucaaagaggaacucgacaaguauuuuaagaaccacaccagcccugacguggaucugggcgacaucagcggcaucaacgcuagcguggugaacauccagaaggaaaucgacagacugaacgagguggccaagaaccugaacgagagccugaucgaccugcaggagcugggcaaguacgagcaguacaucaaguggccuugguacauuuggcugggcuucaucgcagggcugaucgccaucgugauggugacaaucaugcuguguugcaugaccucuuguugcagcugucuuaaaggcugcugcagcuguggaagcugcugcaaguucgacgaggaugauagcgaacccgugcugaagggcgucaagcugcacuacaccugacaaacaccauugucacacuccauuuaaaggcugucaauucauaggucaguuuaaacuuccagucgaggauguuuaca

SEQ ID NO: 68 - RNA, 1738 nt, Artificial Sequence
  source: /note=“Description of Artificial Sequence Synthetic polynucleotide”
auguguccucagaagcuaaccaucuccugguuugccaucguuuugcuggugucuccacucauggccaugugggagcuggagaaagacguuuauguuguagagguggacuggacucccgaugccccuggagaaacagugaaccucaccugugacacgccugaagaagaugacaucaccuggaccucagaccagagacauggagucauaggcucuggaaagacccugaccaucacugucaaagaguuucuagaugcuggccaguacaccugccacaaaggaggcgagacucugagccacucacaucugcugcuccacaagaaggaaaauggaauuugguccacugaaauuuuaaaaaauuucaaaaacaagacuuuccugaagugugaagcaccaaauuacuccggacgguucacgugcucauggcuggugcaaagaaacauggacuugaaguucaacaucaagagcaguagcaguuccccugacucucgggcagugacauguggaauggcgucucugucugcagagaaggucacacuggaccaaagggacuaugagaaguauucaguguccugccaggaggaugucaccugcccaacugccgaggagacccugcccauugaacuggcguuggaagcacggcagcagaauaaauaugagaacuacagcaccagcuucuucaucagggacaucaucaaaccagacccgcccaagaacuugcagaugaagccuuugaagaacucacagguggaggucagcugggaguacccugacuccuggagcacuccccauuccuacuucucccucaaguucuuuguucgaauccagcgcaagaaagaaaagaugaaggagacagaggagggguguaaccagaaaggugcguuccucguagagaagacaucuaccgaaguccaaugcaaaggcgggaaugucugcgugcaagcucaggaucgcuauuacaauuccucaugcagcaagugggcauguguucccugcaggguccgauccgguggcgguggcucgggcgguggugggucggguggcggcggaucuagggucauuccagucucuggaccugccaggugucuuagccagucccgaaaccugcugaagaccacagaugacauggugaagacggccagagaaaaacugaaacauuauuccugcacugcugaagacaucgaucaugaagacaucacacgggaccaaaccagcacauugaagaccuguuuaccacuggaacuacacaagaacgagaguugccuggcuacuagagagacuucuuccacaacaagagggagcugccugcccccacagaagacgucuuugaugaugacccugugccuugguagcaucuaugaggacuugaagauguaccagacagaguuccaggccaucaacgcagcacuucagaaucacaaccaucagcagaucauucuagacaagggcaugcugguggccaucgaugagcugaugcagucucugaaucauaauggcgagacucugcgccagaaaccuccugugggagaagcagacccuuacagagugaaaaugaagcucugcauccugcuucacgccuucagcacccgcgucgugaccaucaacagggugaugggcuaucugagcuccgccuaacaaacaccauugucacacuccauuuaaaauacauacuucuuuacauuccauuuaaacuagugguccuaaacauuucacuuuaaacuuccagucgaggauguuuaca

SEQ ID NO: 69 - RNA, 1497 nt, Influenza A virus
auggcgucucaaggcaccaaacgaucauaugaacaaauggagacugguggggagcgccaggauaccacagaaaucagagcaucuguuggaagaaugauugguggaaucgggagauucuacauccaaaugugcacugaacucaaacucagugauuaugauggacgacuaauccagaauagcauaacaauagagaggauggugcuuucugcuuuugaugagagaagaaauaaauaccuagaagagcauccaagugcugggaaggacccuaagaaaacaggaggacccaucuauagaagaauagacggaaaauggacgagagaacucauccuuuaugacaaagaagaaauaaggagaguuuggcgccaagcaaacaauggcgaagaugcaacagcaggucuuacucauaucaugauuuggcauuccaaccugaaugaugccacauaucagaggacaagagcacuuguucgcacuggaauggaucccagaaugugcucucuaaugcaagguucaacacuucccagaaggucuggugccgcaggugcugcagugaaaggaguuggaacaauagcuauggaguuaaucagaaugaucaaacguggaaucaaugaccgaaauuucuggaggggugaaaauggacgaaggacaagaguugcuuaugaaagaaugugcaauauccucaaaggaaaauuucaaacagcugcccagagggcaaugauggaucaaguaagagaaagucgaaacccaggaaacgcugagauugaagaccucauuuuccuggcacggucagcacucauucugagaggaucaguugcacauaaauccugccugccugcuuguguguaugggcuugcaguagcaaguggccaugacuuugaaagggaaggguacucacuggucgggauagacccauucaaauuacuccaaaacagucaaguggucagccugaugagaccaaaugaaaauccagcucacaagagucaauugguauggauggcaugccacucugcugcauuugaagauuuaagaguaucaaguuucauaagaggaaagaaagugaucccaagaggaaagcuuuccacaagagggguucagauugcuucaaaugagaauguggaaaccauggacuccaauacccuggaacuaagaagcagauacugggccauaagaaccaggaguggaggaaauaccaaucaacagaaggcauccgcaggccagaucagugugcagccuacauucucagugcagcgaaaucucccuuuugaaagagcaaccguuauggcagcauucagcgggaacaaugaaggacggacauccgacaugagaacagaaguuauaagaaugauggaaagugcaaagccagaggauuuguccuuccaggggcggggagucuucgagcucucggacgaaaaggcaacgaacccgaucgugccuuccuuugacaugaguaaugaagggucuuauuucuucggagacaaugcagaggaguaugacaauuga

SEQ ID NO: 70 - RNA, 1410 nt, Influenza A virus
augaauccaaaccaaaagauaauaaccauugguucgaucuguaugacaauuggaauggcuaacuuaauauuacaaauuggaaacauaaucucaauauggguuagccacucaauucaaauuggaaaucaaagccagauugaaacaugcaaucaaagcgucauuacuuaugaaaacaacacuuggguaaaucagacauauguuaacaucagcaacaccaacuuugcugcuggacagucagugguuuccgugaaauuagcgggcaauuccucucucugcccuguuaguggaugggcuauauacaguaaagacaacaguguaagaaucgguuccaagggggauguguuugucauaagggaaccauucauaucaugcucucccuuggaaugcagaaccuucuucuugacucaaggggccuugcuaaaugacaaacauuccaauggaaccauuaaagacaggagcccauaccgaacccuaaugagcuguccuauuggugaaguucccucuccauacaacucaagauuugagucagucgcuuggucagcaagugcuugucaugauggcaucaauuggcuaacaauuggaauuucuggcccagacaguggggcaguggcuguguuaaaguacaauggcauaauaacagacacuaucaagaguuggaggaacaauauauugagaacacaagagucugaaugugcauguguaaaugguucuugcuuuaccauaaugaccgauggaccaagugauggacaggccucauacaaaaucuucagaauagaaaagggaaagauaaucaaaucagucgaaaugaaagccccuaauuaucacuaugaggaaugcuccuguuacccugauucuagugaaaucacaugugugugcagggauaacuggcauggcucgaaucgaccgugggugucuuucaaccagaaucuggaauaucagaugggauacauaugcagugggguuuucggagacaauccacgcccuaaugauaagacaggcaguugugguccaguaucgucuaauggagcaaauggaguaaaaggauuuucauucaaauacggcaaugguguuuggauagggagaacuaaaagcauuaguucaagaaaagguuuugagaugauuugggauccgaauggauggacugggacugacaauaaauucucaauaaagcaagauaucguaggaauaaaugaguggucaggguauagcgggaguuuuguucagcauccagaacuaacagggcuggauuguauaagaccuugcuucuggguugaacuaauaagagggcgacccgaagagaacacaaucuggacuagcgggagcagcauauccuuuugugguguaaacagugacacuguggguuggucuuggccagacggugcugaguugccauuuaccauugacaaguaa

SEQ ID NO: 71 - RNA, 285 nt, Artificial Sequence
  source: /note=“Description of Artificial Sequence Synthetic polynucleotide”
augagucuucuaaccgaggucgaaacgccuaccagaagcgaaugggagugcagaugcagcgguucaagugauccucucgucauuaucauugggaucuugcaccugauauuguggauuacugaucgucuuuuuuucaaaugcauuuaucgucgcuuuaaauacgguuugaaaagagggccuucuacggaaggagugccugaguccaugagggaagaauaucaacaggagcagcagagugcuguggauguugacgauggucauuuugucaacauagagcuagaguaa

SEQ ID NO: 72 - RNA, 798 nt, Artificial Sequence
  source: /note=“Description of Artificial Sequence Synthetic polynucleotide”
augaaagugaagcugcugguccugcugugcaccuucaccgccacauaugccgacaccaucugcaucggcuaccacgccaacaacagcacagauaccguggacaccgugcuggagaagaacgugaccgugacacacagcguuaaucugcuggaaaacggaggcggaggcaaguacgugugcagcgccaagcugagaauggugaccggccugagaaacaaaccuagcaagcagagccaaggccuguucggcgccaucgccggcuucaccgagggcggauggaccggcauggucgacggcugguacggcuaucaccaccagaacgagcagggcagcggcuacgccgcugaucagaagucuacacaaaaugcuauuaacggcaucaccaacaaggugaacagcgugaucgagaagaugaauacccaguacaccgccaucggcugugaauacaacaaguccgagcgguguaugaaacagaucgaagauaagaucgaggagaucgagagcaagaucuggugcuacaacgccgagcugcucgugcugcuggaaaacgagagaacacuggacuuccacgauucuaaugugaagaaccuguacgagaaggugaagagccagcugaagaacaacgcuaaggaaaucggcaacggauguuuugaguucuaccauaagugcaacgacgagugcauggaaucugugaagaauggaacauacgacuaccccaaguacagcgaggaauccaagcugaaccgggaaaaaaucgacggcgugaaacuggaaagcaugggcguguaccagaucuga

SEQ ID NO: 73 - RNA, 1410 nt, Influenza A virus
augaauccaaaccaaaagauaauaaccauugguucuaucuguaugacaauuggaacggcuaacuuaauauuacaaauuggaaacauaaucucaauauggguuagccacucaauucaaauuggaaaucaaagccagauugaaacaugcaauaaaagcgucauuacuuaugaaaacaacacuuggguaaaucagacauuuguuaacaucagcaacacuaacucugcugcuagacagucaguggcuuccgugaaauuagcgggcaauuccucucucugcccuguuaguggaugggcuauauacaguaaagacaacaguguaagaaucgguuccaagggggauguguuugucauaagggaaccauucauaucaugcucucccuuggaaugcagaaccuucuucuugacucaaggggcuuugcuaaaugacaaacauuccaauggaaccauuaaagacagaagcccauaucgaacccuaaugagcuguccuauuggugaaguucccucuccauacaacucaagauuugagucagucgcuuggucagcaagugcuugucaugauggcaccaauuggcuaacaauuggaauuucuggcccagacaguggggcaguggcuguguuaaaauacaauggcauaauaacagacacuaucaagaguuggaggaacaagauauugagaacacaagagucugaaugugcauguguaaaugguucuugcuuuaccauaaugaccgauggaccaagugauggacaggccucauacaaaaucuucagaauagaaaagggaaagauaaucaaaucagucgaaaugaaagccccuaauuaucacuaugaagaaugcuccuguuacccugauucuagugaaaucacaugugugugcagggauaacuggcauggcucgaaucgaccgugggugucuuucaaccagaaucuggaauaucagaugggauacauaugcagugggguuuucggagacaauccacgcccuaaugauaagacaggcaguugugguccaguaucgucuaauggagcaaaugggguaaaaggauuuucauucaaauacggcaaugguguuuggauagggagaacuaagagcauuaguucaagaaaagguuuugagaugauuugggauccgaauggauggacugggacugacaauaaauucucaaaaaagcaagauaucguaggaauaaaugaguggucaggguauagcgggaguuuuguucagcauccagaacuaacagggcugaauuguauaagaccuugcuucuggguugaacuaauaagaggacgacccgaagagaacacaaucuggacuagcgggagcagcauauccuuuugugguguagacagugacauuguggguuggucuuggccagacggugcugaguugccauuuaccauugacaaguaa

SEQ ID NO: 74 - RNA, 1701 nt, Influenza A virus
augaaggcaauacuaguaguuaugcuguauacauuuacaaccgcaaaugcagacacauuauguauagguuaucaugcgaacaauucaacagacacuguggacacaguacuagaaaagaauguaacaguaacacacucugucaaucuucuggaagacaagcauaacggaaaacuaugcaaacuaagagggguagccccauugcauuuggguaaauguaacauugcuggcuggauccugggaaauccagagugugaaucacucuccacagcaagaucaugguccuacauuguggaaacaucuaauucagacaauggaacguguuacccaggagauuucaucaauuaugaggagcuaagagagcaauugagcucagugucaucauuugaaagguuugaaauauuccccaagacaaguucauggccuaaucaugacucggacaaugguguaacggcagcauguccucacgcuggagcaaaaagcuucuacaaaaacuugauauggcugguuaaaaaaggaaaaucauacccaaagaucaaccaaaccuacauuaaugauaaagggaaagaaguccucgugcuguggggcauucaccauccaccuacuauugcugaccaacaaagucucuaucagaaugcagaugcauauguuuuuguggggacaucaagauacagcaagaaguucaagccggaaauagcaacaagacccaaagugagggaucaagaagggagaaugaacuauuacuggacacuaguagaaccgggagacaaaauaacauucgaagcaacugguaaucuaguggcaccgagauaugcauucacaauggaaagagaugcuggaucugguauuaucauuucagauacaccaguccacgauugcaauacaacuugucagacacccgagggugcuauaaacaccagccucccauuucagaauguacauccgaucacaauugggaaauguccaaaguauguaaaaagcacaaaauugagacuggccacaggauugaggaaugucccgucuauucaaucuagaggccuauucggggccauugcuggcuucaucgaagggggguggacagggaugguagauggaugguacgguuaucaccaucaaaaugagcaggggucaggauaugcagccgaucugaagagcacacaaaaugccauugauaagauuacuaacaaaguaaauucuguuauugaaaagaugaauacacaguucacagcaguugguaaagaguucaaccaccuugaaaaaagaauagagaaucuaaauaaaaagguugaugaugguuuccuggacauuuggacuuacaaugccgaacuguugguucuacuggaaaacgaaagaacuuuggacuaucacgauucaaaugugaagaacuuguaugaaaaaguaagaaaccaguuaaaaaacaaugccaaggaaauuggaaacggcugcuuugaauuuuaccacaaaugcgacaacacaugcauggaaagugucaagaaugggacuuaugacuacccaaaauacucagaggaagcaaaauuaaacagagaaaaaauagauggaguaaagcuggacucaacaaggaucuaccagauuuuggcgaucuauucaacuguugccaguucauugguacugguagucucccugggggcaaucagcuucuggaugugcucuaaugggucucuacaguguagaauauguauuuaa

SEQ ID NO: 75 - RNA, 1401 nt, Influenza B virus
augcuaccuucaacuauacaaacguuaacccuauuucucacaucagggggaguauuauuaucacuauaugugucagcuucauuaucauacuuacuauauucggauauauugcuaaaauucucaccaacagaaauaacugcaccaacaaugccauuggauugugcaaacgcaucaaauguucaggcugugaaccguucugcaacaaaaggggugacacuucuucucccagaaccggaguggacauacccgcguuuaucuugcccgggcucaaccuuucagaaagcacuucuaauuagcccucauagauucggagaaaccaaaggaaacucagcucccuugauaauaagggaaccuuuuguagcuuguggaccaaaugaaugcaaacacuuugcuuuaacccauuaugcugcccaaccagggggauacuauaauggaacaagaggagacagaaacaagcugaggcaucuaauuucagucaaauugggcaaaaucccaacaguagagaacuccauuuuccacauggcagcauggagcggguccgcgugccaugaugguaaggaauggacauauaucggaguugauggcccugacaauaaugcauugcucaaaguaaaauauggagaagcauauacugacacauaccauuccuaugcaaacaacauccuaagaacacaagaaagugccugcaauugcaucgggggaaauuguuaucuaaugauaacugauggcucagcuucagguguuagugaaugcagauuucuuaagauucgagagggccgaauaauaaaagaaauauuuccaacaggaagaguaaaacacacugaggagugcacaugcggauuugccagcaauaaaaccauagaaugugccuguagagacaacagguacacagcaaaaagaccuuuugucaaauuaaacguggagacugauacagcagaaauaagguugaugugcacagauacuuauuuggacacccccagaccaaaugauggaagcauaacaggcccuugugaaucugauggggacaaagggaguggaggcaucaagggaggauuuguucaucaaagaaugaaauccaagauuggaaggugguacucucgaacgaugucuaaaacugaaaggauggggaugggacuguaugucaaguaugguggagacccaugggcugacagugaugcccuaacuuuuaguggaguaaugguuucaaugaaagaaccugguugguauuccuuuggcuucgaaauaaaagauaagaaaugcgauguccccuguauugggauagagaugguacaugaugguggaaaagagacuuggcacucagcagcaacagccauuuacuguuuaaugggcucaggacagcugcugugggacacugucacagguguugacauggcucuguaa

SEQ ID NO: 76 - RNA, 1749 nt, Influenza B virus
augaaggcaauaauuguacuacucaugguaguaacauccaaugcagaucgaaucugcacugggauaacaucgucaaacucaccacaugucgucaaaacugcuacucaaggggaggucaacgugaccgguguaauaccacugacaacaacacccaccaaaucucauuuugcaaaucucaaaggaacagaaaccagggggaaacuaugcccaaaaugccucaacugcacagaucuggauguagccuugggcagaccaaaaugcacagggaaaauacccucugcaaggguuucaauacuccaugaagucagaccuguuacaucugggugcuuuccuauaaugcacgauagaacaaaaauuagacagcugccuaaccuucuccgaggauacgaacaugucagguuaucaacucacaacguuaucaaugcagaagaugcaccaggaagacccuacgaaauuggaaccucagggucuugcccuaacauuaccaauggaaacggauucuucgcaacaauggcuugggccgucccaaaaaacaaaacagcaacaaauccauuaacaauagaaguaccauacauuuguacagaaggagaagaccaaauuaccguuuggggguuccacucugacagcgagacccaaauggcaaagcucuauggggacucaaagccccagaaguucaccucaucugccaacggagugaccacacauuacguuucacagauugguggcuucccaaaucaaacagaagacggaggacuaccacaaaguggcagaauuguuguugauuacauggugcagaaaucuggaaaaacaggaacaauuaccuaucaaagagguauuuuauugccucaaaagguguggugcgcaaguggcaggagcaagguaauaaaaggauccuugcccuuaauuggagaagcagauugccuccaugaaaaauacgguggauuaaacaaaagcaagccuuacuacacaggggaacaugcaaaggccauaggaaauugcccaauaugggugaaaacacccuugaagcuggccaauggaaccaaauauagacccccugcaaaacuauuaaaggaaagagguuucuucggagccauugcugguuucuuagagggaggaugggaaggaaugauugcagguuggcacggauacacaucccauggggcacauggaguagcgguggcagcugaccuuaagagcacucaagaggccauaaacaagauaacaaaaaaucucaacucuuugagugagcuggaaguaaagaaucuucaaagacuaagcggugccauggaugaacuccacaacgaaauacuagaacuagaugagaaaguggaugaucucagagcugauacaauaagcucacaaauagaacucgcaguccugcuuuccaaugaaggaauaauaaacagugaagaugaacaucucuuggcgcuugaaagaaagcugaagaaaaugcugggccccucugcuguagagauagggaauggaugcuuugaaaccaaacacaagugcaaccagaccugucucgacagaauagcugcugguaccuuugaugcaggagaauuuucucuccccaccuuugauucacugaauauuacugcugcaucuuuaaaugacgacggauuggacaaucauacuauacugcuuuacuacucaacugcugccuccaguuuggcuguaacacugaugauagcuaucuuuguuguuuauauggucuccagagacaauguuucuugcuccauuugucuauaa

SEQ ID NO: 77 - RNA, 687 nt, Artificial Sequence
  source: /note=“Description of Artificial Sequence Synthetic polynucleotide”
augaagaccaucaucgcccugagcuacauccugugccugguguucgcccagaagcugcccggcaacgacaacagcaccgccacccugugccugggccaccacgccgugcccaacggcaccaucgugaagaccaucaccaacgaccagaucgaggugaccaacgccaccgagcugguguuccccggcugcggcgugcugaagcuggccaccggcaugaggaacgugcccgagaagcagaccaggggcaucuucggcgccaucgccggcuucaucgagaacggcugggagggcaugguggacggcugguacggcuucaggcaccagaacagcgagggcaucggccaggccgccgaccugaagagcacccaggccgccaucaaccagaucaacggcauggugaacagggugaucgagcugauggagcagggcggccccgacugcuaccuggccgagcugcugguggcccugcugaaccagcacaccaucgaccugaccgacagcgagaugaggaagcuguucgagaggaccaagaagcagcugagggagaacgccgaggacaugggcaacggcugcuucaagaucuaccacaagugcgacaacgccugcaucggcagcaucaggaacggcaccuacgaccacgacguguacagggacgaggcccugaacaacagguuccagaucaaguaa

SEQ ID NO: 78 - RNA, 666 nt, Artificial Sequence
  source: /note=“Description of Artificial Sequence Synthetic polynucleotide”
augaacacccagauccugguguucgcccugaucgccaucauccccaccaacgccgacaagaucugccugggccaccacgccgugagcaacggcaccaaggugaacacccugaccgagaggggcguggagguggugaacgccaccgagcugguguuccccggcugcggcgugcugcugcuggccaccggcaugaagaacgugcccgagauccccaagggcaggggccuguucggcgccaucgccggcuucaucgagaacggcugggagggccugaucgacggcugguacggcuucaggcaccagaacgcccagggcgagggcaccgccgccgacuacaagagcacccagagcgccaucgaccagaucaccggcauggugaacagggugaucgagcugauggagcagggcggccccgacugcuaccuggccgagcugcugguggccaugcugaaccagcacaccaucgaccuggccgacagcgagauggacaagcuguacgagagggugaagaggcagcugagggagaacgccgaggaggacggcaccggcugcuucgagaucuuccacaagugcgacgacgacugcauggccagcaucaggaacaacaccuacgaccacagcaaguacagggaggaggccaugcagaacaggauccagaucgacuaa

SEQ ID NO: 79 - RNA, 1725 nt, Artificial Sequence
  source: /note=“Description of Artificial Sequence Synthetic polynucleotide”
auggaguugcuaauccucaaagcaaaugcaauuaccacaauccucacugcagucacauuuuguuuugcuucuggucaaaacaucacugaagaauuuuaucaaucaacaugcagugcaguuagcaaaggcuaucuuagugcucugagaacugguugguauaccaguguuauaacuauagaauuaaguaauaucaaggaaaauaaguguaauggaacagaugcuaagguaaaauugauaaaacaagaauuagauaaauauaaaaaugcuguaacagaauugcaguugcucaugcaaagcacaccaccaacaaacaaucgagccagaagagaacuaccaagguuuaugaauuauacacucaacaaugccaaaaaaaccaauguaacauuaagcaagaaaaggaaaagaagauuucuugguuuuuuguuagguguuggaucugcaaucgccaguggcguugcuguauguaagguccugcaccuagaaggggaagugaacaagaucaaaagugcucuacuauccacaaacaaggcuguagucagcuuaucaaauggaguuagugucuuaaccuucaaaguguuagaccucaaaaacuauauagauaaacaauuguuaccuauucugaacaagcaaagcugcagcauaucaaauauagaaacugugauagaguuccaacaaaagaacaacagacuacuagagauuaccagggaauuuaguguuaaugcagguguaacuacaccuguaagcacuuacauguuaacuaauagugaauuauugucauuaaucaaugauaugccuauaacaaaugaucagaaaaaguuaauguccaacaauguucaaauaguuagacagcaaaguuacucuaucaugugcauaauaaaagaggaagucuuagcauauguaguacaauuaccacuauaugguguuauagauacacccuguuggaaacuacacacauccccucuauguacaaccaacacaaaagaaggguccaacaucuguuuaacaagaacugacagaggaugguacugugacaaugcaggaucaguaucuuucuucccacaagcugaaacauguaaaguucaaucaaaucgaguauuuugugacacaaugaacaguuuaacauuaccaagugaaauaaaucucugcaauguugacauauucaaccccaaauaugauuguaaaauuaugacuucaaaaacagauguaagcagcuccguuaucacaucucuaggagccauugugucaugcuauggcaaaacuaaauguacagcauccaauaaaaaucguggaaucauaaagacauuuucuaacgggugcgauuauguaucaaauaaagggauggacacugugucuguagguaacacauuauauuauguaaauaagcaagaagguaaaagucucuauguaaaaggugaaccaauaauaaauuucuaugacccauuaguauuccccucugaugaauuugaugcaucaauaucucaagucaacgagaagauuaaccagagccuagcauuuauucguaaauccgaugaauuauuacauaauguaaaugcugguaaauccaccacaaauaucaugauaacuacuauaauuauagugauuauaguaauauuguuaucauuaauugcuguuggacugcucuuauacuguaaggccagaagcacaccagucacacuaagcaaagaucaacugagugguauaaauaauauugcauuuaguaacuaa

SEQ ID NO: 80 - RNA, 288 nt, Mycobacterium tuberculosis
augacagagcagcaguggaauuucgcggguaucgaggccgcggcaagcgcaauccagggaaaugucacguccauucauucccuccuugacgaggggaagcagucccugaccaagcucgcagcggccuggggcgguagcgguucggaggcguaccaggguguccagcaaaaaugggacgccacggcuaccgagcugaacaacgcgcugcagaaccuggcgcggacgaucagcgaagccggucaggcaauggcuucgaccgaaggcaacgucacugggauguucgcauag

SEQ ID NO: 81 - RNA, 978 nt, Mycobacterium tuberculosis
augacagacgugagccgaaagauucgagcuuggggacgccgauugaugaucggcacggcagcggcuguaguccuuccgggccugguggggcuugccggcggagcggcaaccgcgggcgcguucucccggccggggcugccggucgaguaccugcaggugccgucgccgucgaugggccgcgacaucaagguucaguuccagagcggugggaacaacucaccugcgguuuaucugcucgacggccugcgcgcccaagacgacuacaacggcugggauaucaacaccccggcguucgagugguacuaccagucgggacugucgauagucaugccggucggcgggcaguccagcuucuacagcgacugguacagcccggccugcgguaaggcuggcugccagacuuacaagugggaaaccuuccugaccagcgagcugccgcaaugguuguccgccaacagggccgugaagcccaccggcagcgcugcaaucggcuugucgauggccggcucgucggcaaugaucuuggccgccuaccacccccagcaguucaucuacgccggcucgcugucggcccugcuggaccccucucaggggauggggccuagccugaucggccucgcgaugggugacgccggcgguuacaaggccgcagacauguggggucccucgagugacccggcaugggagcgcaacgacccuacgcagcagauccccaagcuggucgcaaacaacacccggcuauggguuuauugcgggaacggcaccccgaacgaguugggcggugccaacauacccgccgaguucuuggagaacuucguucguagcagcaaccugaaguuccaggaugcguacaacgccgcgggcgggcacaacgccguguucaacuucccgcccaacggcacgcacagcugggaguacuggggcgcucagcucaacgccaugaagggugaccugcagaguucguuaggcgccggcuga

SEQ ID NO: 82 - RNA, 291 nt, Mycobacterium tuberculosis
augucgcaaaucauguacaacuaccccgcgauguugggucacgccggggauauggccggauaugccggcacgcugcagagcuugggugccgagaucgccguggagcaggccgcguugcagagugcguggcagggcgauaccgggaucacguaucaggcguggcaggcacaguggaaccaggccauggaagauuuggugcgggccuaucaugcgauguccagcacccaugaagccaacaccauggcgaugauggcccgcgacacggccgaagccgccaaauggggcggcuag

SEQ ID NO: 83 - RNA, 432 nt, Mycobacterium tuberculosis
augaccaccgcacgcgacaucaugaacgcaggugugaccuguguuggcgaacacgagacgcuaaccgcugccgcucaauacaugcgugagcacgacaucggcgcguugccgaucugcggggacgacgaccggcugcacggcaugcucaccgaccgcgacauugugaucaaaggccuggcugcgggccuagacccgaauaccgccacggcuggcgaguuggcccgggacagcaucuacuacgucgaugcgaacgcaagcauccaggagaugcucaacgucauggaagaacaucagguccgccguguuccggucaucucagagcaccgcuuggucggaaucgucaccgaagccgacaucgcccgacaccugcccgagcacgccauugugcaguucgucaaggcaaucugcucgcccauggcccucgccagcuag

SEQ ID NO: 84 - RNA, 1074 nt, Artificial Sequence
  source: /note=“Description of Artificial Sequence Synthetic polynucleotide”
augaccgucgacggaaccgcgaugcgggugaccacgaugaaaucgcgggugaucgacaucgucgaagagaacggguucucagucgacgaccgcgacgaccuguaucccgcggccggcgugcagguccaugacgccgacaccaucgugcugcggcguagccguccgcugcagaucucgcuggauggucacgacgcuaagcagguguggacgaccgcgucgacgguggacgaggcgcuggcccaacucgcgaugaccgacacggcgccggccgcggcuucucgcgccagccgcgucccgcuguccgggauggcgcuaccggucgucagcgccaagacggugcagcucaacgacggcggguuggugcgcacggugcacuugccggcccccaaugucgcggggcugcugagugcggccggcgugccgcuguugcaaagcgaccacguggugcccgccgcgacggccccgaucgucgaaggcaugcagauccaggugacccgcaaucggaucaagaaggucaccgagcggcugccgcugccgccgaacgcgcgucgugucgaggacccggagaugaacaugagccgggaggucgucgaagacccggggguuccggggacccaggaugugacguucgcgguagcugaggucaacggcgucgagaccggccguuugcccgucgccaacgucguggugaccccggcccacgaagccguggugcgggugggcaccaagcccgguaccgaggugcccccggugaucgacggaagcaucugggacgccaucgcgcaaugcaaauccggcggcaauugggcggccaacaccgguaacggguuauacgguggucugcagaucagccaggcggcgugggauuccaacgguggugucgggucgccggcggccgcgaguccccagcaacagaucgaggucgcagacaacauuaugaaaaccgcaggcccgggugcguggccgaaauguaguucuuguagucagggagacgcaccgcugggcucgcucacccacauccugacguuccucgcggccgagacuggagguuguucggggagcagggacgauuga

(19) United States
(12) Patent Application Publication
(10) Pub. No.: US 2024/0024451 A1
(43) Pub. Date: Jan. 25, 2024

(54) COMPOSITIONS AND METHODS FOR IMPROVED VACCINATION

(71) Applicant: Combined Therapeutics, Inc., Wilmington, DE (US)

(72) Inventors: Romain Micol, London (GB); Valerie Duval, Wilmington, DE (US)

(21) Appl. No.: 18/018,759

(22) PCT Filed: Jul. 30, 2021
(86) PCT No.: PCT/US2021/043975

§ 371 Date: Jan. 30, 2023

Related U.S. Application Data
(63) Continuation of application No. PCT/US2021/019028, filed on Feb. 22, 2021.
(60) Provisional application No. 63/059,458, filed on Jul. 31, 2020.

(51) Int. Cl. A61K 39/12; C07K 14/54; C12N 15/113; A61K 39/04; A61K 39/00; A61P 31/14; A61K 48/00

(52) U.S. Cl. CPC A61K 39/12; C07K 14/5434; C12N 15/113; A61K 39/04; A61K 39/0011; A61P 31/14; A61K 48/005; A61K 2039/53

(57) ABSTRACT

Provided is a composition comprising a first mRNA construct comprising a first open reading frame (ORF), wherein the first ORF encodes an antigen; wherein the first ORF is operatively linked to at least one untranslated region (UTR), wherein the UTR comprises at least a first organ protection sequence (OPS), and wherein the first OPS comprises at least two micro-RNA (miRNA) target sequences, wherein each of the at least two miRNA target sequences are optimised to hybridise with a corresponding miRNA sequence. Also provided are further compositions comprising mRNA constructs comprising an ORF and an OPS wherein the ORF encodes a proinflammatory cytokine, and methods including one or both of these compositions for the treatment and prevention of disease such as pathogenic disease.

### RELATED APPLICATIONS

This application claims the benefit of priority to U.S. Provisional Patent Application Ser. No. 63/059,458, filed Jul. 31, 2020; and PCT Application serial number PCT/US21/19028, filed Feb. 22, 2021. The contents of each of which are hereby incorporated by reference in their entirety.

### FIELD

The present invention relates to messenger ribonucleic acid (mRNA) delivery technologies, and methods of using these mRNA delivery technologies in a variety of therapeutic, diagnostic and prophylactic indications.

### BACKGROUND

The ability to induce expression of a specified gene product such as a polypeptide in a particular target tissue or organ is frequently desired. In many situations, a target tissue or organ, will comprise more than one type of cell, and in such cases it is also frequently desired to express the gene product to different degrees in the different cell types—that is, to provide differential expression of the gene product between the different cell types in the target tissue. For example, in gene therapy a mutated and/or functionless gene can be replaced in target cells by an intact copy, but it is also useful to minimise off target protein production in neighbouring cells, tissues and organs. Likewise, a gene product for vaccine antigens such as the spike protein for COVID-19 is preferably expressed in or around dendritic cells of the immune system in order to ensure a maximal response.

Gene therapy often relies on viral vectors to introduce coding polynucleotides into target cells, but other techniques exist to deliver polynucleotides to cells without the use of viruses. The advantages of viruses include relatively high possible transfection rates, as well as the ability to target the virus to particular cell types by control of the binding proteins by which viruses enter a target cell. In contrast, non-viral methods of introducing coding polynucleotides into cells can have problems with low transfection rates, as well as having limited options for targeting expression to particular organs and cell types. However, the nature of viral intervention carries risks of toxicity and inflammation, but also has limited control over the duration and degree of the expression of the introduced factor.

Tumor therapies based upon biological approaches have advantages over traditional chemotherapeutics because they can employ numerous diverse mechanisms to target and destroy cancers more precisely—e.g. via direct cell lysis, cytotoxic immune effector mechanisms and vascular collapse amongst others. As a result, there has been a significant increase in the number of clinical studies into the potential of such approaches. However due to the diverse range of therapeutic activities, pre-clinical and clinical study is complex, as multiple parameters may affect their therapeutic potential and, hence, defining reasons for treatment failure or methodologies that might enhance the therapeutic activity can be difficult. Maintaining on-target activities, tumor specificity and reducing side effects is also a major challenge for such experimental and powerful therapies.

Viral based therapies have emerged as a promising approach to address many aspects of disease treatment. Cancer vaccines that are based upon inactivated or attenuated viruses offer considerable potential for hard-to-treat cancers. However, the effectiveness of therapeutic viruses is often thwarted by the body's own immune response thereby limiting applications to avoid systemic administration. Hence, it would be advantageous to provide novel compositions and methods that are able to improve and enhance the range of therapeutic viral approaches currently available.

Vaccines are often a highly effective preventative intervention against infectious disease. However, for some applications and in some situations, vaccine efficacy can be suboptimal. For example, the development of an effective response against a delivered antigen depends on the competency of the subject's immune system. In all subjects, immunity can be lost over time, and/or the immune response against a particular antigen can be insufficient.

Similarly, certain types or classes of pathogen can be difficult to vaccinate against, due to anti-immune adaptations, rapid mutation, or natural history. For example, intracellular parasites such as viruses, intracellular bacteria or single-celled eukaryotes (for example, the malaria parasite) can often be challenging to provide vaccines against.

Often, live attenuated vaccines can provide an improved response, but have attendant risks, primarily the risk of reactivation of the attenuated pathogen. Other shortcomings of existing vaccine technology include the possibility of ‘vaccine escape’, where a pathogen variant evolves which is not combatted as effectively by the immune response triggered by the vaccine (for example, if mutations arise in the genes coding for the targeted antigen); the loss of immunity over time; and incomplete resistance.

For all these reasons, vaccines are therefore often provided with adjuvants to increase immune response, but these have risks of their own, such as the induction of symptoms and the risk of autoimmune attack. There is therefore a need to provide more effective and safer vaccines and/or adjuvants, in particular with regards to pathogens which are more challenging to vaccinate against.

WO-2017/132552-A1 describes recombinant oncolytic virus with an engineered genome that includes micro-RNA binding sites.

US-2013/156849-A1 relates to methods for expressing a polypeptide of interest in a mammalian cell or tissue, the method comprising, contacting said mammalian cell or tissue with a formulation comprising a modified mRNA encoding the polypeptide of interest. WO-2016/011306-A2 describes design, preparation, manufacture and/or formulation of nucleic acids comprising at least one terminal modification that may comprise a micro-RNA binding site. The aforementioned prior art do not address the problems of ensuring effective protection of single or multiple organ types in the body of a subject who is treated with a co-administered therapeutic agent or factor.

WO 2019/051100 A1 and WO 2019/158955 A1 describe compositions and methods for delivery of mRNA sequences for expression of one or more polypeptides within one or more target organs, comprising miRNA binding site sequences which allow for differential expression of the coding sequence in at least a first and a second cell type within the target organ or organs.

There is a need to further develop further improved and optimized methods and compositions for modulating expression of polynucleotide sequences, such as mRNA, in specific organs and/or tissues.

### SUMMARY

In various embodiments, the invention provides compositions and methods suitable for delivering nucleotide-encoded products such as mRNA constructs, for example for use as vaccine and/or adjuvant compositions. In some embodiments, one or more of the delivered compositions are adapted for controlled expression by the inclusion of miRNA binding site sequences, in particular, by the provision of organ protection sequences. In all of the aspects described herein, it is contemplated that ‘mRNA constructs’ include circular or circularised RNA constructs which can be translated to produce protein products.

In a first aspect, there is provided a composition comprising: a first mRNA construct comprising a first open reading frame (ORF), wherein the first ORF encodes an antigen. The first ORF is operatively linked to at least one untranslated region (UTR), wherein the UTR comprises at least a first organ protection sequence (OPS), and wherein the first OPS comprises at least two micro-RNA (miRNA) target sequences, wherein each of the at least two miRNA target sequences are optimised to hybridise with a corresponding miRNA sequence.

The first mRNA construct may be comprised within or adsorbed to an in vivo delivery composition. The antigen may be selected from the group consisting of: a pathogenic microbial protein and a tumor-associated antigen, or an epitope containing fragment thereof. The pathogenic microbial protein may be selected from the group consisting of: a viral protein; a bacterial protein; a fungal protein; a parasite protein; and a prion.

The antigen may comprise a viral protein or an epitope containing fragment thereof. The antigen may comprise a coronavirus spike protein; a variant coronavirus spike protein; suitably a SARS-CoV-2 spike protein. The antigen may comprise an influenza protein or a variant thereof, or an epitope containing fragment thereof; suitably wherein the influenza protein is selected from the group consisting of a hemagglutinin, a neuraminidase, a matrix-2 and/or a nucleoprotein. The influenza protein may be selected from type A influenza, a type B influenza, or a subtype of type A influenza of H1, H2, H3, H4, H5, H6, H7, H8, H9, H10, H11, H12, H13, H14, H15 or H16. The antigen may comprise a respiratory syncytial virus (RSV) protein, or a variant thereof, or an epitope containing fragment thereof; suitably wherein the protein of the respiratory syncytial virus is the F glycoprotein or the G glycoprotein. The antigen may comprise a Human Immunodeficiency Virus (HIV) protein or an epitope containing fragment thereof; suitably wherein the HIV protein is the glycoprotein 120 neutralizing epitope or glycoprotein 145.

The antigen may comprise a protein from the Mycobacterium tuberculosis bacterium or an epitope containing fragment thereof; suitably wherein the protein from the Mycobacterium tuberculosis bacterium is selected from ESAT-6, Ag85B, TB110.4, Rv2626 and/or RpfD-B.

The antigen may be a tumor-associated antigen. The tumor-associated antigen may comprise a colorectal tumour antigen; MUC 1; and/or a neoantigen.

The first mRNA construct may further comprise a further open reading frame (ORF), wherein the further ORF encodes an antigen different to the antigen encoded by the first ORF. The further ORF may be selected from: a bacterial protein, a viral protein, or a tumor-associated antigen, or an epitope containing fragment thereof. The further antigen may be similar to any possibility for the antigen encoded by the first ORF; independently of the identity of the antigen encoded by the first ORF.

In some embodiments, the first mRNA construct may further comprise a further open reading frame (ORF), wherein the further ORF encodes a proinflammatory cytokine. The proinflammatory cytokine may be selected from: IFNγ; IFNα; IFNβ; TNFα; IL-12; IL-2; IL-6; IL-8; and GM-CSF.

The first OPS may comprise at least three, at least four, or at least five miRNA target sequences. The first OPS may comprise at least three miRNA target sequences which are all different from each other. Any of the miRNA sequences of the first OPS may be repeated. In some embodiments, the first OPS comprises miRNA sequences selected to protect one or more organs or tissues selected from the group consisting of muscle, liver, brain, breast, endothelium, pancreas, colon, kidney, lungs, spleen and skin, heart, gastrointestinal organs, reproductive organs, and esophagus, more specifically, from the group consisting of muscle, liver, kidney, lungs, spleen, skin, heart, gastrointestinal organs, reproductive organs, and esophagus.

In some embodiments, the first OPS comprises at least two miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family (for example, miRNA-30 a, b, or c); miRNA-877; miRNA-4300; miRNA-4720; and/or miRNA-6761. In some embodiments, the first OPS comprises at least two miRNA target sequences selected from sequences capable of binding with miRNA-1, miRNA-122, miRNA-30a, miRNA-203a, let7b, miRNA-126, and/or miRNA-192. The first OPS may comprise sequences selected from one or more of SEQ ID NOs: 44-57. The first OPS may comprise at least two miRNA target sequences selected from sequences capable of binding with miRNA-1, miRNA133a, miRNA206, miRNA-122, miRNA203a, miRNA205, miRNA200c, miRNA30a, and/or let7a/b. The first OPS may comprise at least two miRNA target sequences selected from sequences capable of binding with miRNA-1, miRNA-122, miR-30a and/or miR-203a; with miRNA-1, miRNA-122, miRNA-30a and miRNA-203a; with miRNA-122, miRNA-1, miRNA-203a, and miRNA-30a; with let7b, miRNA-126, and miRNA-30a; with miRNA-122, miRNA-192, and miRNA-30a. In some embodiments, the first OPS comprises miRNA target sequences capable of binding with miRNA-192, miRNA-30a, and miRNA-124, and two miRNA target sequences capable of binding with miRNA 122.

In some embodiments, the composition further comprises a second mRNA construct comprising a second open reading frame (ORF), wherein the second ORF encodes a proinflammatory cytokine. The proinflammatory cytokine may be selected from the group consisting of: IL-12; IL-2; IL-6; IL-8; IFNγ; IFNα; IFNβ; TNFα; and GM-CSF. The second mRNA construct may be comprised within or adsorbed to a delivery composition, which may be the same or different to that associated with the first mRNA construct. The delivery composition(s) may comprise a delivery vector selected from the group consisting of: a particle, such as a polymeric particle; a liposome; a lipidoid particle; and a viral vector.

The second ORF may code for an IL-12 protein, or a subunit, derivative, fragment, agonist or homologue thereof. In particular, the second ORF may comprise a sequence at least 90% identical to SEQ ID NO: 59.

In some embodiment, the second ORF is operatively linked to a second untranslated region (UTR), wherein the UTR comprises a second organ protection sequence (OPS) and wherein the second OPS comprises at least two micro-RNA (miRNA) target sequences. The at least two miRNA target sequences may be optimised to hybridise with a corresponding miRNA sequence. The second OPS may be defined similarly to any variation of the first OPS, as discussed above, and can vary independently of the identity of the first OPS.

In some embodiments, the second OPS comprises at least three, at least four, or at least five miRNA target sequences. The second OPS may comprise at least three miRNA target sequences which are all different from each other. The second OPS comprises miRNA sequences selected to protect one or more organs or tissues selected from the group consisting of muscle, liver, brain, breast, endothelium, pancreas, colon, kidney, lungs, spleen and skin; more specifically from the group consisting of muscle, liver, kidney, lungs, spleen, skin, heart, gastrointestinal organs, reproductive organs, and esophagus.

In some embodiments, the second OPS comprises at least two miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family (for example, miRNA-30 a, b, or c) family (for example, miRNA-30 a, b, or c); miRNA-877; miRNA-4300; miRNA-4720; and/or miRNA-6761. In some embodiments, the second OPS comprises at least two miRNA target sequences selected from sequences capable of binding with miRNA-1, miRNA-122, miRNA-30a, miRNA-203a, let7b, miRNA-126, and/or miRNA-192. The second OPS may comprise sequences selected from one or more of SEQ ID NOs: 44-57. The second OPS may comprise at least two miRNA target sequences selected from sequences capable of binding with miRNA-1, miRNA133a, miRNA206, miRNA-122, miRNA203a, miRNA205, miRNA200c, miRNA30a, and/or let7a/b. The second OPS may comprise at least two miRNA target sequences selected from sequences capable of binding with miRNA-1, miRNA-122, miR-30a and/or miR-203a; with miRNA-1, miRNA-122, miRNA-30a and miRNA-203a; with miRNA-122, miRNA-1, miRNA-203a, and miRNA-30a; with let7b, miRNA-126, and miRNA-30a; and/or with miRNA-122, miRNA-192, and miRNA-30a. In some embodiments, the second OPS comprises miRNA target sequences capable of binding with miRNA-192, miRNA-30a, and miRNA-124, and two miRNA target sequences capable of binding with miRNA 122.

The first OPS may comprise includes at least one different miRNA target sequence to the second OPS. The first OPS and the second OPS may include the same miRNA target sequences. In an embodiment, the first OPS comprises miRNA target sequences capable of binding with miRNA-1, miRNA-122, miR-30a and miR-203a; and the second OPS comprises miRNA target sequences capable of binding with miRNA-122, miRNA-192, and/or miRNA 30a.

The composition may further comprise at least a third mRNA construct (in addition to or instead of the second mRNA construct) comprising at least a third open reading frame (ORF), wherein the third ORF encodes an antigen different to the antigen encoded by the first ORF, and selected from: a bacterial protein, a viral protein, or a tumor-associated antigen, or an epitope containing fragment thereof. The third ORF may be operatively linked to at least a third untranslated region (UTR), wherein the UTR comprises at least a third organ protection sequence (OPS), wherein the third OPS protects multiple organs, and wherein the third OPS comprises at least two micro-RNA (miRNA) target sequences, and wherein each of the at least two miRNA target sequences are optimised to hybridise with a corresponding miRNA sequence. The third OPS may be defined similarly to any variation of the first or second OPS, as discussed above, and can vary independently of the identity of the first or second OPS.

In an embodiment, the first ORF codes for a coronavirus spike protein or an epitope containing fragment thereof, and the third ORF codes for a viral protein or an epitope containing fragment thereof that comprises all or a part of an influenza protein, or a variant thereof.

The composition may be suitable for administration intravenously, subcutaneously, intra-muscularly, intranasally, intra-arterially and/or through inhalation.

In a second aspect, there is provided a composition comprising at least a first mRNA construct comprising at least a first open reading frame (ORF); and a second construct comprising at least a second mRNA construct comprising at least one open reading frame (ORF), wherein the ORF encodes a proinflammatory cytokine, and wherein the second ORF is operatively linked to at least one untranslated region (UTR), wherein the UTR comprises at least one OPS that protects multiple organs, and wherein the OPS comprises at least two miRNA target sequences, and wherein each of the at least two miRNA target sequences are optimised to hybridise with a corresponding miRNA sequence.

The components of the composition of this second aspect may be defined similarly to any variation of the corresponding factors of the first aspect, as defined above, and further components such as further ORFs and further mRNA constructs may also be included as described above. For example, the ORF of the first mRNA construct may encode an antigen selected from: a bacterial protein; and/or a viral protein, and/or may be defined as described above for the first aspect. The composition may comprise an in vivo delivery composition, and the first and/or second constructs may be comprised within or adsorbed to the delivery composition. The delivery composition may comprise delivery vectors selected from the group consisting of: a particle, such as a polymeric particle; a liposome; a lipidoid particle; and a viral vector.

The ORF of the second mRNA construct may encode a proinflammatory cytokine selected from: IFNγ; IFNα; IFNβ; TNFα; IL-12; IL-2; IL-6; IL-8; and GM-CSF, and may code for IL-12 protein, or a derivative, agonist or homologue thereof.

The OPS of the second construct may be defined as any of the OPS described for the first aspect above. In some embodiments, the OPS comprises miRNA sequences selected to protect one or more organs selected from the group consisting of muscle, liver, kidney, lungs, spleen and skin. The OPS may comprise sequences selected from one or more of SEQ ID NOs: 44-57. The OPS may comprise at least two miRNA target sequences selected from sequences capable of binding with miRNA-1, miRNA-122, miRNA-30a, miRNA-203a, let7b, miRNA-126, and/or miRNA-192; with miRNA-1, miRNA133a, miRNA206, miRNA-122, miRNA203a, miRNA205, miRNA200c, miRNA30a, and/or let7a/b; with miRNA-1, miRNA-122, miR-30a and/or miR-203a; with miRNA-1, miRNA-122, miRNA-30a and miRNA-203a; with miRNA-122, miRNA-1, miRNA-203a, and miRNA-30a; with let7b, miRNA-126, and miRNA-30a; and/or with miRNA-122, miRNA-192, and miRNA-30a. In an embodiment, the OPS comprises miRNA target sequences capable of binding with miRNA-192, miRNA-30a, and miRNA-124, and two miRNA target sequences capable of binding with miRNA-122.

The antigen encoded by the first mRNA construct may be selected from the group consisting of: a pathogenic microbial protein and a tumor-associated antigen, or an epitope containing fragment thereof. The pathogenic microbial protein may be selected from the group consisting of: a viral protein; a bacterial protein; a fungal protein; a parasite protein; and a prion.

The antigen may comprise a viral protein or an epitope containing fragment thereof. The antigen may comprise a coronavirus spike protein; a variant coronavirus spike protein; suitably a SARS-CoV-2 spike protein. The antigen may comprise an influenza protein or a variant thereof, or an epitope containing fragment thereof; suitably wherein the influenza protein is selected from the group consisting of a hemagglutinin, a neuraminidase, a matrix-2 and/or a nucleoprotein. The influenza protein may be selected from type A influenza, a type B influenza, or a subtype of type A influenza of H1, H2, H3, H4, H5, H6, H7, H8, H9, H10, H11, H12, H13, H14, H15 or H16. The antigen may comprise a respiratory syncytial virus (RSV) protein, or a variant thereof, or an epitope containing fragment thereof; suitably wherein the protein of the respiratory syncytial virus is the F glycoprotein or the G glycoprotein. The antigen may comprise a Human Immunodeficiency Virus (HIV) protein or an epitope containing fragment thereof; suitably wherein the HIV protein is the glycoprotein 120 neutralizing epitope or glycoprotein 145.

The antigen may comprise a protein from the Mycobacterium tuberculosis bacterium or an epitope containing fragment thereof; suitably wherein the protein from the Mycobacterium tuberculosis bacterium is selected from ESAT-6, Ag85B, TB110.4, Rv2626 and/or RpfD-B.

In a further embodiment, the compositions as described in any aspect or variation above is for use in a method of the prevention or treatment of pathogenic disease, which may comprise administering the composition to a subject in need thereof; and/or coadministering the various constructs described to a subject in need thereof. The pathogenic disease may be caused by a coronavirus, which may be the SARS-CoV-2 virus.

In a further embodiment there is provided a method of increasing a Th1 immune response, comprising administering a composition as defined above, in particular wherein an ORF coding for an IL-12 protein, or a subunit, derivative, fragment, agonist or homologue thereof is included.

In a third aspect, there is provided a composition comprising at least one mRNA construct comprising at least one open reading frame (ORF), wherein the at least one ORF encodes a proinflammatory cytokine, and wherein the ORF is operatively linked to at least one untranslated region (UTR), wherein the UTR comprises at least one OPS that protects multiple organs, and wherein the OPS comprises at least two miRNA target sequences, and wherein each of the at least two miRNA target sequences are optimised to hybridise with a corresponding miRNA sequence.

Again, the components of the composition of this third aspect may be defined similarly to any variation of the corresponding factors of the first or second aspect, as defined above, in particular the second mRNA construct(s) so defined. The composition may further comprise an in vivo delivery composition, wherein the mRNA construct is comprised within or adsorbed to the delivery composition. The delivery composition may comprise delivery vectors selected from the group consisting of: a particle, such as a polymeric particle; a liposome; a lipidoid particle; and a viral vector. The proinflammatory cytokine may be selected from: IL-12; IFNγ; IFNα; IFNβ; TNFα; IL-2; IL-6; IL-8; and GM-CSF; and may be an IL-12 protein, or a derivative, agonist or homologue thereof.

The OPS may be defined as any of the OPS described for the aspects above. In some embodiments, the OPS comprises miRNA sequences selected to protect one or more organs selected from the group consisting of muscle, liver, kidney, lungs, spleen and skin. The OPS may comprise sequences selected from one or more of SEQ ID NOs: 44-57.

The OPS may comprise at least two miRNA target sequences selected from sequences capable of binding with miRNA-1, miRNA-122, miRNA-30a, miRNA-203a, let7b, miRNA-126, and/or miRNA-192; with miRNA-1, miRNA133a, miRNA206, miRNA-122, miRNA203a, miRNA205, miRNA200c, miRNA30a, and/or let7a/b; with miRNA-1, miRNA-122, miR-30a and/or miR-203a; with miRNA-1, miRNA-122, miRNA-30a and miRNA-203a; with miRNA-122, miRNA-1, miRNA-203a, and miRNA-30a; with let7b, miRNA-126, and miRNA-30a; with miRNA-122, miRNA-192, and miRNA-30a; with miRNA-192, miRNA-30a, and miRNA-124, and two miRNA target sequences capable of binding with miRNA-122.

In an embodiment, the composition as described is for use in a method of the prevention of pathogenic disease, the method comprising administering the composition to a subject in need thereof; and coadministering a vaccine composition to the subject.

In another embodiment, the composition further comprises a vaccine selected from the group consisting of: a toxoid vaccine, a recombinant vaccine, a conjugated vaccine, an RNA-based vaccine, a DNA-based vaccine, a live-attenuated vaccine, an inactivated vaccine, a recombinant-vector based vaccine, and combinations thereof.

In a fourth aspect, there is provided a method of treating or preventing one or more pathogenic disease or improving an immune response, the method comprising administering to a subject in need thereof a composition comprising at least a first mRNA construct comprising at least a first open reading frame (ORF), wherein the first ORF encodes an antigen selected from: a bacterial protein, or a viral protein, or an epitope containing fragment thereof. The first ORF is operatively linked to at least one untranslated region (UTR), wherein the UTR comprises at least a first organ protection sequence (OPS), wherein the OPS protects multiple organs, and wherein the first OPS comprises at least two micro-RNA (miRNA) target sequences, and wherein each of the at least two miRNA target sequences are optimised to hybridise with a corresponding miRNA sequence; and an in vivo delivery composition; wherein the mRNA construct is comprised within or adsorbed to the delivery composition. The delivery composition may comprise delivery vectors selected from the group consisting of: a particle, such as a polymeric particle; a liposome; a lipidoid particle; and a viral vector.

The components of the composition used in this fourth aspect may be defined similarly to any variation of the corresponding factors of the aspects as defined above, and may comprise further components as described in the above aspects, in particular the first aspect.

In some embodiments, the method further comprises co-administering to the subject a composition comprising at least one mRNA construct comprising at least a second open reading frame (ORF), wherein the second ORF encodes a proinflammatory cytokine, which may be selected from: IL-12; IFNγ; IFNα; IFNβ; TNFα; IL-2; IL-6; IL-8; and GM-CSF. The second ORF may code for an IL-12 protein, or a derivative, agonist or homologue thereof. The second ORF may comprise a sequence at least 90% identical to SEQ ID NO: 59.

The first OPS may include a different set of miRNA target sequences to the second OPS. The first OPS and the second OPS may include the same miRNA target sequences. The first and/or second OPS may be independently defined similarly to those in the aspects described above.

In some embodiments, the pathogenic disease is caused by a coronavirus, which may be the SARS-CoV-2 virus. The antigen may comprise a viral protein or an epitope containing fragment thereof that comprises all or a part of a coronavirus spike protein or a variant coronavirus spike protein. The coronavirus spike protein may be a SARS-CoV-2 spike protein.

In some embodiments, the first mRNA construct further comprises a further open reading frame (ORF), wherein the further ORF encodes an antigen different to the antigen encoded by the first ORF. In some embodiments, the method further comprises co-administering to the subject a third mRNA construct comprising at least a third open reading frame (ORF), wherein the third ORF encodes an antigen different to the antigen encoded by the first ORF.

In a fifth aspect, there is provided a method of preventing one or more pathogenic disease or improving an immune response, the method comprising administering a vaccine composition to a subject in need thereof; and co-administering to the subject an adjuvant composition comprising at least one mRNA construct comprising at least one open reading frame (ORF), wherein the at least one ORF encodes a proinflammatory cytokine, and wherein the ORF is operatively linked to at least one untranslated region (UTR), wherein the UTR comprises at least one OPS that protects multiple organs, and wherein the OPS comprises at least two miRNA target sequences, and wherein each of the at least two miRNA target sequences are optimised to hybridise with a corresponding miRNA sequence; and an in vivo delivery composition; wherein the mRNA construct is comprised within or adsorbed to the delivery composition.

Again, the components of the composition used in this fourth aspect may be defined similarly to any variation of the corresponding factors of the aspects as defined above, and may comprise further components as described in the above aspects.

The proinflammatory cytokine may be selected from: IL-12; IFNγ; IFNα; IFNβ; TNFα; IL-2; IL-6; IL-8; and GM-CSF.

In some embodiments, the vaccine composition is selected from the group consisting of: a toxoid vaccine, a recombinant vaccine, a conjugated vaccine, an RNA-based vaccine, a DNA-based vaccine, a live-attenuated vaccine, an inactivated vaccine, a recombinant-vector based vaccine, and combinations thereof. In some embodiments, the vaccine composition comprises at least a first mRNA construct comprising at least a first open reading frame (ORF), wherein the first ORF encodes an antigen; and an in vivo delivery composition, wherein the mRNA construct is comprised within or adsorbed to the delivery composition. The antigen may be defined as in any preceding aspect.

Coadministering may comprise administering the vaccine composition and the adjuvant composition concurrently or consecutively, in either order. The vaccine composition and/or the adjuvant composition may be administered intravenously, subcutaneously, intra-muscularly, intranasally, intra-arterially and/or through inhalation.

In some embodiments of the fourth or fifth aspect, the pathogenic disease is caused by an intracellular pathogen. The pathogenic disease may be a latent infection, or an active infection. The pathogenic disease may be caused by an influenza virus, a coronavirus, the SARS-CoV-2 virus, the respiratory syncytial virus (RSV), the Human Immunodeficiency Virus (HIV), the Varicella zoster virus (VZV), or the Mycobacterium tuberculosis bacterium.

In a sixth aspect, there is provided a method of treating or preventing cancer, the method comprising administering to a subject in need thereof a first composition comprising at least a first mRNA construct comprising at least a first open reading frame (ORF), wherein the first ORF encodes a tumor-associated antigen, or an epitope containing fragment thereof. The first ORF is operatively linked to at least one untranslated region (UTR), wherein the UTR comprises at least a first organ protection sequence (OPS), wherein the OPS protects multiple organs, and wherein the first OPS comprises at least two micro-RNA (miRNA) target sequences, and wherein each of the at least two miRNA target sequences are optimised to hybridise with a corresponding miRNA sequence; and an in vivo delivery composition, wherein the mRNA construct is comprised within or adsorbed to the delivery composition.

In some embodiments, the method further comprises co-administering to the subject a second composition comprising at least one mRNA construct comprising at least a second open reading frame (ORF), wherein the second ORF encodes a proinflammatory cytokine which may be selected from: IL-12; IFNγ; IFNα; IFNβ; TNFα; IL-2; IL-6; IL-8; and GM-CSF. The second mRNA construct may comprise an OPS as defined in any preceding aspect. Co-administering may comprise administering the first composition and the second composition concurrently or consecutively, in either order.

In a seventh aspect, there is provided a method of treating or preventing cancer, the method comprising administering a cancer therapeutic vaccine composition to a subject in need thereof; and coadministering to the subject a composition comprising at least one mRNA construct comprising at least one open reading frame (ORF), wherein the at least one ORF encodes a proinflammatory cytokine which may be selected from: IL-12; IFNγ; IFNα; IFNβ; TNFα; IL-2; IL-6; IL-8; and GM-CSF. The ORF is operatively linked to at least one untranslated region (UTR), wherein the UTR comprises at least one OPS that protects multiple organs, and wherein the OPS comprises at least two miRNA target sequences, and wherein each of the at least two miRNA target sequences are optimised to hybridise with a corresponding miRNA sequence; and an in vivo delivery composition; wherein the mRNA construct is comprised within or adsorbed to the delivery composition.

In some embodiments, the cancer therapeutic vaccine composition delivers a tumor-associated antigen to the subject. The tumor-associated antigen is delivered to the subject using a viral vector, which may be an adenovirus vector, and in some embodiments is ChAdOx1 or ChAdOx2.

In some embodiments of the sixth or seventh aspect, the tumor-associated antigen comprises a colorectal tumour antigen and/or MUC1. The tumor-associated antigen may be a neoantigen, which may be personalised to the subject.

The invention is further exemplified in a variety of embodiments and examples described herein, the features of which may be further combined to form additional embodiments as would be understood by the skilled addressee.

### DRAWINGS

FIG. 1 shows schematic view (i.e. not to scale) of an mRNA construct incorporating an organ protection sequence (OPS) according to an embodiment of the invention.

FIG. 2 shows a schematic of the protocol carried out to determine the expression of the reporter gene mCherry after administration of compositions as described herein to various cell types, mCherry signal analysis is carried out by fluorescence microscopy (Texas Red and DAPI filters), with cell nuclei stained with Hoechst 33342.

FIG. 3 shows mCherry signal in three liver cell types following the above protocol, and demonstrates significant reduction of cell signal in both normal murine and human hepatocytes when transfected with the mRNAs containing multiple organ protection sequences (MOP), mCherry-3MOP or mCherry-5MOP mRNA, compared to the signal found in human liver cancer cells (Hep3B) or in normal murine hepatocyte (AML12) cells after transfection with control mCherry mRNA. The images are superimposition of images acquired with Texas Red and DAPI filters, showing mCherry fluorescence signal and cell nuclei staining.

FIG. 4 shows quantification of mCherry fluorescence in the transfected cells using a Cytation instrument (Biotek).

FIGS. 5A-5B show reduction in signal in the mCherry-3MOP treated cells. FIG. 5A shows mCherry signal in normal human kidney cells transfected with compositions as described herein, showing a reduction in signal in the mCherry-3MOP treated cells, indicating a reduction in mCherry translation. The images are superimposition of images acquired with Texas Red and DAPI filter cubes, showing mCherry fluorescence signal and cell nuclei staining. FIG. 5B shows quantification of mCherry fluorescence in the transfected normal human kidney cells using a Cytation instrument (Biotek).

FIGS. 6A-6F show comparison of mCherry signal in liver cells transfected with composition as described herein that comprise a perfect matched MOP sequence that binds miRNA-122, miRNA-192, and miRNA-30a, and demonstrates that the MOP sequence suppresses expression in AML12 murine hepatocytes but not in liver cancer cells (Hep3B). For each set of pictures, the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 6A) The top panel shows control Hep3B cells (liver cancer) that are not transfected with mRNA, the bottom panel shows no mCherry signal, (FIG. 6B) Cell nuclei staining and mCherry signal in Hep3B cells transfected with mRNA without a MOP sequence, (FIG. 6C) Cell nuclei staining and mCherry signal in Hep3B cells transfected with mRNA with the MOP sequence (FIG. 6D) Cell nuclei staining in control AML12 cells (normal liver) that are not transfected with mRNA, the bottom panel shows no mCherry signal, (FIG. 6E) Cell nuclei staining and mCherry signal in AML12 cells transfected with mRNA without a MOP sequence, (FIG. 6F) Cell nuclei staining and mCherry signal in AML12 cells transfected with mRNA with the MOP sequence.

FIGS. 7A-7F show comparison of mCherry signal in liver cells transfected with composition as described herein that comprise a perfect matched MOP sequence that binds Let7b, miRNA-126, and miRNA-30a, and demonstrates that the MOP sequence suppresses expression in AML12 murine hepatocytes but not in liver cancer cells (Hep3B). For each set of pictures, the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 7A) The top panel shows control Hep3B cells (liver cancer) that are not transfected with mRNA, the bottom panel shows no mCherry signal, (FIG. 7B) Cell nuclei staining and mCherry signal in Hep3B cells transfected with mRNA without a MOP sequence, (FIG. 7C) Cell nuclei staining and mCherry signal in Hep3B cells transfected with mRNA with the MOP sequence (FIG. 7D) Cell nuclei staining in control AML12 cells (normal liver) that are not transfected with mRNA, the bottom panel shows no mCherry signal, (FIG. 7E) Cell nuclei staining and mCherry signal in AML12 cells transfected with mRNA without a MOP sequence, (FIG. 7F) Cell nuclei staining and mCherry signal in AML12 cells transfected with mRNA with the MOP sequence.

FIGS. 8A-8B show comparison of mCherry signal in liver cells transfected with compositions as described herein that comprise a perfect matched multiplexed MOP sequence that binds to miRNA-122 replicated once (1*), twice (2*) or four times (4*), and shows that there is some dose dependence in suppression of mCherry expression in AML12 normal hepatocytes (FIG. 8A), but far less so in Hep3B cancer cells (FIG. 8B). For each of (FIG. 8A) and (FIG. 8B), the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. A control of no injected mRNA is included as well as mRNA for mCherry but without a MOP sequence.

FIGS. 9A-9D show comparison of mCherry signal in AML12 murine liver hepatocytes transfected with compositions as described herein that comprise an unperfect matched duplex (2*) MOP sequence that binds to miRNA122. For each of (FIG. 9A), (FIG. 9B), (FIG. 9C) and (FIG. 9D), the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 9A) the top panel shows cell nuclei staining in control AML12 cells (normal liver) that are not transfected with mRNA, and the bottom panel shows no mCherry signal, (FIG. 9B) Cell nuclei staining and mCherry signal in AML12 cells transfected with mRNA without a MOP sequence, (FIG. 9C) Cell nuclei staining and mCherry signal in AML12 cells transfected with mRNA with the 2* unperfect matched miRNA122 MOP sequence (nonoptimized), the bottom panel showing that there is detectable expression of mCherry in the AML12 cells, (FIG. 9D) Cell nuclei staining and mCherry signal in AML12 cells transfected with mRNA with the 2* perfect matched miRNA-122 MOP binding sequence (optimized), the bottom panel showing that there is almost no detectable expression of mCherry in the AML12 cells,

FIG. 10A-10F show comparison of mCherry signal in kidney cells transfected with compositions as described herein that comprise a perfect matched MOP sequence that binds to Let7b, miRNA-126, and miRNA-30a, and demonstrates that the MOP sequence suppresses mCherry expression in human kidney cells (hREC) but not in cancer cells (786-0). For each of (FIG. 10A), (FIG. 10B), (FIG. 10C), (FIG. 10D), (FIG. 10E) and (FIG. 10F), the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 10A) the top panel shows cell nuclei staining in control 786-0 human renal cell adenocarcinoma cells that are not transfected with mRNA, the bottom panel shows no mCherry signal, (FIG. 10B) cell nuclei staining and mCherry signal in 786-0 cells transfected with mRNA without a MOP sequence, (FIG. 10C) cell nuclei and mCherry signal in 786-0 cells transfected with mRNA with the MOP sequence, which shows evidence of expression, (FIG. 10D) cell nuclei staining in control hREC cells (normal mixed kidney epithelial cells) that are not transfected with mRNA, the bottom panel shows no mCherry signal, (FIG. 10E) cell nuclei staining and mCherry signal in hREC cells transfected with mRNA without a MOP sequence, (FIG. 10F) cell nuclei staining and mCherry signal in hREC cells transfected with mRNA with the MOP sequence, the mCherry signal alone showing virtually no expression.

FIGS. 11A-11F shows comparison of mCherry signal in kidney cells transfected with compositions as described herein that comprise a perfect matched MOP sequence that binds to miRNA-122, miRNA-192, and miRNA-30a, and demonstrates that the MOP sequence suppresses mCherry expression in human kidney cells (hREC) but not in cancer cells (786-0). For each of (FIG. 11A), (FIG. 11B), (FIG. 11C), (FIG. 11D), (FIG. 11E) and (FIG. 11F), the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 11A) the top panel shows cell nuclei staining in control 786-0 human renal cell adenocarcinoma cells that are not transfected with mRNA, the bottom panel shows no mCherry signal, (FIG. 11B) cell nuclei staining and mCherry signal in 786-0 cells transfected with mRNA without a MOP sequence, (FIG. 11C) cell nuclei and mCherry signal in 786-0 cells transfected with mRNA with the MOP sequence, which shows evidence of expression, (FIG. 11D) cell nuclei staining in control hREC cells (normal mixed kidney epithelial cells) that are not transfected with mRNA, the bottom panel shows no mCherry signal, (FIG. 11E) cell nuclei staining and mCherry signal in hREC cells transfected with mRNA without a MOP sequence, (FIG. 11F) cell nuclei staining and mCherry signal in hREC cells transfected with mRNA with the MOP sequence, the mCherry signal alone showing virtually no expression.

FIGS. 12A-12B show results of an experiment according to one embodiment in which human PBMC cells have been transfected with compositions as described herein that comprise (FIG. 12A) mRNA that expresses human IL-12 at three levels of dosage, expression of IL-12 is recorded six hours after transfection with the following mRNAs: NC (noncoding human recombinant IL-12 from a single chain—no ATG codon), hdcIL-12 (human recombinant IL-12 from a 1:1 mixture of separate IL12A and IL12B mRNAs), hscIL-12 (human recombinant IL-12 from a single chain), and hscIL-12-MOP (human recombinant IL-12 from a single chain), which is an single chain recombinant IL-12 expressing mRNA comprising a perfect matched MOP sequence that binds to miRNA-122-miRNA-203a-miRNA-1-miRNA-30a; (FIG. 12B) mRNA that expresses human GM-CSF at three levels of dosage, expression of GM-CSF is recorded six hours after transfection with the following mRNAs: NC (noncoding GM-CSF mRNA—no ATG codon), hGM-CSF, and hGM-CSF-MOP, which is an hGM-CSF expressing mRNA comprising a perfect matched MOP sequence that binds to miRNA-122-miRNA-203a-miRNA-1-miRNA-30a.

FIGS. 13A-13F shows comparison of mCherry signal in colon epithelial cells transfected with composition as described herein that comprise a perfect matched MOP sequence that binds miRNA-122, miRNA-192, and miRNA-30a, and demonstrates that the MOP sequence suppresses expression in colon epithelial cells but not in colon cancer cells (HCT-116). For each set of pictures, the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 13A) The top panel shows control colon epithelial cells that are not transfected with mRNA, the bottom panel shows no mCherry signal, (FIG. 13B) Cell nuclei staining and mCherry signal in colon cells transfected with mRNA without a MOP sequence, (FIG. 13C) Cell nuclei staining and mCherry signal in colon cells transfected with mRNA with the MOP sequence (FIG. 13D) Cell nuclei staining in control HCT-116 cells (colon cancer) that are not transfected with mRNA, the bottom panel shows no mCherry signal, (FIG. 13E) Cell nuclei staining and mCherry signal in HCT-116 cells transfected with mRNA without a MOP sequence, (FIG. 13F) Cell nuclei staining and mCherry signal in HCT-116 cells transfected with mRNA with the MOP sequence.

FIGS. 14A-14F shows comparison of mCherry signal in colon epithelial cells transfected with composition as described herein that comprise a perfect matched MOP sequence that binds miRNA-Let7b, miRNA-126, and miRNA-30a, and demonstrates that the MOP sequence provides organ protection by suppressing expression in both normal colon cells and colon cancer cells, attributed to presence of miRNA-Let7b binding site. For each set of pictures, the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 14A) The top panel shows control colon epithelial cells that are not transfected with mRNA, the bottom panel shows no mCherry signal, (FIG. 14B) Cell nuclei staining and mCherry signal in colon cells transfected with mRNA without a MOP sequence, (FIG. 14C) Cell nuclei staining and mCherry signal in colon cells transfected with mRNA with the MOP sequence (FIG. 14D) Cell nuclei staining in control HCT-116 cells (colon cancer) that are not transfected with mRNA, the bottom panel shows no mCherry signal, (FIG. 14E) Cell nuclei staining and mCherry signal in HCT-116 cells transfected with mRNA without a MOP sequence, (FIG. 14F) Cell nuclei staining and mCherry signal in HCT-116 cells transfected with mRNA with the MOP sequence.

FIGS. 15A-15C shows mCherry signal in normal healthy lung cells (BEAS-2B) transfected with composition as described herein that comprise a perfect matched MOP sequence that binds miRNA-Let7b, miRNA-126, and miRNA-30a, and demonstrates that the MOP sequence provides organ protection for the lung by suppressing expression in healthy lung cells, attributed to presence of miRNA-Let7b binding site. For each set of pictures, the top panel is a superimposition of images acquired with the Texas Red and DAPI filter cubes, showing cell nuclei staining and mCherry fluorescence. The bottom panel represents an image acquired with the Texas Red filter cube and shows the mCherry fluorescence only. (FIG. 15A) The top panel shows control cells that are not transfected with mRNA, the bottom panel shows no mCherry signal, (FIG. 15B) Cell nuclei staining and mCherry signal in cells transfected with mRNA without a MOP sequence, (FIG. 15C) Cell nuclei staining and mCherry signal in lung cells transfected with mRNA with the MOP sequence.

FIGS. 16A-16B shows results of an in vivo biodistribution experiment in mice, demonstrating that (FIG. 16A) after 3.5 hours post-administration (T3.5 h) with compositions of the invention, high levels of Luciferase expression can be seen in all groups through whole body imaging, including the MOP containing constructs (Group 2 and 3) and the control group with no MOP construct (Group 1); but significant down regulation of Luciferase expression is seen in MOP containing compositions after 24 hours (T24 h). In (FIG. 16B) ex vivo imaging of the organs after 24 hours shows decreased Luciferase expression in the liver, lungs, spleen, and kidney for mice in Group 2 and 3 (MOP containing constructs) as compared to Group 1 (no MOP control).

FIGS. 17A-17B show further results of the biodistribution study in mice carrying a subcutaneous Hep3B tumour (human liver cancer). Each mouse received via intra-tumoral injection compositions according to certain embodiments of the invention. Luciferase expression can be seen in tumour tissue in all groups through ex vivo imaging after 24 hours, while protection of the liver is provided by the MOP sequence for Group 2 and 3. Vehicle group received phosphate-buffered saline. Group 1 received Luciferase (no MOP control).

FIG. 18 shows the results of animal study of an antigen-specific immune response to ovalbumin with or without murine IL-12 adjuvant present. The administered dosages of mRNA are shown in the table underneath the graph. The response is shown in terms of amount of anti-ovalbumin murine IgG detected in serum 14 days after immunisation.

FIG. 19 shows results of an in vivo biodistribution experiment in mice following intra-muscular administration. The ex vivo imaging results demonstrates that after 4 hours post-administration with compositions of certain embodiments of the invention, there is decreased Luciferase expression in multiple organs for mice in groups that had MOP containing constructs (Luc-MOP1, Luc-MOP2, Luc-MOP3), as compared to no MOP control (Luc). Expression at the injection site remained high in all groups but for Luc-MOP1. Vehicle group received phosphate-buffered saline. The results show effective organ protection can be achieved using compositions of the invention via the intra-muscular administration route.

FIG. 20 shows results of an in vivo biodistribution experiment in mice following intravenous administration. The ex vivo imaging results demonstrates that after 6 hours post-administration with compositions of certain embodiments of the invention, there is decreased Luciferase expression in multiple organs for mice in groups that had MOP-containing constructs (Luc-MOP1, Luc-MOP2, Luc-MOP3), as compared to no MOP control (Luc). Vehicle group received phosphate-buffered saline. The results show effective organ protection can be achieved using compositions of the invention via the intravenous administration route.

FIG. 21 shows the results of an animal study of SARS-CoV-2 viral Spike protein-specific immune response in the presence or absence of immunostimulation from a murine IL-12 mRNA adjuvant. FIG. 21 shows the response of Balb/c mice in terms of serum IgG generated 42 days after immunization.

FIGS. 22A-22B shows results of an experiment according to one embodiment in which human PBMC cells have been transfected with compositions as described herein that comprise mRNA that expresses human IL-12 at three levels of dosage. FIG. 22a shows expression of IL-12 quantified 24 h after transfection with the following mRNAs: NC (noncoding human recombinant IL-12 from a single chain—lacking ATG codon), hscIL-12 (human recombinant IL-12 from a single chain), and hscIL-12-MOPV (single chain recombinant IL-12 expressing mRNA comprising a perfect matched MOP sequence that binds to miRNA-122-miRNA-203a-miRNA-1-miRNA-30a), hscIL-12-MOPC (single chain recombinant IL-12 expressing mRNA comprising a perfect matched MOP sequence that binds to miRNA-122-miRNA-192-miRNA-30a). FIG. 22b shows IL-12-mediated induction of interferon-gamma (IFN-γ) in PBMC transfected by mRNA that expresses human IL-12. Human interferon-gamma is measured 72 h after transfection with the mRNAs. The data shows a dose-dependent expression of human IL-12 (FIG. 22A), and an IL-12-mediated induction of interferon-gamma (FIG. 22B), which is an immunostimulatory cytokine critical for both innate and adaptive immunity.

FIG. 23 shows results of an experiment according to one embodiment in which human PBMC cells have been transfected with compositions as described herein that comprise mRNA that expresses SARS-CoV-2 Spike mRNA with MOP in combination with human single chain recombinant IL-12 expressing mRNA with (hscIL-12-MOPV) and without MOP (hscIL-12). The MOP sequences comprise a perfect matched binding sequence for miRNA-122-miRNA-203a-miRNA-1-miRNA-30a). The results show that 120 h post-transfection, interferon-gamma (INF-γ) expression is increased in the presence of mRNA expressing human IL-12 with and without MOP.

### DETAILED DESCRIPTION

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN:1934-3647); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, Pages 2-662 (2011); Synthetic Biology, Part B, Computer Aided Design and DNA Assembly, Methods in Enzymology, Edited by Christopher Voigt, Volume 498, Pages 2-500 (2011); RNA Interference, Methods in Enzymology, David R. Engelke, and John J. Rossi, Volume 392, Pages 1-454 (2005). Each of these general texts is herein incorporated by reference.

Prior to setting forth the invention, a number of definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘isolated’, when applied to a polynucleotide sequence, denotes that the sequence has been removed from its natural organism of origin and is, thus, free of extraneous or unwanted coding or regulatory sequences. The isolated sequence is suitable for use in recombinant DNA processes and within genetically engineered protein synthesis systems. Such isolated sequences include cDNAs, mRNAs and genomic clones. The isolated sequences may be limited to a protein encoding sequence only or can also include 5′ and 3′ regulatory sequences such as promoters and transcriptional terminators, or untranslated sequences (UTRs). Prior to further setting forth the invention, a number of definitions are provided that will assist in the understanding of the invention.

A ‘polynucleotide’ is a single or double stranded covalently-linked sequence of nucleotides in which the 3′ and 5 ends on each nucleotide are joined by phosphodiester bonds. The polynucleotide may be made up of deoxyribonucleotide bases or ribonucleotide bases. Polynucleotides include DNA and RNA, and may be manufactured synthetically in vitro or isolated from natural sources. Sizes of polynucleotides are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called ‘oligonucleotides’. The term ‘nucleic acid sequence’ as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acid sequences may include DNA and RNA, and may be manufactured synthetically in vitro or isolated from natural sources. In specific embodiments of the present invention the nucleic acid sequence comprises messenger RNA (mRNA).

Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA).

According to the present invention, homology to the nucleic acid sequences described herein is not limited simply to 100% sequence identity. In this regard, the term “substantially similar”, relating to two sequences, means that the sequences have at least 70%, 80%, 90%, 95% or 100% similarity. Likewise, the term “substantially complementary”, relating to two sequences, means that the sequences are completely complementary, or that at least 70%, 80%, 90%, 95% or 99% of the bases are complementary. That is, mismatches can occur between the bases of the sequences which are intended to hybridise, which can occur between at least 1%, 5%, 10%, 20% or up to 30% of the bases. However, it may be desired in some cases to distinguish between two sequences which can hybridise to each other but contain some mismatches—an “inexact match”, “imperfect match”, or “inexact complementarity”- and two sequences which can hybridise to each other with no mismatches—an “exact match”, “perfect match”, or “exact complementarity”. Further, possible degrees of mismatch are considered.

As used herein, the term ‘organ protection sequence’ (‘OPS’) refers to a sequence comprised of a plurality of microRNA (miRNA) target sequences of natural or synthetic origin and, optionally, one or more auxiliary sequences. Where an OPS confers protection to multiple organs it may be referred to as a multiple or ‘multi-’organ protection (MOP) sequence. The term ‘target sequence’ refers to a sequence comprised within a mRNA sequence, such as within an untranslated region (UTR), that is targeted for binding by a specified miRNA. Binding occurs by way of nucleic acid hybridisation between complementary base pairs comprised within the miRNA and the corresponding target sequence. The binding interaction may be optimised such that no mismatches between the specified miRNA and the target sequence occur, or mismatches are limited to no more than a single base pair mismatch across the length of the target sequence. In an embodiment of the invention a single base mismatch is limited to the 5′ or 3′ end of the target sequence. Optimised sequences can also be described as being perfectly matched to the target miRNA that is present in the cell and may differ from the wild type binding sequence by two or more base pairs. Wild type sequences that comprise more than two naturally occurring mismatches are deemed to be un-perfectly or imperfectly matched to the corresponding complementary miRNA sequence.

The term ‘operatively linked’, when applied to nucleic acid sequences, for example in an expression construct, indicates that the sequences are arranged so that they function cooperatively in order to achieve their intended purposes. By way of example, in a DNA vector a promoter sequence allows for initiation of transcription that proceeds through a linked coding sequence as far as a termination sequence. In the case of RNA sequences, one or more untranslated regions (UTRs) may be arranged in relation to a linked polypeptide coding sequence referred to as an open reading frame (ORF). A given mRNA as disclosed herein may comprise more than one ORFs, a so-called polycistronic RNA. An mRNA may encode more than one polypeptide, and may as a result include cleavage sites or other sequences necessary to result in the production of multiple functional products, as known in the art. A UTR may be located 5′ or 3′ in relation to an operatively linked coding sequence ORF. UTRs may comprise sequences typically found in mRNA sequences found in nature, such as any one or more of: Kozak consensus sequences, initiation codons, cis-acting translational regulatory elements, cap-independent translation initiator sequences, poly-A tails, internal ribosome entry sites (IRES), structures regulating mRNA stability and/or longevity, sequences directing the localisation of the mRNA, and so on. An mRNA may comprise multiple UTRs that are the same or different. The one or more UTRs may comprise or be located proximate or adjacent to an OPS. UTRs may comprise linear sequences that provide translational or stability control over the mRNA, such as Kozak sequences, or they may also comprise one or more sequences that promote the formation of localised secondary structure, particularly within a 5′ UTR. In one embodiment of the invention, a 5′ UTR that has a lower-than-average GC content may be utilised to promote efficient translation of the mRNA.

The term ‘expressing a polypeptide’ in the context of the present invention refers to production of a polypeptide for which the polynucleotide sequences described herein code. Typically, this involves translation of the supplied mRNA sequence—i.e. the ORF—by the ribosomal machinery of the cell to which the sequence is delivered.

The term ‘diseased’ as used herein, as in ‘diseased cells’ and/or ‘diseased tissue’ indicates tissues and organs (or parts thereof) and cells which exhibit an aberrant, non-healthy or disease pathology. For instance, diseased cells may be infected with a virus, bacterium, prion, fungi or eukaryotic parasite; may comprise deleterious mutations; and/or may be cancerous, precancerous, tumoral or neoplastic. Infection may comprise a pathogen that is internalised and resides within the cell for a significant portion of its life cycle. Diseased cells may comprise an altered intra-cellular miRNA environment when compared to otherwise normal or so-called healthy cells. In certain instances, diseased cells may be pathologically normal but comprise an altered intra-cellular miRNA environment that represents a precursor state to disease. Diseased tissues may comprise healthy tissues that have been infiltrated by diseased cells from another organ or organ system. By way of example, many inflammatory diseases comprise pathologies where otherwise healthy organs are subjected to infiltration with immune cells such as T cells and neutrophils. By way of a further example, organs and tissues subjected to stenotic or cirrhotic lesions may comprise both healthy and diseased cells in close proximity.

The term ‘cancer’ as used herein refers to neoplasms in tissue, including malignant tumors, which may be primary cancer starting in a particular tissue, or secondary cancer having spread by metastasis from elsewhere. The terms cancer, neoplasm and malignant tumors are used interchangeably herein. Cancer may denote a tissue or a cell located within a neoplasm or with properties associated with a neoplasm. Neoplasms typically possess characteristics that differentiate them from normal tissue and normal cells. Among such characteristics are included, but not limited to: a degree of anaplasia, changes in morphology, irregularity of shape, reduced cell adhesiveness, the ability to metastasize, and increased cell proliferation. Terms pertaining to and often synonymous with ‘cancer’ include sarcoma, carcinoma, malignant tumor, epithelioma, leukaemia, lymphoma, transformation, neoplasm and the like. As used herein, the term ‘cancer’ includes premalignant, and/or precancerous tumors, as well as malignant cancers.

The term ‘healthy’ as used herein, as in ‘healthy cells’ and/or ‘healthy tissue’ indicates tissues and organs (or parts thereof) and cells which are not themselves diseased and/or approximate to a typically normal functioning phenotype. It can be appreciated that in the context of the invention the term ‘healthy’ is relative, as, for example, non-neoplastic cells in a tissue affected by tumors may well not be entirely healthy in an absolute sense. Therefore ‘non-healthy cells’ means cells which are not themselves neoplastic, cancerous or pre-cancerous but which may be cirrhotic, inflamed, or infected, or otherwise diseased for example. Similarly, ‘healthy or non-healthy tissue’ means tissue, or parts thereof, without tumors, neoplastic, cancerous or pre-cancerous cells; or other diseases as mentioned above; regardless of overall health. For instance, in the context of an organ comprising cancerous and fibrotic tissue, cells comprised within the fibrotic tissue may be thought of as relatively ‘healthy’ compared to the cancerous tissue. Models used for approximation of normal functioning phenotypes for ‘healthy’ cells may include immortalised cell lines that are otherwise close to the originator cells in terms of cellular function and gene expression.

In an alternative embodiment, the health status of a cell, cell type, tissue and/or organ is determined by the quantification of miRNA expression. In certain disease types, such as cancer, the expression of particular miRNA species is affected, and can be up- or down-regulated compared to unaffected cells. This difference in the miRNA transcriptome can be used to identify relative states of health, and/or to track the progression of healthy cells, cell types, tissues and/or organs towards a disease state. The disease state may include the various stages of transformation into a neoplastic cell. In embodiments of the present invention the differential variations in the miRNA transcriptome of cell types comprised within a given organ or organ system is leveraged in order to control protein expression in the different cell types.

As used herein, the term ‘organ’ is synonymous with an ‘organ system’ and refers to a combination of tissues and/or cell types that may be compartmentalised within the body of a subject to provide a biological function, such as a physiological, anatomical, homeostatic or endocrine function. Suitably, organs or organ systems may mean a vascularized internal organ, such as a liver or pancreas. Typically, organs comprise at least two tissue types, and/or a plurality of cell types that exhibit a phenotype characteristic of the organ. Tissues or tissue systems may cooperate but not formally be considered as an organ. For example, blood is generally considered a tissue, or even a liquid tissue, but depending upon the definition used may not be regarded as an organ in the strict sense. Nevertheless, the compositions and methods of the invention in certain embodiments may serve to exhibit a protective effect in respect of organs, tissues and tissue systems including the blood, haematopoietic and lymphoid tissue.

The term ‘therapeutic virus’ as used herein refers to a virus which is capable of infecting and killing cancer cells, including indirect killing by the stimulation of host anti-tumoral responses. Therapeutic viruses may also include attenuated or modified viruses that are useful in vaccine formulation.

TABLE 1
Examples of therapeutic viruses and subtypes thereof

| Therapeutic virus | Type |
|---|---|
| Rhabdoviridae family (e.g. Maraba virus, Vesicular Somatitis virus) | Enveloped RNA |
| Poxviridae family (e.g. Vaccinia virus) | Enveloped DNA |
| Reoviridae family (e.g. Reovirus) | Non enveloped RNA |
| Paramyxoviridae family (e.g. Measles virus, Newcastle Disease | Enveloped RNA |
| virus) | |
| Picomaviridae family (e.g. Poliovirus, Coxsackie A virus, Seneca | Non enveloped RNA |
| Valley virus) | |
| Togaviridae family (e.g. Semliki Forest Virus, Sindbis Virus) | Enveloped RNA virus |
| Parvoviridae family (e.g. Protoparvovirus) | Non enveloped DNA |
| Herpesviridae family (e.g. Herpes Simplex Virus Type 1) | Enveloped DNA |
| Adenoviridae family (e.g. Adenovirus) | Non enveloped DNA |

In embodiments of the invention viruses may be selected from any one of the Groups I-VII of the Baltimore classification of viruses (Baltimore D (1971). “Expression of animal virus genomes”. Bacteriol Rev. 35 (3): 235-41). In specific embodiments of the invention suitable viruses may be selected from Baltimore Group I, which are characterised as having double stranded DNA viral genomes; Group II, which are characterized as having positive single stranded DNA genomes, Group Ill, which are characterized as having double stranded RNA viral genomes, Group IV, which have single stranded positive RNA genomes; and Group V, which have single stranded negative RNA genomes.

The term ‘polypeptide’ as used herein is a polymer of amino acid residues joined by peptide bonds, whether produced naturally or in vitro by synthetic means. Polypeptides of less than around 12 amino acid residues in length are typically referred to as “peptides” and those between about 12 and about 30 amino acid residues in length may be referred to as “oligopeptides”. The term “polypeptide” as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term “protein” is used herein to refer to a macromolecule comprising one or more polypeptide chains.

The term ‘gene product’ as used herein refers to the peptide or polypeptide encoded by at least one coding sequence or Open Reading Frame (ORF) comprised within an mRNA construct of the invention as described herein. A polycistronic mRNA construct may be used, which results in the production of multiple gene products encoded by multiple ORFs located on the same polynucleic strand. It will be appreciated that multiple ORFs may lead to the production in situ of a variety of products—e.g. proteins, peptides or polypeptides—that may cooperate functionally, or may form complexes and/or multimeric proteins with diverse biological and potentially therapeutic effects.

The gene product encoded by the mRNA is typically a peptide, polypeptide or protein. Where a particular protein consists of more than one subunit, the mRNA may code for one or more than one subunit within one or more ORFs. In alternative embodiments, a first mRNA may code for a first subunit, whilst a second co-administered mRNA may code for a second subunit that, when translated in situ, leads to assembly of a multi-subunit protein gene product. Translation of the gene product within the target cell allows for localised post-translational modification appropriate to the cell type to be applied. Such modifications may regulate folding, localization, interactions, degradation, and activity of the gene product. Typical post translational modifications may include cleavage, refolding and/or chemical modification such as methylation, acetylation or glycosylation.

Where present on separate mRNA constructs, and formulated to be associated with delivery particles (as described elsewhere herein), these may be co-formulated, such that different mRNA constructs may be associated with the same individual delivery particles, or separately formulated, such that different mRNA constructs may be associated with different delivery particles.

Delivery of mRNA directly to cells allows direct and controllable translation of the desired gene products such as polypeptides and/or proteins in the cells. Provision of mRNA specifically allows not only for the use of cell expression modulation mechanisms, such as miRNA mediated control (as detailed in specific embodiments below), but also represents a finite and exhaustible supply of the product, rather than the potentially permanent change to the transcriptome of a target cell, which an episomal or genomically inserted DNA vector might provide.

In embodiments of the present invention, an mRNA sequence is provided that comprises a sequence that codes for at least one polypeptide in operative combination with one or more untranslated regions (UTRs) that may confer tissue specificity, and stability to the nucleic acid sequence as a whole. By ‘tissue specificity’, it is meant that translation of the protein product encoded by the mRNA is modulated according to the presence of the UTRs. Modulation may include permitting, reducing or even blocking detectable translation of the mRNA into a protein. The UTRs may be linked directly to the mRNA in cis—i.e. on the same polynucleotide strand. In an alternative embodiment, a first sequence that codes for a gene product is provided and a further second sequence, that hybridises to a portion of the first sequence, is provided that comprises one or more UTRs that confer tissue specificity to the nucleic acid sequence as a whole. In this latter embodiment, the UTR is operatively linked to the sequence that encodes the gene product in trans.

According to specific embodiments of the invention, an mRNA is provided that comprises such associated nucleic acid sequences operatively linked thereto as are necessary to prevent or reduce expression of a gene product in non-diseased tissue, e.g. in healthy hepatocytes, CNS, muscle, skin etc. The mRNA is hereafter referred to as a ‘coding mRNA’. As such, this coding mRNA construct, or transcript, is provided that comprises a 5′ cap and UTRs necessary for ribosomal recruitment and tissue and/or organ specific expression (typically, but not exclusively positioned 3′ to the ORF), as well as start and stop codons that respectively define one or more ORFs. When the construct is introduced systemically or via localised administration into non-diseased liver, lung, pancreas, breast, brain/CNS, kidney, spleen, muscle, skin and/or colon-GI tract, expression of the gene product is prevented or reduced. In contrast, neoplastic or otherwise diseased cells comprised within the aforementioned organs typically do not conform to normal non-diseased cell expression patterns, possessing a quite different miRNA transcriptome. The polypeptide(s) encoded by the mRNA is translated specifically in these aberrant cells but not—or to a lesser extent—in neighbouring healthy or non-diseased cells. Delivery of the mRNA construct to the organs mentioned above may be achieved via a particulate delivery platform as described herein, or in any suitable way known in the art. Cell type specific expression can be mediated via microRNA modulation mechanisms such as those described in more detail below.

According to further embodiments of the invention, an mRNA is provided that comprises such associated nucleic acid sequences operatively linked thereto as are necessary to prevent or reduce expression of a gene product in tissues or organs not required to generate an immune response to an antigen, e.g. in hepatocytes, CNS, muscle, skin, kidney etc. The coding mRNA construct, or transcript, is provided that may or may not comprise a 5′ cap, as well as one or more UTRs necessary for ribosomal recruitment and tissue and/or organ specific expression (typically, but not exclusively positioned 3′ to the ORF), as well as start and stop codons that respectively define one or more ORFs. When the construct is introduced systemically or via localised administration into a subject, expression of the gene product is prevented or reduced in cells and tissues that are not typically required for an immune response. In contrast, immune cells, such as T cells, B Cells or antigen presenting cells (APCs), including different types of dendritic cells (DCs), comprised within the body or in the aforementioned organs possess a different miRNA transcriptome. The polypeptide(s) encoded by the mRNA is translated specifically in these immune cells but not—or to a lesser extent—in neighbouring healthy cells and tissues. Delivery of the mRNA construct to the cells and tissues mentioned above may be achieved via a particulate delivery platform as described herein, or in any suitable way known in the art.

A ‘therapeutic component’ or ‘therapeutic agent’ as defined herein refers to a molecule, substance, cell or organism that when administered to an individual human or other animal as part of a therapeutic intervention, contributes towards a therapeutic effect upon that individual human or other animal. The therapeutic effect may be caused by the therapeutic component itself, or by another component of the therapeutic intervention. The therapeutic component may be a coding nucleic acid component, in particular an mRNA. The coding nucleic acid component(s) may code for therapeutic enhancement factors, as defined below. A therapeutic component may also comprise a drug, optionally a chemotherapeutic drug such as a small molecule or monoclonal antibody (or fragment thereof). In other embodiments of the invention, the therapeutic agent comprises a therapeutic virus, such as a viral vector.

The term ‘therapeutic effect’ refers to a local or systemic effect in an animal subject, typically a human, caused by a pharmacologically or therapeutically active agent that comprises a substance, molecule, composition, cell or organism that has been administered to the subject, and the term ‘therapeutic intervention’ refers to the administration of such a substance, molecule, composition, cell or organism. The term thus means any agent intended for use in the diagnosis, cure, mitigation, treatment or prevention of disease or in the enhancement of desirable physical or mental development and conditions in an animal or human subject. The phrase ‘therapeutically-effective amount’ means that amount of such an agent that produces a desired local or systemic effect at a reasonable benefit/risk ratio applicable to any treatment. In certain embodiments, a therapeutically effective amount of an agent will depend on its therapeutic index, solubility, and the like. For example, certain therapeutic agents of the present invention may be administered in a sufficient amount to produce a reasonable benefit/risk ratio applicable to such treatment. In the specific context of treatment of disease including infectious disease or cancer, a ‘therapeutic effect’ can be manifested by various means, including but not limited to, a decrease in infectious pathogenic organism titre, an increase in beneficial cellular biomarkers (e.g. an increase in white cell count), a reduction in solid tumor volume, a decrease in the number of cancer cells, a decrease in the number of metastases observed, an increase in life expectancy, decrease in cancer cell proliferation, decrease in cancer cell survival, a decrease in the expression of tumor cell markers, and/or amelioration of various physiological symptoms associated with the condition. In the specific context of the treatment of a viral, bacterial or parasitic infection, such as by prophylaxis through vaccination, a ‘therapeutic effect’ may be shown by full or partial resistance to pathogen challenge, presence of circulating antibodies to the pathogen in the human or animal subject, or other known measures of vaccine efficacy.

In one embodiment, the subject to whom therapy is administered is a mammal (e.g., rodent, primate, non-human mammal, domestic animal or livestock, such as a dog, cat, rabbit, guinea pig, cow, horse, sheep, goat and the like), and is suitably a human. In a further embodiment, the subject is an animal model of disease, such as cancer. For example, the animal model can be an orthotopic xenograft animal model of a human-derived cancer, suitably liver, lung, pancreas, breast, brain, kidney, muscle, skin and/or colon-GI tract cancer. In a further embodiment, the subject is an animal model of infectious disease. For example, the animal model may be infected with one or more viruses, bacteria, fungi, prions or eukaryotic parasites, or is to be infected with such pathogens.

In a specific embodiment of the methods of the present invention, the subject has not yet undergone a therapeutic treatment, such as therapeutic viral therapy, chemotherapy, radiation therapy, targeted therapy, vaccination, and/or anti-immune checkpoint therapy. In still another embodiment, the subject has undergone a therapeutic treatment, such as the aforementioned therapies. In yet a further embodiment, the subject is undergoing a therapeutic treatment, such as the aforementioned therapies.

In further embodiments, the subject has had surgery to remove cancerous or precancerous tissue. In other embodiments, the cancerous tissue has not been removed, for example, the cancerous tissue may be located in an inoperable region of the body, such as in a tissue or organ that if subjected to surgical intervention may compromise the life of the subject, or in a region where a surgical procedure would cause considerable risk of permanent harm or even lethality.

In some embodiments, the provided coding mRNA construct may code fora ‘therapeutic enhancement factor’. According to the present invention therapeutic enhancement factors are gene products or polypeptides that may enhance or facilitate the ability of another, co-administered therapeutic agent, to exert a therapeutic effect upon a given cell, suitably the target cell. When introduced into or in the vicinity of the target cell, expression of the therapeutic enhancement factor may cooperate with a co-administered therapeutic agent thereby enabling or enhancing the therapeutic activity of the agent. In other embodiments the therapeutic enhancement factor may act as an adjuvant for a co- or sequentially administered vaccine. Adjuvants are pharmacological or immunological substances that may be used to activate the innate immune system of a subject. In this way they enable the innate immune system of the subject to respond to infection from a pathogen more rapidly. Adjuvants may also serve to stimulate adaptive immune responses that are specific to particular infectious agents, such as viral or bacterial infections. Some adjuvants may also be effective in directing effective antigen presentation and stimulating and enhancing T helper type-1 (Th1) immune responses. Alternatively, the therapeutic enhancement factor may act as an adjuvant for a co- or sequentially administered attenuated or modified virus, such as a modified adenovirus utilised in a vaccine formulation. Inactivated virus or live attenuated virus vaccines will typically need adjuvants in order to promote immune response. In addition, the inherent immunogenicity of recombinant protein-based subunit vaccines is also relatively low, and co-administered adjuvants are desirable. Hence, in specific embodiments of the invention the role of an adjuvant composition is to increase the level of neutralising antibodies produced by immune cells in response to a presented antigen.

Multiple therapeutic enhancement factors may be combined in compositions according to specific embodiments of the present invention. In such embodiments, the coding sequences for each therapeutic enhancement factor may be present in separate mRNA molecules. In some embodiments, sequences for more than one therapeutic enhancement factor may be present on the same mRNA molecule. In such cases the polycistronic mRNA molecule further comprises sequences as necessary for the expression of all coded sequences, such as internal ribosome entry sites (IRES).

In embodiments where multiple different mRNA molecules are comprised in one or more delivery system, it is contemplated that each delivery system—e.g. particle, liposome, viral vector system—may comprise one or more than one type of mRNA molecule as the ‘payload’; that is, not every delivery payload in a particular embodiment will necessarily comprise all of the mRNA molecules provided in said embodiment. In this way, it is also considered possible to direct different delivery systems and their associated sequences to different target cells, with the targeting agents described herein.

Similarly, in any embodiments where separate mRNA constructs are provided, and in which they are formulated to be associated with delivery particles (as described elsewhere herein), these may be co-formulated (that is, the different mRNA may be packaged with the delivery particles together in the same process), such that different mRNA constructs may be associated with the same delivery particles, or separately formulated, such that different mRNA constructs may be associated with different delivery particles.

The mRNA constructs of certain embodiments of the invention may be synthesised from a polynucleotide expression construct, which may be for example a DNA plasmid. This expression construct may comprise any promoter sequence necessary for the initiation of transcription and a corresponding termination sequence, such that transcription of the mRNA construct can occur. Such polynucleotide expression constructs are contemplated to comprise embodiments of the invention in their own right.

Cytokines

In an embodiment of the invention the mRNA constructs may encode a gene product for a cytokine, for example, a cytokine that acts as an adjuvant.

Cytokines are a broad category of small proteins important in cell signaling. Cytokines have been shown to be involved in autocrine, paracrine and endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. They act through cell surface receptors and are especially important in the immune system; cytokines modulate the balance between humoral and cell-based immune responses, and they regulate the maturation, growth, and responsiveness of particular cell populations. Cytokines have been classed as interleukins, lymphokines, monokines, interferons, colony stimulating factors and chemokines.

Interleukins (ILs) are a group of cytokines (secreted proteins and signal molecules) that were first seen to be expressed by white blood cells (leukocytes). The function of the immune system depends in a large part on interleukins, and rare deficiencies of a number of them have been described, all featuring autoimmune diseases or immune deficiency. The majority of interleukins are synthesized by helper CD4 T lymphocytes, as well as through monocytes, macrophages, and endothelial cells. They promote the development and differentiation of T and B lymphocytes, and hematopoietic cells. Interleukins include interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), interleukin 19 (IL-19), interleukin 20 (IL-20), interleukin 21 (IL-21), interleukin 22 (IL-22), interleukin 23 (IL-23), interleukin 24 (IL-24), interleukin 25 (IL-25), interleukin 26 (IL-26), interleukin 27 (IL-27), interleukin 28 (IL-28), interleukin 29 (IL-29), interleukin 30 (IL-30), interleukin 31 (IL-31), interleukin 32 (IL-32), interleukin 33 (IL-33), interleukin 35 (IL-35) and interleukin 36 (IL-36).

IL-1 alpha and IL-1 beta are cytokines that participate in the regulation of immune responses, inflammatory reactions, and hematopoiesis. IL-2 is a lymphokine that induces the proliferation of responsive T cells. In addition, it acts on some B cells, via receptor-specific binding, as a growth factor and antibody production stimulant. IL-3 is a cytokine that regulates hematopoiesis by controlling the production, differentiation and function of granulocytes and macrophages. IL-4 induces proliferation and differentiation of B cells and T cell proliferation. IL-5 regulates eosinophil growth and activation. IL-6 plays an essential role in the final differentiation of B cells into immunoglobulin-secreting cells, as well as inducing myeloma/plasmacytoma growth, nerve cell differentiation, and, in hepatocytes, acute-phase reactants. IL-7 is a cytokine that serves as a growth factor for early lymphoid cells of both B- and T-cell lineages. IL-8 induces neutrophil chemotaxis. IL-9 is a cytokine that supports IL-2 independent and IL-4 independent growth of helper T cells. IL-10 is a protein that inhibits the synthesis of a number of cytokines, including IFN-gamma, IL-2, IL-3, TNF, and GM-CSF produced by activated macrophages and by helper T cells. IL-11 stimulates megakaryocytopoiesis, leading to an increased production of platelets, as well as activating osteoclasts, inhibiting epithelial cell proliferation and apoptosis, and inhibiting macrophage mediator production. IL-12 is involved in the stimulation and maintenance of Th1 cellular immune responses, including the normal host defence against various intracellular pathogens. IL-13 is a pleiotropic cytokine that may be important in the regulation of the inflammatory and immune responses. IL-14 controls the growth and proliferation of B cells and inhibits Ig secretion. IL-15 induces production of Natural killer cells. IL-16 is a CD4+ chemoattractant. IL-17 is a potent proinflammatory cytokine produced by activated memory T cells. IL-18 induces production of IFNg and increased natural killer cell activity. IL-20 regulates proliferation and differentiation of keratinocytes. IL-21 co-stimulates activation and proliferation of CD8+ T cells, augment NK cytotoxicity, augments CD40-driven B cell proliferation, differentiation and isotype switching, promotes differentiation of Th17 cells. IL-22 stimulates production of defensins from epithelial cells and activates STAT1 and STAT3. IL-23 is involved in the maintenance of IL-17 producing cells and increases angiogenesis but reduces CD8 T-cell infiltration. IL-24 plays important roles in tumor suppression, wound healing and psoriasis by influencing cell survival, inflammatory cytokine expression. IL-25 induces the production IL-4, IL-5 and IL-13, which stimulate eosinophil expansion. IL-26 enhances secretion of IL-10 and IL-8 and cell surface expression of CD54 on epithelial cells. IL-27 regulates the activity of B lymphocyte and T lymphocytes. IL-28 plays a role in immune defense against viruses. IL-29 plays a role in host defenses against microbes. IL-30 forms one chain of IL-27. IL-31 may play a role in inflammation of the skin. IL-32 induces monocytes and macrophages to secrete TNF-α, IL-8 and CXCL2. IL-33 induces helper T cells to produce type 2 cytokines. IL-35 induces suppression of T helper cell activation. IL-36 regulates DC and T cell responses.

Lymphokines are a subset of cytokines that are produced by a type of immune cell known as a lymphocyte. They are protein mediators typically produced by T cells to direct the immune system response by signalling between its cells. Lymphokines have many roles, including the attraction of other immune cells, including macrophages and other lymphocytes, to an infected site and their subsequent activation to prepare them to mount an immune response. Lymphokines aid B cells to produce antibodies. Important lymphokines secreted by the T helper cell include IL2, IL3, IL4, IL5, IL6, granulocyte-macrophage colony-stimulating factor (GM-CSF) and interferon gamma (IFNγ).

GM-CSF stimulates stem cells to produce granulocytes (neutrophils, eosinophils, and basophils) and monocytes. Monocytes exit the circulation and migrate into tissue, whereupon they mature into macrophages and dendritic cells. Thus, it is part of the immune/inflammatory cascade, by which activation of a small number of macrophages can rapidly lead to an increase in their numbers, a process crucial for fighting infection. GM-CSF also enhances neutrophil migration and causes an alteration of the receptors expressed on the cells surface. IFNγ is a cytokine that is critical for innate and adaptive immunity against infections. IFNγ is an activator of macrophages and inducer of major histocompatibility complex class II molecule expression. The importance of IFNγ in the immune system stems in part from its ability to inhibit viral replication directly, and most importantly from its immunostimulatory and immunomodulatory effects.

A monokine is a type of cytokine produced primarily by monocytes and macrophages. Some monokines include IL-1, tumor necrosis factor-alpha, alpha and beta interferon, and colony stimulating factors. Tumor necrosis factor (TNF) is a cytokine—a small protein used by the immune system for cell signaling. TNF is released to recruit other immune system cells as part of an inflammatory response to an infection. Interferons (IFNs) are a group of signalling proteins made and released by host cells in response to the presence of several viruses. IFN-α proteins are produced mainly by plasmacytoid dendritic cells (pDCs) and are mainly involved in innate immunity against viral infection. IFN-β proteins are produced in large quantities by fibroblasts and have antiviral activity that is involved mainly in innate immune response. Colony-stimulating factors (CSFs) are secreted glycoproteins that bind to receptor proteins on the surfaces of hemopoietic stem cells, thereby activating intracellular signalling pathways that can cause the cells to proliferate and differentiate into a blood cell.

Chemokines are a family of small cytokines that have the ability to induce directed chemotaxis in nearby responsive cells. Chemokines are functionally divided into those that are homeostatic and those that are inflammatory. Homeostatic chemokines are constitutively produced in certain tissues and are responsible for basal leukocyte migration and include: CCL14, CCL19, CCL20, CCL21, CCL25, CCL27, CXCL12 and CXCL13. Inflammatory chemokines are formed under pathological conditions and actively participate in the inflammatory response attracting immune cells to the site of inflammation and include CXCL-8, CCL2, CCL3, CCL4, CCL5, CCL11, CXCL10.

Interferons (IFNs) are a group of signaling proteins made and released by host cells in response to the presence of several viruses. IFN-α, IFN-β, IFN-ε, IFN-κ and IFN-ω bind to the IFN-α/β receptor complex and bind to specific receptors on target cells, which leads to expression of proteins that will prevent the virus from producing and replicating its RNA and DNA. IFN-γ is released by cytotoxic T cells and type-1 T helper cells, however, IFN-γ blocks the proliferation of type-2 T helper cells.

A growth factor is a naturally occurring substance capable of stimulating cell proliferation, wound healing, and occasionally cellular differentiation. Growth factors include Adrenomedullin (AM), Angiopoietin (Ang), Autocrine motility factor, Bone morphogenetic proteins (BMP1, BMP2, BMP3, BMP4, BMP5, BMP6, BMP7, BMP8, BMP9, BMP10, BMP11, BMP12, BMP12, BMP14 and BMP15), Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Fetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF) and Wnt Signaling Pathway proteins.

As mentioned above, interleukin 12 (IL-12) is an immune-stimulatory cytokine for immune cells including T cells and NK cells. IL-12 is a heterodimeric cytokine that is produced specifically by phagocytic cells as well as antigen-presenting cells and enhances anti-tumor immune responses. A consequence of the potent immune stimulatory properties of IL-12 is that systemic administration can lead to serious side effects that limit its clinical application in patients. Expression of IL-12 by engineered NK92 at tumor sites has been shown to increase the antitumor activities of chimeric antigen receptor (CAR)-modified T cells (Luo et al. Front Oncol. (2019) Dec. 19; 9:1448). It is believed that IL-12 induced IFNγ accumulation in tumors also promotes the penetration of T-lymphocytes or other host immune cells (e.g. NK cells) into the tumors, thereby enhancing the therapeutic effects (Chinnasamy D. et al. Clin Cancer Res 2012:18/Chmielewski M. et al. Cancer Res 2011; 71/Kerkar S P. Et al. J Clin Invest 2011; 121/Jackson H J. Et al. Nat Rev Clin Oncol 2016; 13).

In embodiments of the present invention the compositions of the invention comprise an mRNA that include at least one ORF that encodes functional IL-12 or an analogue or derivative thereof. Since, wild type IL-12 is comprised of a heterodimer of 35 kDa IL-12A and 40 kDa IL-12B subunits, the ORF may comprise one of these subunits and be administered in combination with another mRNA encoding the other subunit thereby allowing the assembly of functional IL-12 in the cell. Alternatively, functional IL-12 may be in the form of a modified single chain version of IL-12 that comprises both subunits within a single ORF (for example, see SEQ ID NO: 59).

In some embodiments of the invention, the coding mRNA is transiently expressed in a tumor microenvironment. In other embodiments, the coding mRNA encodes a cytokine or other gene product involved in regulating the survival, proliferation, and/or differentiation of APCs or immune cells, such as, for example, activated T cells and NK cells. By way of non-limiting example, the coding mRNA can encode for any cytokine disclosed herein, and more particularly a cytokine such as IL-1, IL-2, IL-4, IL-6, IL-8, IL-10, IL-12, IL-17, IL-33, IL-35, TGF-beta, TNFα, TNFβ, IFNα, IFNβ, IFNgamma, and any combination thereof.

MicroRNAs

MicroRNAs (miRNAs) are a class of noncoding RNAs each containing around 20 to 25 nucleotides, some of which are believed to be involved in post-transcriptional regulation of gene expression by binding to complementary target sequences in the 3′ untranslated regions (3′ UTR) of target mRNAs, leading to their silencing. These miRNA complementary target sequences are also referred to herein as miRNA binding sites, or miRNA binding site sequences. Certain miRNAs are highly tissue-specific in their expression; for example, miRNA-122 and its variants are abundant in the liver and infrequently expressed in other tissues (Lagos-Quintana et al. Current Biology. 2002; 12: 735-739).

The miRNA system therefore provides a robust platform by which nucleic acids introduced into cells can be silenced in selected cell types in a target tissue, and expressed in others. By including a target sequence for a particular given miRNA into an mRNA construct to be introduced into target cells, particularly within a UTR, expression of certain introduced genes can be reduced or substantially eliminated in some cell types, while remaining in others (Brown and Naldini, Nat Rev Genet. 2009; 10(8): 578-585).

In accordance with specific embodiments of the present invention it is contemplated that a plurality of such miRNA target sequences can be comprised within an organ protection sequence, which is then included in the mRNA construct. Where a plurality of miRNA target sequences are present, this plurality may include for example greater than two, greater than three, typically greater than four miRNA target sequences. These miRNA target sequences may be arranged sequentially, in tandem or at predetermined locations within, a specified UTR within the mRNA constructs. Multiple miRNA target sequences may be separated with auxiliary sequences that serve to support or facilitate the functioning of the organ protection sequence as a whole. By way of example, suitable auxiliary sequences may consist of a linker or spacer sequence, which may be randomized, or may comprise a particular sequence, for example, “uuuaaa”, although other spacer sequences can also be used. The length of the spacer can vary, and can comprise repetitions of a spacer sequence, for example the spacer “uuuaaa” can be included once (i.e. “uuuaaa”), twice (i.e. “uuuaaauuuaaa”—SEQ ID NO: 1), three times, four times, five times, or six times between each and any target sequence to be linked. In some embodiments, no spacer sequence may be present between binding site sequences.

miRNA-122, despite its abundance in healthy non-diseased liver tissue, is reduced in the majority of liver cancers as well as in diseased cells (Braconi et al. Semin Oncol. 2011; 38(6): 752-763, Brown and Naldini, Nat Rev Genet. 2009; 10(8): 578-585). By the above-mentioned method, it has been found that when the target tissue is the liver, translation of the introduced mRNA sequences can be facilitated in cancerous liver cells and reduced or substantially eliminated in transfected healthy cells, by including miRNA-122 target sequence (for example, SEQ ID NO: 1) in their 3′ UTRs.

In a similar way, differential translation of such mRNA is also possible between cancer cells and healthy cells in other organs, by using other miRNA target sequences. Suitable candidates include (but are not limited to) target sites for: miRNA-1, miRNA-125, miRNA-199, miRNA-124a, miRNA-126, miRNA-Let7, miRNA-375, miRNA-141, miRNA-142, miRNA-143, miRNA-145, miRNA-148, miRNA-194, miRNA-200c, miRNA-34a, miRNA-192, miRNA-194, miRNA-204, miRNA-215 and miRNA-30 family (for example, miRNA-30 a, b, or c).

Table 2 demonstrates further (non-limiting) examples of miRNA sequences where expression has been demonstrated in particular organs and/or tissues, and in several cases where differential expression is demonstrated between healthy and diseased cells.

miRNA-1, miRNA-133a and miRNA-206 have been described as examples of muscle and/or myocardium-specific miRNAs (Sempere et al. Genome Biology. 2004; 5:R13; Ludwig et al. Nucleic Acids Research. 2016; 44(8): 3865-3877). miRNA-1 has also been demonstrated to be dysregulated in disease, for example downregulation of miRNA-1 has been detected in infarcted heart tissue (Bostjancic E, et al. Cardiology. 2010; 115(3):163-169), while a drastic reduction of miRNA-1 has also been detected in rhabdomyosarcoma cell lines (Rao, Prakash K et al. FASEB J. 2010; 24(9):3427-3437). Use of miRNA-1, miRNA-133a and miRNA-206 may be particularly considered where compositions according to the invention are to be administered intramuscularly, so to reduce expression in local normal myocytes, if desired.

miRNA-125 is expressed in a number of tissues as shown in Table 2, and is downregulated in several solid tumors, such as hepatocellular carcinoma (Coppola et al. Oncotarget 2017; 8); breast (Mattie et al. Mol Cancer 2006; 5), lung (Wang et al. FEBS J 2009), ovarian (Lee et al. Oncotarget 2016; 7), gastric (Xu et al. Mol Med Rep 2014; 10), colon (Tong et al. Biomed Pharmacother 2015; 75), and cervical cancers (Fan et al Oncotarget 2015; 6); neuroblastoma, medulloblastoma (Ferretti et al. Int J Cancer 2009; 124), glioblastoma (Cortez et al. Genes Chromosomes Cancer 2010; 49), and retinoblastoma (Zhang et al; Cell signal 2016; 28).

Several miRNA species are also differentially expressed in glioblastoma multiforme cells (Zhangh et al. J Miol Med 2009; 87/Shi et al. Brain Res 2008; 1236) compared to non-diseased brain cells (e.g. neurons), with miRNA-124a one of the most dysregulated (Karsy et al. Gene Cancer 2012; 3; Riddick et al. Nat Rev Neurol 2011; 7; Gaur et al. Cancer Res 2007; 67/Silber et al. BMC Med 2008; 6).

In lung cancer, a recent meta-analysis confirmed the downregulation of Let-7 (as well as miRNA-148a and miRNA-148b) in non-small-cell lung cancer (Lamichhane et al. Disease Markers 2018).

Similarly, miRNA-375 expression has been found to be downregulated in pancreatic cancer cells, compared to healthy pancreatic cells (Shiduo et al. Biomedical Reports 2013; 1). In the pancreas, miRNA-375 expression has been indicated to be high in normal pancreas cells but significantly lower in diseased and/or cancerous tissues (Song, Zhou et al. 2013). This expression has been shown to relate to the stage of cancer, with expression further reduced with more advanced cancer. It is thought that miRNA-375 is involved with the regulation of glucose-induced biological responses in pancreatic β-cells, by targeting 3-phosphoinositide-dependent protein kinase-1 (PDK1) mRNA and so affecting the PI 3-kinase/PKB cascade (El Ouaamari et al. Diabetes 57:2708-2717, 2008). An anti-proliferative effect of miRNA-375 is implicated by this putative mode of action, which may explain its downregulation in cancer cells.

Table 2 discusses non-limiting examples of miRNAs associated with particular organs and/or tissues, which may be used in embodiments of the present invention. It will be appreciated, that the present invention is not limited only to instances where a given miRNA or class of miRNAs is downregulated in a first cell type versus a second cell type within a given organ or organ system. On the contrary, it is merely required that there exists a differential expression pattern of a regulatory miRNA between cell types, for example those comprised within an organ or organ system, or between different organs or organ systems. The differential expression of the miRNA system can be exploited using the compositions and methods described herein to enable corresponding differential translation of protein products between cells, thereby reducing undesired off-target side effects. This is of particular use in embodiments where differential expression of a mRNA between cell types or tissues is desired. For example, it may be advantageous to express an mRNA encoding a pro-inflammatory cytokine, if used as an adjuvant, primarily in immune cells but not in one or more healthy tissues where an increase in inflammation would not be desired—such as the skin, liver, kidney or colon.

The differential expression of miRNA between cancers and the adjacent healthy tissues represent a model system whereby the use of miRNA silencing of mRNAs can be identified and characterised. Examples of cancers where evidence has been found for similar differential miRNA expression between healthy and cancer cells include breast (Nygaard et al, BMC Med Genomics, 2009 Jun. 9; 2:35), ovarian (Wyman et al, PloS One, 2009; 4(4):e5311), prostate (Watahiki et al, PloS One, 2011; 6(9):e24950), and cervical cancers (Lui et al. Cancer Research, 2007 Jul. 1; 67(13):6031-43). WO 2017/132552 A1 describes a wide range of miRNAs with differing expression levels in various cancer cells. In skin, differential expression miRNA expression between healthy tissue and adjacent melanoma cells is also observed.

TABLE 2
miRNA associated with particular tissue/organ types

| | Implicated | |
|---|---|---|
| Tissue | miRNA | Reference |
| Liver | miRNA-122 | Braconi et al. Seminars in Oncology. 2011; 38(6): 752-763; |
| | | Brown and Naldini. Nature Reviews Genetics. 2009; |
| | | 10: 578-585; |
| | | Fu and Colin. EBioMedicine. 2018; 17-18. |
| Liver | miRNA-125, | Coppola N et al. Oncotarget, 2017; 8(15): 25289-299 |
| | miRNA-199 | Murakami Y. Oncogene 2006; 25 |
| | | Hou et al. Cancer Cell, 2011; 19(2): 232-243. |
| | | Shi et al. Medicine. 2017; 96(32): e7764 |
| Brain | miRNA-124a, | Mazzacurati L. Molecular Therapy 23, 2015; |
| | Let7 family | Gaur et al. Cancer Res. 2007; 67(5): 2456-68. |
| Lung | Let-7 family | Edge RE et al. Mol Ther 2008; 16(8): 1437-43 |
| | miRNA- | Lamichhane et al. Disease Markers. 2018; ID8309015 |
| | 148a/b | |
| | miRNA-30 | |
| | family | |
| | miRNA-126 | |
| Breast | Let7 family | Yu F. Cell 2007; 11431(6): 1109-23 |
| | | Takamizawa J. Cancer Res 2004; 64(11): 3753- |
| | | 3756 |
| Pancreas | miRNA-375 | Song S et al. Biomed Rep. 2013 (3): 393-398; |
| | Let7 family | Dai et al. Cancer Cell Int. 2020. 20: 98. |
| | miRNA-142 | https://doi.org/10.1186/s12935-020-01185-z |
| | miRNA-145 | |
| | miRNA-217 | |
| | miRNA-122 | |
| | Let7 family | |
| Colon | miRNA-143, -145, | Michael M Z. Mol Cancer Res 2003; 334 (1): 882-891; |
| | -194, -34a, | Ding et al. Int. J. Mol. Sci. 2018; 19, 2719 |
| | -126, -192, | |
| | -215, Let7 | |
| | family | |
| Kidney | miRNA-192, -194, | Sempere et al. Genome Biol 2004; 5(3): R13 |
| | -204, -215, | Wu et al. Nat.Commun. Apr 4; 7: 11169.; |
| | -30 family, | Nakada et al. J Pathol. 2008; 216: 418-427; |
| | -141, -200c, | Jiang et al. Oncology Letters (2018) 16, 3038-3044; |
| | Let7 family | Khella et al. Carcinogenesis. 2013. 34(10): 2231-2239; |
| | | Chen. et al. Cell Death and Disease (2017) 8, e2859; |
| | | doi: 10.1038/cddis.2017.252 |
| Skin | miRNA-877, -4300, | Aksenenko et al. BMC Dermatology. 2019; 19: 1. |
| | -4720, -6761 | Liu et al. Laboratory Investigation (2012) 92, |
| | miRNA-203a, | 1084-1096 |
| | -205, -200c, | |
| | Let7 family | |
| Spleen | miRNA-142, | Chen et al. Science (2004); 303(5654): 83-86; |
| | miRNA-126, | Trissal et al. Cancer Res. 2018; 78(13): 3510-3521; |
| | Let7 family | Merkerova et al. Eur J Haematol 2008; 81(4): 304-10 |
| Muscle and | miRNA-1, | Bostjancic E, et al. Cardiology. 2010; 115(3): 163-169; |
| cardiac | miRNA-133a, | Rao, Prakash K et al. FASEB J. 2010; 24(9): |
| muscle | miRNA-206, | 3427-3437; |
| | Let7 family | Ma et al. Int. J. Biol. Sci. 2015; 11: 345-352. |
| Endothelium | miRNA-98, | Harris T A et al. PNAS. 2008.105(5): 1516-1521 |
| | miRNA-126 | Matarese A. et al. Biomedicines. 2020, 8, 462; |
| | | doi: 10.3390/biomedicines8110462 |

Treating patients with immunotherapies may have safety issues due to the possibility of off-target effects. Even the expression of certain polypeptides by the provision of coding mRNA sequences can have negative effects on certain organs. Protecting healthy tissues, for example liver, brain, breast, lung, pancreas, colon/GI-tract, skin, muscle, and kidneys is thus paramount for successful clinical applications. miRNAs such as those described above can be used to reduce the expression of an administered mRNA in particular cell, tissue and/or organ types, to protect those cells, tissues and/or organs from any off-target effects. For instance, target sequences for specific miRNA that are highly expressed in specific tissues can be used to protect healthy cells, such as miRNA-1, miRNA-133a and/or miRNA-206 to protect healthy muscle and/or myocardium tissues. As a result, it may be desired to use miRNA target sequences which are not necessarily associated with differential expression in diseased and healthy cells. For example, miRNA-142 and miRNA 145 have expression in pancreatic tissue, while miRNA-9 can be used for brain and lung protection because of its high expression in these tissues.

If more than one tissue is to be protected, a combination of multiple miRNA target sequences is used. For instance, the target sequence for miRNA-122, miRNA-203a, miRNA-1 and miRNA-30a is used together to protect cells of the liver, skin, muscle and kidney tissues.

Hence, the present compositions may represent an enabling technology platform for enhancing and facilitating the successful adoption of hitherto ‘experimental’ cellular or viral therapies.

As is evident from this disclosure, the present invention is envisioned to relate to a number of possible combinations of therapies, delivery platforms (such as different nanoparticle compositions), therapeutic agents (such as drugs, vaccines and/or viruses), encoded polypeptides and target cells, tissues or organs. Each and all of these possibilities have implications for the optimal expression for the encoded polypeptides supplied by the mRNA sequences.

It has been found that the optimisation of one or more characteristics of the miRNA target sequences can lead to particular efficacy at promoting differential expression and thereby healthy organ protection. By the same token, such characteristics can be controlled to increase or decrease the resultant differential expression in particular organ, tissue or cell types, according to the specific context. There may be situations where a variety of expression levels are desired in various different cell types, and it is intended that target sequences can be modified to allow for such an outcome, by varying one or more characteristics as described herein. Also, an miRNA target site sequence can be modified so it is subject to regulation by more than one miRNA, either within the same tissue or in different tissues.

Sequence matching: the degree to which the target sequences are an exact match with the complementary miRNA sequence (that is, the number of mismatches between the miRNA sequence and the binding site sequence) has been shown to impact the efficacy of resultant expression silencing. For example, an exact or perfect match has been shown to lead to more rapid degradation of the sequence possessing the miRNA binding site sequence (Brown and Naldini, Nat Rev Genet. 2009; 10(8): 578-585. Therefore, if complete, or close to complete silencing of a particular polypeptide product is required in a particular cell type, it may be desired to select an miRNA target sequence which is an exact match, or has at most no more than one base pair mismatch, with an miRNA sequence associated with that cell type. Likewise, if reduced but not absent expression is desired in a particular cell type, an miRNA binding site sequence with an increased number of mismatches can be chosen to allow for this. Examples of several miRNA sequences mentioned herein, including the sequences of the stem-loop pre-miRNA with the eventual processed mature 5P or 3P miRNA and the sequences which form a duplex with the mature miRNA in the pre-miRNA underlined, as well as the mature miRNA sequences and duplex forming sequences themselves, are shown in Table 3 below. The mature miRNA expressed at significant levels in the cell (which can be either or both of the 5P and 3P strands) is marked (*). Table 4 shows the original, imperfectly matched, target sequence which forms the duplex in the pre-miRNA, followed by the mature miRNA sequence and the development of a modified complementary target sequence, which is designed to be a perfect match with the overexpressed mature miRNA sequence. The modified target sequence in the conventional 5′ to 3′ orientation is shown in bold.

TABLE 3
Optimisation of the miRNA target sequences by
testing 5P vs 3P mature binding sequences
(*overexpressed mature miRNA from RNA-Seq
database http://www.mirbase.org)

| | | Nucleotide Sequence |
|---|---|---|
| miRNA-122 | Pre-miRNA | 5-CCUUAGCAGAGCUGUGGAGUGUGACAAUGGUGUUUGUGU |
| | [SEQ ID | CUAAACUAUCAAACGCCAUUAUCACACUAAAUAGCUACUGC |
| | NO: 2] | UAGGC-3 |
| | 5P mature* | 5-UGGAGUGUGACAAUGGUGUUUG-3 |
| | [SEQ ID | |
| | NO: 3] | |
| | 3P mature | 5-AACGCCAUUAUCACACUAAAUA-3 |
| | [SEQ ID | |
| | NO: 4] | |
| miRNA-199a | Pre-miRNA | 5-GCCAACCCAGUGUUCAGACUACCUGUUCAGGAGGCUCUC |
| | [SEQ ID | AAUGUGUACAGUAGUCUGCACAUUGGUUAGGC-3 |
| | NO: 5] | |
| | 5P mature | 5-CCCAGUGUUCAGACUACCUGUUC-3 |
| | [SEQ ID | |
| | NO: 6] | |
| | 3P mature* | 5-ACAGUAGUCUGCACAUUGGUUA-3 |
| | [SEQ ID | |
| | NO: 7] | |
| miRNA-125a | Pre-miRNA | 5-UGCCAGUCUCUAGGUCCCUGAGACCCUUUAACCUGUGAG |
| | [SEQ ID | GACAUCCAGGGUCACAGGUGAGGUUCUUGGGAGCCUGGCGU |
| | NO: 8] | CUGGCC-3 |
| | 5P mature* | 5-UCCCUGAGACCCUUUAACCUGUGA-3 |
| | [SEQ ID | |
| | NO: 9] | |
| | 3P mature | 5-ACAGGUGAGGUUCUUGGGAGCC-3 |
| | [SEQ ID | |
| | NO: 10] | |
| miRNA-192 | Pre-miRNA | 5-GCCGAGACCGAGUGCACAGGGCUCUGACCUAUGAAUUGA |
| | [SEQ ID | CAGCCAGUGCUCUCGUCUCCCCUCUGGCUGCCAAUUCCAUA |
| | NO: 11] | GGUCACAGGUAUGUUCGCCUCAAUGCCAGC-3 |
| | 5P mature* | 5-UCUGACCUAUGAAUUGACAGCC-3 |
| | [SEQ ID | |
| | NO: 12] | |
| | 3P mature | 5-CUGCCAAUUCCAUAGGUCACAG-3 |
| | [SEQ ID | |
| | NO: 13] | |
| miRNA-let7b | Pre-miRNA | 5-CGGGGUGAGGUAGUAGGUUGUGUGGUUUCAGGGCAGUGA |
| | [SEQ ID | UGUUGCCCCUCGGAAGAUAACUAUACAACCUACUGCCUUCC |
| | NO: 14] | CUG-3 |
| | 5P mature* | 5-UGAGGUAGUAGGUUGUGUGGUU-3 |
| | [SEQ ID | |
| | NO: 15] | |
| | 3P mature | 5-CUAUACAACCUACUGCCUUCCC-3 |
| | [SEQ ID | |
| | NO: 16] | |
| miRNA-375 | Pre-miRNA | 5-CCCCGCGACGAGCCCCUCGCACAAACCGGACCUGAGCGU |
| | [SEQ ID | UUUGUUCGUUCGGCUCGCGUGAGGC-3 |
| | NO: 17] | |
| | 5P mature | 5-GCGACGAGCCCCUCGCACAAACC-3 |
| | [SEQ ID | |
| | NO: 18] | |
| | 3P mature* | 5-UUUGUUCGUUCGGCUCGCGUGA-3 |
| | [SEQ ID | |
| | NO: 19] | |
| miRNA-124a | Pre-miRNA | 5-AGGCCUCUCUCUCCGUGUUCACAGCGGACCUUGAUUUAA |
| | [SEQ ID | AUGUCCAUACAAUUAAGGCACGCGGUGAAUGCCAAGAAUGG |
| | NO: 20] | GGCUG-3 |
| | 5P mature | 5-CGUGUUCACAGCGGACCUUGAU-3 |
| | [SEQ ID | |
| | NO: 21] | |
| | 3P mature* | 5-UAAGGCACGCGGUGAAUGCCAA-3 |
| | [SEQ ID | |
| | NO: 22] | |
| miRNA-143 | Pre-miRNA | 5-GCGCAGCGCCCUGUCUCCCAGCCUGAGGUGCAGUGCUGC |
| | [SEQ ID | AUCUCUGGUCAGUUGGGAGUCUGAGAUGAAGCACUGUAGCU |
| | NO: 23] | CAGGAAGAGAGAAGUUGUUCUGCAGC-3 |
| | 5P mature | 5-GGUGCAGUGCUGCAUCUCUGGU-3 |
| | [SEQ ID | |
| | NO: 24] | |
| | 3P mature* | 5-UGAGAUGAAGCACUGUAGCUC-3 |
| | [SEQ ID | |
| | NO: 25] | |
| miRNA-142 | Pre-miRNA | 5-GACAGUGCAGUCACCCAUAAAGUAGAAAGCACUACUAAC |
| | [SEQ ID | AGCACUGGAGGGUGUAGUGUUUCCUACUUUAUGGAUGAGUG |
| | NO: 26] | UACUGUG-3 |
| | 5P mature* | 5-CAUAAAGUAGAAAGCACUACU-3 |
| | [SEQ ID | |
| | NO: 27] | |
| | 3P mature* | 5-UGUAGUGUUUCCUACUUUAUGGA-3 |
| | [SEQ ID | |
| | NO: 28] | |
| miRNA-203a | Pre-miRNA | 5-GUGUUGGGGACUCGCGCGCUGGGUCCAGUGGUUCUUAAC |
| | [SEQ ID | AGUUCAACAGUUCUGUAGCGCAAUUGUGAAAUGUUUAGGAC |
| | NO: 29] | CACUAGACCCGGCGGGCGCGGCGACAGCGA-3 |
| | 5P mature | 5-AGUGGUUCUUAACAGUUCAACAGUU-3 |
| | [SEQ ID | |
| | NO: 30] | |
| | 3P mature* | 5-GUGAAAUGUUUAGGACCACUAG-3 |
| | [SEQ ID | |
| | NO: 31] | |
| Let7a | Pre-miRNA | 5-UGGGAUGAGGUAGUAGGUUGUAUAGUUUUAGGGUCACAC |
| | [SEQ ID | CCACCACUGGGAGAUAACUAUACAAUCUACUGUCUUUCCU |
| | NO: 32] | A-3 |
| | 5P mature* | 5-UGAGGUAGUAGGUUGUAUAGUU-3 |
| | [SEQ ID | |
| | NO: 33] | |
| | 3P mature | 5-CUAUACAAUCUACUGUCUUUC-3 |
| | [SEQ ID | |
| | NO: 34] | |
| miRNA-30a | Pre-miRNA | 5-GCGACUGUAAACAUCCUCGACUGGAAGCUGUGAAGCCAC |
| | [SEQ ID | AGAUGGGCUUUCAGUCGGAUGUUUGCAGCUGC-3 |
| | NO: 35] | |
| | 5P mature* | 5-UGUAAACAUCCUCGACUGGAAG-3 |
| | [SEQ ID | |
| | NO: 36] | |
| | 3P mature | 5-CUUUCAGUCGGAUGUUUGCAGC-3 |
| | [SEQ ID | |
| | NO: 37] | |
| miRNA-1b | Pre-miRNA | 5-UGGGAAACAUACUUCUUUAUAUGCCCAUAUGGACCUGCU |
| | [SEQ ID | AAGCUAUGGAAUGUAAAGAAGUAUGUAUCUCA-3 |
| | NO: 38] | |
| | 5P mature | 5-ACAUACUUCUUUAUAUGCCCAU-3 |
| | [SEQ ID | |
| | NO: 39] | |
| | 3P mature* | 5-UGGAAUGUAAAGAAGUAUGUAU-3 |
| | [SEQ ID | |
| | NO: 40] | |
| miRNA-126 | Pre-miRNA | 5-CGCUGGCGACGGGACAUUAUUACUUUUGGUACGCGCUGU |
| | [SEQ ID | GACACUUCAAACUCGUACCGUGAGUAAUAAUGCGCCGUCCA |
| | NO: 41] | CGGCA-3 |
| | 5P mature | 5-CAUUAUUACUUUUGGUACGCG |
| | [SEQ ID | |
| | NO: 42] | |
| | 3P mature* | 5-UCGUACCGUGAGUAAUAAUGCG |
| | [SEQ ID | |
| | NO: 43] | |

TABLE 4
Optimization of the miRNA target sequences by modifying
the nucleotides sequence to obtain a perfect match with
the miRNA (*overexpressed mature miRNA from RNA-Seq
database http://www.mirbase.org)

| | | Nucleotide Sequence |
|---|---|---|
| miRNA- | 3P = sequence within pre- | 5-AACGCCAUUAUCACACUAAAUA-3 |
| 122 | miRNA [SEQ ID NO: 4] | |
| | Perfect matching [SEQ ID | 5P* 5-UGGAGUGUGACAAUGGUGUUUG-3 |
| | NO: 3] | |
| | Target Sequence [SEQ ID | TS 3-ACCUCACACUGUUACCACAAAC-5 |
| | NO: 44] | |
| | Orientated 5′ -> 3′ | TS 5-CAAACACCAUUGUCACACUCCA-3 |
| | [SEQ ID NO: 44] | |
| miRNA- | 5P = Target sequence | 5-CCCAGUGUUCAGACUACCUGUUC-3 |
| 199a | within pre-miRNA [SEQ ID | |
| | NO: 6] | |
| | Perfect matching [SEQ ID | 3P* 5-ACAGUAGUCUGCACAUUGGUUA-3 |
| | NO: 7] | |
| | TS [SEQ ID NO: 45] | TS 3-UGUCAUCAGACGUGUAACCAAU-5 |
| | Orientated 5′ -> 3′ | TS 5-UAACCAAUGUGCAGACUACUGU-3 |
| | [SEQ ID NO: 45] | |
| miRNA- | 3P = sequence within pre- | 5-ACAGGUGAGGUUCUUGGGAGCC-3 |
| 125a | miRNA [SEQ ID NO: 10] | |
| | Perfect matching | 5P* 5-UCCCUGAGACCCUUUAACCUGUGA-3 |
| | [SEQ ID NO: 9] | |
| | TS [SEQ ID NO: 46] | TS 3-AGGGACUCUGGGAAAUUGGACACU-5 |
| | Orientated 5′ -> 3′ | TS 5-UCACAGGUUAAAGGGUCUCAGGGA-3 |
| | [SEQ ID NO: 46] | |
| miRNA- | 3P = sequence within pre- | 5-CUGCCAAUUCCAUAGGUCACAG-3 |
| 192 | miRNA [SEQ ID NO: 13] | |
| | Perfect matching [SEQ ID | 5P* 5-UCUGACCUAUGAAUUGACAGCC-3 |
| | NO: 12] | |
| | TS [SEQ ID NO: 47] | TS 3-AGACUGGAUACUUAACUGUCGG-5 |
| | Orientated 5′ -> 3′ | TS 5-GGCUGUCAAUUCAUAGGUCAGA-3 |
| | [SEQ ID NO: 47] | |
| miRNA- | 3P = sequence within pre- | 5-CUAUACAACCUACUGCCUUCCC-3 |
| let7b | miRNA [SEQ ID NO: 6] | |
| | Perfect matching [SEQ ID | 5P* 5-UGAGGUAGUAGGUUGUGUGGUU-3 |
| | NO: 15] | |
| | TS [SEQ ID NO: 48] | TS 3-ACUCCAUCAUCCAACACACCAA-5 |
| | Orientated 5′ -> 3′ | TS 5-AACCACACAACCUACUACCUCA-3 |
| | [SEQ ID NO: 48] | |
| miRNA- | 5P = sequence within pre- | 5-GCGACGAGCCCCUCGCACAAACC-3 |
| 375 | miRNA [SEQ ID NO: 18] | |
| | Perfect matching [SEQ ID | 3P* 5-UUUGUUCGUUCGGCUCGCGUGA-3 |
| | NO: 19] | |
| | TS [SEQ ID NO: 49] | TS 3-AAACAAGCAAGCCGAGCGCACU-5 |
| | Orientated 5′ -> 3′ | TS 5-UCACGCGAGCCGAACGAACAAA-3 |
| | [SEQ ID NO: 49] | |
| miRNA- | 5P = sequence within pre- | 5-CGUGUUCACAGCGGACCUUGAU-3 |
| 124a | miRNA [SEQ ID NO: 21] | |
| | Perfect matching [SEQ ID | 3P* 5-UAAGGCACGCGGUGAAUGCCAA-3 |
| | NO: 22] | |
| | TS SEQ ID NO: 50] | TS 3-AUUCCGUGCGCCACUUACGGUU-5 |
| | Orientated 5′ -> 3′ | TS 5-UUGGCAUUCACCGCGUGCCUUA-3 |
| | [SEQ ID NO: 50] | |
| miRNA- | 5P = sequence within pre- | 5-GGUGCAGUGCUGCAUCUCUGGU-3 |
| 143 | miRNA [SEQ ID NO: 24] | |
| | Perfect matching [SEQ ID | 3P* 5-UGAGAUGAAGCACUGUAGCUC-3 |
| | NO: 25] | |
| | TS SEQ ID NO: 51] | TS 3-ACUCUACUUCGUGACAUCGAG-5 |
| | Orientated 5′ -> 3′ | TS 5-GAGCUACAGUGCUUCAUCUCA-3 |
| | [SEQ ID NO: 51] | |
| miRNA- | 5P = sequence within pre- | 5-CAUAAAGUAGAAAGCACUACU-3 |
| 142 | miRNA [SEQ ID NO: 27] | |
| | Perfect matching | 3P* 5-UGUAGUGUUUCCUACUUUAUGGA-3 |
| | [SEQ ID NO: 28] | |
| | TS [SEQ ID NO: 52] | TS 3-ACAUCACAAAGGAUGAAAUACCU-5 |
| | Orientated 5′ -> 3′ | TS 5-UCCAUAAAGUAGGAAACACUACA -3 |
| | [SEQ ID NO: 52] | |
| miRNA- | 5P = sequence within pre- | 5-AGUGGUUCUUAACAGUUCAACAGUU-3 |
| 203a | miRNA [SEQ ID NO: 30] | |
| | Perfect matching [SEQ ID | 3P*5-GUGAAAUGUUUAGGACCACUAG-3 |
| | NO: 31] | |
| | TS [SEQ ID NO: 53] | TS 3-CACUUUACAAAUCCUGGUGAUC-5 |
| | Orientated 5′ -> 3′ | TS 5-CUAGUGGUCCUAAACAUUUCAC-3 |
| | [SEQ ID NO: 53] | |
| Let7a | 3P = sequence within | 5-CUAUACAAUCUACUGUCUUUC-3 |
| | miRNA [SEQ ID NO: 34] | |
| | Perfect matching [SEQ ID | 5P* 5-UGAGGUAGUAGGUUGUAUAGUU-3 |
| | NO: 33] | |
| | TS [SEQ ID NO: 54] | TS 3-ACUCCAUCAUCCAACAUAUCAA-5 |
| | Orientated 5′ -> 3′ | TS 5-AACUAUACAACCUACUACCUCA-3 |
| | [SEQ ID NO: 54] | |
| miRNA- | 3P = sequence within pre- | 5-CUUUCAGUCGGAUGUUUGCAGC-3 |
| 30a | miRNA SEQ ID NO: 37] | |
| | Perfect matching [SEQ ID | 5P* 5-UGUAAACAUCCUCGACUGGAAG-3 |
| | NO: 36] | |
| | TS [SEQ ID NO: 55] | TS 3-ACAUUUGUAGGAGCUGACCUUC-5 |
| | Orientated 5′ -> 3′ | TS 5-CUUCCAGUCGAGGAUGUUUACA-3 |
| | [SEQ ID NO: 55] | |
| miRNA- | 5P = sequence within pre- | 5-ACAUACUUCUUUAUAUGCCCAU-3 |
| 1b | miRNA [SEQ ID NO: 39] | |
| | Perfect matching [SEQ ID | 3P* 5-UGGAAUGUAAAGAAGUAUGUAU-3 |
| | NO: 40] | |
| | TS [SEQ ID NO: 56] | TS 3-ACCUUACAUUUCUUCAUACAUA-5 |
| | Orientated 5′ -> 3′ | TS 5-AUACAUACUUCUUUACAUUCCA-3 |
| | [SEQ ID NO: 56] | |
| miRNA- | 5P = sequence within pre- | 5-CAUUAUUACUUUUGGUACGCG-3 |
| 126 | miRNA [SEQ ID NO: 39] | |
| | Perfect matching [SEQ ID | 3P* 5-UCGUACCGUGAGUAAUAAUGCG -3 |
| | NO: 40] | |
| | TS [SEQ ID NO: 57] | TS 3-AGCAUGGCACUCAUUAUUACGC -5 |
| | Orientated 5′ -> 3′ | TS 5-CGCAUUAUUACUCACGGUACGA-3 |
| | [SEQ ID NO: 57] | |

It is known that variants and polymorphisms of miRNA sequences can be found, and that miRNA families exist with similar properties. In the present invention, it is envisioned that all suitable variants and family members of particular miRNA sequences and associated binding sites can be used where appropriate. On the other hand, apparently closely related miRNA sequences can have different expression profiles (Sun et al, World J Gastroenterol. 2017 Nov. 28), so in some situations it will be necessary to determine whether a specific substitution is appropriate, by reference to the literature. For example, Let-7 is part of a wider family with a number of related variants, which can be denoted as Let-7a to Let-7k, and so on. As discussed above, such variants and polymorphisms may vary in their efficacy at allowing for miRNA-mediated silencing, and it is intended that particular selections can therefore be made to allow for the desired level of silencing in a particular cell type.

The presence of a plurality of miRNA target sequences in the mRNA construct enables improved efficacy of the differential expression of the supplied polypeptide or polypeptides. Without being bound by theory, it is thought that with an increased number of target sites, the likelihood of translation inhibition by the miRNA is increased. Multiple miRNA target sites can comprise multiple copies of substantially the same target sequence, thereby introducing redundancy. Alternatively or additionally, the multiple target sequences can comprise substantially different sequences, thereby allowing the mRNA construct to be targeted by more than one species of miRNA. In this way, differential expression of a supplied mRNA construct can be achieved for more than one cell type, and/or in more than one organ, as is evident from the discussion of organs and their associated specific miRNA expression above. Both approaches are considered to be possible within the same sequence or multiple sequences. An intermediate approach is also envisioned, wherein target sites are included which are intended to be targets for the same miRNA sequence, but have differences in order to bind different miRNA variants of the same family, e.g. Let7.

Some advantages associated with the use of multiple target sites include an increase in the efficiency of differential expression of polypeptides supplied by the mRNA sequences of the present invention, within a single organ. Use of different binding site sequences, or sequences which are applicable to more than one tissue or organ type can enable differential expression to be achieved in different cell types in more than one organ or tissue. This may be desirable when systemic administration of compositions according to the invention is used, and it is necessary to avoid off-target effects in more than one organ.

Even with localised or targeted administration, it is possible that supplied mRNA constructs may encounter or accumulate in organs, tissues, and/or cells for which they were not intended. In particular, liver and spleen tissue may accumulate administered compositions, due to the physiological function of these organs. In these cases, to avoid off-target effects, it may be advantageous for the supplied constructs to comprise miRNA target sequences which would enable reduced expression in these tissues. Conversely, it may be desirable for expression to be encouraged in some organs, tissues and/or cell types but not others, which can be achieved by the selection of miRNA target sequences accordingly.

Particular combinations of miRNA target sites can relate to particular combinations of target organs, which may be especially effective in different contexts. For example, administered compositions may accumulate in the liver and spleen, and therefore the use of miRNA target sequences associated with those organs can give directed protection to healthy cells which may be contacted with the compositions. For example, the binding site sequences can provide one or more targets for each of miRNA-122 and miRNA-142, or any other combination of liver and spleen-associated miRNA sequences, for example any combination of those listed for these organs in Table 2. Such combinations could include, for example, at least one copy of at least one target site selected from miRNA-122, miRNA-125, and miRNA-199 (liver); at least one copy of at least one binding site sequence selected from miRNA-192, miRNA-194, miRNA-204, miRNA-215, and miRNA-30 a,b,c (kidney); and at least one copy of a binding site for miRNA-142 (spleen).

Such an approach may be especially advantageous for certain varieties of delivery nanoparticles. For instance, liposome-based nanoparticles may be prone to accumulate in the liver, kidneys and spleen. Other nanoparticle types or alternative administration approaches may accumulate in different organs or tissues, or the targeting of the compositions may cause particular organs or tissues to be in particular need of modulation of expression. For example, intramuscular administration may lead to accumulation in muscle tissue, and subcutaneous administration may lead to accumulation in skin tissue, with effects on which cell types would benefit from protection. It is therefore possible to select generic, likely longer, sequences comprising miRNA binding site sequences which give broad protection from unwanted expression in multiple organs, or to select particular miRNA binding site sequences to allow specific protection in one or more organs as required in a particular situation, which may allow for shorter sequences, and/or the inclusion of repeated binding site sequences (see below). In such a way, the delivered mRNA sequence can be optimised with respect to the mode of delivery (or vice versa).

In some cases, the miRNA target sequences used in the organ protection sequence may not be associated with the tissues or organs to be treated, and may not be designed to lead to differential expression between healthy and diseased cells within said tissues and organs. The miRNA binding sequences may rather be chosen to prevent off-target effects in organs which are not intended to be treated. For example, compositions and methods according to the invention could be designed for the treatment of skin, for instance, for the treatment of melanoma. Application of the compositions to the skin could be topical, or intratumoral (IT), such as by injection directly into the tumor or into blood supply leading directly into the tumor. In such cases, however, the composition could be taken up by the bloodstream, lymphatic system, or by these means or otherwise contact and/or accumulate in organs other than the skin, such as the liver, kidneys and/or spleen. In such cases, the miRNA target sequences may be chosen to accommodate for undesirable biodistribution and to prevent expression of the encoded mRNA within such off-target organs. For instance, the use of miRNA target sequences associated with the liver, kidneys and spleen may be chosen, and so prevent expression within healthy cells comprised within these organs. Examples of potential combinations of miRNA target sequences which could allow for this are set out above.

It is also envisioned that since a perfect match between a binding site sequence and an miRNA sequence is not required for miRNA-mediated silencing to occur, and since some miRNA sequences (especially sequences which are present within similar cell types) have considerable similarity, it is possible that sequences could be devised that could provide a target for more than one miRNA sequence. For example, miRNA-122 and miRNA-199 have similar binding site sequences, and a sequence which is substantially complementary to both miRNA could be designed and included as a miRNA target sequence, for example by slightly modifying a miRNA-122 binding site sequence. In this way, both miRNA-122 and miRNA-199 could bind to such a sequence, increasing degradation of the mRNA. Similarly, a target sequence for the Let-7 miRNA could serve as a target sequence for other members of the Let-7 family. Binding site sequences for different miRNAs can be aligned with any suitable alignment technique and compared for shared nucleotides, whereupon a binding site sequence comprising those shared nucleotides can be designed.

In specific embodiments of the invention, the number of times a particular target site sequence is repeated within an mRNA may impact the efficacy of silencing mediated by the binding site sequences. For instance, an increased number of repeats of one miRNA target site can increase the likelihood of the relevant miRNA binding to it, and so the likelihood of translation inhibition or degradation before translation occurs. As a result, if more complete miRNA-mediated silencing is required in a particular cell type, more repeats of a suitable target sequence for an miRNA expressed in those cells can be used. Likewise, reduced but not absent expression can be achieved by including fewer binding site sequences, with or without any of the other approaches discussed herein. Therefore, the same binding site sequence can be provided in the mRNA once, twice, three times, four times, five times, or more, and can be provided alone or in combination with target site sequences for other miRNAs.

According to certain embodiments, the order of the miRNA target sites comprised within the mRNA sequence may affect the resultant organ protection efficacy. For example, the target sequences for miRNA-122, let 7b, miRNA-375, miRNA-192, miRNA-142, (present in liver, lung, breast, pancreas, kidney, and spleen cells) can be presented in this order, or in a number of other permutations, for example:
miRNA-122-miRNA-375-Let 7-miRNA-192-miRNA-142;
miRNA-122-miRNA-375-Let 7-miRNA-142-miRNA-192; or
miRNA-122-Let 7-miRNA-375-miRNA-142-miRNA-192.

As another example, the target sequences for miRNA-122, Let 7a, miRNA-142, miRNA-30a, miRNA-143, (present in liver, lung/colon, spleen/haematopoietic cells, kidney, and colon cells) can be presented in this order, or in a number of other permutations, for example:
miRNA-122-Let7a-miRNA-142-miRNA-30a-miRNA-143;
miRNA-122-miRNA-142-Let7a-miRNA-143-miRNA-30a; or
miRNA-122-miRNA-30a-Let7a-miRNA-143-miRNA-142

In specific embodiments of the invention described in more detail below the target sequences for miRNA-122, miRNA-192 and miRNA-30a (present in liver, colon and kidney) can be presented in a variety of combinations such as:
miRNA-122-miRNA-192-miRNA-30a;
miRNA-122-miRNA-30a-miRNA-192; or
miRNA-192-miRNA-122-miRNA-30a

In further embodiments of the invention described in more detail below the target sequences for Let7b, miRNA-126 and miRNA-30a (present in liver, colon, spleen, lung and kidney) can be presented in a variety of combinations such as:
Let7b-miRNA-126-miRNA-30a;
Let7b-miRNA-30a-miRNA-126; or
miRNA-126-Let7b-miRNA-30a

Such combinations can be useful in protecting tissues likely to be affected by administration of compositions designed to be used in treatments for certain cancers or in vaccine or adjuvant expression systems, as discussed herein.

As a further example, the target sequences for miRNA-122, miRNA-203a, miRNA-1, miRNA-30a (present in liver, skin, muscle/myocardium, and kidney) can be presented in this order, or in a number of other permutations, for example:
miRNA-122-miRNA-203a-miRNA-1-miRNA-30a;
miRNA-122-miRNA-1-miRNA-203a-miRNA-30a; or
miRNA-122-miRNA-30a-miRNA-1-miRNA-203a

Such a combination can be useful in protecting tissues likely to be affected by administration of compositions designed to induce an immune response, as discussed below in relation to vaccines, adjuvants and similar approaches.

The present invention therefore allows different approaches to be selected which are tuneable to the coding sequence being delivered by the mRNA, and in which cell types. In other words, the differential expression allowed by the present invention is ‘configurable’ in order to allow for whatever level of expression or reduced expression is required.

In another embodiment, the delivered mRNA may code for a immunostimulatory or anti-immunosuppressive protein, or in another way may act to induce an immune response. In such cases, it may be desired to have maximal expression of the encoded product in the target diseased cells, but also to have reduced but still present expression in surrounding healthy tissue of the target organ. On the other hand, it may be desirable for expression of such immune-stimulating products in certain tissues (such as brain or other neural tissue) to be avoided completely, and/or for expression to be reduced in cells, tissues and organs where the composition is likely to accumulate, to prevent off-target immune responses and possible systemic reaction. Therefore, in one example the miRNA target sequences can be determined by one or more of the approaches discussed above to allow full expression in target diseased cells, partially reduce expression in healthy cells in the target organ, while more completely reducing expression in neural tissue and sites of accumulation.

In some embodiments, more than one different mRNA sequence may be provided in a single composition. These different sequences can encode different polypeptides, and/or different miRNA target sites. In this way, a single composition can allow for multiple different polypeptides to be expressed. By using different combinations of miRNA target sequences in the separate mRNA sequences, different cell types or target organs can express, or be protected from the expression of certain polypeptides, according to the desired objective. For instance, if healthy cells in liver and brain must be protected from the expression of a polypeptide ‘A’, but it is desired to express a polypeptide ‘B’ in healthy brain, but not liver, a first mRNA sequence could comprise the sequence of ‘A’, with target sites for miRNA-122, miRNA-125a and miRNA-124a, while a second mRNA sequence could comprise the sequence of ‘B’, with binding sites for miRNA-122 and miRNA-125a.

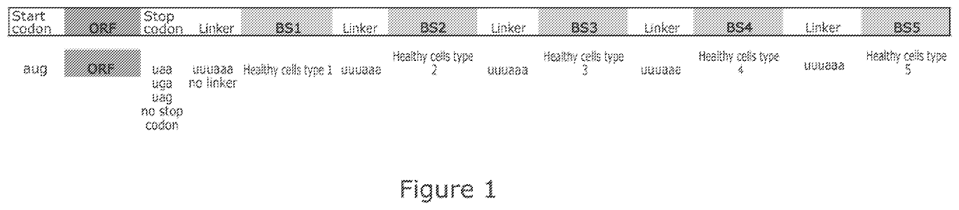

It can be appreciated that the person of skill in the art will be able to devise combinations of miRNA target sites, polypeptide sequences and multiple mRNA sequences in order to achieve any combination of expression in a given set of organ and cell types. The relevant organs and tissue types relating to these sequences are discussed above and in Table 2. FIG. 1 shows schematic views of mRNA constructs according to some embodiments of the invention. An ORF is preceded by a start codon and terminated with a stop codon, and a subsequent series of up to five or more binding site sequences are present in the 3′UTR. As shown in FIG. 1, the miRNA target sites (BS1 to BS5) that define the OPS may be separated by spacers, or no spacer at all if preferred. The ORF can code for example for a polypeptide as described herein. Variability in the stop codon is envisioned in any embodiment, and there may in all embodiments be no stop codon between the ORF and the binding site sequences.

The UTR of the mRNA sequences supplied by the present invention can be selected to have similarity, for example greater than 90% similarity, to part or all of a UTR sequence expressed in one of the cell types within the target organ. Particular cell types can have genes which are up- or down-regulated in expression, and the UTR sequence can mediate this regulation, for instance through encouraging the stability or degradation of the relevant mRNA sequences.

As an example, UTRs associated with genes which are known to be upregulated in cancer cells may have one or more features, such as miRNA binding site sequences, which encourage their stability and translation in these cancer cells. By incorporating similar sequences into supplied mRNA sequences, stability and translation can be improved in cancerous cells but not non-cancerous or healthy cells.

In certain situations, it is possible that more than one candidate for an miRNA sequence which exhibits differential expression in different cell types in a target tissue may exist. In such cases, it may be advantageous that a plurality of miRNA target sequences are included in the mRNA construct, and that these sequences may be substantially different sequences. However, it is also envisaged that each of the plurality of miRNA target sequences may be substantially the same sequence.

Combinations with Cytokines

It is contemplated that the compositions and methods as described herein may act to induce an immune response against disease or infection from a pathogenic organism. In particular, immune responses may be induced against cancer cells. The process of carcinogenesis frequently involves ways in which the cancer cells attempt to evade the immune system, involving changes to the antigens produced and displayed by these cells,

In some embodiments, the mRNA provided by the invention comprises at least one polynucleotide encoding a protein that is a bispecific T-cell engager (BiTE), an anti-immunosuppressive protein, or an immunogenic agent. The term “anti-immunosuppressive protein” as used herein is a protein that inhibits an immunosuppressive pathway.

The term “immunogenic agent” as used herein refers to a protein that increases an inflammatory or immunogenic immune response. In particular embodiments, the anti-immunosuppressive and immunogenic agents induce an anti-tumor immune response. Examples of such agents include antibody or antigen binding fragments thereof that bind to and inhibit immune checkpoint receptors (e.g. CTLA4, LAG3, PD1, PDL1, and others), proinflammatory cytokines (e.g., IFNγ, IFNα, IFNβ, TNFα, IL-12, IL-2, IL-6, IL-8, GM-CSF, and others), or proteins that binding to and activate an activating receptor (e.g., FcγRI, FcγIIa, FcγIIIa, costimulatory receptors, and others). In particular embodiments, the protein is selected from EpCAM, IFNβ, anti-CTLA-4, anti-PD1, anti-PDL1, A2A, anti-FGF2, anti-FGFR/FGFR2b, anti-SEMA4D, CCL5, CD137, CD200, CD38, CD44, CSF-1R, CXCL10, CXCL13, endothelin B Receptor, IL-12, IL-15, IL-2, IL-21, IL-35, ISRE7, LFA-1, NG2 (also known as SPEG4), SMADs, STING, TGFβ, VEGF and VCAM1.

The invention encompasses compositions supplying mRNA coding for functional macromolecules to targeted cell populations used in cell-based therapies. In some embodiments, the targeted cell population is a genetically engineered T cell population.

The coding mRNA may be used to attract a population of immune cells or a combination of immune cell populations to a particular site in a subject. In some embodiments, the coding mRNA and the delivery particles are used to attract immune cells to the tumor microenvironment. In some embodiments, the coding mRNA and the delivery particles are used to overcome insufficient migration of an immune cell to the tumor microenvironment. In some embodiments, the immune cell is a T cell, a natural killer (NK) cell, a B cell, an antigen-presenting cell (APC) such as a macrophage or dendritic cell, or any combination thereof. In some embodiments, the coding mRNA and the delivery particles are used to attract T cells to the tumor microenvironment.

The coding mRNA may be used to overcome insufficient migration of T cells to the tumor microenvironment. In some embodiments, the delivery particles specifically target the tumor microenvironment, and the coding mRNA encodes a gene product that attracts or otherwise recruits T cells to the tumor microenvironment. In some embodiments, the coding mRNA expresses a chemokine. By way of non-limiting example, the coding mRNA can encode a chemokine that attracts T-cells such as CCL2, CCL3, CCL4, CCL5, CCL20, CCL22, CCL28, CXCL8, CXCL9, CXCL10, CXCL11, CXCL12, XCL1, and any combination thereof. In situations where the reverse effect is desired, such as in autoimmune disease, the coding mRNA can express blockers, antagonists and/or inhibitors of the above-mentioned factors.

The coding mRNA may be delivered to and transiently expressed within the tumor microenvironment. In some embodiments, the coding mRNA encodes a cytokine or other gene product involved in regulating the survival, proliferation, and/or differentiation of immune cells in the tumor response, such as, for example, activated T cells and NK cells. By way of non-limiting example, the coding mRNA can encode for a cytokine such as IL-1, IL-2, IL-3, IL-4, IL-5, IL-6, IL7, IL-8, IL-9, IL-10, IL-11, IL-12, IL-17, IL-33, IL-35, TGF-beta, and any combination thereof. Again, in situations where the reverse effect is desired, such as in autoimmune disease, the coding mRNA can express blockers, antagonists and/or inhibitors of the above-mentioned factors, for example, inhibitors of TGF-beta.

The compositions supplying mRNA may be designed to target particular cell subtypes and, upon binding to them, stimulate receptor-mediated endocytosis, thereby introducing the synthetic mRNA they carry to the cells, which can now express the synthetic mRNA. Because nuclear transport and transcription of the transgene are not required, this process is fast and efficient.

In some embodiments, the coding mRNA may code for receptors or other cell surface proteins associated with immune cells (such as costimulators) or immune pathways, or for molecules which target such receptors. For example, the coding mRNA may code for molecules targeting the following cellular receptors and their ligands selected from one or more of: CD40, CD40L, CD160, 2B4, Tim-3, GP-2, B7H3 and B7H4. Similarly, the coding mRNA may code for dendritic cell activators selected from one or more of GM-CSF, TLR7 and TLR9. In one embodiment, the coding mRNA codes for one or more T-cell membrane protein 3 inhibitors. In one embodiment, the coding mRNA codes for one or more inhibitors of NF-κB.

The Toll-like receptor (TLR) family are involved in pathogen recognition and the activation of innate immunity. TLR8 in particular can recognise single stranded RNA and therefore plays a role in the recognition of ssRNA viruses by the activation of the transcription factor NF-κB and an antiviral response. Therefore, embodiments where the coding mRNA encodes a member of the TLR family, for example TLR8, are considered where an antiviral response is desired.

In some embodiments, the mRNA delivery systems may be used to deliver an mRNA that codes for one or more agents that program T cells toward a desired phenotype. In some embodiments, the mRNA nanoparticle delivery compositions may be used to induce markers and transcriptional patterns that are characteristic of a desired T cell phenotype. In some embodiments, the mRNA nanoparticle delivery compositions may be used to promote development of CD26L+ central memory T cells (Tcm). In some embodiments, compositions supply mRNA encoding one or more transcription factors to control cell differentiation in a target cell population. In some embodiments, the transcription factor is Foxo1, which controls development effector-to-memory transition in CD8 T-cells.

In some embodiments, the mRNA delivery compositions include a surface-anchored targeting domain that is specific for a T cell marker, such as, for example, a surface antigen found on T cells. In some embodiments, the surface-anchored targeting domain is specific for an antigen that selectively binds the nanoparticle to T-cells and initiates receptor-induced endocytosis to internalize the mRNA nanoparticle delivery compositions. In some embodiments, the surface-anchored targeting domain selectively binds CD3, CD8, or a combination thereof. In some embodiments, surface-anchored targeting domain is or is derived from an antibody that selectively binds CD3, CD8, or a combination thereof.

Delivery Platforms

The introduction of coding nucleotide sequences into a target cell often requires the use of a delivery agent or ‘in vivo delivery composition’ to transfer the desired substance from the extracellular space to the intracellular environment. Frequently, such delivery agents/compositions may comprise delivery particles. Delivery particles may undergo phagocytosis and/or fuse with a target cell. Delivery particles may contain the desired substance by encapsulation or by comprising the substance within a matrix or structure.

The term ‘delivery particle’ as used herein refers to drug or biological molecule delivery systems that comprise particles which can comprise therapeutic components by encapsulation, holding within a matrix, the formation of complex, surface adsorption or by other means. These systems can deliver a therapeutic component such as a coding nucleic acid sequence into a target cell. Compared to direct administration of a molecule or substance, the use of delivery particles may improve not only the efficacy of delivery, but also safety, by controlling the amount, time and/or release kinetics of the substance to be delivered at the site of action. Delivery particle systems are also adept at crossing biologic membranes to enable the substance or drug to get to the desired therapeutic target location. Delivery particles may on the micro-scale, but in specific embodiments may typically be on the nanoscale—i.e. nanoparticles. Nanoparticles are typically sized at least 50 nm (nanometres), suitably at least approximately 100 nm and typically at most 150 nm, 200 nm, although optionally up to 300 nm in diameter. In one embodiment of the invention the nanoparticles have a mean diameter of approximately at least 60 nm. An advantage of these sizes is that this means that the particles are below the threshold for reticuloendothelial system (mononuclear phagocyte system) clearance, i.e. the particle is small enough not to be destroyed by phagocytic cells as part of the body's defence mechanism. This facilitates the use of intravenous delivery routes for the compositions of the invention. The routes used to administer and deliver active substances comprised within delivery particles to their target tissue are a highly relevant factor when treating a disease, particularly an infectious disease. These routes may have different levels of efficacy depending on how they are applied. In specific embodiments of the present invention the administration of the delivery particles is normally systemic, such as via subcutaneous, intravenous or intra-arterial administration. Occasionally, due to the type or severity of the disease delivery particles may be applied directly to an affected organ or tissue such via intra-tumoral administration.

Alternative possibilities for the composition of the nanoparticles include polylactic acid (PLA), poly(lactic-co-glycolic acid) (PLGA), polycaprolactones, lipid- or phospholipid-based particles such as liposomes or exosomes; particles based on proteins and/or glycoproteins such as collagen, albumin, gelatin, elastin, gliadin, keratin, legumin, zein, soy proteins, milk proteins such as casein, and others (Lohcharoenkal et al. BioMed Research International; Volume 2014 (2014)); colloidal nanoparticles; and particles based on metals or metallic compounds such as gold, silver, aluminium, copper oxides, metal-organic cycles and cages (MOCs) and so on. In specific embodiments poly(lactic-co-glycolic acid) (PLGA) may be used in delivery particles of the invention due to its high biocompatibility and biodegradability. PLGA was approved for clinical use in 1989, by the US Food and Drug Administration (FDA). It has been favoured for sustained release formulations of a wide range of drugs and biomolecules since that time. PLGA may be co-formulated with polyvinyl alcohol (PVA) in order to create micelle based nanoparticles as well. Micelles may also be prepared using a diblock copolymer of PLGA and PEG, or a PEG-PLGA-PEG triblock copolymer.

In particular, polymers comprising polyethyleneimine (PEI) have been investigated for the delivery of nucleic acids. Nanoparticle vectors composed of poly(β-amino esters) (PBAEs) have also been shown to be suitable for nucleic acid delivery, especially in coformulation with polyethylene glycol (PEG) (Kaczmarek J C et al Angew Chem Int Ed Engl. 2016; 55(44): 13808-13812). Dendrimers are also contemplated for use. Particles of such coformulations have been used to deliver mRNA to the lung.

Also considered are particles based on polysaccharides and their derivatives, such as cellulose, chitin, cyclodextrin, and chitosan. Chitosan is a cationic linear polysaccharide obtained by partial deacetylation of chitin, with nanoparticles comprising this substance possessing promising properties for drug delivery such as biocompatibility, low toxicity and small size (Felt et al., Drug Development and Industrial Pharmacy, Volume 24, 1998—Issue 11). It is envisioned that combinations between the above constituents may be used. In specific embodiments of the invention the nanoparticles comprise chitosan which exhibits excellent mucoadhesion and penetration properties that make it ideal for sustained release biomolecule delivery in mucosa.

Delivery particles may include lipid-based, such as niosomal or liposomal, nanoparticle delivery systems. Lipid nanoparticles are multicomponent lipid systems typically containing a phospholipid, an ionizable lipid, cholesterol, and a PEGylated lipid. The PEGylated lipids on the particle surface can help to reduce particle aggregation and prolong the circulation time in vivo. Suitable liposomal formulations may include L-α-phosphatidylcholine and PEG-DMG (1,2-dimyristoyl-rac-glycero-3-methoxypolyethylene glycol). Alternative liposomal formulations comprising an ionizable lipid that are particularly, suitable for delivery of a nucleic acid may comprise DSPC (1,2-distearoyl-sn-glycero-3-phosphocholine) and Dlin-MC3-DMA (6Z, 9Z, 28Z, 31Z)-heptatriacont-6,9,28,31-tetraene-19-yl 4-(dimethylamino)butanoate. Another determinant for the potency of lipid-based nanoparticle is the lipid pKa. An optimal lipid pKa for the delivery of mRNA cargo is in the range of 6.6-6.8.

The delivery particles may comprise aminoalcohol lipidoids. These compounds may be used in the formation of particles including nanoparticles, liposomes and micelles, which are particularly suitable for the delivery of nucleic acids. An illustrative example for the production of nanoformulations comprising aminoalcohol lipidoid particles according to some embodiments of the invention may be found in the Examples. In embodiments of the invention, lipid nanoparticles (LNPs) comprised of dipalmitoylphosphatidylcholine (DPPC), cholesterol, and dioleoylglycerophosphate-diethylenediamine conjugate (DOP-DEDA) are positively charged at pH of 6.0, neutral at pH of 7.4 and negatively charged at pH of 8.0. This delivery system is neutral in the bloodstream to minimize degradation by plasma proteins and protect the encapsulated mRNA cargo. When delivered in vivo these LNP vehicles bind to apolipoproteins (e.g., apoE3) at their hydrophobic lipid regions, which can promote cellular uptake, especially by tumor cells.

The delivery particles may be targeted to the cells of the target tissue. This targeting may be mediated by a targeting agent on the surface of the delivery particles, which may be a protein, peptide, carbohydrate, glycoprotein, lipid, small molecule, nucleic acid, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, aptamers etc. Targeted liposomes, for example, modified by active targeting ligands can significantly improve liposome capacity by increasing accumulation at the target tissues/organs/cells without releasing the cargo, such as mRNA, to other sites.

Lipid-based nanoparticles may also act advantageously as an adjuvant in themselves. a broad range of lipids are reported to possess the strong inherent adjuvant activity. Cationic lipids such as dimethyldioctadecylammonium bromide (DDA) show the deposition of antigen at the injection site as well as the enhancement of a cellular antigen internalization. Solid lipid nanoparticles structured by DDA demonstrate high antigen adsorption efficiency, in vitro antigen trafficking, in vivo distribution, and high antibody response (Anderluzzi et al. J. Control Release 2020, 330, 933-944). As a result, efforts to improve adjuvancy in mRNA delivery vaccines that utilise LNPs as a delivery system tend to focus on engineering the lipids used in the nanoparticles. As mentioned above, however, there is a trade-off between lipid properties and suitability for encapsulation of mRNA as a cargo as well as in terms of biodistribution, release kinetics and cellular uptake.

When administered to a subject, a therapeutic component is suitably administered as part of the in vivo delivery composition and may further comprise a pharmaceutically acceptable vehicle in order to create a pharmaceutical composition. Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. Pharmaceutical compositions, if desired, can also contain minor amounts of wetting or emulsifying agents, or buffering agents.

The medicaments and pharmaceutical compositions of the invention can take the form of liquids, solutions, suspensions, gels, modified-release formulations (such as slow or sustained-release), emulsions, capsules (for example, capsules containing liquids or gels), liposomes, microparticles, nanoparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

For any compound or composition described herein, the therapeutically effective amount can be initially determined from in vitro cell culture assays. Target concentrations will be those concentrations of active component(s) that are capable of achieving the methods described herein, as measured using the methods described herein or known in the art.

As is well known in the art, therapeutically effective amounts for use in human subjects can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

It is contemplated that embodiments of the invention may include compositions formulated for use in medicine. As such, the composition of the invention may be suspended in a biocompatible solution to form a composition that can be targeted to a location on a cell, within a tissue or within the body of a patient or animal (i.e. the composition can be used in vitro, ex vivo or in vivo). Suitably, the biocompatible solution may be phosphate buffered saline or any other pharmaceutically acceptable carrier solution. One or more additional pharmaceutically acceptable carriers (such as diluents, adjuvants, excipients or vehicles) may be combined with the composition of the invention in a pharmaceutical composition. Suitable pharmaceutical carriers are described in ‘Remington's Pharmaceutical Sciences’ by E. W. Martin. Pharmaceutical formulations and compositions of the invention are formulated to conform to regulatory standards and can be administered orally, intravenously, topically, intratumorally, or subcutaneously, or via other standard routes. Administration can be systemic or local or intranasal or intrathecal. In particular, compositions according to the invention can be administered intravenously, intralesionally, intratumorally, subcutaneously, intra-muscularly, intranasally, intrathecally, intra-arterially and/or through inhalation.

Further intended are embodiments wherein the composition of some embodiments of the invention is administered separately to or in combination with alternative antitumoral or otherwise anti-cancer therapeutic components. These components can include oncolytic viruses, small molecule drugs, chemotherapeutics, radiotherapeutics, therapeutic vaccines or biologicals. The components may be administered concurrently with the composition of the invention, and may be comprised within delivery particles, or may be administered separately, before or after administration of the composition of the invention, by any means suitable.

It is also contemplated that the composition of some embodiments of the invention may be used in in vitro and/or ex vivo methods, for example in a laboratory setting. An example of an in vitro method is wherein a composition including a delivery system comprising an mRNA sequence as described herein is administered to target in vitro cells, and the miRNA binding site sequences comprised in the mRNA sequence allow for differential expression of the coding sequence of the mRNA in different cell types within the target in vitro cells. Similarly, a method is contemplated wherein a composition comprising a delivery system and an mRNA sequence as described herein is administered to a target ex vivo sample taken from an animal, and the miRNA binding site sequences comprised in the mRNA sequence allow for differential expression of the coding sequence of the mRNA in different cell types within the target sample.

Vaccines

mRNA constructs and compositions as described herein can be used in vaccine therapy, in the enhancement of the efficacy of a conventional vaccine, and/or as a novel vaccine form for use against infectious pathogens, such as viruses, bacteria, fungi, protozoa, prions, and helminths (worms); or for use in treating diseases such as cancer. It is contemplated that mRNA constructs as described can be circularised by the (direct or indirect) linkage of the 5′ and 3′ ends and such circular or circularised RNA constructs are considered to be included by the term ‘mRNA construct’ as used herein; such constructs have been shown to be potentially effective as RNA-based vaccines, for example against SARS-CoV-2 (Qu L. et al, bioRxiv 2021.03.16.435594; https://doi.org/10.1101/2021.03.16.435594). As a result, mRNA constructs as described herein include circular or circularised RNA constructs which can be translated in cells.

Hence, the compositions of the present invention may be used in the prophylaxis or treatment of infectious pathogenic disease, either by way of inclusion within vaccine formulations or in the form of adjuvants (e.g. with an appropriate cytokine) that is administered in combination with a vaccine.

Examples of infectious bacterial agents include Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Prevotella melaninogenica, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacian, Calymmatobacterium granulomatis, Campylobacter coli, Campylobacter fetus, Campylobacter jejuni, Campylobacter pylori, Chlamydia, Chlamydia trachomatis, Chlamydophila pneumoniae, Chlamydia pneumoniae, Chlamydophila psittaci, Chlamydia psittaci, Clostridium botulinum, Clostridium difficile, Clostridium perfringens, Clostridium welchii, Clostridium tetani, Corynebacterium diphtheriae, Corynebacterium fusiforme, Coxiella burnetiid, Ehrlichia chaffeensis, Ehrlichia ewingii, Eikenella corrodens, Enterobacter cloacae, Enterococcus avium, Enterococcus durans, Enterococcus faecalis, Enterococcus faecium, Enterococcus gallinarum, Enterococcus maloratus, Escherichia coli, Fusobacterium necrophorum, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leishmania donovani, Leptospira interrogans, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Peptostreptococcus, Porphyromonas gingivalis, Prevotella melaninogenica, Bacteroides melaninogenicus, Pseudomonas aeruginosa, Rhizobium radiobacter, Rickettsia prowazekii, Rickettsia psittaci, Rickettsia quintana, Rickettsia, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Ureaplasma urealyticum, Vibrio cholerae, Vibrio comma, Vibrio parahaemolyticus, Vibrio vulnificus, Yersinia enterocolitica, Yersinia pestis and Yersinia pseudotuberculosis.

Examples of viral infectious agents include Adeno-associated virus; Aichi virus, Australian bat lyssavirus; BK polyomavirus; Banna virus; Barmah forest virus; Bunyamwera virus; Bunyavirus La Crosse; Bunyavirus snowshoe hare; Cercopithecine herpesvirus; Chandipura virus; Chikungunya virus; Cosavirus A; Cowpox virus; Coxsackievirus; Crimean-Congo hemorrhagic fever virus; Dengue virus; Dhori virus; Dugbe virus; Duvenhage virus; Eastern equine encephalitis virus; Ebolavirus; Echovirus; Encephalomyocarditis virus; Epstein-Barr virus; European bat lyssavirus; GB virus C/Hepatitis G virus; Hantaan virus; Hendra virus; Hepatitis A virus; Hepatitis B virus; Hepatitis C virus; Hepatitis E virus; Hepatitis delta virus; Horsepox virus; Human adenovirus; Human astrovirus; Human coronavirus; Human cytomegalovirus; Human enterovirus 68, 70; Human herpesvirus 1; Human herpesvirus 2; Human herpesvirus 6; Human herpesvirus 7; Human herpesvirus 8; Human immunodeficiency virus; Human papillomavirus 1; Human papillomavirus 2; Human papillomavirus 16,18; Human parainfluenza; Human parvovirus B19; Human respiratory syncytial virus; Human rhinovirus; Human SARS coronavirus; Human spumaretrovirus; Human T-lymphotropic virus; Human torovirus; Influenza A virus; Influenza B virus; Influenza C virus; Isfahan virus; JC polyomavirus; Japanese encephalitis virus; Junin arenavirus; KI Polyomavirus; Kunjin virus; Lagos bat virus; Lake Victoria Marburgvirus; Langat virus; Lassa virus; Lordsdale virus; Louping ill virus; Lymphocytic choriomeningitis virus; Machupo virus; Mayaro virus, MERS coronavirus; Measles virus; Mengo encephalomyocarditis virus; Merkel cell polyomavirus; Mokola virus; Molluscum contagiosum virus; Monkeypox virus; Mumps virus; Murray valley encephalitis virus; New York virus; Nipah virus; Norwalk virus; O'nyong-nyong virus; Orf virus; Oropouche virus; Pichinde virus; Poliovirus; Punta toro phlebovirus; Puumala virus; Rabies virus; Respiratory syncytial virus; Rift valley fever virus; Rosavirus A; Ross river virus; Rotavirus A; Rotavirus B; Rotavirus C; Rubella virus; Sagiyama virus; Salivirus A; Sandfly fever sicilian virus; Sapporo virus; SARS coronavirus 2 (COVID); Semliki forest virus; Seoul virus; Simian foamy virus; Simian virus 5; Sindbis virus; Southampton virus; St. louis encephalitis virus; Tick-borne powassan virus; Torque teno virus; Toscana virus; Uukuniemi virus; Vaccinia virus; Varicella-zoster virus; Variola virus; Venezuelan equine encephalitis virus; Vesicular stomatitis virus; Western equine encephalitis virus; WU polyomavirus; West Nile virus; Yaba monkey tumor virus; Yaba-like disease virus; Yellow fever virus; and Zika virus.

Examples of fungal infectious agents include: Gymnopus spp., Rhodocollybia butyracea, Hypholoma fasciculare, Saccharomyces cerevisiae, Tuber spp., Bothia castanella, Rhizosphere spp., Herpotrichiellaceae spp., Verrucariaceae spp., Marchandiomyces spp., Minimedusa spp., Marchandiobasidium aurantiacum, Marchandiomyces corallinus, Marchandiomyces lignicola, Burgoa spp., Athelia arachnoidea, Alternaria alternata, Alternaria spp., Boletus edulis, Leccinum aurantiacum, Trametes versicolor, Trametes spp., Sympodiomycopsis spp., Flavocetraria nivalis, Ampelomyces spp., Gymnopus biformis, Gymnopus spp., Gymnopus confluens, Gymnopus spongiosus, Collybia readii, Marasmiellus stenophyllus, Marasmiellus ramealis, Marasmius scorodonius, Collybia marasmioides, Micromphale brassicolens, Caripia montagnei, Rhodocollybia spp., Anthracophyllum lateritium, Anthracophyllum archeri, Anthracophyllum spp., Phanerochaete spp., Schizosaccharomyces pombe, Saccharomyces cerevisiae, Aspergillus fumigatus, Aspergillus flavus, Aspergillus niger, Aspergillus spp., Tricholoma imbricatum, Tricholoma flavovirens, Tomentella sublilacina, Rhizopogon spp., Laccaria spp., Inocybe spp., Hebeloma spp., Cortinarius spp., Clavulina spp., Xerocomus spp., Amanita spp., Eurotium herbariorum, Edyuillia athecia, Warcupiella spinulosa, Hemicarpenteles paradoxus, Hemicarpenteles acanthosporus, Hemicarpenteles spp., Chaetosartorya cremea, Petromyces spp., Graphium tectonae, Di plolaimelloides spp., Rhabdolaimus spp., Hohenbuehelia petalodes, Glomerella graminicola, Cryptococcus arboriformis, Cryptococcus neoformans, Cryptococcus spp., Gamsylella parvicollis, Monacrosporium haptotylum, Monacrosporium sichuanense, Monacrosporium Spp., Monacrosporium gephyropagum, Monacrosporium spp., Drechslerella coelobrocha, Drechslerella dactyloides, Drechslerella spp., Arthrobotrys musiformis, Arthrobotrys flagrans, Arthrobotrys hertziana, Arthrobotrys oligospora, Arthrobotrys vermicola, Arthrobotrys spp., Monacrosporium drechsleri, Vermispora spp., Pseudallescheria boydii (Scedosporium apiospermum), Scedosporium inflatum, Geosmithia spp., Glomerella cingulata, Lophodermium piceae, Fusarium asiaticum, Fusarium spp., Pleurotus eryngii, Cintractia sorghi-vulgaris, Cantharocybe gruberi, Bourdotia spp., Auricularia spp., Puccinia bartholomaei, Puccinia spp., Diaporthe phaseolorum, Melanconis stilbostoma, Xylaria spp., Trichophyton equinum, Trichophyton tonsurans, Trichophytum violaceum, Trichophytum rubrum, Trichophytum interdigitale, Trichophytum schoenleinii Trichophyton spp., Chlorophyllum agaricoides, Cenococcum geophilum, Helotiales spp., Rhizoscyphus ericae, Lactarius pubescens, Lactarius spp., Piloderma fallax, Suillus luteus, Amanita muscaria, Tricholoma spp., Laccaria cf bicolour, Cortinarius purpurascens, Seiridium spp., Apiospora montagnei, Chondrostereum purpureum, Botryobasidium subcoronatum, Boletellus shichianus, Boletellus spp., Hypocrea farinose, Hypocrea spp., Sarcostroma restionis, Sarcostroma spp., Truncatella betulae, Truncatella spp., Pestalotiopsis matildae, Paraconiothyrium spp., Phoma spp., Cunninghamella bainieri, Cunninghamella bertholletiae, Cantharellus cibarius, Apiospora bambusae, Apiospora spp., Discostroma botan, Cercophora caudate, Gnomonia ribicola, Faurelina elongate, Mycorrhiza fungi, Geomyces pannorum, Coprinus spp., Acremonium spp., Clonostachys spp., Phoma eupyrena, Tetracladium spp., Mortierella spp., Tulasnella calospora, Epulorhiza spp., Tulasnella calospora, Antarctomyces psychrotrophicus, Amphisphaeriaceae spp., Phomopsis spp., Trichoderma spp., Pestalotiopsis spp., Pestalotiopsis spp., Trichocomaceae spp., Coniochaetales spp., Tremellales spp., Dothideales spp., Phyllachoraceae spp., Saccharomycetales spp., Herpotrichiellaceae spp., Liliopsida spp., Trichosporonales spp., Trichosporon mycotoxinivorans, Trichosporon spp., Dothioraceae spp., Hypocreales spp., Mycosphaerellaceae spp., Sporidiobolales spp., Clavicipitaceae spp., Pleosporales spp., Ustilaginaceae spp., Phyllachoraceae spp., Mucoraceae spp., Sordariales spp., Filobasidiales spp., Calosphaeriaceae spp., Clavicipitaceae spp., Mucorales spp., Herpotrichiellaceae spp., Microdochium spp., Phyllachoraceae spp., Zopfiaceae spp., Botryosphaeriaceae spp., Helotiaceae spp., Bionectriaceae spp., Lachnocladiaceae spp., Di podascaceae spp., Caulerpaceae spp., Microstromatales spp., Aphyllophorales spp., Montagnulaceae spp., Gymnoascaceae spp., Cryphonectriaceae spp., Xylariales spp., Montagnulaceae spp., Chaetomiaceae spp., Xanthoria elegans, Rhizopus spp., Penicillium spp., Cetraria aculeate, Nephromopsis laureri, Tuckermannopsis chlorophylla, Cetraria ericetorum, Cetraria spp., Flavocetraria cucullata, Kaernefeltia merrillii, Amorosia littoralis, Quambalaria cyanescens, Cordyceps roseostromata, Cordyceps spp., Russula spp., Clavulina spp., Tuber quercicola, Gymnomyces spp., Tetrachaetum elegans, Anguillospora longissima, Hypocrea spp., Sirococcus conigenus, Rhizopogon roseolus, Rhizopogon olivaceotinctus, Rhizopogon spp., Pisolithus microcarpus, Rhizoscyphus ericae, Cortinarius glaucopus, Paxillus spp., Suillus variegates, Pyrobaculum aerophilum, Tulasnella spp., Hohenbuehelia spp., Cochliobolus lunatus, Plicaturopsis crispa, Bondarcevomyces taxi, Tapinella panuoides, Tapinella spp., Austropaxillus spp., Gomphidius roseus, Gyrodon lividus, Phylloporus pelletieri, Chamonixia caespitose, Porphyrellus porphyrosporus, Truncocolumella citrina, Tapinella atrotomentosa, Scleroderma leave, Suillus variegates, Suillus spp., Porphyrellus porphyrosporus, Pisolithus arrhizus, Phaeogyroporus portentosus, Melanogaster variegates, Leucogyrophana mollusca, Hydnomerulius pinastri, Gomphidius roseus, Gyrodon lividus, Gyroporus cyanescens, Chalciporus piperatus, Chamonixia caespitose, Bondarcevomyces taxi, Dendryphiella triticicola, Guignardia spp., Shiraia spp., Cladosporium spp., Phomopsis spp., Diaporthales spp., Pestalotiopsis spp., Lophiostoma spp., Verticillium chlamydosporium, Paecilomyces lilacinus, Paecilomyces varioti, Paecilomyces spp., Ceratorhiza oryzae-sativae, Geosmithia pallida, Geosmithia spp., Geosiphon pyriformis, Agonimia spp., Pyrgillusjavanicus, Exophiala dermatitidis, Exophiala pisciphila, Exophiala spp., Ramichloridium anceps, Ramichloridium spp., Capronia pilosella, Isaria farinose, Pochonia suchlasporia, Lecanicillium psalliotae, Dothideomycete spp., Leotiomycete spp., Ustilaginoidea vixens, Hyphozyma lignicola, Coniochaeta malacotricha, Coniochaeta spp., Torrubiella confragosa, Isaria tenuipes, Microsporum canis, Microsporum audouinii, Microsporum spp., Epicoccum floccosum, Gigaspora rosea, Gigaspora spp., Ganoderma spp., Pseudoperonospora cubensis, Hyaloperonospora parasitica, Plectophomella spp., Aureobasidium pullulans, Gloeophyllum sepiarium, Gloeophyllum spp., Donkioporia expansa, Antrodia sinuosa, Phaeoacremonium rubrigenum, Phaeoacremonium spp., Albertiniella polyporicola, Cephalotheca sulfurea, Fragosphaeria renifbrmis, Fragosphaeria spp., Phialemonium dimorphosporum, Phialemonium spp., Pichia norvegensis, Pichia spp., Candida albicans, Candida tropicalis, Candida glabrata, Candida parapsilosis, Candida spp., Gondawanamyces spp., Graphium spp., Ambrosiella spp., Microglossum spp., Neobulgaria pura, Holwaya mucida, Chlorovibrissea spp., Chlorociboria spp., Thaxterogaster spp., Cortinarius spp., Setchelliogaster spp., Timgrovea spp., Descomyces spp., Hymenogaster arenarius, Quadrispora tubercularis, Quadrispora spp., Protoglossum violaceum, Ceratostomella pyrenaica, Ceratosphaeria lampadophora, Fonsecaea pedrosoi, Phlebia acerina, Phlebia spp., Pestalotiopsis disseminata, Paracoccidioides brasiliensis, Racospermyces koae, Endoraecium acaciae, Uromycladium tepperianum, Uromycladium spp., Agaricus bisporus, Agaricus spp., Psilocybe quebecensis, Psilocybe merdaria, Psilocybe spp., Gymnopilus luteofolius, Gymnopilus Iiquiritiae, Gymnopilus spp., Hypholoma tuberosum, Melanotus hartii, Panaeolus uliginosus, Stropharia rugosoannulata, Dermocybe semisanguinea, Dermocybe spp., Helicoma month* pes, Helicoma spp., Tubeufia helicomyces, Tubeufia spp., Leohumicola verrucosa, Leptosphaerulina chartarum, Macrophoma spp., Marssonina rosae, Botryotinia fuckeliana, Pestalotiopsis spp., Chrysosporium carmichaelii, Chrysosporium spp., Dactylella oxyspora, Dactylellina lobatum, Cucurbitaceae spp., Chrysophyllum sparsiflorum, Chrysophyllum spp., Blumeria graminis, Sawadaea polyfida, Sawadaea spp., Parauncinula septata, Erysiphe mori, Erysiphe spp., Typhulochaeta japonica, Golovinomyces orontii, Golovinomyces spp., Podosphaera xanthii, Podosphaera spp., Arthrocladiella mougeotii, Neoerysiphe galeopsidis, Phyllactinia kakicola, Phyllactinia spp., Cyphellophora laciniata, Sphaerographium tenuirostrum, Microsphaera trifolii, Sphaerotheca spiraeae, Sphaerotheca spp., Uncinuliella australiana, Absidia corymbifera, Absidia spp., Geotrichum spp., Nectria curta, Anamika lactariolens, Hebeloma velutipes, Stropharia ambigua, Agrocybe praecox, Hydnum rufescens, Hydnum spp., Meliniomyces variabilis, Rhizoscyphus ericae, Cryptosporiopsis ericae, Hyalodendron spp., Leptographium lundbergii, Leptographium spp., Termitomyces spp., Coccidioides posadasii, Coccidioides immitis, Sclerotinia sclerotiorum, Phomopsis spp., Metarhizium anisopliae, Cordyceps spp., Tilletiopsis washingtonensis, Cerrena unicolor, Stachybotrys chartarum, Phaeococcomyces nigricans, Ganoderma philippii, Ganoderma spp., Gloeophyllum sepiarium, Cystotheca lanestris, Leveillula taurica, Phyllactinia fraxini, Varicosporium elodeae, Rhinocladiella basitonum, Melanchlenus oligospermus, Clavispora lusitaniae, Rhizopus spp., Phizomucor spp., Mucor spp., Conidiobolus coronatus, Conidobolus spp., Basidiobolus ranarum, basidiobolus spp., Ochronis spp., Histoplasma capsulatum, histoplasma spp., Wilcoxina mikolae, Lasiodiplodia spp., Physcia caesia, Physcia spp., Brachyconidiellopsis spp., Conocybe lacteal, Gastrocybe lateritia, Gastrocybe spp., Agrocybe semiorbicularis, Taphrina pruni, Taphrina spp., Asterophora parasitica, Asterophora spp., Eremothecium ashbyi, Tricladium splendens, Ramaria flava, Ramaria spp., Laccaria fraternal, Scutellospora spp., Illosporium carneum, Hobsonia christiansenii, Marchandiomyces corallinus, Fusicoccum luteum, Botryosphaeria ribis, Pseudozyma aphidis, Pseudozyma spp., Pesotum erubescens, Battarrea stevenii, Battarrea spp., Harposporium Janus, Harposporium spp., Hirsutella rhossiliensis, Arthroderma ciferrii, Arthroderma spp., Pucciniastrum goeppertianum, Cronartium occidentale, Cronartium arizonicum, Cronartium spp., Peridermium harknessii, Peridermium spp., Chrysomyxa arctostaphyli, Holleya sinecauda, Holleya spp., Zoophthora radicans, Smittium culisetae, Auxarthron zuffianum, Renispora flavissima, Ctenomyces serratus, and Sporothrix schenckii.

Examples of parasitic species as infectious agents may include helmiths (worms) that may be selected from: cestodes: e.g. Anaplocephala spp.; Dipylidium spp.; Diphyllobothrium spp.; Echinococcus spp.; Moniezia spp.; Taenia spp.; trematodes e.g. Dicrocoelium spp.; Fasciola spp.; Paramphistomum spp.; Schistosoma spp.; or nematodes, e.g.; Ancylostoma spp.; Anecator spp.; Ascaridia spp.; Ascaris spp.; Brugia spp.; Bunostomum spp.; Capillaria spp.; Chabertia spp.; Cooperia spp.; Cyathostomum spp.; Cylicocyclus spp.; Cylicodontophorus spp.; Cylicostephanus spp.; Craterostomum spp.; Dictyocaulus spp.; Dipetalonema spp; Dirofilaria spp.; Dracunculus spp.; Enterobius spp.; Filaroides spp.; Habronema spp.; Haemonchus spp.; Heterakis spp.; Hyostrongylus spp.; Metastrongylus spp.; Meullerius spp. Necator spp.; Nematodirus spp.; Nippostrongylus spp.; Oesophagostomum spp.; Onchocerca spp.; Ostertagia spp.; Oxyuris spp.; Parascaris spp.; Stephanurus spp.; Strongylus spp.; Syngamus spp.; Toxocara spp.; Strongyloides spp.; Teladorsagia spp.; Toxascaris spp.; Trichinella spp.; Trichuris spp.; Trichostrongylus spp.; Triodontophorous spp.; Uncinaria spp., and/or Wuchereria spp.

Examples of parasitic species as infectious agents may include protozoa that are selected from: Leishmania species including Trypanosoma, Donovan Leishmania, Plasmodium spp. including, but not limited to, Plasmodium falciparum; Pneumocystis carini, Cryptosporidium parum, Rumble flagellate, Shigella amoeba, and Cyclosporanga canetenensis.

Vaccine compositions and methods as discussed herein are non-exclusively contemplated for the treatment and prevention of diseases which may already be known to be susceptible to vaccination, particularly where an effective immunogenic protein is known.

Table 5 (below) provides an illustrative example of antigens that are selected to use in the compositions and methods of the present invention, for which an immune response is desired. One of skill in the art could readily obtain similar antigens/targets from public databases and publications and generate compositions of the invention. It should be understood that more than one antigen may be delivered to a subject depending on the state of disease, e.g., prophylactic prior to infection versus an active infection. By way of example, for a subject with an active tuberculosis disease, one might deliver the TB antigen that codes for a TB protein from the active phase (e.g., ESAT6 Ag85B), from latent phase (Rv2626), and/or from the resuscitation phase (RPfB-D). In this way, an active tuberculosis can be treated, particularly when it is desired to administer an adjuvant that elicits a Th1 response.

In one aspect of the invention, the compositions described herein are administered in combination with standard therapies, e.g., for an active bacterial or viral infection, antimicrobial agents or antiviral agents known in the field to treat such diseases can be administered. Such agents can be administered prior to, simultaneously with (either alone or as a fixed dose combination) or following treatment with a composition of the invention.

In some embodiments, the coding mRNA can code for an antigen against which an immune response is desired. Delivery of such antigens can be used to induce a local immune response as discussed above, or in order to provoke an adaptive immune response to the antigen itself—that is, to induce immunity against that antigen, similar to a vaccine. In such cases, the compositions according to the invention may be combined with adjuvants to encourage the generation of an immune response. Suitably, one or more proinflammatory cytokines may be utilised as an adjuvant—e.g. selected from: IFNγ; IFNα; IFNβ; TNFα; IL-12; IL-2; IL-6; IL-8; and GM-CSF, or agonists and homologues thereof. Optionally, the one or more proinflammatory cytokines is particularly selected from IL-2; IL-12; IFNγ; TNFα and GM-CSF. In specific embodiments, the one or more proinflammatory cytokines is selected from: IL-12, IFNγ and GM-CSF. In specific embodiments the proinflammatory cytokine acts as an adjuvant for a co- or serially administered vaccine composition.

Prophylactic Vaccine to Prevent Infectious Diseases or to Prevent Pathogen Infection

For example, the coding mRNA can encode a bacterial, viral or otherwise microbial protein against which an immune reaction is desired, in whole or part. Such encoded products are referred to for this discussion as ‘antigen products’ or ‘antigen’. In some cases, immunity can be generated against only part of a bacterial, viral or otherwise microbial protein (an ‘epitope’ or ‘antigenic determinant’), so the encoding of only those parts is also envisaged. In particular, parts of a microbial protein which are displayed externally can be selected as likely targets for immune recognition. As a result, an encoded antigen can be a bacterial, viral or otherwise microbial protein, but can be a partial sequence, part or fragment thereof, in particular, an ‘epitope containing fragment’ thereof. It is envisioned that more than one antigen for a particular microbe or pathogen can be provided, in the same or different mRNA constructs.

Vaccine compositions and methods as discussed herein are non-exclusively contemplated for the treatment and prevention of diseases which are already known to be susceptible to vaccination, particularly where an effective immunogenic protein is known, such as described Table 5. As a result, compositions and methods herein can use mRNA constructs which encode one or more of the below-described immunogenic proteins, or variants thereof.

TABLE 5
Exemplary vaccine antigens for infectious diseases

| Pathogen | Antigens/targets | Reference |
|---|---|---|
| Chickenpox (varicella) | whole varicella-zoster virus (VZV) antigen, | Bergen, R. E., Diaz, P. |
| | VZV glycoprotein I (gpl), immediate early | S., & Arvin, A. M. |
| | protein (IE-62) | (1990). |
| | VZV gE: varicella-zoster virus glycoproteinE | https://academic.oup.com/ |
| | | jid/article- |
| | | abstract/162/5/1049/ |
| | | 829640 |
| | | https://doi.org/10.1517/ |
| | | 14712598.2016.1134481 |
| Chlamydia spp. | PmpG, PmpI, PmpE, MOMP, PmpD, PmpH, | US10953080B2 |
| | OmcB, OmpH and HtrA | |
| Cholera | rCTB | Price, G. A., McFann, |
| | LT | K., & Holmes, R. K. |
| | inactivated Vibro Cholerae O1 bacteria, | (2013). |
| | enterotoxigenic Escherichia coli (ETEC) | https://journals.plos.org/ |
| | CFA/I, CS3, CS5 and CS6, LCTBBA | plosone/article?id=10.1371/ |
| | | journal.pone.0057269 |
| | | Dukoral, INN-cholera |
| | | vaccine (inactivated |
| | | oral) Annex I: |
| | | Summary of product |
| | | Characteristics |
| | | https://www.ema.europa.eu/ |
| | | en/documents/ |
| | | product- |
| | | information/dukoral- |
| | | epar-product- |
| | | information_en.pdf |
| | | Mottram, L., Lundgren, |
| | | A., Svennerholm, A. |
| | | M., & Leach, S. (2021) |
| | | https://www.frontiersin.org/ |
| | | articles/10.3389/ |
| | | fimmu.2021.647873/ |
| | | full?utm_source=S- |
| | | TWT&utm_medium= |
| | | SNET&utm_campaign= |
| | | ECO_FIMMU_XXXXX |
| | | XXX_auto-dlvrit |
| Cryptococcus | Cda1, Cda2, Cda3, Fpd1, MP88, and Sod1 | Specht, C. A., et al., |
| neoformans | | (2017). |
| | | https://journals.asm.org/ |
| | | doi/full/10.1128/mBio.01872-17 |
| Diphtheria | diphtheria toxoid | Stratton, K., Ford, A., |
| | Antigens for Corynebacterium diphtheriae | Rusch, E., Clayton, E. |
| | | W., & Committee to |
| | | Review Adverse |
| | | Effects of Vaccines. |
| | | (2011). |
| | | https://www.ncbi.nlm.nih.gov/ |
| | | books/NBK190028/ |
| | | US20210023199A1 |
| Haemophilus | PRP | Kelly, D. F., Moxon, E. |
| Influenzae type b | | R., & Pollard, A. J. |
| | | (2004). |
| | | https://www.ncbi.nlm.nih.gov/ |
| | | pmc/articles/PMC1782565/ |
| Hepatitis A | inactivated hepatitis A virus (strain HM175) | https://www.rxlist.com/ |
| | | twinrix-drug.htm |
| Hepatitis B | noninfectious hepatitis B virus surface | See above |
| | antigen (HBsAg) | |
| Histoplasma | Hsp60 from Histoplasma capsulatum | Deepe, Jr., G. S., & |
| | | Gibbons, R. S. (2002). |
| | | https://journals.asm.org/ |
| | | doi/10.1128/IAI.70.7.3759- |
| | | 3767.2002 |
| Human | gag, pol, env, and nef | I P Nascimento and |
| Immunodeficiency | | L C C Leite, Braz J Med |
| Virus (HIV) | | Biol Res. 2012 |
| | | doi: 10.1590/S0100- |
| | | 879X2012007500142 |
| Human | L1 major capsid protein of HPV | https://www.cdc.gov/ |
| Papillomavirus | | vaccines/pubs/pinkbook/ |
| Infection | | hpv.html |
| | L2 major capsid protein of HPV | https://doi.org/10.1128/ |
| | | JVI.75.19.9201- |
| | | 9209.2001 |
| Influenza | Influenza A virus (HA, NA, NP, M2, M1 | US20210023199A1 |
| | antigens), influenza B virus (HA, NA | |
| | antigens), respiratory syncytial virus (F, G, | |
| | M, SH antigens), parainfluenza virus | |
| | (glycoprotein antigens) | |
| Leishmania spp. | Tagatose-6-phosphate kinase-like protein | John, L., John, G. J., & |
| | (XP_822202.1) | Kholia, T. (2012). |
| | Phosphatidylinositol 3-kinase-like protein | https://link.springer.com/ |
| | (XP_822211.1) | article/10.1007/ |
| | XP_001687567.1 is a surface antigen | s12010-012-9649-0 |
| | protein and is predicted to be an | https://doi.org/10.1371/ |
| | extracellular/secreted protein | journal.pntd.0005527 |
| | Phosphoglycan beta 1,3 | https://doi.org/10.1089/ |
| | galactosyltransferase 4 (XP_822217.1, | hum.1998.9.13-1899 |
| | XP_822221.1, and XP_001686570.1) and | https://doi.org/10.1084/ |
| | glycosomal membrane protein | jem.186.7.1137 |
| | (XP_843475.1) | https://doi.org/10.1016/ |
| | Proteophosphoglycan ppg4 (XP_843162.1), | j.micinf.2007.05.012 |
| | proteophosphoglycan ppg5 (XP_843163.1), | https://doi.org/10.5812/ |
| | and proteophosphoglycan ppg1 | jm.8974 |
| | (XP_843164.1) | https://doi.org/10.1016/ |
| | Tuzin protein (XP_001686384.1) | j.vaccine.2011.02.096 |
| | Receptor-type adenylate cyclase a-like | |
| | protein (XP_001686897.1) | |
| | KMP11: kinetoplastid membrane protein-11 | |
| | HASPB: hydrophilic acylated surface | |
| | protein B | |
| | GP63: glycoprotein 63 | |
| | LACK: Leishmania homologue for receptors | |
| | of activated C kinase | |
| | A2: Amastigote-specific A2 proteins | |
| | NH: nucleoside hydrolase | |
| | TSA: Thiol-specific antioxidant | |
| | LeIF: Leishmania elongation initiation factor | |
| | LmSTI1: Leishmania major stress-inducible | |
| | protein 1 | |
| Listeria | listeriolysin O (LLO) | Calderón-González, |
| monocytogenes | glyceraldehyde-3-phosphate- | R., Frande-Cabanes, |
| | dehydrogenase (GAPDH) | E., Bronchalo-Vicente, |
| | | L., Lecea-Cuello, M. |
| | | J., Pareja, E., Bosch- |
| | | Martinez, A., . . . & |
| | | Álvarez-Dominguez, |
| | | C. (2014). |
| | | https://www.frontiersin.org/ |
| | | articles/10.3389/ |
| | | fcimb.2014.00022/full |
| Measles | measles virus antigens (e.g., live attenuated | US9107831B2 |
| | virus antigens) | |
| Meningococcal | meningococcal (Neisseria meningitidis) | US9107831B2 |
| Disease | antigens, polysaccharide and conjugate | |
| | antigens (e.g., meningitis A, meningitis B, | |
| | meningitis C, meningitis W, meningitis Y) | |
| Mumps | mumps virus antigens (e.g., live, attenuated | US9107831B2 |
| | virus antigens) | |
| Mycobacteria | Early secretory antigenic target-6 (ESAT6) | Kwon, B. E., Ahn, J. |
| | | H., Park, E. K., Jeong, |
| | | H., Lee, H. J., Jung, Y. |
| | | J., . . . & Ko, H. J. |
| | | (2019). |
| | | https://www.frontiersin.org/ |
| | | articles/10.3389/ |
| | | fimmu.2019.02542/full |
| Pertussis (whooping | pertussis toxin (PT), filamentous | US20200405839A1 |
| cough) | hemagglutinin (FHA), fimbriae (FIM), | |
| | pertactin (PRN), the siderophore receptor | |
| | protein FauA, the xenosiderophore receptor | |
| | protein BfeA, the hemophore receptor | |
| | protein BfuR | |
| Poliomyelitis | inactivated poliovirus serotypes, D-antigen | Bandyopadhyay, A. S., |
| | (D-Ag) | Garon, J., Seib, K., & |
| | polio virus (VP1-4) | Orenstein, W. A. |
| | | (2015). |
| | | https://www.futuremedicine.com/ |
| | | doi/10.2217/fmb.15.19 |
| | | US20210023199A1 |
| Pneumococcal | pneumococcal (Streptococcus pneumoniae) | US9107831B2 |
| Disease | antigens (e.g., polysaccharide and | US20210023199A1 |
| | conjugate antigens) | https://doi.org/10.5863/ |
| | Streptococcus pneumoniae (Pht, PcsB, | 1551-6776-21.1.27 |
| | StkP antigens) | https://doi.org/10.1016/ |
| | PspA: Pneumococcal surface protein A | j.vaccine.2012.11.005 |
| | PlyD1: genetically detoxified pneumolysin | https://doi.org/10.1128/ |
| | protein with 3 mutations | IAI.05780-11 |
| | RrgB321: Fusion Protein of the Three | https://doi.org/10.5863/ |
| | Variants of the Pneumococcal Pilus | 1551-6776-21.1.27 |
| | Backbone RrgB | https://doi.org/10.3390/ |
| | PcsB | vaccines7010009 |
| | StkP: serine-threonine protein kinase | https://doi.org/10.3390/ |
| | PsaA: pneumococcal surface adhesion | vaccines7010009 |
| | protein A | |
| | PspC: Pneumococcal surface protein C | |
| | PhtD: pneumococcal histidine triad D | |
| | PcpA: pneumococcal-choline binding | |
| | protein A | |
| Legionella | recombinant peptidoglycan-associated | Mobarez, A. M., |
| pneumophila | lipoprotein (rPAL) | Rajabi, R. A., |
| | | Salmanian, A. H., |
| | | Khoramabadi, N., & |
| | | Doust, S. R. H. (2019). |
| | | https://pubmed.ncbi.nlm.nih.gov/ |
| | | 30550844/ |
| Protozoa | Fucose-Mannose-Ligand glycoprotein | Mcallister, M. M. |
| | antigen | (2014). |
| | Leishmania amastigote antigen (A2) | https://www.ncbi.nlm.n |
| | Excreted Secreted Proteins (ESP) of | ih.gov/pmc/articles/PM |
| | L. infantum | C3961066/ |
| Rickettsia spp. | rickettsial outer membrane protein B | Chan, Y. G. Y., Riley, |
| | (rOmpB) | S. P., Chen, E., & |
| | | Martinez, J. J. (2011). |
| | | https://www.ncbi.nlm.nih.gov/ |
| | | pmc/articles/PMC3125829/ |
| Rotavirus | VP6 | Svensson, L., |
| | | Sheshberadaran, H., |
| | | Vesikari, T., Norrby, |
| | | E., & Wadell, G. |
| | | (1987). |
| | | https://www.microbiologyresearch.org/ |
| | | content/journal/jgv/ |
| | | 10.1099/0022-1317-68-7- |
| | | 1993?crawler=true |
| Rubella | rubella virus antigen (e.g., live, attenuated | US9107831B2 |
| | virus antigens) | |
| Salmonella | lipid-A free lipopolysaccharide (LFPS) | Chiu, T. W., Peng, C. |
| typhimurium | | J., Chen, M. C., Hsu, |
| | | M. H., Liang, Y. H., |
| | | Chiu, C. H., . . . & Lee, |
| | | Y. C. (2020). |
| | | https://jbiomedsci.biomedcentral.com/ |
| | | articles/10.1186/ |
| | | s12929-020-00681-8 |
| Shingles (Herpes | lyophilized varicella zoster virus | Monslow, M. A., |
| Zoster) | glycoprotein E (gE) | Elbashir, S., Sullivan, |
| | | N. L., Thiriot, D. S., |
| | | Ahl, P., Smith, J., . . . & |
| | | Vora, K. A. (2020). |
| | | https://www.sciencedirect.com/ |
| | | science/article/pii/ |
| | | S0264410X20308483 |
| Smallpox | lyophilized preparation of infectious vaccinia | Walsh, S. R., & Dolin, |
| | virus | R. (2011). |
| | | https://www.tandfonline.com/ |
| | | doi/abs/10.1586/ |
| | | erv.11.79 |
| Tetanus | tetanus toxoid | Stratton, K., Ford, A., |
| | Antigens for clostridium tetani | Rusch, E., Clayton, E. |
| | 8MTT (tetanus toxin genetically inactivated | W., & Committee to |
| | with 8 aa mutations) | Review Adverse |
| | TeNT-Hc (tetanus toxin fragment C) | Effects of Vaccines. |
| | | (2011). |
| | | https://www.ncbi.nlm.nih.gov/ |
| | | books/NBK190028/ |
| | | US20210023199A1 |
| | | Przedpelski et al. |
| | | mBio. 2020 Aug |
| | | 11; 11(4): e01668-20. |
| | | doi: 10.1128/mBio.01668-20. |
| | | Yu et al. |
| | | Toxins 2016, 8(7), |
| | | 194; doi: |
| | | 10.3390/toxins8070194 |
| Trypanosoma spp. | VSG | Akhoon, B. A., Slathia, |
| | IFX: invariant flagellum antigen from T. | P. S., Sharma, P., |
| | vivax | Gupta, S. K., & Verma, |
| | TcG2: Trypanosoma cruzi protein G2 | V. (2011). |
| | TcG4: Trypanosoma cruzi protein G4 | https://www.sciencedirect.com/ |
| | Enolase | science/article/ |
| | TSA1: trypomastigote surface antigen | abs/pii/S0882401011000222 |
| | Tc24: trypomastigote excretory-secretory | https://doi.org/10.1038/ |
| | protein 24 | s41586-021-03597-x |
| | Tc52: trypomastigote excretory-secretory | https://doi.org/10.3389/ |
| | protein 52 | fimmu.2019.01456 |
| | ASP1: amastigote surface protein 1 | https://doi.org/10.1155/ |
| | ASP2: amastigote surface protein 2 | 2018/8964085 |
| | ASP9: amastigote surface protein 9 | https://doi.org/10.1016 |
| | | j.actatropica.2019.105/168 |
| | | https://doi.org/10.1111/ |
| | | j.1574- |
| | | 695X.2007.00251.x |
| | | https://doi.org/10.1016/ |
| | | j.actatropica.2019.105168 |
| Mycobacterium | ESAT6, Ag85B, peptide 190-198 of MPT64, | Liu, Xun, et al., (2016). |
| tuberculosis | Mtb8.4, latency antigen Rv2626c | https://pubmed.ncbi.nlm.nih.gov/ |
| | resuscitation phase (RPfB-D) | 26901244/ |
| | Rv2041c | Shin, S. J., et al., |
| | ESAT-6; Ag85b; TB10.4; RpfB-D; Rv2626 | (2009). |
| | | https://pubmed.ncbi.nlm.nih.gov/ |
| | | 19874550/ |
| | | WO2014009438 A2 |
| Typhoid Fever | Vi polysaccharide or the live attenuated | Ni, Y., Springer, M. J., |
| | strain Ty21a | Guo, J., Finger-Baker, |
| | steD: fimbrial subunit | I., Wilson, J. P., Cobb, |
| | T2544 gene: possible outer membrane | R. R., . . . & Tizard, I. |
| | adhesin | (2017). |
| | OmpC: Outer membrane porin C | https://www.ncbi.nlm.nih.gov/ |
| | C-ter of sopB (last 261aa): inositol | pmc/articles/PMC5754192/ |
| | phosphatase | https://doi.org/10.1016/ |
| | SseB: Secreted effector protein | j.ygeno.2020.06.022 |
| | Flagellin | https://doi.org/10.1016/ |
| | Mig-14 | j.vaccine.2017.07.035 |
| | LptD: LPS-assembly protein D | https://doi.org/10.5402/ |
| | LptE: LPS-assembly protein E | 2012/512848 |
| | | https://doi.org/10.1016/ |
| | | j.vaccine.2009.02.092 |
| | | https://doi.org/10.1111/ |
| | | imm.12327 |
| | | https://doi.org/10.4049/ |
| | | jimmunol.1601357 |
| | | https://doi.org/10.1073/ |
| | | pnas.0401283101 |
| | | https://doi.org/10.1101/ |
| | | 521518 |
| Vaccines for Sexually | Gag, gp120, Pol, Nef, Env, Tat, Rev, Vpr, | US10894078B2 |
| Transmitted Diseases | Vif, Vpu | |
| Yersinia pestis | Caf1 | Chalton, D. A., |
| | low calcium response protein V (LcrV) | Musson, J. A., Flick- |
| | rV10 | Smith, H., Walker, N., |
| | recombinant F1 and V proteins | McGregor, A., Lamb, |
| | rF1-V: recombinant fusion protein of the F1 | H. K., . . . & Lakey, J. H. |
| | capsular protein and the LcrV protein | (2006). |
| | YadC: Yersinia adhesin A | https://journals.asm.org/ |
| | | doi/10.1128/iai.00437- |
| | | 06?permanently=true |
| | | Sun, W., & Singh, A. |
| | | K. (2019). |
| | | https://www.nature.com/ |
| | | articles/s41541-019- |
| | | 0105-9 |
| | | https://doi.org/10.1016/ |
| | | j.vaccine.2019.07.103 |
| | | https://doi.org/10.1007/ |
| | | 978-0-387-72124- |
| | | 8_37 |
| Yellow Fever | Live attenuated yellow fever antigens | Gibney, K. B., |
| | NS1: Non-structural protein 1 | Edupuganti, S., |
| | YFE: Yellow Fever Envelope | Panella, A. J., Kosoy, |
| | | O. I., Delorey, M. J., |
| | | Lanciotti, R. S., . . . & |
| | | Staples, J. E. (2012). |
| | | https://www.ncbi.nlm.nih.gov/ |
| | | pmc/articles/PMC3516084/ |
| | | https://doi.org/10.3390/ |
| | | vaccines9060622 |
| | | https://doi.org/10.4269/ |
| | | ajtmh.16-0293 |
| Other | Antigens for human papillomavirus (L1, L2, | US20210023199A1 |
| | E6, E7), SARS CoV (spike protein), | |
| | Staphylococcus aureus (IsdA, IsdB, toxin | |
| | antigens), Bordetella pertussis (toxin), | |
| | Plasmodium (NANP, CSP protein, ssp2, | |
| | ama1, msp142 antigens), Rabies virus (G, | |
| | N antigens), Staphylococcus aureus (toxin | |
| | antigen), Clostridium difficile (toxin antigen), | |
| | Candida albicans | |

Vaccines as discussed herein are suitably (although not exclusively), envisioned for direction towards intracellular pathogens, whether cytoplasmic or vesicular. Examples in this regard of intracellular cytoplasmic pathogens are viruses, Chlamydia spp., Rickettsia spp., Listeria monocytogenes, and protozoal parasites such as Plasmodium spp. Examples of vesicular intracellular pathogens include mycobacteria, Salmonella typhimurium, Leishmania spp., Listeria spp., Trypanosoma spp., Legionella pneumophila, Cryptococcus neoformans, Histoplasma, and Yersinia pestis.

In some embodiments, the coding mRNA can encode one or more viral proteins of the Severe acute respiratory syndrome coronavirus, like the severe acute respiratory syndrome coronavirus 2 virus (SARS-CoV-2), that is, the virus responsible for the Covid-19 pandemic. This virus has four structural proteins, the S (spike), E (envelope), M (membrane), and N (nucleocapsid) proteins. In some embodiments, the coding mRNA encodes all or part of the spike protein of SARS-CoV-2. In some embodiments, the mRNA encodes the prefusion form of the S protein ectodomain (amino acids 1 to 1208 with proline substitutions at residues 986 and 987; GenBank MN908947). In some embodiments, the mRNA encodes the Spike protein's Receptor Binding Domain or RBD (residues 319 to 591; GenBank MN908947). As an external part of this protein, this is a likely location for epitopes which could be recognised by the immune system. In some embodiments, the mRNA encodes all or part of the spike protein of a variant of SARS-CoV-2, for example, that of the Alpha, Beta, Gamma, Epsilon, Delta, Kappa, or Eta variants. In some embodiments, the mRNA includes one or more of the sequences recited in Table 6A below (SEQ ID NOs: 62 to 67), or a sequence with at least 90%, at least 95%, at least 98%, or at least 99% similarity thereto. In some embodiments, the coding mRNA for the spike protein or part thereof has been codon-optimised for expression in human or other mammalian cells. In some embodiments, one or more of the nucleosides used in the mRNA are been replaced by an isomer thereof. As example one, more or all of the uridine nucleosides in the mRNA construct are replaced by pseudouridine nucleosides. In one embodiment, the mRNA encodes the spike protein of the SARS-CoV-2 Delta variant, and the organ protecting MOP sequence of the mRNA comprises target sites for each of miRNA 122, miRNA 192 and miRNA 30a, and in another embodiment further comprises a target site for miRNA let7b. In other embodiments of the invention described in more detail below, the mRNA encodes a prefusion spike protein of the SARS-CoV-2 selected from non-codon optimized or human codon optimized Wuhan strain, beta variant or alpha variant, with or without a MOP sequence. The MOP sequence may be selected from one that comprises the following combinations of miRNA binding sequences: miRNA 122, miRNA 192 and miRNA 30a; and let7b, miRNA 126, and miRNA 30a; miRNA 122, miRNA 1, miRNA 203a, and miRNA 30a. It will be appreciated that other MOP sequences may be selected depending upon the particular context in which organ protection is required. As described herein, the selected MOP sequences may comprise miRNA binding sequences that are further optimised to ensure perfect match hybridisation with the respective target miRNA sequence in the body.

TABLE 6A
Exemplary mRNA constructs for a range of SARS-COV-2 spike
protein variants suitable for use in vaccine compositions
Prefusion Spike mRNA sequence from Wuhan strain with K986P
and V987P mutation
[SEQ ID NO: 62]
The codons are not optimized for human cellular expression
This mRNA does not contain a MOP sequence
AUGUUUGUUUUUCUUGUUUUAUUGCCACUAGUCUCUAGUCAGUGUGUUAAUCUUACAACCA
GAACUCAAUUACCCCCUGCAUACACUAAUUCUUUCACACGUGGUGUUUAUUACCCUGACAA
AGUUUUCAGAUCCUCAGUUUUACAUUCAACUCAGGACUUGUUCUUACCUUUCUUUUCCAAU
GUUACUUGGUUCCAUGCUAUACAUGUCUCUGGGACCAAUGGUACUAAGAGGUUUGAUAACC
CUGUCCUACCAUUUAAUGAUGGUGUUUAUUUUGCUUCCACUGAGAAGUCUAACAUAAUAAG
AGGCUGGAUUUUUGGUACUACUUUAGAUUCGAAGACCCAGUCCCUACUUAUUGUUAAUAAC
GCUACUAAUGUUGUUAUUAAAGUCUGUGAAUUUCAAUUUUGUAAUGAUCCAUUUUUGGGUG
UUUAUUACCACAAAAACAACAAAAGUUGGAUGGAAAGUGAGUUCAGAGUUUAUUCUAGUGC
GAAUAAUUGCACUUUUGAAUAUGUCUCUCAGCCUUUUCUUAUGGACCUUGAAGGAAAACAG
GGUAAUUUCAAAAAUCUUAGGGAAUUUGUGUUUAAGAAUAUUGAUGGUUAUUUUAAAAUAU
AUUCUAAGCACACGCCUAUUAAUUUAGUGCGUGAUCUCCCUCAGGGUUUUUCGGCUUUAGA
ACCAUUGGUAGAUUUGCCAAUAGGUAUUAACAUCACUAGGUUUCAAACUUUACUUGCUUUA
CAUAGAAGUUAUUUGACUCCUGGUGAUUCUUCUUCAGGUUGGACAGCUGGUGCUGCAGCUU
AUUAUGUGGGUUAUCUUCAACCUAGGACUUUUCUAUUAAAAUAUAAUGAAAAUGGAACCAU
UACAGAUGCUGUAGACUGUGCACUUGACCCUCUCUCAGAAACAAAGUGUACGUUGAAAUCC
UUCACUGUAGAAAAAGGAAUCUAUCAAACUUCUAACUUUAGAGUCCAACCAACAGAAUCUA
UUGUUAGAUUUCCUAAUAUUACAAACUUGUGCCCUUUUGGUGAAGUUUUUAACGCCACCAG
AUUUGCAUCUGUUUAUGCUUGGAACAGGAAGAGAAUCAGCAACUGUGUUGCUGAUUAUUCU
GUCCUAUAUAAUUCCGCAUCAUUUUCCACUUUUAAGUGUUAUGGAGUGUCUCCUACUAAAU
UAAAUGAUCUCUGCUUUACUAAUGUCUAUGCAGAUUCAUUUGUAAUUAGAGGUGAUGAAGU
CAGACAAAUCGCUCCAGGGCAAACUGGAAAGAUUGCUGAUUAUAAUUAUAAAUUACCAGAU
GAUUUUACAGGCUGCGUUAUAGCUUGGAAUUCUAACAAUCUUGAUUCUAAGGUUGGUGGUA
AUUAUAAUUACCUGUAUAGAUUGUUUAGGAAGUCUAAUCUCAAACCUUUUGAGAGAGAUAU
UUCAACUGAAAUCUAUCAGGCCGGUAGCACACCUUGUAAUGGUGUUGAAGGUUUUAAUUGU
UACUUUCCUUUACAAUCAUAUGGUUUCCAACCCACUAAUGGUGUUGGUUACCAACCAUACA
GAGUAGUAGUACUUUCUUUUGAACUUCUACAUGCACCAGCAACUGUUUGUGGACCUAAAAA
GUCUACUAAUUUGGUUAAAAACAAAUGUGUCAAUUUCAACUUCAAUGGUUUAACAGGCACA
GGUGUUCUUACUGAGUCUAACAAAAAGUUUCUGCCUUUCCAACAAUUUGGCAGAGACAUUG
CUGACACUACUGAUGCUGUCCGUGAUCCACAGACACUUGAGAUUCUUGACAUUACACCAUG
UUCUUUUGGUGGUGUCAGUGUUAUAACACCAGGAACAAAUACUUCUAACCAGGUUGCUGUU
CUUUAUCAGGAUGUUAACUGCACAGAAGUCCCUGUUGCUAUUCAUGCAGAUCAACUUACUC
CUACUUGGCGUGUUUAUUCUACAGGUUCUAAUGUUUUUCAAACACGUGCAGGCUGUUUAAU
AGGGGCUGAACAUGUCAACAACUCAUAUGAGUGUGACAUACCCAUUGGUGCAGGUAUAUGC
GCUAGUUAUCAGACUCAGACUAAUUCUCCUCGGCGGGCACGUAGUGUAGCUAGUCAAUCCA
UCAUUGCCUACACUAUGUCACUUGGUGCAGAAAAUUCAGUUGCUUACUCUAAUAACUCUAU
UGCCAUACCCACAAAUUUUACUAUUAGUGUUACCACAGAAAUUCUACCAGUGUCUAUGACC
AAGACAUCAGUAGAUUGUACAAUGUACAUUUGUGGUGAUUCAACUGAAUGCAGCAAUCUUU
UGUUGCAAUAUGGCAGUUUUUGUACACAAUUAAACCGUGCUUUAACUGGAAUAGCUGUUGA
ACAAGACAAAAACACCCAAGAAGUUUUUGCACAAGUCAAACAAAUUUACAAAACACCACCA
AUUAAAGAUUUUGGUGGUUUUAAUUUUUCACAAAUAUUACCAGAUCCAUCAAAACCAAGCA
AGAGGUCAUUUAUUGAAGAUCUACUUUUCAACAAAGUGACACUUGCAGAUGCUGGCUUCAU
CAAACAAUAUGGUGAUUGCCUUGGUGAUAUUGCUGCUAGAGACCUCAUUUGUGCACAAAAG
UUUAACGGCCUUACUGUUUUGCCACCUUUGCUCACAGAUGAAAUGAUUGCUCAAUACACUU
CUGCACUGUUAGCGGGUACAAUCACUUCUGGUUGGACCUUUGGUGCAGGUGCUGCAUUACA
AAUACCAUUUGCUAUGCAAAUGGCUUAUAGGUUUAAUGGUAUUGGAGUUACACAGAAUGUU
CUCUAUGAGAACCAAAAAUUGAUUGCCAACCAAUUUAAUAGUGCUAUUGGCAAAAUUCAAG
ACUCACUUUCUUCCACAGCAAGUGCACUUGGAAAACUUCAAGAUGUGGUCAACCAAAAUGC
ACAAGCUUUAAACACGCUUGUUAAACAACUUAGCUCCAAUUUUGGUGCAAUUUCAAGUGUU
UUAAAUGAUAUCCUUUCACGUCUUGACCCGCCGGAGGCUGAAGUGCAAAUUGAUAGGUUGA
UCACAGGCAGACUUCAAAGUUUGCAGACAUAUGUGACUCAACAAUUAAUUAGAGCUGCAGA
AAUCAGAGCUUCUGCUAAUCUUGCUGCUACUAAAAUGUCAGAGUGUGUACUUGGACAAUCA
AAAAGAGUUGAUUUUUGUGGAAAGGGCUAUCAUCUUAUGUCCUUCCCUCAGUCAGCACCUC
AUGGUGUAGUCUUCUUGCAUGUGACUUAUGUCCCUGCACAAGAAAAGAACUUCACAACUGC
UCCUGCCAUUUGUCAUGAUGGAAAAGCACACUUUCCUCGUGAAGGUGUCUUUGUUUCAAAU
GGCACACACUGGUUUGUAACACAAAGGAAUUUUUAUGAACCACAAAUCAUUACUACAGACA
ACACAUUUGUGUCUGGUAACUGUGAUGUUGUAAUAGGAAUUGUCAACAACACAGUUUAUGA
UCCUUUGCAACCUGAAUUAGACUCAUUCAAGGAGGAGUUAGAUAAAUAUUUUAAGAAUCAU
ACAUCACCAGAUGUUGAUUUAGGUGACAUCUCUGGCAUUAAUGCUUCAGUUGUAAACAUUC
AAAAAGAAAUUGACCGCCUCAAUGAGGUUGCCAAGAAUUUAAAUGAAUCUCUCAUCGAUCU
CCAAGAACUUGGAAAGUAUGAGCAGUAUAUAAAAUGGCCAUGGUACAUUUGGCUAGGUUUU
AUAGCUGGCUUGAUUGCCAUAGUAAUGGUGACAAUUAUGCUUUGCUGUAUGACCAGUUGCU
GUAGUUGUCUCAAGGGCUGUUGUUCUUGUGGAUCCUGCUGCAAAUUUGAUGAAGACGACUC
UGAGCCAGUGCUCAAAGGAGUCAAAUUACAUUACACAUAA
Prefusion Spike mRNA sequence from Wuhan strain with MOPV in
3′ UTR
[SEQ ID NO: 63]
Codons are not optimized for human cellular expression
MOPV sequence is shown at 3′ end with underlining comprises
binding sequences for miRNA-122-5P, miRNA-1-3P, miRNA-203a-
3P, miRNA-30a-5P
AUGUUUGUUUUUCUUGUUUUAUUGCCACUAGUCUCUAGUCAGUGUGUUAAUCUUACAACCA
GAACUCAAUUACCCCCUGCAUACACUAAUUCUUUCACACGUGGUGUUUAUUACCCUGACAA
AGUUUUCAGAUCCUCAGUUUUACAUUCAACUCAGGACUUGUUCUUACCUUUCUUUUCCAAU
GUUACUUGGUUCCAUGCUAUACAUGUCUCUGGGACCAAUGGUACUAAGAGGUUUGAUAACC
CUGUCCUACCAUUUAAUGAUGGUGUUUAUUUUGCUUCCACUGAGAAGUCUAACAUAAUAAG
AGGCUGGAUUUUUGGUACUACUUUAGAUUCGAAGACCCAGUCCCUACUUAUUGUUAAUAAC
GCUACUAAUGUUGUUAUUAAAGUCUGUGAAUUUCAAUUUUGUAAUGAUCCAUUUUUGGGUG
UUUAUUACCACAAAAACAACAAAAGUUGGAUGGAAAGUGAGUUCAGAGUUUAUUCUAGUGC
GAAUAAUUGCACUUUUGAAUAUGUCUCUCAGCCUUUUCUUAUGGACCUUGAAGGAAAACAG
GGUAAUUUCAAAAAUCUUAGGGAAUUUGUGUUUAAGAAUAUUGAUGGUUAUUUUAAAAUAU
AUUCUAAGCACACGCCUAUUAAUUUAGUGCGUGAUCUCCCUCAGGGUUUUUCGGCUUUAGA
ACCAUUGGUAGAUUUGCCAAUAGGUAUUAACAUCACUAGGUUUCAAACUUUACUUGCUUUA
CAUAGAAGUUAUUUGACUCCUGGUGAUUCUUCUUCAGGUUGGACAGCUGGUGCUGCAGCUU
AUUAUGUGGGUUAUCUUCAACCUAGGACUUUUCUAUUAAAAUAUAAUGAAAAUGGAACCAU
UACAGAUGCUGUAGACUGUGCACUUGACCCUCUCUCAGAAACAAAGUGUACGUUGAAAUCC
UUCACUGUAGAAAAAGGAAUCUAUCAAACUUCUAACUUUAGAGUCCAACCAACAGAAUCUA
UUGUUAGAUUUCCUAAUAUUACAAACUUGUGCCCUUUUGGUGAAGUUUUUAACGCCACCAG
AUUUGCAUCUGUUUAUGCUUGGAACAGGAAGAGAAUCAGCAACUGUGUUGCUGAUUAUUCU
GUCCUAUAUAAUUCCGCAUCAUUUUCCACUUUUAAGUGUUAUGGAGUGUCUCCUACUAAAU
UAAAUGAUCUCUGCUUUACUAAUGUCUAUGCAGAUUCAUUUGUAAUUAGAGGUGAUGAAGU
CAGACAAAUCGCUCCAGGGCAAACUGGAAAGAUUGCUGAUUAUAAUUAUAAAUUACCAGAU
GAUUUUACAGGCUGCGUUAUAGCUUGGAAUUCUAACAAUCUUGAUUCUAAGGUUGGUGGUA
AUUAUAAUUACCUGUAUAGAUUGUUUAGGAAGUCUAAUCUCAAACCUUUUGAGAGAGAUAU
UUCAACUGAAAUCUAUCAGGCCGGUAGCACACCUUGUAAUGGUGUUGAAGGUUUUAAUUGU
UACUUUCCUUUACAAUCAUAUGGUUUCCAACCCACUAAUGGUGUUGGUUACCAACCAUACA
GAGUAGUAGUACUUUCUUUUGAACUUCUACAUGCACCAGCAACUGUUUGUGGACCUAAAAA
GUCUACUAAUUUGGUUAAAAACAAAUGUGUCAAUUUCAACUUCAAUGGUUUAACAGGCACA
GGUGUUCUUACUGAGUCUAACAAAAAGUUUCUGCCUUUCCAACAAUUUGGCAGAGACAUUG
CUGACACUACUGAUGCUGUCCGUGAUCCACAGACACUUGAGAUUCUUGACAUUACACCAUG
UUCUUUUGGUGGUGUCAGUGUUAUAACACCAGGAACAAAUACUUCUAACCAGGUUGCUGUU
CUUUAUCAGGAUGUUAACUGCACAGAAGUCCCUGUUGCUAUUCAUGCAGAUCAACUUACUC
CUACUUGGCGUGUUUAUUCUACAGGUUCUAAUGUUUUUCAAACACGUGCAGGCUGUUUAAU
AGGGGCUGAACAUGUCAACAACUCAUAUGAGUGUGACAUACCCAUUGGUGCAGGUAUAUGC
GCUAGUUAUCAGACUCAGACUAAUUCUCCUCGGCGGGCACGUAGUGUAGCUAGUCAAUCCA
UCAUUGCCUACACUAUGUCACUUGGUGCAGAAAAUUCAGUUGCUUACUCUAAUAACUCUAU
UGCCAUACCCACAAAUUUUACUAUUAGUGUUACCACAGAAAUUCUACCAGUGUCUAUGACC
AAGACAUCAGUAGAUUGUACAAUGUACAUUUGUGGUGAUUCAACUGAAUGCAGCAAUCUUU
UGUUGCAAUAUGGCAGUUUUUGUACACAAUUAAACCGUGCUUUAACUGGAAUAGCUGUUGA
ACAAGACAAAAACACCCAAGAAGUUUUUGCACAAGUCAAACAAAUUUACAAAACACCACCA
AUUAAAGAUUUUGGUGGUUUUAAUUUUUCACAAAUAUUACCAGAUCCAUCAAAACCAAGCA
AGAGGUCAUUUAUUGAAGAUCUACUUUUCAACAAAGUGACACUUGCAGAUGCUGGCUUCAU
CAAACAAUAUGGUGAUUGCCUUGGUGAUAUUGCUGCUAGAGACCUCAUUUGUGCACAAAAG
UUUAACGGCCUUACUGUUUUGCCACCUUUGCUCACAGAUGAAAUGAUUGCUCAAUACACUU
CUGCACUGUUAGCGGGUACAAUCACUUCUGGUUGGACCUUUGGUGCAGGUGCUGCAUUACA
AAUACCAUUUGCUAUGCAAAUGGCUUAUAGGUUUAAUGGUAUUGGAGUUACACAGAAUGUU
CUCUAUGAGAACCAAAAAUUGAUUGCCAACCAAUUUAAUAGUGCUAUUGGCAAAAUUCAAG
ACUCACUUUCUUCCACAGCAAGUGCACUUGGAAAACUUCAAGAUGUGGUCAACCAAAAUGC
ACAAGCUUUAAACACGCUUGUUAAACAACUUAGCUCCAAUUUUGGUGCAAUUUCAAGUGUU
UUAAAUGAUAUCCUUUCACGUCUUGACCCGCCGGAGGCUGAAGUGCAAAUUGAUAGGUUGA
UCACAGGCAGACUUCAAAGUUUGCAGACAUAUGUGACUCAACAAUUAAUUAGAGCUGCAGA
AAUCAGAGCUUCUGCUAAUCUUGCUGCUACUAAAAUGUCAGAGUGUGUACUUGGACAAUCA
AAAAGAGUUGAUUUUUGUGGAAAGGGCUAUCAUCUUAUGUCCUUCCCUCAGUCAGCACCUC
AUGGUGUAGUCUUCUUGCAUGUGACUUAUGUCCCUGCACAAGAAAAGAACUUCACAACUGC
UCCUGCCAUUUGUCAUGAUGGAAAAGCACACUUUCCUCGUGAAGGUGUCUUUGUUUCAAAU
GGCACACACUGGUUUGUAACACAAAGGAAUUUUUAUGAACCACAAAUCAUUACUACAGACA
ACACAUUUGUGUCUGGUAACUGUGAUGUUGUAAUAGGAAUUGUCAACAACACAGUUUAUGA
UCCUUUGCAACCUGAAUUAGACUCAUUCAAGGAGGAGUUAGAUAAAUAUUUUAAGAAUCAU
ACAUCACCAGAUGUUGAUUUAGGUGACAUCUCUGGCAUUAAUGCUUCAGUUGUAAACAUUC
AAAAAGAAAUUGACCGCCUCAAUGAGGUUGCCAAGAAUUUAAAUGAAUCUCUCAUCGAUCU
CCAAGAACUUGGAAAGUAUGAGCAGUAUAUAAAAUGGCCAUGGUACAUUUGGCUAGGUUUU
AUAGCUGGCUUGAUUGCCAUAGUAAUGGUGACAAUUAUGCUUUGCUGUAUGACCAGUUGCU
GUAGUUGUCUCAAGGGCUGUUGUUCUUGUGGAUCCUGCUGCAAAUUUGAUGAAGACGACUC

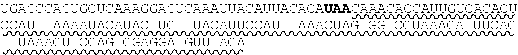

Prefusion Spike mRNA sequence from Wuhan strain with MOPC in
3′ UTR
[SEQ ID NO: 64]
3′ MOP for miRNA-122-5P, miRNA-192-5P, miRNA-30a-5P MOPC,
underlined portion)
Codons optimized for human cellular expression
AUGUUCGUUUUCUUGGUCCUGCUUCCCCUGGUGUCUUCACAGUGCGUGAAUCUGACCACCA
GAACACAGCUGCCUCCAGCAUACACCAACAGCUUCACCAGAGGCGUGUAUUAUCCUGACAA
GGUGUUUCGCUCCAGCGUGCUGCACAGCACCCAGGACCUUUUUCUGCCUUUUUUCUCCAAC
GUGACAUGGUUCCACGCAAUCCACGUGAGCGGAACCAACGGAACGAAGAGAUUCGACAACC
CUGUGCUGCCCUUCAACGACGGAGUGUACUUCGCCAGCACAGAGAAGAGCAACAUCAUCCG
GGGCUGGAUCUUCGGAACCACCCUGGACAGCAAAACCCAAUCUCUGCUUAUCGUGAACAAC
GCAACCAACGUGGUGAUCAAGGUGUGUGAAUUCCAAUUUUGUAACGACCCAUUCCUGGGAG
UGUACUACCAUAAGAACAACAAGAGCUGGAUGGAAAGCGAGUUCCGGGUGUACAGCAGCGC
CAACAACUGCACCUUCGAGUACGUGAGUCAGCCCUUUCUGAUGGACCUGGAAGGCAAGCAG
GGAAAUUUCAAGAAUCUGAGAGAGUUCGUGUUCAAAAACAUCGAUGGCUAUUUCAAGAUCU
AUAGCAAGCACACCCCUAUCAACCUGGUGAGAGAUCUGCCCCAGGGCUUCAGCGCCCUGGA
GCCUCUGGUAGACCUACCUAUCGGCAUCAACAUAACGAGAUUUCAGACCCUGUUGGCUCUU
CAUAGGAGCUACCUGACCCCCGGCGAUUCUAGCAGCGGAUGGACAGCCGGCGCCGCUGCCU
ACUACGUUGGCUACCUGCAACCUCGGACAUUCCUGCUGAAAUACAAUGAGAACGGCACUAU
CACCGAUGCCGUGGACUGUGCCCUGGAUCCUCUGAGCGAAACCAAGUGCACCCUGAAGAGC
UUUACCGUGGAAAAGGGCAUCUACCAGACCAGCAAUUUCCGGGUGCAGCCUACAGAGAGCA
UCGUGAGAUUCCCCAACAUCACCAAUCUGUGUCCUUUCGGCGAGGUGUUCAACGCUACAAG
AUUCGCAAGCGUGUACGCCUGGAAUCGGAAGCGGAUCAGUAACUGUGUGGCCGAUUAUUCG
GUGCUGUAUAAUUCUGCCAGCUUUAGCACCUUCAAGUGCUACGGUGUGAGCCCUACCAAAC
UCAACGACCUGUGCUUCACCAACGUGUAUGCCGACUCUUUCGUGAUCCGGGGCGACGAGGU
GCGGCAGAUCGCCCCCGGACAGACAGGCAAAAUCGCCGACUACAACUACAAGCUGCCUGAC
GACUUCACAGGGUGCGUUAUCGCCUGGAACAGCAACAAUCUGGAUUCAAAGGUGGGCGGAA
ACUACAACUACCUGUACAGACUGUUCAGAAAGUCCAACCUGAAGCCCUUUGAGAGAGACAU
CUCUACAGAAAUCUACCAGGCCGGCUCCACCCCAUGCAACGGCGUGGAAGGCUUCAACUGC
UACUUCCCCCUGCAGAGCUACGGCUUUCAGCCUACCAAUGGCGUCGGUUACCAGCCUUACC
GCGUGGUCGUUCUAUCCUUCGAGCUGCUGCACGCCCCUGCUACAGUGUGCGGACCUAAGAA
GAGCACAAACCUGGUCAAAAACAAGUGUGUCAACUUCAACUUCAACGGCCUGACCGGCACA
GGCGUACUGACAGAAAGCAACAAGAAGUUCCUGCCUUUCCAGCAGUUUGGCAGAGAUAUCG
CUGAUACCACAGACGCCGUGCGGGAUCCUCAGACACUGGAAAUCCUGGACAUCACACCCUG
CAGCUUUGGCGGCGUGUCUGUCAUCACCCCAGGCACCAACACGUCCAACCAAGUGGCCGUG
CUGUACCAGGACGUCAACUGCACCGAGGUGCCCGUUGCUAUCCACGCCGAUCAGCUAACUC
CUACCUGGCGGGUUUAUAGCACCGGAUCUAACGUGUUCCAGACCAGGGCCGGAUGUCUGAU
CGGCGCUGAACACGUAAACAAUAGCUACGAGUGUGAUAUCCCUAUCGGAGCCGGCAUCUGU
GCCAGCUACCAGACCCAGACAAAUUCUCCACGGAGAGCUAGAAGCGUCGCCUCUCAGAGCA
UCAUCGCCUACACCAUGAGCCUGGGCGCCGAAAACUCUGUGGCCUACAGCAACAACAGCAU
CGCCAUUCCCACCAACUUUACAAUCAGCGUGACAACAGAGAUCCUGCCUGUGAGCAUGACC
AAAACCAGCGUGGACUGCACCAUGUACAUCUGCGGCGACAGCACCGAAUGCUCUAACCUUC
UGCUGCAAUACGGCAGCUUCUGCACUCAGCUGAACAGAGCCCUGACCGGCAUCGCCGUGGA
GCAGGAUAAGAACACCCAGGAGGUGUUCGCCCAGGUGAAACAAAUCUACAAGACACCUCCC
AUCAAGGACUUCGGCGGAUUUAACUUCAGCCAGAUCCUGCCUGACCCAUCUAAGCCUAGCA
AGCGGUCCUUUAUCGAGGACUUGCUGUUCAACAAGGUGACCCUGGCCGAUGCCGGCUUUAU
CAAGCAGUACGGCGACUGCCUUGGCGACAUCGCCGCCAGAGACCUGAUCUGCGCCCAGAAG
UUUAACGGCCUGACAGUGCUGCCUCCUCUGCUGACCGACGAAAUGAUCGCCCAGUAUACCA
GCGCUCUGCUGGCGGGUACCAUCACCAGCGGCUGGACCUUCGGCGCCGGCGCUGCUCUGCA
GAUCCCUUUCGCCAUGCAGAUGGCCUACCGGUUCAACGGCAUUGGCGUGACCCAGAACGUG
CUGUACGAGAAUCAGAAGCUGAUCGCCAACCAGUUCAACAGCGCUAUCGGAAAAAUCCAGG
ACUCUCUGAGCUCUACUGCCUCUGCUCUUGGGAAACUGCAGGACGUGGUUAAUCAGAACGC
CCAGGCCCUCAAUACCCUGGUGAAGCAACUGUCGAGCAAUUUUGGCGCCAUCAGCAGCGUG
CUGAAUGACAUUCUGUCUAGACUGGACCCUCCAGAAGCUGAGGUGCAGAUUGACAGACUGA
UCACAGGCAGACUGCAGAGCCUGCAGACCUACGUGACCCAACAACUGAUCAGAGCCGCCGA
GAUUAGGGCCUCUGCCAAUCUGGCCGCCACGAAAAUGUCCGAGUGCGUCCUGGGCCAGUCA
AAGAGAGUGGAUUUCUGCGGCAAGGGAUACCACCUGAUGAGCUUCCCCCAGAGCGCUCCGC
ACGGCGUGGUGUUUCUGCACGUGACCUACGUGCCAGCCCAGGAGAAGAACUUCACCACGGC
CCCUGCCAUCUGCCACGACGGCAAGGCCCACUUCCCCAGAGAAGGAGUGUUCGUGAGCAAU
GGCACACACUGGUUCGUGACACAAAGAAACUUCUACGAGCCUCAGAUCAUCACAACCGAUA
ACACCUUCGUGAGCGGCAAUUGCGACGUGGUGAUCGGCAUUGUGAACAACACCGUGUACGA
CCCCCUGCAGCCCGAGCUGGAUAGCUUCAAAGAGGAACUGGAUAAGUACUUCAAGAACCAC
ACCAGCCCUGAUGUGGAUCUGGGCGACAUUUCUGGCAUCAACGCCUCUGUCGUGAACAUCC
AGAAAGAGAUAGAUAGACUGAACGAGGUUGCAAAGAACCUGAACGAAAGCCUGAUCGACUU
GCAGGAGCUCGGCAAGUACGAGCAGUACAUCAAGUGGCCUUGGUACAUUUGGCUGGGCUUU
AUCGCCGGACUGAUCGCCAUCGUGAUGGUCACAAUCAUGCUGUGCUGCAUGACAAGUUGCU
GUUCCUGCCUGAAGGGCUGCUGUAGCUGUGGAAGCUGCUGUAAAUUCGACGAAGAUGACAG

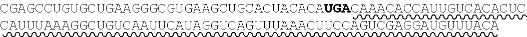

Prefusion Spike mRNA sequence of the South Africa variant
(B.1.351 or Beta variant)with MOPC in 3′ UTR
[SEQ ID NO: 65]
3′ MOP for miRNA-122-5P, miRNA-192-5P, miRNA-30a-5P MOPC,
underlined portion)
Codons optimized for human cellular expression
AUGUUCGUGUUCCUGGUGUUACUGCCCCUGGUGUCUUCUCAGUGCGUCAACUUCACCACAA
GAACACAGCUGCCUCCUGCCUAUACAAACAGCUUUACCCGGGGAGUGUACUACCCCGAUAA
AGUGUUCCGGAGCUCUGUGCUGCACAGCACACAGGACUUGUUCCUGCCUUUCUUCAGCAAU
GUGACAUGGUUCCACGCCAUCCACGUCUCCGGCACAAACGGCACCAAGAGGUUCGCCAAUC
CUGUGCUGCCAUUCAAUGAUGGCGUGUAUUUCGCCAGCACAGAGAAGUCUAACAUCAUCAG
AGGCUGGAUCUUCGGCACCACCCUCGACUCUAAAACCCAGAGCCUGCUGAUAGUGAACAAC
GCCACAAACGUGGUGAUCAAGGUCUGUGAAUUCCAGUUCUGCAACGACCCAUUCCUGGGCG
UGUACUACCACAAGAAUAACAAGAGCUGGAUGGAAUCUGAGUUCAGAGUGUAUUCAUCAGC
CAACAACUGCACAUUCGAGUACGUGUCUCAGCCAUUCCUGAUGGACCUGGAAGGCAAGCAG
GGCAAUUUCAAGAAUCUCAGAGAGUUCGUCUUCAAGAACAUCGACGGCUACUUUAAGAUCU
AUAGCAAGCACACCCCUAUCAACCUGGUUCGGGGCCUGCCCCAGGGCUUUAGCGCCCUGGA
ACCUCUGGUGGAUCUGCCAAUUGGCAUCAACAUCACCCGGUUUCAGACACUGCACAUCAGC
UACCUGACACCUGGCGACAGCAGCAGCGGCUGGACCGCCGGCGCCGCCGCCUACUACGUCG
GCUACCUGCAGCCCCGGACCUUCCUGCUGAAGUACAAUGAAAAUGGCACCAUCACAGACGC
CGUGGAUUGCGCCCUGGACCCUCUGUCUGAAACAAAGUGCACCCUGAAAAGCUUCACCGUG
GAAAAGGGCAUAUACCAGACCUCCAACUUCCGGGUGCAGCCUACAGAGUCUAUCGUGAGAU
UCCCCAACAUCACCAAUCUGUGUCCUUUUGGCGAGGUGUUCAACGCCACCAGAUUCGCAAG
CGUGUACGCCUGGAACCGGAAGAGGAUCAGCAACUGCGUGGCAGAUUACAGCGUGCUCUAC
AACAGCGCCAGUUUCUCUACCUUUAAGUGCUACGGCGUCAGCCCUACAAAACUGAACGAUC
UGUGCUUCACCAACGUGUACGCCGAUUCCUUUGUGAUACGGGGCGACGAAGUUAGACAGAU
CGCCCCUGGACAGACAGGAAAUAUCGCCGACUACAACUAUAAGCUGCCUGACGACUUCACC
GGCUGCGUCAUCGCUUGGAACUCCAACAACCUGGAUUCCAAGGUGGGCGGAAACUACAACU
ACCUGUACAGACUGUUCAGAAAGAGCAACCUGAAACCUUUCGAGAGGGACAUCAGCACAGA
GAUCUACCAGGCCGGCAGCACCCCCUGUAAUGGAGUCAAAGGCUUCAAUUGCUACUUCCCU
CUGCAGUCUUACGGCUUCCAGCCAACAUACGGCGUGGGCUACCAGCCCUACCGGGUGGUUG
UGCUGUCCUUCGAGCUGCUGCAUGCCCCAGCCACAGUAUGCGGUCCUAAGAAAAGCACCAA
CCUGGUGAAGAACAAAUGUGUGAACUUUAACUUUAACGGCCUGACCGGCACCGGCGUGCUG
ACCGAAUCCAAUAAGAAGUUCCUGCCGUUCCAGCAGUUUGGCAGAGAUAUCGCCGACACCA
CAGACGCCGUGAGAGACCCCCAGACCCUGGAAAUCCUGGACAUCACCCCUUGCUCCUUUGG
AGGGGUGAGCGUGAUCACCCCGGGCACAAACACCAGCAACCAGGUGGCCGUGCUGUACCAG
GGCGUGAAUUGUACCGAGGUGCCUGUGGCGAUCCACGCCGAUCAGCUGACCCCUACCUGGC
GGGUGUACAGCACCGGAUCUAACGUGUUCCAAACAAGAGCCGGCUGUCUGAUCGGAGCUGA
ACACGUGAACAACUCUUACGAGUGUGACAUUCCUAUCGGCGCCGGCAUCUGCGCCUCUUAU
CAGACCCAGACCAACAGCCCCAGACGUGCCAGAUCUGUGGCCUCUCAGAGCAUCAUCGCCU
ACACCAUGUCUCUGGGAGUGGAAAACUCCGUGGCUUACAGCAACAAUUCUAUCGCCAUCCC
CACCAACUUUACAAUCAGCGUGACCACCGAGAUACUGCCUGUGUCCAUGACAAAGACCAGC
GUGGACUGCACUAUGUACAUCUGCGGCGACAGCACAGAAUGCAGCAACCUGCUGCUGCAGU
ACGGAAGCUUUUGUACUCAGCUGAACAGAGCCCUGACUGGCAUCGCUGUUGAGCAGGAUAA
GAAUACUCAGGAGGUCUUCGCUCAAGUGAAGCAGAUCUACAAGACCCCUCCAAUCAAGGAC
UUCGGCGGCUUCAACUUCAGCCAAAUUCUGCCUGAUCCUAGCAAGCCCAGCAAGCGGAGCU
UCAUCGAGGACCUGCUGUUUAACAAAGUGACACUUGCCGACGCCGGAUUCAUUAAGCAGUA
UGGCGACUGCCUGGGCGACAUCGCCGCGAGAGAUUUGAUCUGCGCCCAAAAGUUCAACGGC
CUCACCGUGCUGCCUCCUCUUCUGACCGACGAGAUGAUCGCUCAGUACACCAGCGCUCUUC
UGGCCGGCACAAUCACCAGCGGCUGGACAUUUGGCGCUGGUGCCGCCCUCCAGAUCCCUUU
CGCCAUGCAGAUGGCCUACAGAUUCAACGGCAUCGGCGUCACCCAAAACGUGCUCUAUGAG
AACCAGAAACUUAUCGCUAAUCAGUUCAACUCUGCCAUCGGCAAGAUCCAAGAUAGCCUGU
CCUCCACCGCUAGCGCCCUGGGAAAGCUCCAGGACGUGGUGAAUCAGAACGCCCAAGCCCU
GAACACCCUGGUGAAACAGCUGAGCAGCAACUUCGGCGCUAUCAGCUCCGUUCUGAACGAC
AUUCUGUCUAGACUGGACCCUCCUGAGGCCGAGGUCCAGAUCGAUAGACUGAUCACUGGAC
GCCUGCAAUCACUGCAAACAUACGUGACCCAGCAGCUGAUUAGAGCCGCCGAGAUCAGAGC
CUCAGCAAAUCUGGCCGCCACGAAGAUGAGCGAGUGCGUGCUGGGCCAGAGCAAGAGAGUC
GACUUUUGCGGCAAAGGCUACCACCUGAUGAGCUUCCCUCAGAGCGCCCCACACGGCGUGG
UGUUCCUGCAUGUGACCUACGUGCCCGCCCAGGAAAAGAACUUUACCACCGCCCCUGCUAU
CUGUCACGACGGCAAGGCCCACUUCCCUCGCGAGGGCGUGUUCGUCAGCAACGGCACCCAC
UGGUUCGUGACACAACGUAACUUCUACGAGCCUCAGAUCAUAACCACCGAUAACACAUUCG
UGAGCGGCAAUUGCGAUGUGGUGAUCGGAAUCGUGAACAACACCGUGUACGACCCGCUGCA
GCCCGAGCUGGACAGCUUCAAAGAGGAACUGGAUAAGUACUUUAAGAACCACACUUCUCCA
GACGUGGACCUGGGCGAUAUCAGCGGAAUCAACGCUUCCGUGGUGAACAUCCAGAAGGAAA
UCGACAGACUGAACGAGGUGGCUAAAAACCUGAAUGAGAGCCUGAUCGACCUGCAGGAGCU
GGGAAAAUACGAACAGUACAUCAAGUGGCCUUGGUACAUCUGGCUGGGCUUUAUCGCUGGC
CUGAUCGCCAUCGUGAUGGUGACCAUCAUGCUGUGCUGUAUGACCAGCUGUUGUAGCUGCC
UGAAGGGUUGCUGUUCCUGCGGAAGCUGCUGCAAGUUCGACGAGGAUGACAGCGAGCCCGU

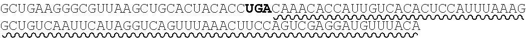

Prefusion Spike mRNA sequence of the Delta variant B.1.617.2
first identified in India) with MOPV in 3′ UTR
[SEQ ID NO: 66]
3′ MOP for miRNA-122-5P, miRNA-1-3P, miRNA-203a-3P, miRNA-
30a-5P (MOPV, underlined portion)
Codons optimized for human cellular expression
AUGUUCGUCUUCCUGGUGCUGCUGCCCCUGGUGAGCUCUCAGUGCGUGAAUCUGAGAACCC
GGACACAGCUGCCUCCUGCCUACACAAACAGCUUUACAAGAGGCGUCUACUACCCUGACAA
GGUGUUCCGGUCGAGCGUGCUGCAUUCUACCCAGGAUUUGUUUCUUCCUUUUUUCAGUAAC
GUGACAUGGUUCCACGCCAUCCACGUGUCCGGAACCAACGGCACCAAGAGAUUCGACAACC
CUGUGCUGCCUUUCAACGACGGAGUGUAUUUCGCCUCUACCGAGAAGAGCAACAUCAUUCG
GGGAUGGAUCUUCGGAACCACCCUCGACAGCAAGACACAGAGCCUGCUAAUAGUCAACAAC
GCUACCAACGUGGUGAUUAAGGUGUGCGAGUUCCAAUUUUGUAACGAUCCUUUCCUGGGAG
UUUAUUACCAUAAGAACAAUAAAAGCUGGAUGGAAAGCGAGGUGUACAGCAGCGCAAACAA
CUGCACAUUCGAGUAUGUGAGCCAACCUUUCCUGAUGGACCUGGAAGGCAAGCAGGGGAAC
UUCAAGAACCUGAGGGAAUUCGUGUUUAAGAACAUCGACGGCUACUUCAAGAUCUACAGCA
AGCACACACCAAUUAACCUCGUUAGAGAUCUGCCACAGGGCUUCAGUGCCCUGGAACCCCU
GGUGGAUCUGCCCAUCGGAAUCAACAUCACCAGAUUCCAGACCCUCCUGGCCCUGCACAGA
AGCUAUCUGACCCCUGGCGAUUCUAGCUCUGGCUGGACAGCUGGCGCCGCUGCUUACUACG
UGGGCUACCUGCAGCCUAGAACAUUCCUGCUCAAGUACAACGAGAAUGGCACAAUCACCGA
CGCCGUUGACUGCGCCCUGGAUCCUUUGUCUGAGACAAAGUGCACUCUGAAGAGCUUCACC
GUGGAAAAGGGCAUCUACCAGACAUCUAACUUCAGAGUGCAGCCUACAGAGAGCAUCGUGC
GGUUCCCCAACAUAACAAACCUGUGUCCAUUCGGAGAAGUGUUUAAUGCCACCAGAUUCGC
UAGCGUGUACGCCUGGAACCGGAAGAGAAUCAGCAACUGCGUCGCCGACUACUCCGUGCUG
UACAAUAGCGCCUCUUUCAGCACCUUUAAGUGUUACGGCGUCUCUCCAACAAAGCUGAACG
ACCUGUGCUUCACAAACGUGUACGCCGACAGCUUCGUGAUCCGGGGCGACGAAGUGCGGCA
GAUUGCACCUGGUCAGACUGGGAAAAUCGCAGAUUACAACUACAAGCUGCCAGAUGAUUUU
ACCGGCUGUGUGAUCGCCUGGAAUAGCAAUAACCUGGACAGCAAAGUGGGCGGCAAUUACA
ACUACCGGUACAGACUGUUCCGGAAGAGCAAUCUGAAGCCUUUUGAGAGAGACAUCUCCAC
AGAGAUCUACCAGGCCGGCUCUAAGCCUUGCAACGGCGUGGAGGGGUUUAAUUGCUACUUC
CCUCUGCAGUCUUACGGGUUCCAGCCCACCAACGGCGUGGGCUAUCAGCCUUACAGAGUGG
UGGUGCUGUCUUUCGAACUGCUGCACGCCCCUGCUACCGUGUGCGGGCCUAAGAAGUCCAC
CAACCUUGUGAAGAACAAGUGUGUGAACUUCAACUUCAAUGGCCUGACCGGAACCGGCGUG
UUGACCGAAUCUAACAAGAAAUUCCUGCCGUUCCAACAGUUCGGCAGAGAUAUUGCCGACA
CCACCGAUGCCGUGCGGGACCCCCAAACCCUGGAAAUCCUGGAUAUCACCCCAUGCAGCUU
CGGCGGCGUGUCUGUUAUCACCCCCGGCACAAACACGAGCAACCAGGUCGCCGUGCUCUAC
CAGGGCGUGAACUGCACAGAAGUGCCCGUGGCUAUCCACGCCGAUCAGCUGACACCCACAU
GGCGGGUGUACAGCACAGGAUCUAACGUUUUCCAGACAAGAGCUGGCUGCCUUAUGGCGCU
UGAACACGUGAAUAACAGCUACGAGUGUGACAUCCCAAUCGGCGCCGGCAUCUGCGCCUCC
UACCAGACCCAGACCAACAGCAGAAGGAGAGCCCGGAGCGUGGCCAGCCAGUCUAUCAUCG
CCUACACAAUGAGCCUGGGCGCCGAAAACUCCGUGGCCUAUAGCAACAACUCCAUCGCUAU
CCCUACCAACUUCACCAUCAGCGUGACAACGGAAAUUCUGCCUGUGAGCAUGACCAAGACC
UCUGUGGACUGUACAAUGUACAUCUGCGGCGACUCUACAGAAUGCAGCAACCUGCUGCUGC
AGUACGGCAGCUUUUGCACCCAGCUUAAUAGAGCCCUGACCGGAAUCGCCGUGGAACAGGA
CAAGAACACCCAGGAGGUCUUCGCCCAGGUGAAACAGAUCUACAAGACCCCUCCUAUUAAG
GACUUCGGCGGAUUUAACUUCAGCCAGAUCCUGCCUGACCCUAGCAAGCCCAGCAAAAGAA
GCUUCAUCGAGGACCUCCUGUUCAACAAAGUGACCCUGGCCGACGCUGGCUUUAUCAAGCA
GUAUGGCGACUGCCUGGGCGACAUCGCUGCUAGGGACCUGAUCUGUGCCCAGAAGUUCAAC
GGCCUGACAGUGCUGCCUCCUCUGCUGACCGAUGAAAUGAUCGCCCAGUACACAAGCGCCC
UGCUGGCCGGCACCAUCACCAGCGGCUGGACCUUUGGAGCCGGCGCCGCCCUGCAGAUCCC
CUUUGCCAUGCAGAUGGCCUAUCGGUUCAACGGAAUCGGCGUGACCCAAAACGUACUGUAC
GAGAACCAGAAGCUGAUCGCCAAUCAAUUUAAUAGCGCCAUCGGUAAAAUCCAGGAUAGCC
UGAGCUCCACUGCCAGCGCCCUGGGCAAACUGCAGAACGUGGUGAACCAGAACGCCCAAGC
UCUGAACACCCUGGUGAAGCAGCUGUCUUCCAACUUUGGUGCUAUCUCUAGCGUCCUGAAU
GAUAUCCUGAGCAGACUGGACCCCCCCGAGGCCGAGGUGCAGAUCGAUAGACUGAUCACCG
GCAGACUGCAAUCGCUGCAAACUUACGUGACCCAGCAGCUGAUCAGAGCCGCCGAGAUCAG
AGCUAGCGCCAACCUGGCCGCCACUAAGAUGAGCGAGUGCGUUCUGGGCCAGAGUAAGCGG
GUGGACUUCUGUGGCAAGGGAUACCACCUGAUGUCUUUUCCACAGAGCGCCCCUCACGGCG
UGGUGUUCCUGCACGUUACCUACGUGCCAGCCCAGGAGAAGAACUUCACCACAGCCCCUGC
CAUCUGCCACGACGGCAAGGCCCACUUCCCUAGAGAGGGCGUGUUCGUCAGCAACGGCACC
CACUGGUUCGUGACGCAAAGAAACUUCUACGAGCCCCAGAUCAUUACCACCGACAAUACCU
UCGUAUCCGGCAACUGCGACGUGGUGAUCGGCAUCGUGAACAACACAGUGUACGACCCUCU
GCAGCCUGAGCUGGACUCUUUCAAGGAAGAGCUGGACAAGUAUUUCAAGAACCACACCAGC
CCUGAUGUGGACCUGGGCGACAUCAGCGGAAUCAAUGCCUCAGUGGUGAACAUCCAGAAAG
AGAUCGACAGACUGAACGAGGUCGCCAAGAACCUGAAUGAGAGCCUGAUCGACCUGCAGGA
GCUGGGCAAGUACGAGCAAUACAUCAAGUGGCCUUGGUACAUCUGGCUGGGCUUCAUCGCC
GGCCUGAUUGCCAUCGUGAUGGUGACCAUCAUGCUGUGUUGCAUGACCAGUUGCUGUAGUU
GCCUGAAAGGCUGCUGUUCUUGCGGCAGCUGCUGCAAAUUCGAUGAGGACGACUCCGAGCC

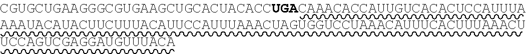

Prefusion Spike mRNA sequence of the UK/Kent variant
(B.1.1.7 or Alpha variant) with MOPC in 3′ UTR
[SEQ ID NO: 67]
3′ MOP for miRNA-122-5P, miRNA-192-5P, miRNA-30a-5P MOPC,
underlined portion)
Codons optimized for human cellular expression
AUGUUCGUGUUUCUGGUCCUGCUGCCCCUGGUGUCCUCCCAGUGCGUGAACCUGACGACCA
GAACACAACUGCCUCCUGCCUACACCAACAGCUUUACAAGAGGCGUCUAUUACCCCGACAA
GGUGUUCCGGAGCUCCGUCCUGCACUCUACCCAGGACCUUUUCCUGCCUUUUUUCAGCAAC
GUGACAUGGUUCCACGCCAUCAGCGGUACCAACGGCACCAAGCGCUUCGACAACCCUGUGC
UGCCAUUUAACGACGGAGUGUAUUUCGCCUCCACAGAAAAGUCGAACAUCAUCAGGGGCUG
GAUCUUCGGCACCACACUGGACAGCAAGACCCAGAGCCUGCUGAUCGUGAAUAACGCCACA
AACGUGGUCAUCAAAGUCUGCGAGUUCCAGUUCUGUAAUGACCCCUUCCUGGGCGUUCAUA
AAAACAACAAAAGCUGGAUGGAAAGCGAGUUCAGAGUGUAUUCUAGCGCCAAUAACUGUAC
AUUUGAGUACGUGUCCCAGCCCUUCCUGAUGGACCUGGAAGGCAAGCAAGGAAAUUUUAAG
AACCUGCGUGAGUUUGUGUUCAAGAACAUCGAUGGUUAUUUCAAAAUCUACAGCAAGCACA
CCCCCAUUAACCUGGUCAGAGACCUGCCCCAGGGCUUCUCUGCCCUGGAACCUCUGGUGGA
CCUGCCGAUCGGAAUCAACAUCACACGGUUCCAGACCCUGCUAGCCCUGCAUAGAUCUUAC
CUGACCCCCGGCGACAGCUCUUCCGGCUGGACAGCCGGCGCCGCUGCUUACUACGUGGGCU
ACCUGCAGCCUAGAACCUUCCUGCUCAAGUACAACGAAAAUGGCACCAUCACCGACGCCGU
GGACUGCGCCCUGGACCCUCUUAGCGAGACAAAGUGCACACUGAAGAGCUUCACCGUGGAA
AAGGGCAUCUACCAGACAUCGAACUUCAGAGUGCAGCCUACCGAGUCCAUCGUGAGGUUUC
CUAACAUCACCAACCUGUGUCCUUUCGGCGAGGUGUUCAACGCCACCAGAUUCGCCAGCGU
GUACGCCUGGAAUAGAAAGAGAAUCUCUAACUGUGUGGCCGAUUACAGCGUGCUGUACAAC
UCUGCCAGCUUUAGCACCUUUAAAUGUUACGGCGUGAGCCCUACAAAGCUGAACGAUCUGU
GCUUCACCAAUGUGUACGCCGAUUCUUUCGUGAUCCGGGGCGAUGAGGUGCGGCAGAUCGC
CCCAGGCCAGACAGGCAAGAUCGCCGACUACAAUUACAAGCUGCCUGAUGACUUCACCGGC
UGCGUGAUUGCCUGGAACAGCAACAAUCUGGACAGCAAGGUGGGAGGCAACUACAACUACC
UGUACAGACUCUUCCGGAAGAGCAACCUGAAACCUUUCGAGAGAGAUAUCUCAACUGAAAU
CUACCAGGCCGGCUCAACACCCUGCAAUGGAGUUGAGGGCUUCAAUUGCUACUUCCCCCUG
CAGUCUUACGGCUUUCAGCCUACAUACGGCGUGGGCUACCAGCCUUACCGGGUGGUUGUGU
UGAGCUUCGAACUGCUGCACGCCCCUGCUACCGUGUGCGGUCCUAAGAAAAGCACCAACCU
GGUGAAGAACAAGUGCGUAAACUUCAACUUCAACGGCCUGACUGGAACAGGCGUCCUGACC
GAAAGCAACAAGAAGUUCCUGCCUUUUCAACAAUUUGGCAGAGAUAUUGAUGAUACAACAG
AUGCUGUGCGGGAUCCUCAGACACUGGAAAUCCUGGACAUCACCCCCUGCUCCUUCGGCGG
AGUCAGCGUGAUAACCCCUGGCACUAACACCAGCAAUCAGGUGGCCGUGCUCUACCAGGGC
GUCAACUGCACCGAAGUCCCUGUUGCUAUCCACGCUGACCAGCUGACACCUACCUGGAGAG
UGUAUAGCACCGGUUCUAACGUCUUCCAGACCCGCGCCGGCUGUCUGAUCGGCGCCGAGCA
CGUGAACAACAGCUACGAGUGCGACAUCCCCAUCGGCGCUGGCAUCUGCGCCUCUUAUCAG
ACACAGACCAACAGCCACCGGAGAGCUAGAAGCGUGGCCUCUCAGUCGAUCAUUGCCUACA
CCAUGUCCCUGGGCGCCGAGAACUCGGUGGCCUACAGCAACAAUUCUAUCGCCAUCCCCAU
CAACUUCACCAUCAGCGUGACAACCGAAAUUCUGCCAGUGUCCAUGACGAAGACAUCCGUG
GACUGCACAAUGUACAUCUGCGGCGAUAGCACAGAAUGUUCUAAUCUGCUGCUUCAAUAUG
GAUCUUUCUGCACCCAGCUGAACCGGGCCCUGACAGGCAUCGCCGUGGAACAGGACAAAAA
UACCCAGGAGGUGUUUGCCCAGGUGAAGCAGAUCUACAAGACCCCACCAAUCAAGGACUUC
GGAGGGUUUAAUUUCAGCCAGAUCCUGCCCGAUCCUAGCAAGCCUUCCAAGCGGAGUUUCA
UCGAGGACCUGCUGUUCAACAAAGUGACCCUGGCUGAUGCCGGCUUCAUCAAGCAGUACGG
CGACUGCCUGGGCGACAUCGCCGCCAGAGAUCUGAUCUGCGCCCAGAAAUUUAACGGGCUG
ACCGUGCUGCCUCCACUGCUGACCGACGAGAUGAUCGCACAGUACACCAGCGCUUUGCUGG
CGGGCACCAUCACGAGCGGCUGGACCUUCGGGGCCGGCGCCGCCCUGCAAAUUCCUUUCGC
CAUGCAGAUGGCCUACCGGUUUAACGGCAUCGGCGUGACACAGAACGUGCUAUACGAGAAC
CAGAAGCUGAUAGCUAAUCAGUUUAACUCUGCCAUCGGCAAGAUCCAGGACAGCCUCUCCA
GCACCGCCAGCGCCCUGGGUAAGCUGCAGGACGUGGUGAACCAGAACGCCCAAGCCCUGAA
CACCCUGGUUAAGCAGCUGUCCAGCAAUUUCGGCGCUAUUAGCAGCGUUCUGAAUGACAUC
CUGGCCAGACUGGACCCACCUGAGGCCGAGGUGCAGAUCGAUAGACUGAUCACAGGAAGAC
UGCAGAGCCUGCAGACCUACGUCACCCAACAACUCAUCCGGGCCGCCGAAAUCCGGGCCAG
CGCCAACCUUGCAGCCACCAAGAUGAGCGAGUGCGUGCUCGGCCAGAGCAAAAGAGUGGAC
UUUUGCGGCAAAGGCUACCACCUGAUGUCCUUCCCUCAGAGCGCCCCACACGGCGUGGUGU
UCCUGCACGUGACAUAUGUGCCCGCACAGGAGAAGAACUUCACGACUGCUCCCGCCAUCUG
CCACGACGGCAAGGCCCACUUCCCCAGAGAAGGCGUGUUCGUGAGUAACGGGACCCACUGG
UUCGUGACCCAGAGAAACUUCUACGAGCCUCAGAUCAUCACAACCCACAACACAUUCGUGA
GCGGAAACUGCGAUGUGGUGAUCGGAAUCGUGAACAAUACCGUGUACGACCCUCUGCAGCC
UGAGCUGGACAGCUUCAAAGAGGAACUCGACAAGUAUUUUAAGAACCACACCAGCCCUGAC
GUGGAUCUGGGCGACAUCAGCGGCAUCAACGCUAGCGUGGUGAACAUCCAGAAGGAAAUCG
ACAGACUGAACGAGGUGGCCAAGAACCUGAACGAGAGCCUGAUCGACCUGCAGGAGCUGGG
CAAGUACGAGCAGUACAUCAAGUGGCCUUGGUACAUUUGGCUGGGCUUCAUCGCAGGGCUG
AUCGCCAUCGUGAUGGUGACAAUCAUGCUGUGUUGCAUGACCUCUUGUUGCAGCUGUCUUA
AAGGCUGCUGCAGCUGUGGAAGCUGCUGCAAGUUCGACGAGGAUGAUAGCGAACCCGUGCU
GAAGGGCGUCAAGCUGCACUACACCUGACAAACACCAUUGUCACACUCCAUUUAAAGGCUG
UCAAUUCAUAGGUCAGUUUAAACUUCCAGUCGAGGAUGUUUACA

In some embodiments, the coding mRNA can encode one or more viral proteins of the Human alpha-herpesvirus 3 (HHV-3), also known as the varicella-zoster virus (VZV). In particular embodiments, the coding mRNA can encode one or more glycoproteins of VZV, for example, glycoprotein E (VZVgE).

In some embodiments, the coding mRNA can encode one or more immunogenic viral proteins of the influenza virus (type A and B that cause epidemic seasonal flu) such as the hemagglutinin, the neuraminidase, the matrix-2 and/or the nucleoprotein. Hemagglutinin is highly variable between groups, types, and even subtypes of influenza, which is a factor in the difficulty of developing a universal flu vaccine. The Head domain of the Hemagglutinin is highly variable, but the membrane proximal stalk-domain of the Hemagglutinin is relatively well conserved within a group, but is immunosubdominant. Some vaccine strategies therefore use a reduced HA without the Head domain and it is accordingly contemplated that such a reduced HA may be provided in embodiments of the present invention.
It is considered to provide one or more immunogenic viral proteins from any group, type or subtype of influenza, for example, from influenza A Group 1: H1, H2, H5, H6, H8, H9, H11, H12, H13, H16, H17, H18 subtypes and N1, N4, N5, N8 subtypes; from Influenza A Group 2: H3, H4, H7, H10, H14, H15 subtypes+N2, N3, N6, N7, N9 subtypes; or from Influenza B. Influenza B viruses are not divided into subtypes, but instead are further classified into two lineages: B/Yamagata and B/Victoria.
Neuraminidase drifts more slowly than Hemagglutinin, and antibodies against Neuraminidase have been shown to be cross-protective within a subtype. Neuraminidase is immunosubdominant compared to Hemagglutinin. The matrix-2 and/or the nucleoprotein are more conserved than Hemagglutinin but are immunosubdominant.
Each year, the WHO recommends quadrivalent or trivalent influenza vaccines based on predictions. As a result, it is particularly envisioned to provide compositions and constructs which encode more than one influenza antigen, in order to provide broad protection.

In some embodiments, the coding mRNA can encode one or more immunogenic viral proteins of the respiratory syncytial virus such as the F glycoprotein and/or the G glycoprotein. The F glycoprotein from A2 strain can be stabilized in prefusion conformation using the modification described by McLellan et al., 2013, which induces cross-protection against RSV A (Long) and RSV B (18537) strains.

In some embodiments, the coding mRNA can encode one or more immunogenic viral proteins of the human immunodeficiency virus such as the full length or part of the glycoprotein 120 neutralizing epitope (such as CD4BS 421-433 epitope) or the glycoprotein 145. Antigens from HIV such as gag, pol, env, and nef have been expressed in various vectors as possible vaccine candidates (IP Nascimento and LCC Leite, Braz J Med Biol Res. 2012 doi: 10.1590/S0100-879X2012007500142).

In some embodiments, the coding mRNA can encode one or more immunogenic bacterial proteins, or parts thereof, of bacteria from the Mycobacterium genus. In particular, the coding mRNA may encode one or more bacterial proteins from the Mycobacterium tuberculosis and/or Mycobacterium leprae bacteria. In some embodiments, the coding mRNA may encode one or more proteins from the active and/or latent and/or resuscitation phase of M. tuberculosis. For example, the mRNA may encode one or more of the M. tuberculosis proteins selected from ESAT-6, Ag85B, TB10.4, Rv2626 and/or RpfD-B, or a part thereof.

Table 6B below shows examples of ORFs encoding antigen for a number of different potential pathogens, which can be used in the present invention. These ORFs can be present with further RNA sequences, most particularly OPS, as described herein, and/or used in combination with further mRNA constructs. Similar to the discussion above, in some embodiments, the RNA includes one or more of the sequences recited in Table 6 below (SEQ ID NOs: 69 to 84), or an epitope-containing fragment thereof, or a sequence with at least 90%, at least 95%, at least 98%, or at least 99% similarity thereto. In some embodiments, the coding mRNA for the antigen or part thereof has been codon-optimised for expression in human or other mammalian cells. In some embodiments, one or more of the nucleosides used in the mRNA are been replaced by an isomer thereof. As example one, more or all of the uridine nucleosides in the mRNA construct are replaced by pseudouridine nucleosides.

TABLE 6B
Example ORF for antigen for several pathogens
suitable for use in vaccine compositions, not
being optimised for human cellular expression
or containing MOP sequences.
INFLUENZA MRNA ORF for Nucleoprotein (NP) from
A/Michigan/45/2015(H1N1) [SEQ ID NO: 69]
AUGGCGUCUCAAGGCACCAAACGAUCAUAUGAACAAAUGGAGACUGGUGGGGAGCGCCAGGAU
ACCACAGAAAUCAGAGCAUCUGUUGGAAGAAUGAUUGGUGGAAUCGGGAGAUUCUACAUCCAA
AUGUGCACUGAACUCAAACUCAGUGAUUAUGAUGGACGACUAAUCCAGAAUAGCAUAACAAUA
GAGAGGAUGGUGCUUUCUGCUUUUGAUGAGAGAAGAAAUAAAUACCUAGAAGAGCAUCCAAGU
GCUGGGAAGGACCCUAAGAAAACAGGAGGACCCAUCUAUAGAAGAAUAGACGGAAAAUGGACG
AGAGAACUCAUCCUUUAUGACAAAGAAGAAAUAAGGAGAGUUUGGCGCCAAGCAAACAAUGGC
GAAGAUGCAACAGCAGGUCUUACUCAUAUCAUGAUUUGGCAUUCCAACCUGAAUGAUGCCACA
UAUCAGAGGACAAGAGCACUUGUUCGCACUGGAAUGGAUCCCAGAAUGUGCUCUCUAAUGCAA
GGUUCAACACUUCCCAGAAGGUCUGGUGCCGCAGGUGCUGCAGUGAAAGGAGUUGGAACAAUA
GCUAUGGAGUUAAUCAGAAUGAUCAAACGUGGAAUCAAUGACCGAAAUUUCUGGAGGGGUGAA
AAUGGACGAAGGACAAGAGUUGCUUAUGAAAGAAUGUGCAAUAUCCUCAAAGGAAAAUUUCAA
ACAGCUGCCCAGAGGGCAAUGAUGGAUCAAGUAAGAGAAAGUCGAAACCCAGGAAACGCUGAG
AUUGAAGACCUCAUUUUCCUGGCACGGUCAGCACUCAUUCUGAGAGGAUCAGUUGCACAUAAA
UCCUGCCUGCCUGCUUGUGUGUAUGGGCUUGCAGUAGCAAGUGGCCAUGACUUUGAAAGGGAA
GGGUACUCACUGGUCGGGAUAGACCCAUUCAAAUUACUCCAAAACAGUCAAGUGGUCAGCCUG
AUGAGACCAAAUGAAAAUCCAGCUCACAAGAGUCAAUUGGUAUGGAUGGCAUGCCACUCUGCU
GCAUUUGAAGAUUUAAGAGUAUCAAGUUUCAUAAGAGGAAAGAAAGUGAUCCCAAGAGGAAAG
CUUUCCACAAGAGGGGUUCAGAUUGCUUCAAAUGAGAAUGUGGAAACCAUGGACUCCAAUACC
CUGGAACUAAGAAGCAGAUACUGGGCCAUAAGAACCAGGAGUGGAGGAAAUACCAAUCAACAG
AAGGCAUCCGCAGGCCAGAUCAGUGUGCAGCCUACAUUCUCAGUGCAGCGAAAUCUCCCUUUU
GAAAGAGCAACCGUUAUGGCAGCAUUCAGCGGGAACAAUGAAGGACGGACAUCCGACAUGAGA
ACAGAAGUUAUAAGAAUGAUGGAAAGUGCAAAGCCAGAGGAUUUGUCCUUCCAGGGGCGGGGA
GUCUUCGAGCUCUCGGACGAAAAGGCAACGAACCCGAUCGUGCCUUCCUUUGACAUGAGUAAU
GAAGGGUCUUAUUUCUUCGGAGACAAUGCAGAGGAGUAUGACAAUUGA
INFLUENZA MRNA Neuraminidase (NA) from
A/Michigan/45/2015(H1N1) [SEQ ID NO: 70]
AUGAAUCCAAACCAAAAGAUAAUAACCAUUGGUUCGAUCUGUAUGACAAUUGGAAUGGCUAAC
UUAAUAUUACAAAUUGGAAACAUAAUCUCAAUAUGGGUUAGCCACUCAAUUCAAAUUGGAAAU
CAAAGCCAGAUUGAAACAUGCAAUCAAAGCGUCAUUACUUAUGAAAACAACACUUGGGUAAAU
CAGACAUAUGUUAACAUCAGCAACACCAACUUUGCUGCUGGACAGUCAGUGGUUUCCGUGAAA
UUAGCGGGCAAUUCCUCUCUCUGCCCUGUUAGUGGAUGGGCUAUAUACAGUAAAGACAACAGU
GUAAGAAUCGGUUCCAAGGGGGAUGUGUUUGUCAUAAGGGAACCAUUCAUAUCAUGCUCUCCC
UUGGAAUGCAGAACCUUCUUCUUGACUCAAGGGGCCUUGCUAAAUGACAAACAUUCCAAUGGA
ACCAUUAAAGACAGGAGCCCAUACCGAACCCUAAUGAGCUGUCCUAUUGGUGAAGUUCCCUCU
CCAUACAACUCAAGAUUUGAGUCAGUCGCUUGGUCAGCAAGUGCUUGUCAUGAUGGCAUCAAU
UGGCUAACAAUUGGAAUUUCUGGCCCAGACAGUGGGGCAGUGGCUGUGUUAAAGUACAAUGGC
AUAAUAACAGACACUAUCAAGAGUUGGAGGAACAAUAUAUUGAGAACACAAGAGUCUGAAUGU
GCAUGUGUAAAUGGUUCUUGCUUUACCAUAAUGACCGAUGGACCAAGUGAUGGACAGGCCUCA
UACAAAAUCUUCAGAAUAGAAAAGGGAAAGAUAAUCAAAUCAGUCGAAAUGAAAGCCCCUAAU
UAUCACUAUGAGGAAUGCUCCUGUUACCCUGAUUCUAGUGAAAUCACAUGUGUGUGCAGGGAU
AACUGGCAUGGCUCGAAUCGACCGUGGGUGUCUUUCAACCAGAAUCUGGAAUAUCAGAUGGGA
UACAUAUGCAGUGGGGUUUUCGGAGACAAUCCACGCCCUAAUGAUAAGACAGGCAGUUGUGGU
CCAGUAUCGUCUAAUGGAGCAAAUGGAGUAAAAGGAUUUUCAUUCAAAUACGGCAAUGGUGUU
UGGAUAGGGAGAACUAAAAGCAUUAGUUCAAGAAAAGGUUUUGAGAUGAUUUGGGAUCCGAAU
GGAUGGACUGGGACUGACAAUAAAUUCUCAAUAAAGCAAGAUAUCGUAGGAAUAAAUGAGUGG
UCAGGGUAUAGCGGGAGUUUUGUUCAGCAUCCAGAACUAACAGGGCUGGAUUGUAUAAGACCU
UGCUUCUGGGUUGAACUAAUAAGAGGGCGACCCGAAGAGAACACAAUCUGGACUAGCGGGAGC
AGCAUAUCCUUUUGUGGUGUAAACAGUGACACUGUGGGUUGGUCUUGGCCAGACGGUGCUGAG
UUGCCAUUUACCAUUGACAAGUAA
INFLUENZA MRNA Matrix-2 (M2) from A/Michigan/45/2015(H1N1)
with deleted amino acid residues 29-31 to inactivate
ion-channel activity and reduce cytotoxicity
[SEQ ID NO: 71]
AUGAGUCUUCUAACCGAGGUCGAAACGCCUACCAGAAGCGAAUGGGAGUGCAGAUGCAGCGGU
UCAAGUGAUCCUCUCGUCAUUAUCAUUGGGAUCUUGCACCUGAUAUUGUGGAUUACUGAUCGU
CUUUUUUUCAAAUGCAUUUAUCGUCGCUUUAAAUACGGUUUGAAAAGAGGGCCUUCUACGGAA
GGAGUGCCUGAGUCCAUGAGGGAAGAAUAUCAACAGGAGCAGCAGAGUGCUGUGGAUGUUGAC
GAUGGUCAUUUUGUCAACAUAGAGCUAGAGUAA
INFLUENZA MRNA mini Hemagglutinin (HA) for Group1
#4900 based on the HA protein sequence from
A/Brisbane/59/2007 (H1N1) (ACA28844) [SEQ ID NO: 72]
AUGAAAGUGAAGCUGCUGGUCCUGCUGUGCACCUUCACCGCCACAUAUGCCGACACCAUCUGC
AUCGGCUACCACGCCAACAACAGCACAGAUACCGUGGACACCGUGCUGGAGAAGAACGUGACC
GUGACACACAGCGUUAAUCUGCUGGAAAACGGAGGCGGAGGCAAGUACGUGUGCAGCGCCAAG
CUGAGAAUGGUGACCGGCCUGAGAAACAAACCUAGCAAGCAGAGCCAAGGCCUGUUCGGCGCC
AUCGCCGGCUUCACCGAGGGCGGAUGGACCGGCAUGGUCGACGGCUGGUACGGCUAUCACCAC
CAGAACGAGCAGGGCAGCGGCUACGCCGCUGAUCAGAAGUCUACACAAAAUGCUAUUAACGGC
AUCACCAACAAGGUGAACAGCGUGAUCGAGAAGAUGAAUACCCAGUACACCGCCAUCGGCUGU
GAAUACAACAAGUCCGAGCGGUGUAUGAAACAGAUCGAAGAUAAGAUCGAGGAGAUCGAGAGC
AAGAUCUGGUGCUACAACGCCGAGCUGCUCGUGCUGCUGGAAAACGAGAGAACACUGGACUUC
CACGAUUCUAAUGUGAAGAACCUGUACGAGAAGGUGAAGAGCCAGCUGAAGAACAACGCUAAG
GAAAUCGGCAACGGAUGUUUUGAGUUCUACCAUAAGUGCAACGACGAGUGCAUGGAAUCUGUG
AAGAAUGGAACAUACGACUACCCCAAGUACAGCGAGGAAUCCAAGCUGAACCGGGAAAAAAUC
GACGGCGUGAAACUGGAAAGCAUGGGCGUGUACCAGAUCUGA
INFLUENZA MRNA Neuramidinase (NA) from WHO recommended
Influenza A virus (A/Wisconsin/588/2019(H1N1))
[SEQ ID NO: 73]
AUGAAUCCAAACCAAAAGAUAAUAACCAUUGGUUCUAUCUGUAUGACAAUUGGAACGGCUAAC
UUAAUAUUACAAAUUGGAAACAUAAUCUCAAUAUGGGUUAGCCACUCAAUUCAAAUUGGAAAU
CAAAGCCAGAUUGAAACAUGCAAUAAAAGCGUCAUUACUUAUGAAAACAACACUUGGGUAAAU
CAGACAUUUGUUAACAUCAGCAACACUAACUCUGCUGCUAGACAGUCAGUGGCUUCCGUGAAA
UUAGCGGGCAAUUCCUCUCUCUGCCCUGUUAGUGGAUGGGCUAUAUACAGUAAAGACAACAGU
GUAAGAAUCGGUUCCAAGGGGGAUGUGUUUGUCAUAAGGGAACCAUUCAUAUCAUGCUCUCCC
UUGGAAUGCAGAACCUUCUUCUUGACUCAAGGGGCUUUGCUAAAUGACAAACAUUCCAAUGGA
ACCAUUAAAGACAGAAGCCCAUAUCGAACCCUAAUGAGCUGUCCUAUUGGUGAAGUUCCCUCU
CCAUACAACUCAAGAUUUGAGUCAGUCGCUUGGUCAGCAAGUGCUUGUCAUGAUGGCACCAAU
UGGCUAACAAUUGGAAUUUCUGGCCCAGACAGUGGGGCAGUGGCUGUGUUAAAAUACAAUGGC
AUAAUAACAGACACUAUCAAGAGUUGGAGGAACAAGAUAUUGAGAACACAAGAGUCUGAAUGU
GCAUGUGUAAAUGGUUCUUGCUUUACCAUAAUGACCGAUGGACCAAGUGAUGGACAGGCCUCA
UACAAAAUCUUCAGAAUAGAAAAGGGAAAGAUAAUCAAAUCAGUCGAAAUGAAAGCCCCUAAU
UAUCACUAUGAAGAAUGCUCCUGUUACCCUGAUUCUAGUGAAAUCACAUGUGUGUGCAGGGAU
AACUGGCAUGGCUCGAAUCGACCGUGGGUGUCUUUCAACCAGAAUCUGGAAUAUCAGAUGGGA
UACAUAUGCAGUGGGGUUUUCGGAGACAAUCCACGCCCUAAUGAUAAGACAGGCAGUUGUGGU
CCAGUAUCGUCUAAUGGAGCAAAUGGGGUAAAAGGAUUUUCAUUCAAAUACGGCAAUGGUGUU
UGGAUAGGGAGAACUAAGAGCAUUAGUUCAAGAAAAGGUUUUGAGAUGAUUUGGGAUCCGAAU
GGAUGGACUGGGACUGACAAUAAAUUCUCAAAAAAGCAAGAUAUCGUAGGAAUAAAUGAGUGG
UCAGGGUAUAGCGGGAGUUUUGUUCAGCAUCCAGAACUAACAGGGCUGAAUUGUAUAAGACCU
UGCUUCUGGGUUGAACUAAUAAGAGGACGACCCGAAGAGAACACAAUCUGGACUAGCGGGAGC
AGCAUAUCCUUUUGUGGUGUAGACAGUGACAUUGUGGGUUGGUCUUGGCCAGACGGUGCUGAG
UUGCCAUUUACCAUUGACAAGUAA
INFLUENZA MRNA Hemagglutinin (HA) from WHO recommended
Influenza A virus (A/Wisconsin/588/2019(H1N1))
[SEQ ID NO: 74]
AUGAAGGCAAUACUAGUAGUUAUGCUGUAUACAUUUACAACCGCAAAUGCAGACACAUUAUGU
AUAGGUUAUCAUGCGAACAAUUCAACAGACACUGUGGACACAGUACUAGAAAAGAAUGUAACA
GUAACACACUCUGUCAAUCUUCUGGAAGACAAGCAUAACGGAAAACUAUGCAAACUAAGAGGG
GUAGCCCCAUUGCAUUUGGGUAAAUGUAACAUUGCUGGCUGGAUCCUGGGAAAUCCAGAGUGU
GAAUCACUCUCCACAGCAAGAUCAUGGUCCUACAUUGUGGAAACAUCUAAUUCAGACAAUGGA
ACGUGUUACCCAGGAGAUUUCAUCAAUUAUGAGGAGCUAAGAGAGCAAUUGAGCUCAGUGUCA
UCAUUUGAAAGGUUUGAAAUAUUCCCCAAGACAAGUUCAUGGCCUAAUCAUGACUCGGACAAU
GGUGUAACGGCAGCAUGUCCUCACGCUGGAGCAAAAAGCUUCUACAAAAACUUGAUAUGGCUG
GUUAAAAAAGGAAAAUCAUACCCAAAGAUCAACCAAACCUACAUUAAUGAUAAAGGGAAAGAA
GUCCUCGUGCUGUGGGGCAUUCACCAUCCACCUACUAUUGCUGACCAACAAAGUCUCUAUCAG
AAUGCAGAUGCAUAUGUUUUUGUGGGGACAUCAAGAUACAGCAAGAAGUUCAAGCCGGAAAUA
GCAACAAGACCCAAAGUGAGGGAUCAAGAAGGGAGAAUGAACUAUUACUGGACACUAGUAGAA
CCGGGAGACAAAAUAACAUUCGAAGCAACUGGUAAUCUAGUGGCACCGAGAUAUGCAUUCACA
AUGGAAAGAGAUGCUGGAUCUGGUAUUAUCAUUUCAGAUACACCAGUCCACGAUUGCAAUACA
ACUUGUCAGACACCCGAGGGUGCUAUAAACACCAGCCUCCCAUUUCAGAAUGUACAUCCGAUC
ACAAUUGGGAAAUGUCCAAAGUAUGUAAAAAGCACAAAAUUGAGACUGGCCACAGGAUUGAGG
AAUGUCCCGUCUAUUCAAUCUAGAGGCCUAUUCGGGGCCAUUGCUGGCUUCAUCGAAGGGGGG
UGGACAGGGAUGGUAGAUGGAUGGUACGGUUAUCACCAUCAAAAUGAGCAGGGGUCAGGAUAU
GCAGCCGAUCUGAAGAGCACACAAAAUGCCAUUGAUAAGAUUACUAACAAAGUAAAUUCUGUU
AUUGAAAAGAUGAAUACACAGUUCACAGCAGUUGGUAAAGAGUUCAACCACCUUGAAAAAAGA
AUAGAGAAUCUAAAUAAAAAGGUUGAUGAUGGUUUCCUGGACAUUUGGACUUACAAUGCCGAA
CUGUUGGUUCUACUGGAAAACGAAAGAACUUUGGACUAUCACGAUUCAAAUGUGAAGAACUUG
UAUGAAAAAGUAAGAAACCAGUUAAAAAACAAUGCCAAGGAAAUUGGAAACGGCUGCUUUGAA
UUUUACCACAAAUGCGACAACACAUGCAUGGAAAGUGUCAAGAAUGGGACUUAUGACUACCCA
AAAUACUCAGAGGAAGCAAAAUUAAACAGAGAAAAAAUAGAUGGAGUAAAGCUGGACUCAACA
AGGAUCUACCAGAUUUUGGCGAUCUAUUCAACUGUUGCCAGUUCAUUGGUACUGGUAGUCUCC
CUGGGGGCAAUCAGCUUCUGGAUGUGCUCUAAUGGGUCUCUACAGUGUAGAAUAUGUAUUUAA
INFLUENZA MRNA Neuramidinase (NA) from recommended
Influenza B virus (B/Washington/02/2019 (B/Victoria
lineage)) [SEQ ID NO: 75]
AUGCUACCUUCAACUAUACAAACGUUAACCCUAUUUCUCACAUCAGGGGGAGUAUUAUUAUCA
CUAUAUGUGUCAGCUUCAUUAUCAUACUUACUAUAUUCGGAUAUAUUGCUAAAAUUCUCACCA
ACAGAAAUAACUGCACCAACAAUGCCAUUGGAUUGUGCAAACGCAUCAAAUGUUCAGGCUGUG
AACCGUUCUGCAACAAAAGGGGUGACACUUCUUCUCCCAGAACCGGAGUGGACAUACCCGCGU
UUAUCUUGCCCGGGCUCAACCUUUCAGAAAGCACUUCUAAUUAGCCCUCAUAGAUUCGGAGAA
ACCAAAGGAAACUCAGCUCCCUUGAUAAUAAGGGAACCUUUUGUAGCUUGUGGACCAAAUGAA
UGCAAACACUUUGCUUUAACCCAUUAUGCUGCCCAACCAGGGGGAUACUAUAAUGGAACAAGA
GGAGACAGAAACAAGCUGAGGCAUCUAAUUUCAGUCAAAUUGGGCAAAAUCCCAACAGUAGAG
AACUCCAUUUUCCACAUGGCAGCAUGGAGCGGGUCCGCGUGCCAUGAUGGUAAGGAAUGGACA
UAUAUCGGAGUUGAUGGCCCUGACAAUAAUGCAUUGCUCAAAGUAAAAUAUGGAGAAGCAUAU
ACUGACACAUACCAUUCCUAUGCAAACAACAUCCUAAGAACACAAGAAAGUGCCUGCAAUUGC
AUCGGGGGAAAUUGUUAUCUAAUGAUAACUGAUGGCUCAGCUUCAGGUGUUAGUGAAUGCAGA
UUUCUUAAGAUUCGAGAGGGCCGAAUAAUAAAAGAAAUAUUUCCAACAGGAAGAGUAAAACAC
ACUGAGGAGUGCACAUGCGGAUUUGCCAGCAAUAAAACCAUAGAAUGUGCCUGUAGAGACAAC
AGGUACACAGCAAAAAGACCUUUUGUCAAAUUAAACGUGGAGACUGAUACAGCAGAAAUAAGG
UUGAUGUGCACAGAUACUUAUUUGGACACCCCCAGACCAAAUGAUGGAAGCAUAACAGGCCCU
UGUGAAUCUGAUGGGGACAAAGGGAGUGGAGGCAUCAAGGGAGGAUUUGUUCAUCAAAGAAUG
AAAUCCAAGAUUGGAAGGUGGUACUCUCGAACGAUGUCUAAAACUGAAAGGAUGGGGAUGGGA
CUGUAUGUCAAGUAUGGUGGAGACCCAUGGGCUGACAGUGAUGCCCUAACUUUUAGUGGAGUA
AUGGUUUCAAUGAAAGAACCUGGUUGGUAUUCCUUUGGCUUCGAAAUAAAAGAUAAGAAAUGC
GAUGUCCCCUGUAUUGGGAUAGAGAUGGUACAUGAUGGUGGAAAAGAGACUUGGCACUCAGCA
GCAACAGCCAUUUACUGUUUAAUGGGCUCAGGACAGCUGCUGUGGGACACUGUCACAGGUGUU
GACAUGGCUCUGUAA
INFLUENZA MRNA Hemagglutinin (HA) from WHO recommended
Influenza B virus (B/Washington/02/2019 (B/Victoria
lineage)) [SEQ ID NO: 76]
AUGAAGGCAAUAAUUGUACUACUCAUGGUAGUAACAUCCAAUGCAGAUCGAAUCUGCACUGGG
AUAACAUCGUCAAACUCACCACAUGUCGUCAAAACUGCUACUCAAGGGGAGGUCAACGUGACC
GGUGUAAUACCACUGACAACAACACCCACCAAAUCUCAUUUUGCAAAUCUCAAAGGAACAGAA
ACCAGGGGGAAACUAUGCCCAAAAUGCCUCAACUGCACAGAUCUGGAUGUAGCCUUGGGCAGA
CCAAAAUGCACAGGGAAAAUACCCUCUGCAAGGGUUUCAAUACUCCAUGAAGUCAGACCUGUU
ACAUCUGGGUGCUUUCCUAUAAUGCACGAUAGAACAAAAAUUAGACAGCUGCCUAACCUUCUC
CGAGGAUACGAACAUGUCAGGUUAUCAACUCACAACGUUAUCAAUGCAGAAGAUGCACCAGGA
AGACCCUACGAAAUUGGAACCUCAGGGUCUUGCCCUAACAUUACCAAUGGAAACGGAUUCUUC
GCAACAAUGGCUUGGGCCGUCCCAAAAAACAAAACAGCAACAAAUCCAUUAACAAUAGAAGUA
CCAUACAUUUGUACAGAAGGAGAAGACCAAAUUACCGUUUGGGGGUUCCACUCUGACAGCGAG
ACCCAAAUGGCAAAGCUCUAUGGGGACUCAAAGCCCCAGAAGUUCACCUCAUCUGCCAACGGA
GUGACCACACAUUACGUUUCACAGAUUGGUGGCUUCCCAAAUCAAACAGAAGACGGAGGACUA
CCACAAAGUGGCAGAAUUGUUGUUGAUUACAUGGUGCAGAAAUCUGGAAAAACAGGAACAAUU
ACCUAUCAAAGAGGUAUUUUAUUGCCUCAAAAGGUGUGGUGCGCAAGUGGCAGGAGCAAGGUA
AUAAAAGGAUCCUUGCCCUUAAUUGGAGAAGCAGAUUGCCUCCAUGAAAAAUACGGUGGAUUA
AACAAAAGCAAGCCUUACUACACAGGGGAACAUGCAAAGGCCAUAGGAAAUUGCCCAAUAUGG
GUGAAAACACCCUUGAAGCUGGCCAAUGGAACCAAAUAUAGACCCCCUGCAAAACUAUUAAAG
GAAAGAGGUUUCUUCGGAGCCAUUGCUGGUUUCUUAGAGGGAGGAUGGGAAGGAAUGAUUGCA
GGUUGGCACGGAUACACAUCCCAUGGGGCACAUGGAGUAGCGGUGGCAGCUGACCUUAAGAGC
ACUCAAGAGGCCAUAAACAAGAUAACAAAAAAUCUCAACUCUUUGAGUGAGCUGGAAGUAAAG
AAUCUUCAAAGACUAAGCGGUGCCAUGGAUGAACUCCACAACGAAAUACUAGAACUAGAUGAG
AAAGUGGAUGAUCUCAGAGCUGAUACAAUAAGCUCACAAAUAGAACUCGCAGUCCUGCUUUCC
AAUGAAGGAAUAAUAAACAGUGAAGAUGAACAUCUCUUGGCGCUUGAAAGAAAGCUGAAGAAA
AUGCUGGGCCCCUCUGCUGUAGAGAUAGGGAAUGGAUGCUUUGAAACCAAACACAAGUGCAAC
CAGACCUGUCUCGACAGAAUAGCUGCUGGUACCUUUGAUGCAGGAGAAUUUUCUCUCCCCACC
UUUGAUUCACUGAAUAUUACUGCUGCAUCUUUAAAUGACGACGGAUUGGACAAUCAUACUAUA
CUGCUUUACUACUCAACUGCUGCCUCCAGUUUGGCUGUAACACUGAUGAUAGCUAUCUUUGUU
GUUUAUAUGGUCUCCAGAGACAAUGUUUCUUGCUCCAUUUGUCUAUAA
INFLUENZA MRNA mini Hemagglutinin (HA) for Group2
H3ssF_C based on the HA protein sequence from
A/Finland/486/2004 (H3N2) [SEQ ID NO: 77]
AUGAAGACCAUCAUCGCCCUGAGCUACAUCCUGUGCCUGGUGUUCGCCCAGAAGCUGCCCGGC
AACGACAACAGCACCGCCACCCUGUGCCUGGGCCACCACGCCGUGCCCAACGGCACCAUCGUG
AAGACCAUCACCAACGACCAGAUCGAGGUGACCAACGCCACCGAGCUGGUGUUCCCCGGCUGC
GGCGUGCUGAAGCUGGCCACCGGCAUGAGGAACGUGCCCGAGAAGCAGACCAGGGGCAUCUUC
GGCGCCAUCGCCGGCUUCAUCGAGAACGGCUGGGAGGGCAUGGUGGACGGCUGGUACGGCUUC
AGGCACCAGAACAGCGAGGGCAUCGGCCAGGCCGCCGACCUGAAGAGCACCCAGGCCGCCAUC
AACCAGAUCAACGGCAUGGUGAACAGGGUGAUCGAGCUGAUGGAGCAGGGCGGCCCCGACUGC
UACCUGGCCGAGCUGCUGGUGGCCCUGCUGAACCAGCACACCAUCGACCUGACCGACAGCGAG
AUGAGGAAGCUGUUCGAGAGGACCAAGAAGCAGCUGAGGGAGAACGCCGAGGACAUGGGCAAC
GGCUGCUUCAAGAUCUACCACAAGUGCGACAACGCCUGCAUCGGCAGCAUCAGGAACGGCACC
UACGACCACGACGUGUACAGGGACGAGGCCCUGAACAACAGGUUCCAGAUCAAGUAA
INFLUENZA MRNA mini Hemagglutinin (HA) for Group2
H7ssF_C based on the HA protein sequence from
A/Shanghai/2/2013 (H7N9) [SEQ ID NO: 78]
AUGAACACCCAGAUCCUGGUGUUCGCCCUGAUCGCCAUCAUCCCCACCAACGCCGACAAGAUC
UGCCUGGGCCACCACGCCGUGAGCAACGGCACCAAGGUGAACACCCUGACCGAGAGGGGCGUG
GAGGUGGUGAACGCCACCGAGCUGGUGUUCCCCGGCUGCGGCGUGCUGCUGCUGGCCACCGGC
AUGAAGAACGUGCCCGAGAUCCCCAAGGGCAGGGGCCUGUUCGGCGCCAUCGCCGGCUUCAUC
GAGAACGGCUGGGAGGGCCUGAUCGACGGCUGGUACGGCUUCAGGCACCAGAACGCCCAGGGC
GAGGGCACCGCCGCCGACUACAAGAGCACCCAGAGCGCCAUCGACCAGAUCACCGGCAUGGUG
AACAGGGUGAUCGAGCUGAUGGAGCAGGGCGGCCCCGACUGCUACCUGGCCGAGCUGCUGGUG
GCCAUGCUGAACCAGCACACCAUCGACCUGGCCGACAGCGAGAUGGACAAGCUGUACGAGAGG
GUGAAGAGGCAGCUGAGGGAGAACGCCGAGGAGGACGGCACCGGCUGCUUCGAGAUCUUCCAC
AAGUGCGACGACGACUGCAUGGCCAGCAUCAGGAACAACACCUACGACCACAGCAAGUACAGG
GAGGAGGCCAUGCAGAACAGGAUCCAGAUCGACUAA
RESPIRATORY SYNCYTIAL VIRUS MRNA RSV Prefusion
F Glycoprotein from A2 strain (S155C, S190F,
V207L, S290C mutations that stabilize the
prefusion conformation) [SEQ ID NO: 79]
AUGGAGUUGCUAAUCCUCAAAGCAAAUGCAAUUACCACAAUCCUCACUGCAGUCACAUUUUGU
UUUGCUUCUGGUCAAAACAUCACUGAAGAAUUUUAUCAAUCAACAUGCAGUGCAGUUAGCAAA
GGCUAUCUUAGUGCUCUGAGAACUGGUUGGUAUACCAGUGUUAUAACUAUAGAAUUAAGUAAU
AUCAAGGAAAAUAAGUGUAAUGGAACAGAUGCUAAGGUAAAAUUGAUAAAACAAGAAUUAGAU
AAAUAUAAAAAUGCUGUAACAGAAUUGCAGUUGCUCAUGCAAAGCACACCACCAACAAACAAU
CGAGCCAGAAGAGAACUACCAAGGUUUAUGAAUUAUACACUCAACAAUGCCAAAAAAACCAAU
GUAACAUUAAGCAAGAAAAGGAAAAGAAGAUUUCUUGGUUUUUUGUUAGGUGUUGGAUCUGCA
AUCGCCAGUGGCGUUGCUGUAUGUAAGGUCCUGCACCUAGAAGGGGAAGUGAACAAGAUCAAA
AGUGCUCUACUAUCCACAAACAAGGCUGUAGUCAGCUUAUCAAAUGGAGUUAGUGUCUUAACC
UUCAAAGUGUUAGACCUCAAAAACUAUAUAGAUAAACAAUUGUUACCUAUUCUGAACAAGCAA
AGCUGCAGCAUAUCAAAUAUAGAAACUGUGAUAGAGUUCCAACAAAAGAACAACAGACUACUA
GAGAUUACCAGGGAAUUUAGUGUUAAUGCAGGUGUAACUACACCUGUAAGCACUUACAUGUUA
ACUAAUAGUGAAUUAUUGUCAUUAAUCAAUGAUAUGCCUAUAACAAAUGAUCAGAAAAAGUUA
AUGUCCAACAAUGUUCAAAUAGUUAGACAGCAAAGUUACUCUAUCAUGUGCAUAAUAAAAGAG
GAAGUCUUAGCAUAUGUAGUACAAUUACCACUAUAUGGUGUUAUAGAUACACCCUGUUGGAAA
CUACACACAUCCCCUCUAUGUACAACCAACACAAAAGAAGGGUCCAACAUCUGUUUAACAAGA
ACUGACAGAGGAUGGUACUGUGACAAUGCAGGAUCAGUAUCUUUCUUCCCACAAGCUGAAACA
UGUAAAGUUCAAUCAAAUCGAGUAUUUUGUGACACAAUGAACAGUUUAACAUUACCAAGUGAA
AUAAAUCUCUGCAAUGUUGACAUAUUCAACCCCAAAUAUGAUUGUAAAAUUAUGACUUCAAAA
ACAGAUGUAAGCAGCUCCGUUAUCACAUCUCUAGGAGCCAUUGUGUCAUGCUAUGGCAAAACU
AAAUGUACAGCAUCCAAUAAAAAUCGUGGAAUCAUAAAGACAUUUUCUAACGGGUGCGAUUAU
GUAUCAAAUAAAGGGAUGGACACUGUGUCUGUAGGUAACACAUUAUAUUAUGUAAAUAAGCAA
GAAGGUAAAAGUCUCUAUGUAAAAGGUGAACCAAUAAUAAAUUUCUAUGACCCAUUAGUAUUC
CCCUCUGAUGAAUUUGAUGCAUCAAUAUCUCAAGUCAACGAGAAGAUUAACCAGAGCCUAGCA
UUUAUUCGUAAAUCCGAUGAAUUAUUACAUAAUGUAAAUGCUGGUAAAUCCACCACAAAUAUC
AUGAUAACUACUAUAAUUAUAGUGAUUAUAGUAAUAUUGUUAUCAUUAAUUGCUGUUGGACUG
CUCUUAUACUGUAAGGCCAGAAGCACACCAGUCACACUAAGCAAAGAUCAACUGAGUGGUAUA
AAUAAUAUUGCAUUUAGUAACUAA
MYCOBACTERIUM TUBERCULOSIS MRNA Tuberculosis
ESAT-6 (Rv3875) - Active phase (sequence from
H37Rv strain) [SEQ ID NO: 80]
AUGACAGAGCAGCAGUGGAAUUUCGCGGGUAUCGAGGCCGCGGCAAGCGCAAUCCAGGGAAAU
GUCACGUCCAUUCAUUCCCUCCUUGACGAGGGGAAGCAGUCCCUGACCAAGCUCGCAGCGGCC
UGGGGCGGUAGCGGUUCGGAGGCGUACCAGGGUGUCCAGCAAAAAUGGGACGCCACGGCUACC
GAGCUGAACAACGCGCUGCAGAACCUGGCGCGGACGAUCAGCGAAGCCGGUCAGGCAAUGGCU
UCGACCGAAGGCAACGUCACUGGGAUGUUCGCAUAG
MYCOBACTERIUM TUBERCULOSIS MRNA Tuberculosis
Ag85B (Rv1886) - Active phase (sequence from
H37Rv strain) [SEQ ID NO: 81]
AUGACAGACGUGAGCCGAAAGAUUCGAGCUUGGGGACGCCGAUUGAUGAUCGGCACGGCAGCG
GCUGUAGUCCUUCCGGGCCUGGUGGGGCUUGCCGGCGGAGCGGCAACCGCGGGCGCGUUCUCC
CGGCCGGGGCUGCCGGUCGAGUACCUGCAGGUGCCGUCGCCGUCGAUGGGCCGCGACAUCAAG
GUUCAGUUCCAGAGCGGUGGGAACAACUCACCUGCGGUUUAUCUGCUCGACGGCCUGCGCGCC
CAAGACGACUACAACGGCUGGGAUAUCAACACCCCGGCGUUCGAGUGGUACUACCAGUCGGGA
CUGUCGAUAGUCAUGCCGGUCGGCGGGCAGUCCAGCUUCUACAGCGACUGGUACAGCCCGGCC
UGCGGUAAGGCUGGCUGCCAGACUUACAAGUGGGAAACCUUCCUGACCAGCGAGCUGCCGCAA
UGGUUGUCCGCCAACAGGGCCGUGAAGCCCACCGGCAGCGCUGCAAUCGGCUUGUCGAUGGCC
GGCUCGUCGGCAAUGAUCUUGGCCGCCUACCACCCCCAGCAGUUCAUCUACGCCGGCUCGCUG
UCGGCCCUGCUGGACCCCUCUCAGGGGAUGGGGCCUAGCCUGAUCGGCCUCGCGAUGGGUGAC
GCCGGCGGUUACAAGGCCGCAGACAUGUGGGGUCCCUCGAGUGACCCGGCAUGGGAGCGCAAC
GACCCUACGCAGCAGAUCCCCAAGCUGGUCGCAAACAACACCCGGCUAUGGGUUUAUUGCGGG
AACGGCACCCCGAACGAGUUGGGCGGUGCCAACAUACCCGCCGAGUUCUUGGAGAACUUCGUU
CGUAGCAGCAACCUGAAGUUCCAGGAUGCGUACAACGCCGCGGGGGGGCACAACGCCGUGUUC
AACUUCCCGCCCAACGGCACGCACAGCUGGGAGUACUGGGGCGCUCAGCUCAACGCCAUGAAG
GGUGACCUGCAGAGUUCGUUAGGCGCCGGCUGA
MYCOBACTERIUM TUBERCULOSIS MRNA Tuberculosis
TB10.4 (Rv0288) - Active phase (sequence from
H37Rv strain) [SEQ ID NO: 82]
AUGUCGCAAAUCAUGUACAACUACCCCGCGAUGUUGGGUCACGCCGGGGAUAUGGCCGGAUAU
GCCGGCACGCUGCAGAGCUUGGGUGCCGAGAUCGCCGUGGAGCAGGCCGCGUUGCAGAGUGCG
UGGCAGGGCGAUACCGGGAUCACGUAUCAGGCGUGGCAGGCACAGUGGAACCAGGCCAUGGAA
GAUUUGGUGCGGGCCUAUCAUGCGAUGUCCAGCACCCAUGAAGCCAACACCAUGGCGAUGAUG
GCCCGCGACACGGCCGAAGCCGCCAAAUGGGGGGCUAG
MYCOBACTERIUM TUBERCULOSIS MRNA Tuberculosis
Hrp1 (Rv2626)- Latent phase (sequence from
H37Rv strain) [SEQ ID NO: 83]
AUGACCACCGCACGCGACAUCAUGAACGCAGGUGUGACCUGUGUUGGCGAACACGAGACGCUA
ACCGCUGCCGCUCAAUACAUGCGUGAGCACGACAUCGGCGCGUUGCCGAUCUGCGGGGACGAC
GACCGGCUGCACGGCAUGCUCACCGACCGCGACAUUGUGAUCAAAGGCCUGGCUGCGGGCCUA
GACCCGAAUACCGCCACGGCUGGCGAGUUGGCCCGGGACAGCAUCUACUACGUCGAUGCGAAC
GCAAGCAUCCAGGAGAUGCUCAACGUCAUGGAAGAACAUCAGGUCCGCCGUGUUCCGGUCAUC
UCAGAGCACCGCUUGGUCGGAAUCGUCACCGAAGCCGACAUCGCCCGACACCUGCCCGAGCAC
GCCAUUGUGCAGUUCGUCAAGGCAAUCUGCUCGCCCAUGGCCCUCGCCAGCUAG
MYCOBACTERIUM TUBERCULOSIS MRNA Tuberculosis
RpfB-D hybrid - Resuscitation phase (sequences
from H37Rv strain). [SEQ ID NO: 84]
AUGACCGUCGACGGAACCGCGAUGCGGGUGACCACGAUGAAAUCGCGGGUGAUCGACAUCGUC
GAAGAGAACGGGUUCUCAGUCGACGACCGCGACGACCUGUAUCCCGCGGCCGGCGUGCAGGUC
CAUGACGCCGACACCAUCGUGCUGCGGCGUAGCCGUCCGCUGCAGAUCUCGCUGGAUGGUCAC
GACGCUAAGCAGGUGUGGACGACCGCGUCGACGGUGGACGAGGCGCUGGCCCAACUCGCGAUG
ACCGACACGGCGCCGGCCGCGGCUUCUCGCGCCAGCCGCGUCCCGCUGUCCGGGAUGGCGCUA
CCGGUCGUCAGCGCCAAGACGGUGCAGCUCAACGACGGCGGGUUGGUGCGCACGGUGCACUUG
CCGGCCCCCAAUGUCGCGGGGCUGCUGAGUGCGGCCGGCGUGCCGCUGUUGCAAAGCGACCAC
GUGGUGCCCGCCGCGACGGCCCCGAUCGUCGAAGGCAUGCAGAUCCAGGUGACCCGCAAUCGG
AUCAAGAAGGUCACCGAGCGGCUGCCGCUGCCGCCGAACGCGCGUCGUGUCGAGGACCCGGAG
AUGAACAUGAGCCGGGAGGUCGUCGAAGACCCGGGGGUUCCGGGGACCCAGGAUGUGACGUUC
GCGGUAGCUGAGGUCAACGGCGUCGAGACCGGCCGUUUGCCCGUCGCCAACGUCGUGGUGACC
CCGGCCCACGAAGCCGUGGUGCGGGUGGGCACCAAGCCCGGUACCGAGGUGCCCCCGGUGAUC
GACGGAAGCAUCUGGGACGCCAUCGCGCAAUGCAAAUCCGGCGGCAAUUGGGCGGCCAACACC
GGUAACGGGUUAUACGGUGGUCUGCAGAUCAGCCAGGCGGCGUGGGAUUCCAACGGUGGUGUC
GGGUCGCCGGCGGCCGCGAGUCCCCAGCAACAGAUCGAGGUCGCAGACAACAUUAUGAAAACC
GCAGGCCCGGGUGCGUGGCCGAAAUGUAGUUCUUGUAGUCAGGGAGACGCACCGCUGGGCUCG
CUCACCCACAUCCUGACGUUCCUCGCGGCCGAGACUGGAGGUUGUUCGGGGAGCAGGGACGAU
UGA

It is particularly envisioned to provide compositions, including pharmaceutical compositions, comprising mRNA which encode more than one antigen, for example, encoding the spike protein from more than one SARS-CoV-2 spike protein. Multiple antigen may be provided by the same, or different mRNA constructs, as described elsewhere herein. In one embodiment, a composition is provided comprising mRNA constructs encoding the spike protein from at least two, suitably all three of the wild type SARS-CoV-2, the Beta (South African) variant SARS-CoV-2, and the Delta variant SARS-CoV-2. These may be present on the same or different mRNA constructs. The mRNA construct(s) encoding these antigen may lack OPS, or one or more, suitably all of them have OPS as described elsewhere herein. In some embodiments, the OPS can comprise sequences capable of binding with miRNA-122, miRNA-1, miRNA-203a, and miRNA-30a; or sequences capable of binding with miRNA-122, miRNA-192, and miRNA-30a. In any of these embodiments, the composition may also comprise mRNA coding for an immunomodulator, as further discussed below. In particular, the composition may also comprise mRNA encoding IL-12, as discussed elsewhere herein. The immunomodulator mRNA may lack an OPS, or may comprise an OPS as described elsewhere herein. In some embodiments, the OPS can comprise sequences capable of binding with miRNA-122, miRNA-1, miRNA-203a, and miRNA-30a; or sequences capable of binding with miRNA-122, miRNA-192, and miRNA-30a. In specific embodiments, the mRNA construct(s) encoding the antigen (for example, two or more variant SARS-CoV-2 spike protein) may lack OPS, while the mRNA construct(s) encoding the immunomodulator (for example, IL-12) may include an OPS, as described.

In some embodiments, it is envisioned that a composition may be provided which comprises mRNA encoding viral proteins from each of SARS-CoV-2 (or a variant thereof) and influenza, for example, in order to provide a multivalent or joint vaccination against a seasonal, new, or emerging variant of one or both of these viruses. As described elsewhere, the different antigen may be provided on the same or different mRNA constructs, and these mRNA construct(s) may lack an OPS, or may comprise an OPS/MOP as described elsewhere. The compositions may further comprise mRNA coding for an immunomodulator, such as IL-12, as further discussed below. This mRNA may also comprise an OPS, as described.

In various embodiments, the mRNA coding for an antigen product additionally comprises at least one OPS that protects multiple organs (i.e. a multi-organ protection sequence or “MOP”), wherein the OPS sequence comprises at least three (for example, at least a first, a second and a third) micro-RNA (miRNA) target sequences. One of the target sequences can be a sequence capable of binding with miRNA-1. The target sequences can comprise sequences capable of binding with one or more of miRNA-1, miRNA-133a, miRNA-206, miRNA-122, miRNA-192, miRNA-203a, miRNA-205, miRNA-200c, miRNA-30a/b/c, and/or Let7a/b, suitably with all of these.

In various embodiments of any antigen-encoding mRNA, the OPS can comprise sequences capable of binding with miRNA-122, miRNA-1, miRNA-203a, and miRNA-30a; sequences capable of binding with Let7b, miRNA-126, and miRNA-30a; sequences capable of binding with miRNA-122, miRNA-192, and miRNA-30a; or sequences capable of binding with miRNA-192, miRNA-30a, and miRNA-124, with two sequences capable of binding with miRNA 122. Any OPS such as those described here may further include a sequence capable of binding with miRNA-124, for the protection of brain tissue, and/or a sequence capable of binding with Let7b. The order of the target sequences within an OPS (that is, their 5′ to 3′ arrangement) is not considered to be important, and any permutation may be considered.

It can be appreciated that the above-mentioned approach is particular suitable for preparing vaccine therapeutic compositions similar to typical ‘toxoid’ vaccines, where an immune response is induced against an inactivated toxin produced by a bacterium or other organism, or ‘subunit’ vaccines, where an immune response is induced against a fragment of a target micro-organism.

While any embodiment of the invention described herein may have, as a proposed target tissue, the blood or subdivisions thereof (such as hematopoietic cells, lymphoid cells, and so on), it is particularly considered that the blood and subdivisions thereof may be particularly appropriate in embodiments where the aim is to induce an immune response, where an immune response is to be induced against the product encoded by the coding mRNA, and/or optionally where the aim is to provide a vaccine therapy. Peripheral blood mononuclear cells (PBMC) are particularly contemplated as targets for such approaches, and suitably, antigen presenting cells (APC).

Conventional vaccines function, at least in part, by presenting pathogen-specific antigen to the immune system (exogenous antigen), so that an immune reaction can be induced against it, and so that this exogenous antigen can be recognised and rapidly countered when it is next encountered. So-called antigen presenting cells (APC) are key to this process. While all nucleated cells can present endogenous antigen to cytotoxic T cells (CD8+), certain cells are ‘professional’ APC, including dendritic cells, macrophages and B cells, with the ability to detect and present exogenous antigens. These cells internalise and process exogenous antigens, and present them or fragments of them (immunodominant epitopes) on the surface, in association with major histocompatibility complexes type II (MHC-II), and often co-stimulatory molecules, to shape an enhanced T cells responses, such as CD4+ helper T cells, that play pivotal role in initiating B cells driven antibody production (adaptive immunity).

Previous efforts have demonstrated that mRNA encoding influenza proteins can be administered in lipid nanoparticles, leading to recruitment of immune cells and the translation of the mRNA by monocytes and dendritic cells (Liang et al Efficient Targeting and Activation of Antigen-Presenting Cells In Vivo after Modified mRNA Vaccine Administration in Rhesus Macaques. Mol Ther. 2017). Therefore, transfection of professional APCs (such as monocytes and dendritic cells) with mRNA constructions or compositions as described herein encoding exogenous antigens or epitopes thereof is contemplated to allow for antigen presentation, and the induction of long-lasting adaptive immunity against that antigen. However, expression of antigen within the professional APC is not necessary, and expression of antigen by othertissue can be effective in inducing a desired immune response, as produced antigen can be taken up and processed by professional APC in the normal way after production.

In addition or instead of this, mRNA constructions or compositions as described herein can be used to deliver and express products associated with the process of vaccine-induced immunity, such as cytokines, chemokines, co-stimulatory molecules, or major histocompatibility complexes. Such encoded products are referred to for this discussion as ‘immunostimulators’, ‘immunomodulatory products’ or ‘immunomodulators’, or, when used to stimulate a response to a coadministered vaccine composition, an ‘adjuvant’. If mRNA coding for both antigen and further (immunomodulatory) components are administered, these can be formulated as separate mRNA constructs, or together on the same, polycistronic mRNA, as described above. Where separate mRNA constructs are used for these products, the separate constructs can each comprise the same set of miRNA binding site sequences (that is, they may each comprise the same OPS), or may comprise different sets of miRNA binding site sequences (different OPS), as further discussed below. In some cases, one or other of the mRNA constructs may entirely lack miRNA binding site sequences. It can be appreciated that mRNA encoding products associated with the process of vaccine-induced immunity can be used in combination with any type of vaccine as known to the person of skill in the art, i.e. combination with protein-based (toxoid, recombinant, conjugated vaccines), RNA, mRNA and DNA-based vaccines (including circular or circularised RNA constructs as described above), live-attenuated vaccines, inactivated vaccines, or recombinant-vector based vaccines (e.g. MVA or adenovirus platform).

In this way, the immune response to a co-administered mRNA-encoded antigen or other type of vaccine can be enhanced in a controllable, versatile way. Another advantage with this approach is the expectation that with the administration of the immunomodulator to enhance the immune response, there is the potential to provide multiple polypeptides in a single composition.

For example, macrophages require activation by T-cell secretion of interferon gamma (IFN-γ) in order to express MHC-II. Therefore, induction of IFN-γ expression by transfection with mRNA constructs and compositions as described can enhance the induction of a vaccine-induced immune response, whether resulting from a conventional vaccine approach or an approach using mRNA constructs and compositions as described herein to induce antigen expression. Similarly, the induction of cell receptors involved in immunogenic processes such as TLR, suitably TLR8 as discussed above, can also be carried out using mRNA constructs and compositions as described herein.

The main difference between Th1 and Th2 immune response is that a Th1 immune response is a proinflammatory response, which kills intracellular parasites and perpetuates autoimmune responses, whereas Th2 immune response promotes IgE and eosinophilic responses in atopy and produces anti-inflammatory responses, which kill large, extracellular parasites such as helminths. Furthermore, the key Th1 cytokine is the interferon gamma (IFN-γ) while Th2 cytokines include interleukin 4, 5, 6, 10, and 13. Th1 immune response is the immune response generated by Th1 cells against intracellular parasites like bacteria and viruses. Generally, the cytokine IL-12 is responsible for triggering the Th1 immune response by activating Th1 cells. Furthermore, the activated Th1 cells secrete cytokines such as interferon-gamma (IFN-γ) and interleukin-2 (IL-2). Th1 immune response is a proinflammatory response that leads to cell-mediated immunity. Therefore, it activates macrophages as well as CD8 T cells, IgG B cells, and IFN-γ CD4 T cells. Cytokines produced by Th1 cells including interferon-gamma (INF-γ), interleukin-2 (IL-2), and tumor necrosis factor-beta (TNF-β) mediate Th1 immune responses, while cytokines produced by Th2 cells such as interleukins (IL-4, IL-5, IL-6, IL-10, and IL-13) mediate Th2 immune responses.

IL-12 is produced by dendritic cells, macrophages, neutrophils, and human B-lymphoblastoid cells in response to antigenic stimulation, and is involved in the stimulation and growth of T cells. IL-12 is a pro-stimulatory and pro-inflammatory cytokine with key roles in the development of the Th1 subset of helper T cells. IL-12 was originally discovered because of its ability to induce interferon-gamma (IFN-γ) production, cell proliferation, and cytotoxicity mediated by natural killer cells and T cells. It is now established that IL-12 also plays a key role in the development of Th1 responses, as described above, leading to IFN-γ and IL-2 production. These cytokines can in turn promote cytotoxic T-cell responses and macrophage activation

In another embodiment, it may be desired to administer mRNA constructs and/or compositions leading to IL-12 expression in order to enhance vaccine potency or enhance a vaccine-induced immune response, whether resulting from a conventional vaccine approach or an approach using mRNA constructs and compositions as described herein to induce exogenous antigen expression. In this way IL-12 is present as an adjuvant in order to provide an immunostimulatory response in a recipient that is particularly targeted towards an antigen that is delivered at the same or around the same time. As mentioned previously, the advantageous biological activity of IL-12 to induce a Th1 response promotes IFN-γ and IL-2 production. Together, these cytokines can in turn promote cytotoxic T-cell immunity in response to the administered antigen. An immunogenic response in a recipient of a vaccine therapy of this type is particularly suitable for treatment or prophylaxis of infectious diseases resulting from intra-cellular pathogens such as viruses including SARS-CoV-2, influenza, HIV and RSV to name a few; or even intracellular bacterial pathogens such as Mycobacterium tuberculosis.

Granulocyte-macrophage colony-stimulating factor (GM-CSF or CSF2; GenBank AAA52578) is an immunomodulator produced by various cell types, including T cells, B cells, macrophages, mastocytes cells, endothelial cells, fibroblasts, and adipocytes. GM-CSF also modulates the function of antigen presenting cells and is involved in the enhancement of dendritic cell activation, and the enhancement of mononuclear phagocyte maturation. GM-CSF has been previously used in vaccines to stimulate a response (Yu et al. Novel GM-CSF-based vaccines: One small step in GM-CSF gene optimization, one giant leap for human vaccines. Hum Vaccin Immunother. 2016). In particular, GM-CSF has been shown to improve vaccine response for bacterial disease or infection including but not limited to diphtheria prevention (Grasse M et al. GM-CSF improves the immune response to the diphtheria-component in a multivalent vaccine. Vaccine. 2018), and tuberculosis prevention (Wang et al, Enhanced immunogenicity of BCG vaccine by using a viral-based GM-CSF transgene adjuvant formulation. Vaccine. 2002). Similar improvements have been found or theorised using GM-CSF in vaccine approaches for viral disease or viral infection including but not limited to coronaviruses, influenzae viruses (Liu et al Influenza virus-like particles composed of conserved influenza proteins and GPI-anchored CCL28/GM-CSF fusion proteins enhance protective immunity against homologous and heterologous viruses. Int Immunopharmacol. 2018), and porcine reproductive and respiratory syndrome virus (Yu et al Construction and in vitro evaluation of a recombinant live attenuated PRRSV expressing GM-CSF. Virol J. 2014).

Therefore, introduction of coding mRNA for GM-CSF using mRNA constructs or compositions as described herein can be used to enhance vaccine immunogenicity, through both antibody and cellular immune responses. Such approaches can therefore be used as vaccine adjuvants, enhancers, or immunological boosters in human and other recipients, and in both preventive and therapeutic vaccine types. Similar effects may be seen for other CSF type proteins such as macrophage colony stimulating factor (M-CSF or CSF1; GenBank BC021117) and granulocyte colony stimulating factor (G-CSF or CSF3; GenBank BC033245).

As discussed above, IFN-α and IFN-β are mainly involved in innate immunity against viral infection, and introduction of one or both of these agents using mRNA constructs or compositions as described herein can be used to increase immunogenicity, as described.

IFN-γ synthesis is known to influence the strength and quality of the adaptive immune response. Early synthesis of IFN-γ after immunisation, which develops before the appearance of adaptive immune responses, is a sign of high-quality immune response against a vaccine. This early release of IFN-γ by innate immune cells influences dendritic cell maturation and consequently the polarization of CD4+ T cells to Th1 lineage.

IFN-γ and IL-2 are also produced by activated CD4+Th1 cells as discussed, and the introduction of one or both of these agents is contemplated to be able to increase related responses. Similarly, TNFα as discussed elsewhere herein is released to recruit other immune system cells as part of an inflammatory response to an infection, and therefore its provision using mRNA constructs or compositions as described herein can be used to enhance antiviral immunogenicity.

IL-6 is involved in the final differentiation of B cells into immunoglobulin-secreting cells, and its introduction using mRNA constructs or compositions as described herein is envisioned to improve immunogenicity.

The introduction of IL-8 when administered as described herein is contemplated to improve neutrophil chemotaxis and so to improve immunogenicity.

Other examples of products associated with the process of vaccine-induced immunity which can be induced using mRNA constructs or compositions as described herein include modulators of the Nuclear Factor NF-κB pathway, which has been implicated in the development of vaccine response to the tuberculosis (BCG) vaccine (Shey et al. Maturation of innate responses to mycobacteria over the first nine months of life. J Immunol. 2014).

The ability to choose particular immunomodulators for co-administration such as those described above permits the promotion of particular types of immune response, which can be beneficial for inducing effective immunity against particular pathogens. As an example, since IL-12 as discussed plays a key role in the development of Th1 responses, leading to IFN-γ and IL-2 production, it can be beneficial to co-administer this cytokine when vaccinating against such an intracellular pathogen. Other Th1-associated cytokines may also or alternatively be useful in promoting such reactions, such as IFN-γ, TNF-β, IL-2 and IL-10.

mRNA constructs and compositions according to the above discussion, whether encoding antigen or immunomodulators, can comprise any organ protection sequences as described herein. However, in particular embodiments, the organ protection sequences are selected to protect one or more of muscle, liver, kidney, lungs, spleen, and skin (for example, using target sequences for miRNA-1, miRNA-122, miRNA-192, miRNA-30a and/or miRNA-203a). In some embodiments, target sequences for all four of miRNA-1, miRNA-122, miRNA-30a and miRNA-203a are included in the organ protection sequences. Such a combination is thought to be effective in protecting muscle tissue (as compositions may be administered intramuscularly), as well as liver and kidney tissue. It is particularly considered in any embodiment where the protection of muscle tissue is desired, that target sequences for miRNA 133a and/or for miRNA 206 may be included instead of or in addition to miRNA 1, in accordance with Table 2. For example, such OPS could include target sequences for miRNA-133a, miRNA-122, miRNA-192, and miRNA-30a; or for miRNA-206, miRNA-122, miRNA-192, and miRNA-30a. Subcutaneous or intradermal administration is also common, and one or more of the miRNA target sequences associated with the skin (see Table 2) may also be used to protect cells of the skin.

It is thought that certain vaccines can have side-effects linked to interactions with endothelial tissue. In Goldman M, Hermans C (2021) PLoS Med 18(5): e1003648. https://doi.org/10.1371/journal.pmed.1003648, the following mechanism was suggested: After intramuscular injection, vaccine adenoviruses infect endothelial cells, inducing their production of the SARS-CoV-2 Spike protein. Heparan sulfate PG could bind the spike protein on the luminal side of endothelial cells or be released by damaged cells. Spike proteins would activate platelets via ACE2-dependent and ACE2-independent mechanisms. PF4 released by activated platelets would become immunogenic after binding heparan sulfate PG shed from endothelial cells.

In some embodiments, it may therefore be desired to include miRNA target sequences to protect endothelial tissue. As discussed in Table 2, miRNA-98 and/or miRNA-126 target sequences may therefore be included in OPS. This type of protection is thought to be of use with any mode of administration, and particularly where administration into blood vessels (intravenous, intraarterial, etc.) or intramuscular administration is used.

In other embodiments target sequences may include any appropriate combinations of one or more sequences from Table 3 or 4 above. In specific embodiments, the OPS comprised within mRNA constructs encoding the immunomodulators can comprise sequences capable of binding with: miRNA-122, miRNA-1, miRNA-203a, and miRNA-30a; Let7b, miRNA-126, and miRNA-30a; miRNA-122, miRNA-192, and miRNA-30a; or sequences capable of binding with miRNA-192, miRNA-30a, and miRNA-124, with two sequences capable of binding with miRNA 122.

It is also considered advantageous to avoid the use of miRNA-142 target sequences in such constructs and compositions, as this miRNA is abundant in cells of haematopoietic origin and immune cells, and therefore could lead to a reduction in expression in the cells anticipated to mediate the vaccine-mediated response.

In embodiments where mRNA coding for both antigen and immunomodulatory components are administered, these can be provided as separate mRNA constructs, which may be coformulated, or separately formulated. In some embodiments one or other of the mRNA constructs may entirely lack miRNA binding site sequences. In other cases each mRNA construct may comprise one or more organ protection sequences as described herein. These organ protection sequences may be the same for each mRNA construct, or may be different. It is considered that given the different purposes and potential for off-target effects of antigen and immunomodulator products, use of different organ protection sequences for each of these products may be beneficial, in order to support a different pattern of differential expression for these products, and/or to extend protection to different tissues or cell types for each product.

For example, it may be advantageous for antigen components to be expressed primarily by the myocytes as well as APC, so the organ protection sequences comprised in mRNA encoding these products may be selected to enable expression in these cell types, while protecting other healthy tissue. In some cases, it may be preferred for the antigen component to have organ protection sequences comprising target sequences for miRNA-122, miRNA-192, and/or miRNA 30a, or all three of these.

Immunomodulators, such as IL-12, have the potential of producing off-target effects, so mRNA encoding these factors may be chosen to provide maximum protection to muscle, liver, kidney, lung, spleen and/or skin as discussed above (for example with target sequences for miRNA-1, miRNA-122, miRNA-30a and/or miRNA-203a, or all four of these), while the mRNA encoding the antigen component may comprise fewer miRNA binding site sequences, in order to increase the breadth of expression.

In one embodiment, the immunomodulators administered according to the embodiments of the present invention improve protein-based vaccine immunogenicity.

In one embodiment, the immunomodulators administered according to the embodiments of the present invention improve virus-based vaccine immunogenicity.

Therapeutic Vaccine (or Active Immunotherapy)

In addition to the conventional preventive or prophylactic vaccinations, a newer field is that of therapeutic vaccines which aim to provoke an immune response against targets which are already present in the body, for example, against persistent infections or cancer. This has proven much more challenging, because in such cases the immune response has often been downregulated or otherwise restrained by tolerance mechanisms which act to protect the disease from the normal immune response (Melief et al Therapeutic cancer vaccines JCI 2015).

Therefore, in one embodiment, mRNA constructs as described herein coding for tumoral antigen are provided, for translation in tumour cells. This aims to induce an immune response against the cancer cells as discussed previously. By selective use of the organ protection sequences according to the invention as will by now be evident, expression can be reduced in cell types, tissues and/or organs other than the target tumour tissue, whether that be healthy cells in the tissue surrounding the tumour of the same or different tissue type, or other organs which may be affected by administration use or systemic dispersal.

Such administration can occur in combination with therapeutic vaccines, in order to improve the immune response generated, or can themselves be the therapeutic vaccines, as the immune system reacts to the introduced enhancers in order to mount a response against the tumour.

Combinations with Therapeutic Viruses as Vaccine to Treat Cancer (Therapeutic Cancer Vaccine as Viral Based Immunotherapies)

Cancer treatment vaccines, also called therapeutic vaccines, are a type of immunotherapy that boost the immune system to recognize and destroy cells carrying cancer-specific antigen (tumor-associated antigen, and/or neoantigen), which are not found on healthy cells. For instance, colorectal neoantigen include MUC1, which is commonly found on colorectal tumour cells. Other neoantigens may be specific to the patient tumour. In this later case, the cancer treatment vaccine will be a personalised neoantigen vaccine. Cancer treatment vaccines are used in patients which are already diagnosed with cancer. The therapy can destroy cancer cells, stop tumour growth and spreading, or prevent the cancer from coming back after other treatments have ended. Cancer vaccines may contain the antigen against which an immune response is desired, as well as adjuvants, which strengthen the immune response.

A typical cancer vaccination strategy may involve selecting a suitable vector to deliver the tumor-associated antigen to the main antigen presentation cells of the immune system, e.g. dendritic cells, which are able to generate a long lasting anti-tumoral immune response. In certain embodiments, an adenovirus (Ad) vector, may be used as a vehicle for the delivery of neoantigen genes due to its high efficiency and its low risk for insertional mutagenesis. Adenovirus vectors, such as the ChAdOx1 or ChAdOx2 vectors, are a promising genetic vaccine platform as they rapidly evoke strong humoral and cellular immune responses against the transgene product and the Ad capsid proteins. This has been demonstrated by the generation of anti-tumor T-cell responses, both in vitro and in vivo through dendritic cells infected by tumoral neoantigen-encoding Ad vectors. Therefore, in one embodiment, mRNA constructs as described herein coding for one or more immunomodulators can be used to attract and activate the cellular response generated by the therapeutic cancer vaccine. Suitable immunomodulators as described herein may include IL-12, as well as derivatives (e.g. single chain forms), and homologues thereof. Such mRNA constructs may comprise one or more organ protection sequences, which may be selected, for example, to protect one or more of muscle, liver, kidney, lung, spleen, and skin (for example, using target sequences for miRNANA-1, miRNANA-122, miRNA-30a and/or miRNA-203a; let7b, miRNANA-126, and/or miRNA-30a; or miRNA-122, miRNA-192, and/or miRNA-30a).

Similar to its potential role in preventive/prophylactic vaccines as discussed previously, GM-CSF has also been identified as a potential adjuvant for therapeutic vaccines (Yan et al Recent progress in GM-CSF-based cancer immunotherapy. Immunotherapy. 2017; Zhao et al Revisiting GM-CSF as an adjuvant for therapeutic vaccines. Cell Mol Immunol. 2018). Similarly, CD40 ligand (CD40L), delivered as part of a virus-based vaccine to enhance antigen-specific immunity against cancer, has been shown to improve immune response and the induction of natural killer (NK) cell activation and expansion (Medina-Echeverz et al Synergistic cancer immunotherapy combines MVA-CD40L induced innate and adaptive immunity with tumor targeting antibodies. Nat Commun. 2019). mRNA constructs and compositions as described herein can therefore be used to induce the expression of GM-CSF or CD40L, to enhance anti-tumoral immune response before, during or after cancer treatment vaccines.

It can also be desired to induce an immune response against patient-specific antigen, including ‘neoantigen’, novel antigen produced by cancer cells as the result of mutations (Lichty et al Going viral with cancer immunotherapy. Nat Rev Cancer. 2014). In another embodiment, therefore, mRNA constructs and/or compositions as described herein can be designed comprising mRNA coding for tumor-associated antigen and/or the neoantigen of a patient. In some embodiments, the mRNA constructs of the invention may encode a tumor-associated antigen selected from one or more of alphafetoprotein (AFP), Carcinoembryonic antigen (CEA), CA-125, MUC-1, Epithelial tumor antigen (ETA), Tyrosinase, Melanoma-associated antigen (MAGE), Prostate-Specific Antigen (PSA), human epidermal growth factor receptor 2 (HER2), abnormal products of ras, or p53.

These can be in conjunction with any other mRNA coding for immunomodulators, immune enhancers, and other effector compounds, as discussed above, either in the same or different mRNA constructs. Such approaches aim to induce tumor cells to produce antigenic protein of enhanced effect, allowing the immune system to better recognise those tumor cells. This cellular response against the tumor cells can also be enhanced by further inducing the expression of immunomodulators by the cancer cells. As in the above discussion on prophylactic vaccines, where separate mRNA constructs are used to provide both tumor-associated antigen (or neoantigen) and immunomodulatory components, in some embodiments one or other of the mRNA constructs may entirely lack miRNA binding site sequences. In other cases each mRNA construct may comprise one or more organ protection sequences as described herein. These organ protection sequences may be the same for each mRNA construct, or may be different. Where the organ protection sequences are different, these may be chosen in order to support a different pattern of differential expression for these products, and/or to extend protection to different tissues or cell types for each product.

Hence, according to specific embodiments of the present invention there is provided an mRNA as described herein that encodes a therapeutic enhancement factor, such as an immunostimulatory or immune-modulatory protein or polypeptide, for use in combination with a cancer immunotherapeutic such as a cancer vaccine. The cancer vaccine may comprise a therapeutic virus, such as a modified human or primate adenovirus, and the immunostimulatory or immune-modulatory protein or polypeptide may comprise biologically active IL-12 and/or GM-CSF.

In another embodiment, mRNA constructs and/or compositions as described herein coding for a modulator and/or inhibitor of the NF-kB pathway are provided, for expression in or by tumoral cells, as discussed throughout.

The compositions and methods of the invention are exemplified by, but in no way limited to, the following Examples.

### EXAMPLES

mRNA Constructs

All mRNA constructs are synthetized by Trilink Biotechnologies (San Diego, CA) from a generated DNA sequence. These mRNAs resemble fully processed, capped and polyadenylated mRNAs and are ready for translation by the ribosome.

Formulation

All mRNA constructs are formulated into a multi-component nanoparticle of ionizable lipid-like material C12-200, phospholipid DOPE, cholesterol and lipid-anchored polyethylene glycol C14-PEG2000-DMPE mixture. This particular composition and specific weight ratio (10:1) of C12-200:mRNA and molar [%] composition of lipid-like material, phospholipid, cholesterol and PEG was optimized for high transfection efficiency in vivo (Kauffman K. J., Nano Letter. 2015, 15, 7300-7306) and is referred to as DMP^{CTx}-mRNA. To make the formulations, lipid components were dissolved in ethanol and mixed at 1:3 ratio with mRNA diluted in 10 mM citrate buffer (pH 3) using a T junction mixing apparatus. Formulations were dialyzed in 20 kDa membrane dialysis cassettes against phosphate buffered saline (PBS, pH 7.4) for 4 hours at room temperature. When necessary, formulations were then concentrated using Amicon Ultra centrifugal filtration units (100 kDa cutoff). Subsequently, formulations were transferred to a new tube ready for characterization. Efficacy of mRNA encapsulation and concentration is measured using the Ribogreen RNA assay (Invitrogen) according to the manufacturer's protocol. Polydispersity Index (PDI) and size (Zₐᵥₑ) of the lipid nanoparticles are measured using dynamic light scattering (Zetasizer Nano-ZS, Malvern).

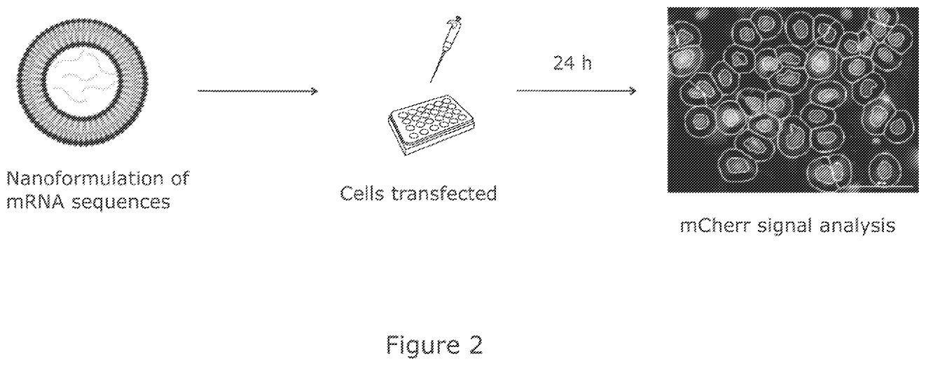

Formulations are used to transfect cells in a multi-well plate assay as depicted in FIG. 2. Formulations are suitably diluted to around 1.5 pmol/well of DMP^{CTx}-mRNA. The readout of the assay is to detect expression of the mRNA via mCherry fluorescence, with Hoechst 33342 staining (NucBlue Liver ReadyProbes Reagent, Life Technologies) to stain cell nuclei and determine cell density. mCherry fluorescence was quantified 24 h after transfection using fluorescence microscopy (Cytation Systems from BIOTEK or EVOS FL Auto from Thermofisher Scientific).

These formulations are also used to transfect cells in in vivo animal studies.

Cell Cultures and Transfection

All cells were grown at 37° C. in the presence of 5% CO₂. In vitro single transfections of cultured cells (Hep3B, AML12, 786-0, hREC, HCT-116) were performed as follows: one day prior to transfection, cells were seeded in a 96-well tissue culture treated microwell plate in recommended complete media and cell density listed in Table 7. The next day, cells were transfected with 1.5 pmol of DMP^{CTx}-mRNAs in 200 uL of reduced serum medium (Opti-MEM medium, Gibco) by direct addition of mRNA-DMP^{CTx} to the medium in the well, with gentle mixture of the cultured cells as needed. After 4 hours of incubation, the Opti-MEM medium was removed and substituted by complete media.

TABLE 7
medium and cell density used for in vitro transfection of DMP^{CTx}-mRNA

| | | Complete | Cell density for |
|---|---|---|---|
| Cell Type | | Medium | seeding microwells |
| Hep3B | Human hepatocellular | Eagle's Minimum Essential | 60,000 cells |
| | carcinoma (ATCC HB- | Medium, 10% Fetal Bovine | per mL |
| | 8064) | Serum | |
| AML12 | Normal murine liver | DMEM:F12, 10 ug/mL | 60,000 cells |
| | cells (ATCC CRL- | insulin, 5.5 ug/mL | per mL |
| | 2254) | transferrin, 5 ng/selenium, | |
| | | 40 ng/ml dexamethasone, | |
| | | 10% Fetal Bovine Serum | |
| 786-0 | Human renal | RPMI-1640, 10% Fetal | 60,000 cells |
| | adenocarcinoma | Bovine Serum | per mL |
| | (ATCC 1932) | | |
| HREC | Normal human renal | Renal Epithelial Cell Basal | 60,000 cells |
| | mixed cells (ATCC | Medium (ATCC PCS-400- | per mL |
| | PCS-400-012) | 030), Renal Epithelial Cell | |
| | | Growth kit (ATCC PCS- | |
| | | 400-040) | |
| HCT-116 | Human colorectal | McCoy's 5A medium, 10% | 400,000 cells |
| | carcinoma | Fetal Bovine Serum | per mL |

For transfection of human normal hepatocytes (Sigma Product Reference No MTOXH1000), cells were plated in 24-well Collagen coated plates at a cell density of 250,000 cells per mL using Sigma recommended thawing, fully supplemented plating and culture media (references MED-HHTM, MED-HHPM, MED-HHPMSP, MED-HHCM, MED-HHCMSP). Transfection of mRNA-DMP^{CTx} was performed in culture medium containing 5% FBS. After 4 hours of incubation, the mRNA mixture was removed, and fully supplemented medium was added back to the wells.

For transfection of normal epithelial adult colonic cells (Cell Applications, Inc., reference 732Cn-05a), cells were thawed, plated and cultured using GI Epithelial Cell Thawing Solution and GI Epithelial Cell Defined Culture Medium (Cell Applications Inc., references 716DC-50 and 716T-20). 96-well microplate wells were pretreated with GI Epithelial Cell Coating Solution (Cell Applications, Inc., reference 025-05) and 60,000 cells per well were seeded. The next day, cells were transfected with 1.5 pmol of DMP^{CTx}-mRNAs in 200 uL of reduced serum medium (Opti-MEM medium, Gibco) by direct addition of mRNA-DMP^{CTx} to the medium in the well, with gentle mixture of the cultured cells as needed. After 4 hours of incubation, the Opti-MEM medium was removed and substituted by the culture medium.

For transfection of normal human lung/bronchial cells (BAES-2B cells, ATCC CRL-9609), cells were grown in BEGM medium (Lonza) supplemented with BEGM Bronchial Epithelial SingleQuots kit (Lonza). Cells were seeded in Collagen I coated microwell plates at a density of 75,000 cells per mL. The next day, cells were transfected with 1.5 pmol of DMP^{CTx}-mRNAs in 200 uL of reduced serum medium (Opti-MEM medium, Gibco) by direct addition of DMP^{CTx}-mRNA to the medium in the well, with gentle mixture of the cultured cells as needed. After 4 hours of incubation, the Opti-MEM medium was removed and substituted by the culture medium.

Fluorescence Microscopy

24 hours following transfection, cells nuclei were stained using the Hoechst 33342 dye (NucBlue™ Live ReadyProbes™ Reagent from Invitrogen). Nuclei staining and mCherry fluorescence were detected in live cells using a fluorescence microscope (Cytation instrument from Biotek or EVOS® FL Imaging Systems from Thermofisher Scientific). Images were acquired with filter cubes Texas Red and DAPI and a 20× objective.

### Example 1: Unoptimized Vs Optimized miRNA Target Sequence (Unmatched Vs Matched)

To investigate the potential of the present invention to successfully transfect target cells with construct mRNA and subsequently drive better protein differential expression than the unmodified miRNA target sequence, the DMP^{CTx} mRNA platform, modified with miRNA binding sites, is first evaluated in an in vitro model using human cancer cell lines and normal primary cells for each organ. Purified mCherry mRNA is used for tracking transfection and translation efficiency in cultured cells.

For instance, miRNA-122 is an abundant, liver-specific miRNA, the expression of which is significantly decreased in human primary hepatocarcinoma (HCC) and HCC derived cell lines such as Hep3B. The objective of this example study is to demonstrate that modification of the 3′-untranslated region (UTR) of an mRNA sequence by the insertion of an optimized miRNA-122 targeted sequences (for example, variant 2) may result in a higher translational repression of exogenous mRNA in normal hepatocytes, but not in tested HCC cell lines. For that purpose, an mCherry mRNA construct was modified to include at least one unoptimized miRNA-122 target sequence in the 3′-UTR (variant 1) or at least one optimized perfect matching target sequence (variant 2). The mRNA construct is transfected into murine AML12 normal hepatocytes, known to express high level of miRNA-122. An mCherry mRNA construct with no miRNA target sequence was used as a positive control. mCherry fluorescence was detected 24 h after single transfection of the mCherry mRNA constructs using fluorescence microscopy (EVOS FL Auto from Thermofisher Scientific). Alternative quantification methodology can be used to verify the expression of the delivered construct, including Western blot or proteome analysis techniques such as mass spectrometry.

Variant 1 [SEQ ID NO: 4]:
5′-AACGCCAUUAUCACACUAAAUA-3′
(unmatched miRNA-122 target sequence)
Variant 2 [SEQ ID NO: 44]:
5′-CAAACACCAUUGUCACACUCCA-3′
(perfect matched miRNA-122 target
sequence)

Results

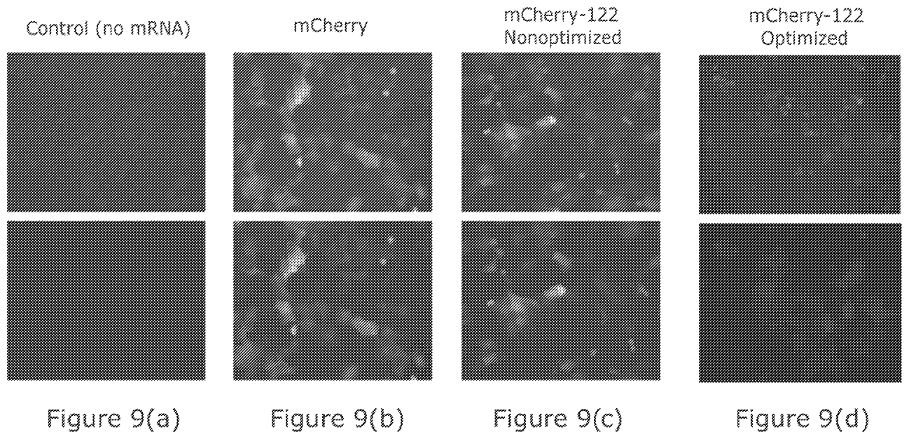

As shown in FIG. 9B the expression of mCherry mRNA without MOP is strong in AML12 cells. When a single imperfectly matched miRNA-122 target sequence (Variant 1) is included in the 3′ UTR, expression of mCherry remains evident (see FIG. 9C. The effect of perfect matching using Variant 2 is clear with much reduced mCherry expression apparent in FIG. 9D, right hand panel.

### Example 2: Comparing the Effect of Repeat Numbers of miRNA Target Sequences

To investigate the potential of the present invention to drive better differential expression by increasing the number of target sequences in the mRNA construct, mCherry mRNA was modified to include one, two or four optimized miRNA-122 miRNA target sequences in the 3′-UTR and translation efficiency was evaluated and compared in vitro in human Hep3B cancer cell lines and corresponding normal AML12 primary cells. An mCherry mRNA construct with no miRNA target sequence was used as a positive control. miRNA-122 target sequences are linked using specific nucleotide (like uuuaaa) as shown in FIG. 1. mCherry fluorescence was detected 24 h after single transfection of the mCherry mRNA constructs by fluorescence microscopy (EVOS FL Auto from Thermofisher Scientific). Alternative quantification methodology can be used to verify the expression of the delivered construct, including Western blot or proteome analysis techniques such as mass spectrometry also.

Results

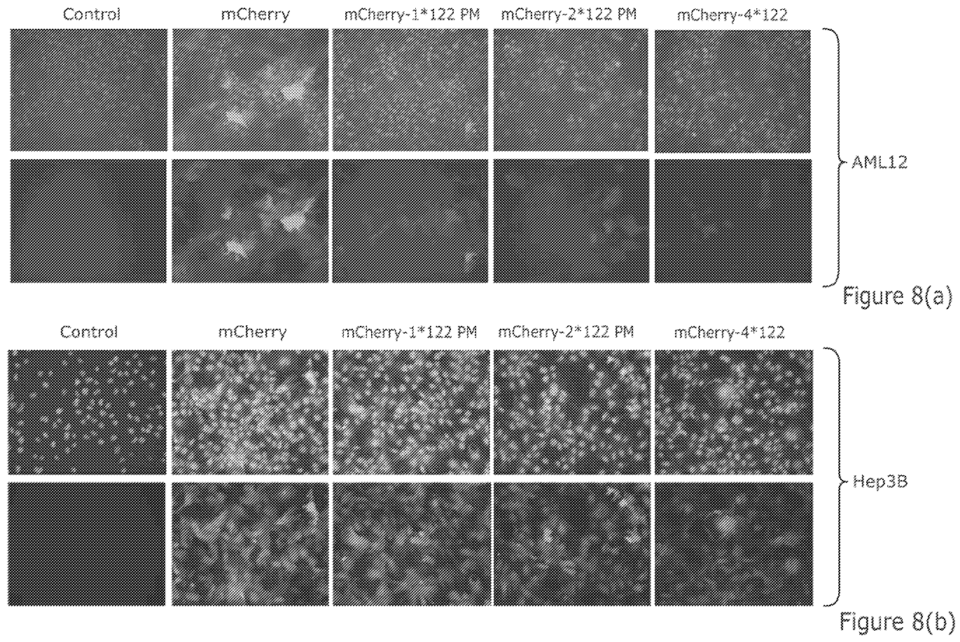

The results of multiplexing a binding site sequence are shown in FIG. 8. There is some dose dependence in suppression of mCherry expression in AML12 normal hepatocytes (a), with two and four repetitions of the binding site sequence showing high levels of suppression of mCherry. However, the effect is less evident for Hep3B cancer cells (b), where the levels of mCherry expression remain largely consistent for one or two repetitions of the miRNA binding site, with only a slight reduction in expression for the four-fold multiplexed sequence.

### Example 3: Proof of Concept of Multi-Organ Protection Methods In Vitro

To investigate the potential of the present invention to demonstrate differential expression of a particular ORF in multiple different recipient cell types, mCherry mRNA was modified to include three or five miRNA target sequences in the 3′UTR. In a first mRNA sequence target sequences for miRNA-122, Let7b and miRNA-192 are provided (mCherry-3MOP), and in a second mRNA sequence target sequences for miRNA-122, miRNA-124a, Let7b, miRNA-375, and miRNA-192 are provided (mCherry-5MOP). A control mCherry mRNA sequence was also used without miRNA target sequences.

The prepared mRNA sequences were nanoformulated as described above. The prepared nanoparticles were transfected into cell lines (FIG. 2) corresponding to human normal hepatocytes (Sigma Product Reference No MTOXH1000); murine normal hepatocytes (AML12 from ATCC), and human hepatocarcinoma cells (Hep3B from ATCC). In addition, a cell line corresponding to normal human kidney cells (hREC from ATCC) was transfected with mCherry-3MOP mRNA, and control mCherry RNA. Cells were seeded in a 24-well plate. 0.5 ug of mRNA was transfected per well and imaging was performed 24 h post-transfection with a Cytation 5 instrument (Biotek).

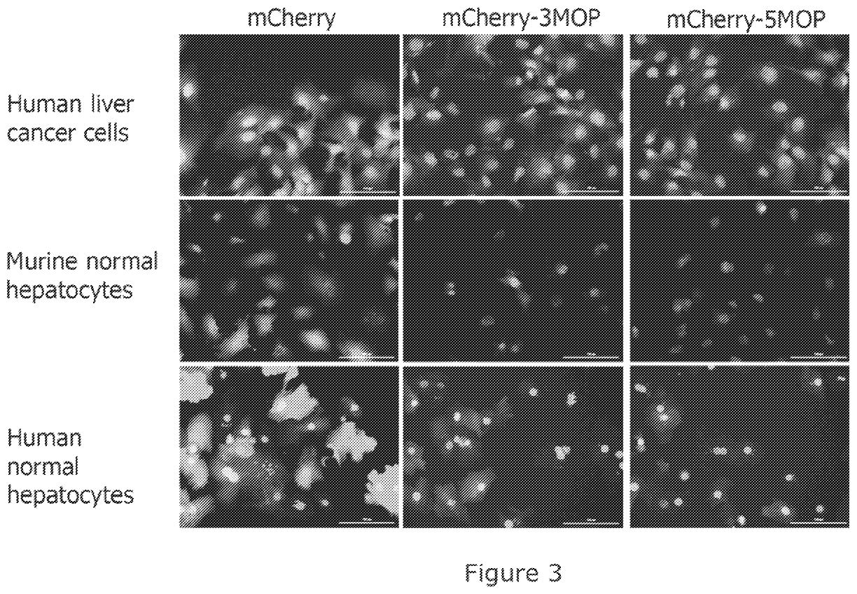

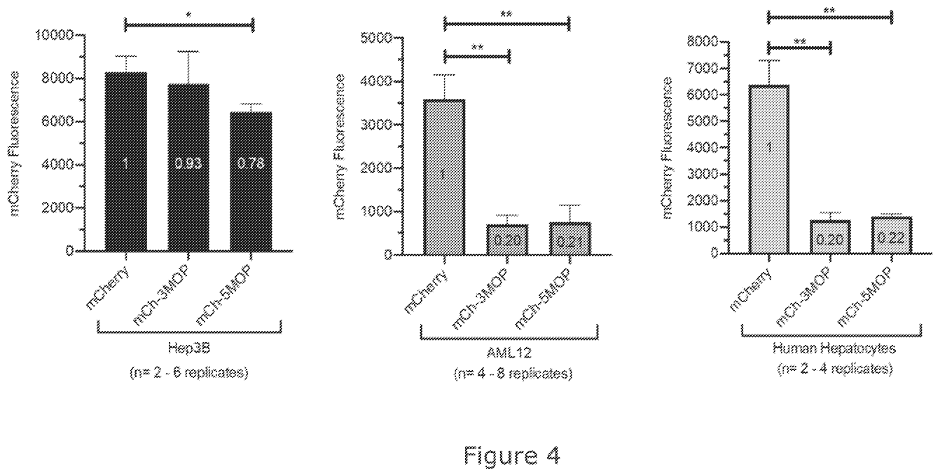

FIG. 3 shows mCherry signal in the three liver cell types, and demonstrates significant reduction of cell signal in both normal murine and human hepatocytes when transfected with mCherry-3MOP or mCherry-5MOP mRNA, compared to the signal found in the human liver cancer cells (Hep3B) or in the normal cells after transfection with control mCherry mRNA. This indicates a reduction in mCherry translation in normal cells as a result of the inclusion of the miRNA target sequences. FIG. 4 shows quantification of mCherry fluorescence in the transfected cells using the Gen5 Imaging Software from Biotek. Background signal has been subtracted. Values represent the mean and standard deviation of fluorescence signal per cell. Statistically significant differences for assessed mRNA compared to control are shown as * P<0.05, ** P<0.005. The results demonstrate approximately 80% reduction in protein expression in normal liver cells (human and murine) when 3MOP or 5MOP miRNA target sequences are used, while less reduction is seen in tumoral cells.

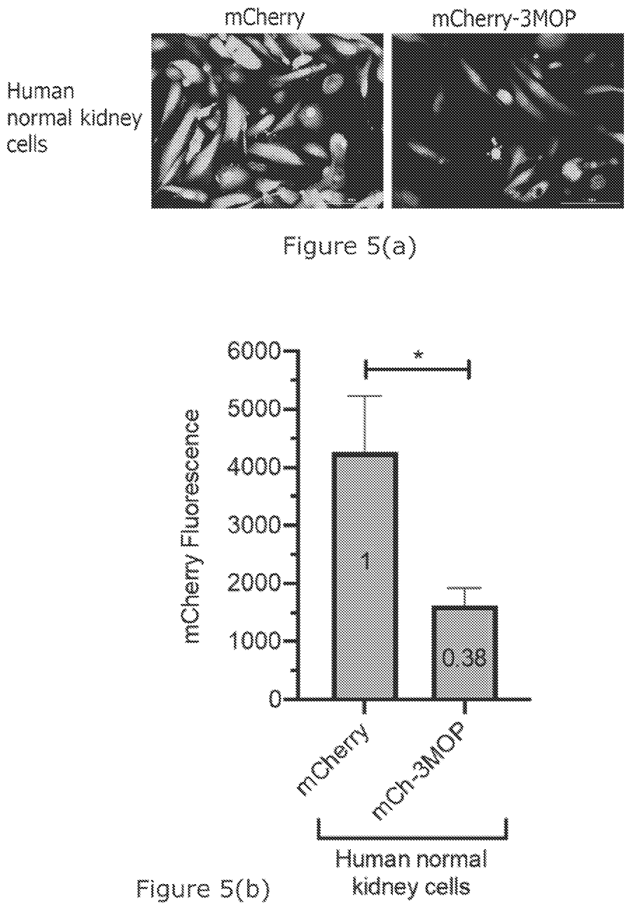

FIG. 5A shows mCherry signal in transfected normal human kidney cells (hREC ATCC-PCS-400-012). A reduction in signal is visible in the mCherry-3MOP treated cells, indicating a reduction in mCherry translation. This is quantified in FIG. 5B using the Gen5 Imaging Software from Biotek, which likewise shows around a 60% reduction in mCherry signal in normal kidney cells after transfection with mCherry-3MOP. In FIG. 5B, background signal has been subtracted, and values represent the mean and standard deviation of fluorescence signal per cell. Statistically significant differences for assessed mRNA compared to control are shown as * P<0.05.

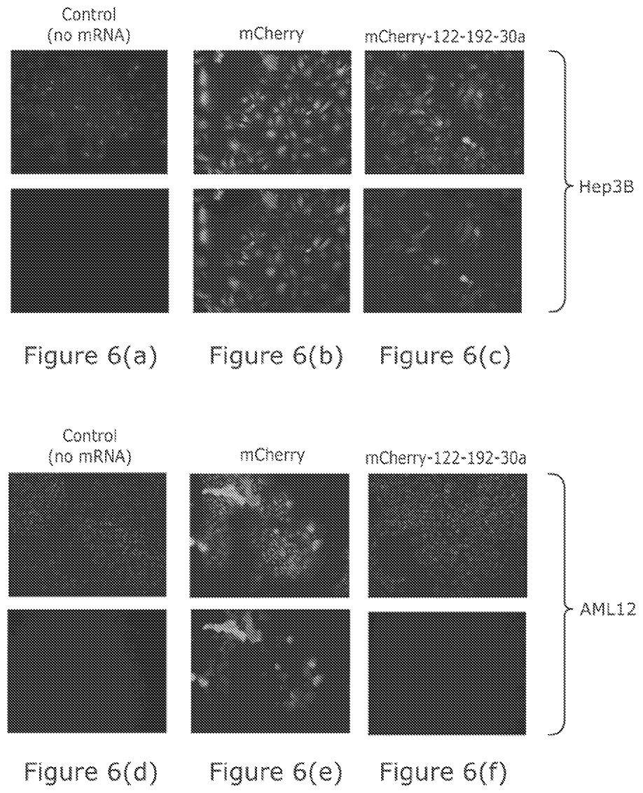

In FIG. 6 the results of an experiment in liver cells using an alternative configuration of a 3MOP sequence are shown. In this instance the 3MOP sequence comprises miRNA binding sites that have been perfectly matched to miRNA122, miRNA192 and miRNA30a. The expression in Hep3B cancer cells is clearly seen in FIG. 6(c) when compared to murine AML12 hepatocytes in FIG. 6(f), where no discernible expression is visible.

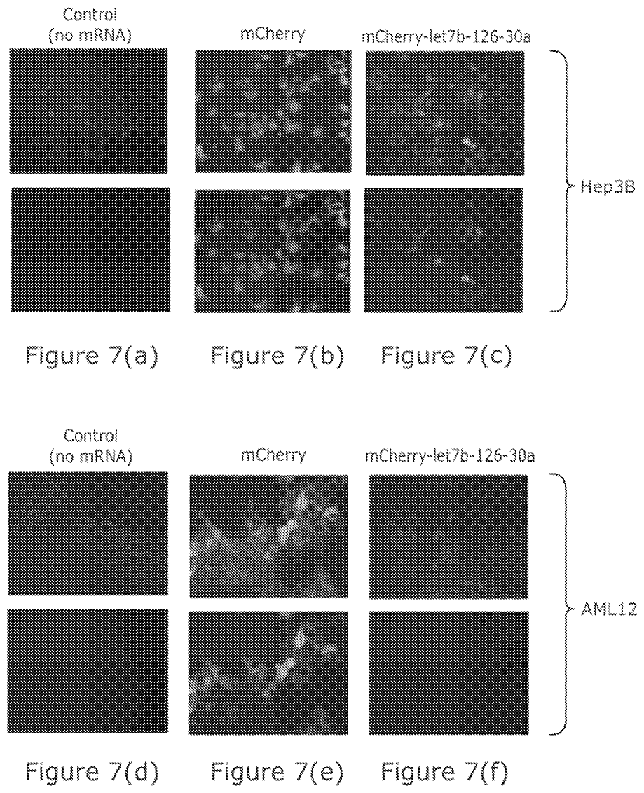

FIG. 7 shows the results of another alternative configuration of the 3MOP sequence. In this instance the 3MOP sequence comprises miRNA binding sites that have been perfectly matched to Let7b, miRNA126 and miRNA30a. Again, the mCherry expression in Hep3B cancer cells is clearly seen in FIG. 7(c) when compared to murine AML12 hepatocytes in FIG. 7(f), where no discernible expression is visible.

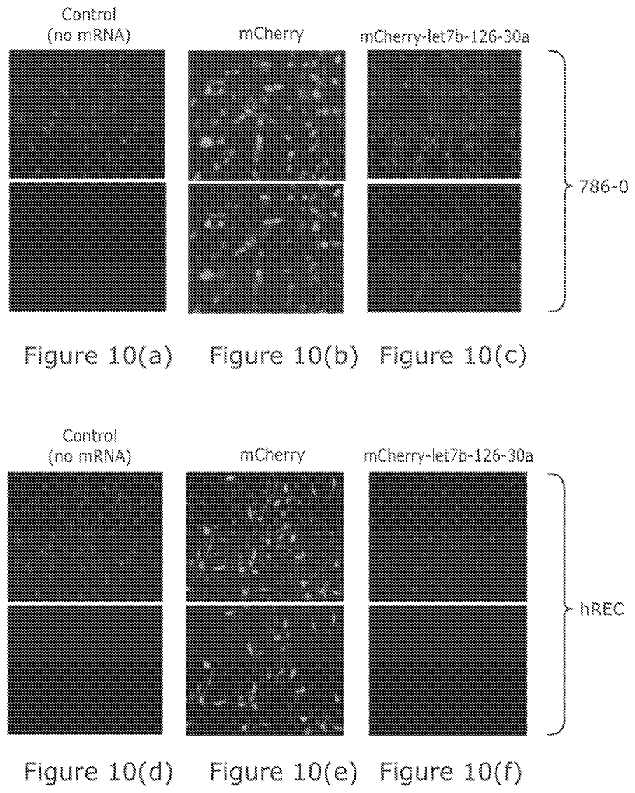

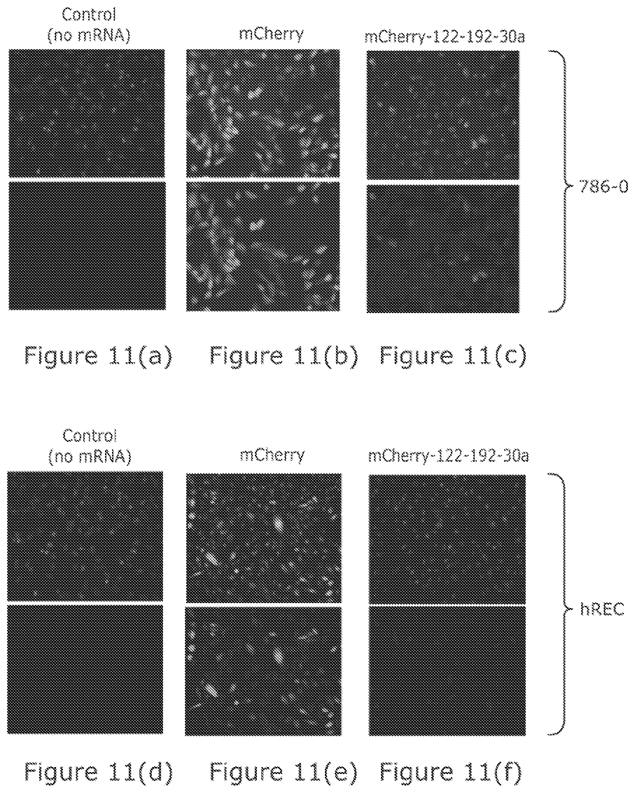

FIG. 10 demonstrates the effects of tissue and organ specific protection in the kidney for the same 3MOP sequence as used in the experiments for FIG. 7 (miRNAlet7b-miRNA126-miRNA30a). The expression of mCherry is almost completely suppressed in hREC human kidney cells (FIG. 10(f)) but not in 786-0 renal adenocarcinoma cells (FIG. 10(c)). In FIG. 11 the alternative 3MOP sequence is tested (miRNA122-miRNA192-miRNA30a). Both the MOP sequences tested for FIGS. 10 and 11 comprise perfect match binding sequence for miRNA30a, which is protective of kidney, however, the latter 3MOP further comprises a perfect match miRNA-192 binding site which provides a putative double layer of kidney protection (see Table 2, above). In FIG. 11 the expression of mCherry is not visible in hREC human kidney cells (FIG. 11(f)) but is clearly evident in 786-0 renal adenocarcinoma cells (FIG. 11(c)).

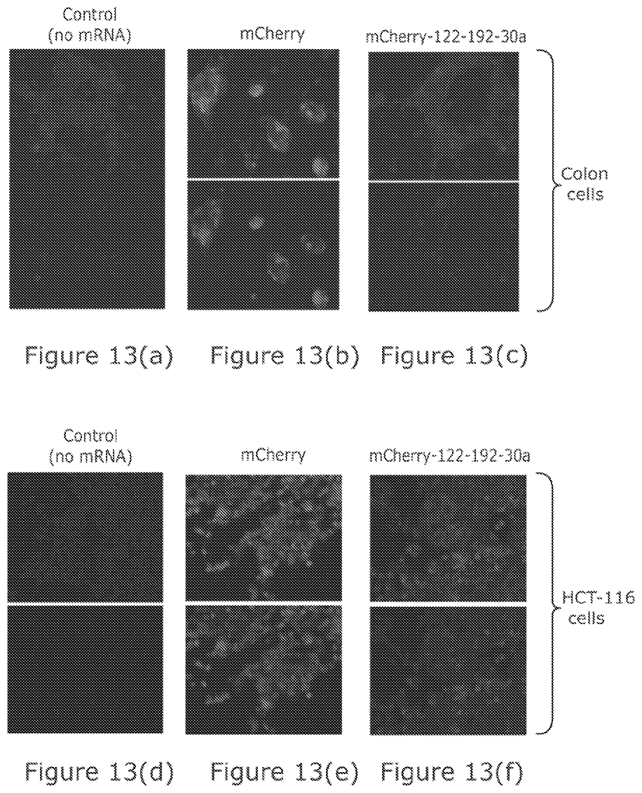

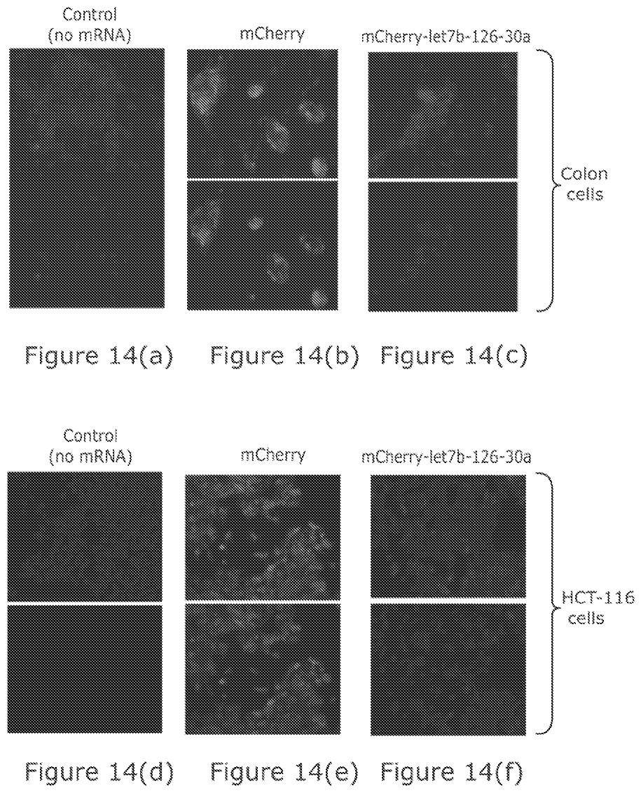

FIG. 13 demonstrates the effects of tissue and organ specific protection in the colon for the same 3MOP sequence as used in the experiments for FIG. 11 (miRNA122-miRNA192-miRNA30a). The expression of mCherry is almost completely suppressed in human colon epithelial cells (FIG. 13(c)) but not in HCT-116 cells (FIG. 13(f)). In FIG. 14 the alternative 3MOP sequence is tested (miRNAlet7b-miRNA126-miRNA30a). The MOP sequence tested for FIG. 14 comprises a perfect match binding sequence for Let7b, which is broadly protective of colon tissue. In FIG. 14 the expression of mCherry is highly reduced in colon epithelial cells (FIG. 14(c)) and also in HCT-116 cells (FIG. 14(f).

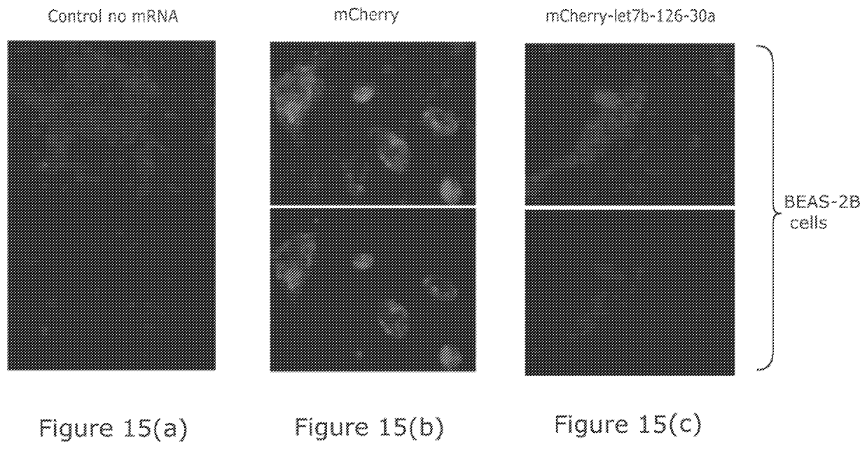

FIG. 15 demonstrates the effects of tissue and organ specific protection in the lung using the MOP sequence comprising binding sites for miRNAlet7b, miRNA126, and miRNA30a. Similarly to the other assays described above, a bronchial epithelial cell line was selected that represents the closest approximation of healthy non-cancerous human lung tissue: the BEAS-2B cell line. Whilst BEAS-2B cells are immortalized via infection with replication-defective SV40/adenovirus 12 hybrid, this is to enable improved handling and cloning. The cells are used in assays to study differentiation of squamous cells as a model of normal functioning lung epithelium. In FIG. 15 (c), presence of the MOP sequence results in very high levels of suppression of mCherry expression compared to the absence of MOP.

The results shown in FIGS. 7(f), 10(f), 14(c) and 15(c) show that inclusion of the miRNAlet7b-miRNA126-miRNA30a MOP sequences provides effective protection from associated ORF expression in healthy liver, kidney, colon and lung. The results for FIGS. 6(f), 11(f), and 13(c) show that the alternative MOP comprising miRNA122-miRNA192-miRNA30a binding sequences provides effective protection for healthy liver, kidney and colon tissue.

This example demonstrates that organ protection sequences comprising multiple different miRNA target sequences can also act to drive differential expression in cells derived from multiple different organs, and can differentiate between normal and tumoral cells in multiple tissues.

### Example 4: Transfection of Human PBMCs with IL-12 and GM-CSF mRNAs

IL-12 and/or GM-CSF are immunomodulatory cytokines that may be utilised in combination with anti-tumour therapies such as in combination with therapeutic viruses, or as adjuvants co-administered with vaccine compositions. In this experiment, DMP^{CTx} hGM-CSF (human GM-CSF) and hdcIL-12 (double chain human IL-12 p70) or hscIL-12 (single chain human IL-12 p70), with or without a MOP sequence were administered in vitro to human PBMCs at a range of dosages. Noncoding mRNA for hscIL-12 p70 and hGMCSF we also used as negative control (NC). The expression of protein within the cell was shown to be linked to the dose of mRNA administered.

In Vitro Transfection Efficiency and Toxicity of LNP-Immunomodulators IL-12 and GM-CSF

PBMC cells from 5 different donors (18-55 years old) were obtained from AllCells and cultured in suspension in AIM V medium (Gibco) at 37° C. in an atmosphere of 5% CO2. 300,000 PBMC cells were seeded per well in a round-bottom 96-well plate and transfected with DMP^{CTx} formulated mRNAs encoding either IL-12 single or double chain variants or GM-CSF, with or without MOP sequence (see Table 8, below). Positive control to validate PBMCs are functioning normally was performed with wells containing 300,000 PBMC cells with LPS (ThermoFisher, 00-4976) added to the media at a final concentration of 100 ng/mL. Negative controls were performed in parallel, with wells without PBMC cells nor LNP transfection (BG-C), wells with only 150,000 non-transfected PBMC cells (LC), and wells with 300,000 non-transfected PBMC cells (HC).

4 hours after transfection, human AB heat-inactivated serum (Sigma) was added to a final concentration of 1%. 6 hours after transfection, 60 μL of the supernatant of each well were transferred to a new 96-well plate, cells were removed by centrifugation and the supernatants were frozen at −80° C. for MSD assay. 21 hours after transfection, Tween-20 was added to HC wells at a final concentration of 1.1%. 24 hours after transfection, all supernatants from each well were collected by centrifugation. 60 μL of supernatant were frozen at −80° C. for MSD assay and the remaining 130 μL were frozen at −80° C. for LDH assay.

MSD assay for human cytokines IL-12p70 and GM-CSF analysis was performed using a U-PLEX assay (Meso Scale Discovery) and following the manufacturer's instructions. The data were plotted into a bar graph using Graph Pad Prism.

LDH assay was performed using the Cytotoxicity Detection KitPLUS (LDH) from Roche (4744926001) and following the manufacturer's instructions.

Results

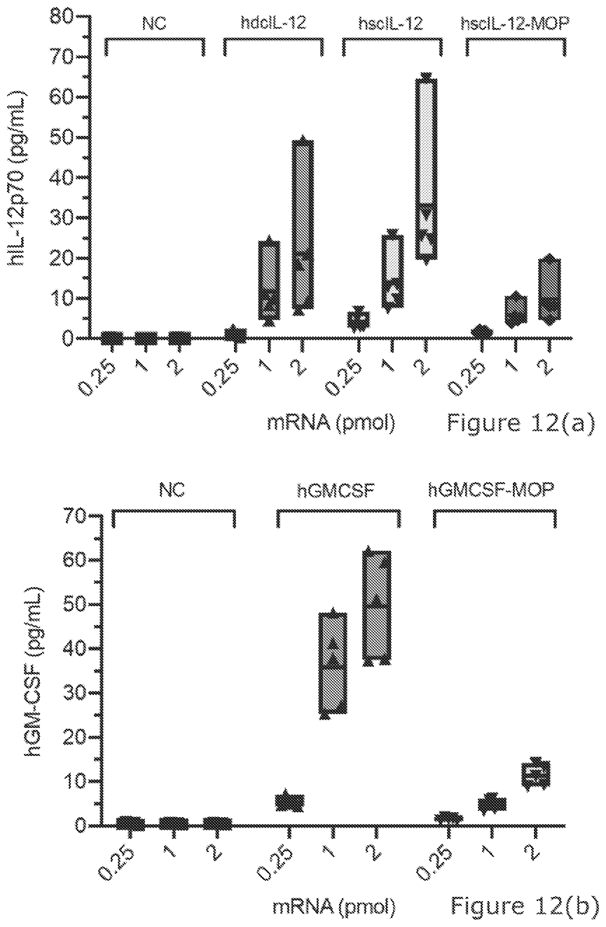

FIG. 12 shows detectable levels of both human IL-12 p70 and GM-CSF from MOP containing constructs 6 h after transfection. The presence of the MOP in the mRNA minimises off-target expression in liver, skin, muscle and kidney tissues. The LDH assay showed that DMP^{CTx} mRNAs encoding for either IL-12 single or double chain variants or GM-CSF did not induce significant cellular cytotoxicity in the PBMCs above the negative control. The results were consistent when checked again after 24 h (data not shown).

TABLE 8
ORF and 3′ UTR for IL-12 MOP and GM-CSF MOP
Human single chain IL-12 MOP (hscIL12-MOP)
with 3′ UTR perfect matching complementary
sequence to miRNA-122-5P, miRNA-1-3P,
miRNA-203a-3P, miRNA-30a-5P are underlined
(MOPV) [SEQ ID NO: 58]
AUGUGUCACCAGCAGUUGGUCAUCUCUUGGUUUUCCCUGGUUUUUCUGGC
AUCUCCCCUCGUGGCCAUAUGGGAACUGAAGAAAGAUGUUUAUGUCGUAG
AAUUGGAUUGGUAUCCGGAUGCCCCUGGAGAAAUGGUGGUCCUCACCUGU
GACACCCCUGAAGAAGAUGGUAUCACCUGGACCUUGGACCAGAGCAGUGA
GGUCUUAGGCUCUGGCAAAACCCUGACCAUCCAAGUCAAAGAGUUUGGAG
AUGCUGGCCAGUACACCUGUCACAAAGGAGGCGAGGUUCUAAGCCAUUCG
CUCCUGCUGCUUCACAAAAAGGAAGAUGGAAUUUGGUCCACUGAUAUUUU
AAAGGACCAGAAAGAACCCAAAAAUAAGACCUUUCUAAGAUGCGAGGCCA
AGAAUUAUUCUGGACGUUUCACCUGCUGGUGGCUGACGACAAUCAGUACU
GAUUUGACAUUCAGUGUCAAAAGCAGCAGAGGCUCUUCUGACCCCCAAGG
GGUGACGUGCGGAGCUGCUACACUCUCUGCAGAGAGAGUCAGAGGGGACA
ACAAGGAGUAUGAGUACUCAGUGGAGUGCCAGGAGGACAGUGCCUGCCCA
GCUGCUGAGGAGAGUCUGCCCAUUGAGGUCAUGGUGGAUGCCGUUCACAA
GCUCAAGUAUGAAAACUACACCAGCAGCUUCUUCAUCAGGGACAUCAUCA
AACCUGACCCACCCAAGAACUUGCAGCUGAAGCCAUUAAAGAAUUCUCGG
CAGGUGGAGGUCAGCUGGGAGUACCCUGACACCUGGAGUACUCCACAUUC
CUACUUCUCCCUGACAUUCUGCGUUCAGGUCCAGGGCAAGAGCAAGAGAG
AAAAGAAAGAUAGAGUCUUCACGGACAAGACCUCAGCCACGGUCAUCUGC
CGCAAAAAUGCCAGCAUUAGCGUGCGGGCCCAGGACCGCUACUAUAGCUC
AUCUUGGAGCGAAUGGGCAUCUGUGCCCUGCAGUGGUGGCGGUGGCGGCG
GAUCUAGAAACCUCCCCGUGGCCACUCCAGACCCAGGAAUGUUCCCAUGC
CUUCACCACUCCCAAAACCUGCUGAGGGCCGUCAGCAACAUGCUCCAGAA
GGCCAGACAAACUCUAGAAUUUUACCCUUGCACUUCUGAAGAGAUUGAUC
AUGAAGAUAUCACAAAAGAUAAAACCAGCACAGUGGAGGCCUGUUUACCA
UUGGAAUUAACCAAGAAUGAGAGUUGCCUAAAUUCCAGAGAGACCUCUUU
CAUAACUAAUGGGAGUUGCCUGGCCUCCAGAAAGACCUCUUUUAUGAUGG
CCCUGUGCCUUAGUAGUAUUUAUGAAGACUUGAAGAUGUACCAGGUGGAG
UUCAAGACCAUGAAUGCAAAGCUUCUGAUGGAUCCUAAGAGGCAGAUCUU
UCUAGAUCAAAACAUGCUGGCAGUUAUUGAUGAGCUGAUGCAGGCCCUGA
AUUUCAACAGUGAGACUGUGCCACAAAAAUCCUCCCUUGAAGAACCGGAU
UUUUAUAAAACUAAAAUCAAGCUCUGCAUACUUCUUCAUGCUUUCAGAAU
UCGGGCAGUGACUAUUGAUAGAGUGAUGAGCUAUCUGAAUGCUUCCUAAC
AAACACCAUUGUCACACUCCAUUUAAAAUACAUACUUCUUUACAUUCCAU
UUAAACUAGUGGUCCUAAACAUUUCACUUUAAACUUCCAGUCGAGGAUGU
UUACA
Human single chain IL-12 without MOP
[SEQ ID NO: 59]
AUGUGUCACCAGCAGUUGGUCAUCUCUUGGUUUUCCCUGGUUUUUCUGGC
AUCUCCCCUCGUGGCCAUAUGGGAACUGAAGAAAGAUGUUUAUGUCGUAG
AAUUGGAUUGGUAUCCGGAUGCCCCUGGAGAAAUGGUGGUCCUCACCUGU
GACACCCCUGAAGAAGAUGGUAUCACCUGGACCUUGGACCAGAGCAGUGA
GGUCUUAGGCUCUGGCAAAACCCUGACCAUCCAAGUCAAAGAGUUUGGAG
AUGCUGGCCAGUACACCUGUCACAAAGGAGGCGAGGUUCUAAGCCAUUCG
CUCCUGCUGCUUCACAAAAAGGAAGAUGGAAUUUGGUCCACUGAUAUUUU
AAAGGACCAGAAAGAACCCAAAAAUAAGACCUUUCUAAGAUGCGAGGCCA
AGAAUUAUUCUGGACGUUUCACCUGCUGGUGGCUGACGACAAUCAGUACU
GAUUUGACAUUCAGUGUCAAAAGCAGCAGAGGCUCUUCUGACCCCCAAGG
GGUGACGUGCGGAGCUGCUACACUCUCUGCAGAGAGAGUCAGAGGGGACA
ACAAGGAGUAUGAGUACUCAGUGGAGUGCCAGGAGGACAGUGCCUGCCCA
GCUGCUGAGGAGAGUCUGCCCAUUGAGGUCAUGGUGGAUGCCGUUCACAA
GCUCAAGUAUGAAAACUACACCAGCAGCUUCUUCAUCAGGGACAUCAUCA
AACCUGACCCACCCAAGAACUUGCAGCUGAAGCCAUUAAAGAAUUCUCGG
CAGGUGGAGGUCAGCUGGGAGUACCCUGACACCUGGAGUACUCCACAUUC
CUACUUCUCCCUGACAUUCUGCGUUCAGGUCCAGGGCAAGAGCAAGAGAG
AAAAGAAAGAUAGAGUCUUCACGGACAAGACCUCAGCCACGGUCAUCUGC
CGCAAAAAUGCCAGCAUUAGCGUGCGGGCCCAGGACCGCUACUAUAGCUC
AUCUUGGAGCGAAUGGGCAUCUGUGCCCUGCAGUGGUGGCGGUGGCGGCG
GAUCUAGAAACCUCCCCGUGGCCACUCCAGACCCAGGAAUGUUCCCAUGC
CUUCACCACUCCCAAAACCUGCUGAGGGCCGUCAGCAACAUGCUCCAGAA
GGCCAGACAAACUCUAGAAUUUUACCCUUGCACUUCUGAAGAGAUUGAUC
AUGAAGAUAUCACAAAAGAUAAAACCAGCACAGUGGAGGCCUGUUUACCA
UUGGAAUUAACCAAGAAUGAGAGUUGCCUAAAUUCCAGAGAGACCUCUUU
CAUAACUAAUGGGAGUUGCCUGGCCUCCAGAAAGACCUCUUUUAUGAUGG
CCCUGUGCCUUAGUAGUAUUUAUGAAGACUUGAAGAUGUACCAGGUGGAG
UUCAAGACCAUGAAUGCAAAGCUUCUGAUGGAUCCUAAGAGGCAGAUCUU
UCUAGAUCAAAACAUGCUGGCAGUUAUUGAUGAGCUGAUGCAGGCCCUGA
AUUUCAACAGUGAGACUGUGCCACAAAAAUCCUCCCUUGAAGAACCGGAU
UUUUAUAAAACUAAAAUCAAGCUCUGCAUACUUCUUCAUGCUUUCAGAAU
UCGGGCAGUGACUAUUGAUAGAGUGAUGAGCUAUCUGAAUGCUUCCUAA
Human GMCSF MOP mRNA (hGMCSF-MOP) perfect
matching complementary sequence to miRNA-
122-5P, miRNA-1-3P, miRNA-203a-3P, miRNA-
30a-5P are underlined (MOPV) [SEQ ID NO: 60]
AUGUGGCUGCAGAGCCUGCUGCUCUUGGGCACUGUGGCCUGCAGCAUCUC
UGCACCCGCCCGCUCGCCCAGCCCCAGCACGCAGCCCUGGGAGCAUGUGA
AUGCCAUCCAGGAGGCCCGGCGUCUCCUGAACCUGAGUAGAGACACUGCU
GCUGAGAUGAAUGAAACAGUAGAAGUCAUCUCAGAAAUGUUUGACCUCCA
GGAGCCGACCUGCCUACAGACCCGCCUGGAGCUGUACAAGCAGGGCCUGC
GGGGCAGCCUCACCAAGCUCAAGGGCCCCUUGACCAUGAUGGCCAGCCAC
UACAAGCAGCACUGCCCUCCAACCCCGGAAACUUCCUGUGCAACCCAGAU
UAUCACCUUUGAAAGUUUCAAAGAGAACCUGAAGGACUUUCUGCUUGUCA
UCCCCUUUGACUGCUGGGAGCCAGUCCAGGAGUGACAAACACCAUUGUCA
CACUCCAUUUAAAAUACAUACUUCUUUACAUUCCAUUUAAACUAGUGGUC
CUAAACAUUUCACUUUAAACUUCCAGUCGAGGAUGUUUACA
Human GM-CSF without MOP
[SEQ ID NO: 61]
AUGUGGCUGCAGAGCCUGCUGCUCUUGGGCACUGUGGCCUGCAGCAUCUC
UGCACCCGCCCGCUCGCCCAGCCCCAGCACGCAGCCCUGGGAGCAUGUGA
AUGCCAUCCAGGAGGCCCGGCGUCUCCUGAACCUGAGUAGAGACACUGCU
GCUGAGAUGAAUGAAACAGUAGAAGUCAUCUCAGAAAUGUUUGACCUCCA
GGAGCCGACCUGCCUACAGACCCGCCUGGAGCUGUACAAGCAGGGCCUGC
GGGGCAGCCUCACCAAGCUCAAGGGCCCCUUGACCAUGAUGGCCAGCCAC
UACAAGCAGCACUGCCCUCCAACCCCGGAAACUUCCUGUGCAACCCAGAU
UAUCACCUUUGAAAGUUUCAAAGAGAACCUGAAGGACUUUCUGCUUGUCA
UCCCCUUUGACUGCUGGGAGCCAGUCCAGGAGUGA

### Example 5: In Vivo Biodistribution of DMP^{CTx} of Luciferase Expressing mRNAs with MOP Sequences

To investigate the potential of the present invention to demonstrate differential expression of a particular ORF in vivo, firefly luciferase (FLuc) mRNA was modified as in Example 3 to include two different combinations of three miRNA target sequences in the 3′ UTR of the mRNA construct. The first mRNA MOP sequence contains the target sequences for Let7b, miRNA-126 and miRNA-30a (Group 2). The second mRNA MOP sequence contains the target sequences for miRNA-122, miRNA-192, miRNA-30a (Group 3). All MOP constructs contained perfect match target sequences to the corresponding miRNAs. A control FLuc mRNA sequence was also used without MOP sequences in the construct (Group1). Vehicle group received phosphate-buffered saline.

Formulations were made as described above and had the following characteristics:

TABLE 9
Delivery formulations for in vivo biodistribution

| | | Conc | Encapsulation | Zₐᵥₑ | |
|---|---|---|---|---|---|
| Group | mRNA | (mg/mL) | Efficiency (%) | (nm) | PDI |
| 1 | FLuc | 0.319 | 95.2 | 70.8 | 0.117 |
| 2 | FLuc-let7b-126-30a PM | 0.284 | 92.8 | 73.6 | 0.116 |
| 3 | FLuc-122-192-30a PM | 0.359 | 95.6 | 73.7 | 0.099 |

Animals. All experiments were performed at Crown Biosciences, Nottingham UK in accordance with all local rules and regulations. All mice were obtained from Charles River.

Non-Tumoral Biodistribution Studies. Healthy, female balb/c mice 7-9 weeks old were injected with 1 mg/kg formulation (DMP^{CTx}-mRNA) encoding for firefly luciferase (FLuc) either with or without MOP sequences through a bolus tail vein injection. Whole body Images were taken pre-dosing (0 h), and 3.5 h, and 24 h post dose and the amount of luciferase signal was quantified using Living Image Software (Caliper LS, US). 15 min prior to imaging, mice were injected (subcutaneous) with 150 mg/kg d-Luciferin, then anesthetized 10 min later and placed in an imaging chamber for luminescence detection (ventral and dorsal views). At the 24 h time point, the liver, kidneys, spleen, and lungs were removed and imaged ex vivo.

Tumoral Biodistribution studies. Human liver cancer cells (Hep3B cells) (2×10⁶ cells) were implanted subcutaneously in the left flank of 8-10 week old Fox Chase SCID mice. Mice were sorted into study groups based on caliper measurements of tumor burden, with tumor sizes of approximately 100 mm³ chosen. Formulation (DMP^{CTx}-mRNA) encoding for firefly luciferase (FLuc) either with or without MOP sequences was then injected intratumorally at a dose of 1 mg/kg. Whole body Images were taken pre-dosing (0 h), and 3.5 h, and 24 h post dose and the amount of luciferase signal was quantified using Living Image Software (Caliper LS, US). 15 min prior to imaging, mice were injected (subcutaneous) with 150 mg/kg d-Luciferin, then anesthetized 10 min later and placed in an imaging chamber for luminescence detection (ventral and dorsal views). At the 24 h time point, the tumor, liver, kidneys, spleen, and lungs were removed and imaged ex vivo.

Results

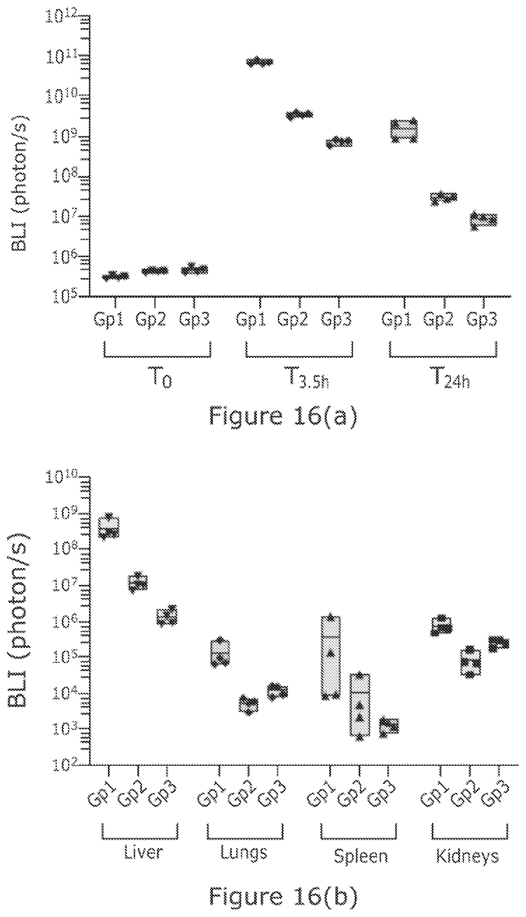

FIG. 16(a) shows that after 3.5 hours post-dosing via intravenous administration, high levels of Luciferase expression can be seen in all groups through whole body imaging, including the MOP containing constructs (Group 2 and 3) and the control group with no MOP construct (Group 1). However, there are 1-2 orders of magnitude less protein expression with the two MOP containing constructs. This trend remains after 24 hours, where there is slightly less overall protein expression in all groups.

The presence of the MOP is surprisingly effective in minimizing off-target expression in vivo in tissues of the liver, lungs, spleen, and kidneys. In FIG. 16(b), ex vivo imaging of the organs show decreased Luciferase expression in the liver (miRNA-122), lungs (let7b, miRNA-126, miR30a), spleen (Let7b, miRNA-126), and kidney (miRNA-192, miRNA-30a) for mice in Group 2 and 3 (MOP containing constructs) as compared to Group 1 (no MOP control), confirming that both MOP constructs provide valuable multi organ protection from expression of the ORF.

While it is important to minimize off target effects in healthy tissue, it is also important to ensure that protein expression still occurs in targeted tissues such as in a tumor. Table 10 shows that protein expression was maintained at the same order of magnitude for all three groups, when a Hep3B liver tumor was present. Additionally, Luciferase expression in the healthy liver decreases 2-3 orders of magnitude when either MOP is present as was seen in the non-tumor bearing in vivo study.

TABLE 10
BLI values (photon/S) obtained in ex vivo imaging

Ex vivo imaging (photon/S)

| Group | Tumor | Liver |
|---|---|---|
| 1 FLuc | 4.02 × 10⁷ | 2.09 × 10⁶ |
| 2 FLuc-let7b-126-30a PM | 3.33 × 10⁷ | 3.75 × 10⁴ |
| 3 FLuc-122-192-30a PM | 1.81 × 10⁷ | 2.13 × 10³ |

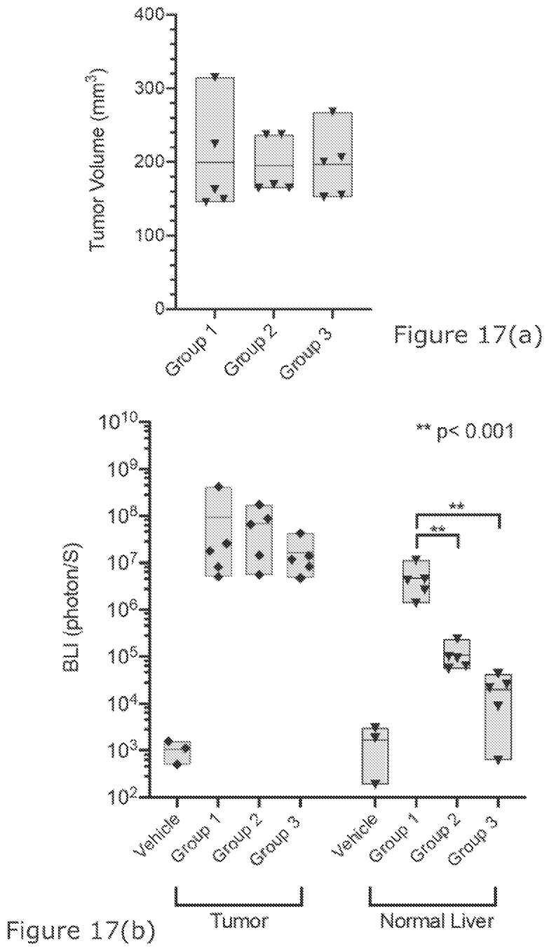

FIG. 17 shows that after 24 hours post-administration, high levels of Luciferase expression can be seen in tumour tissue in all groups through ex vivo imaging, including the MOP containing constructs (Group 2 and 3) and the control group with no MOP construct (Group 1). FIG. 17 (a) shows that the tumour volume in mice was similar in each group. FIG. 17 (b) shows that after 24 hours post-dosing, high Luciferase expression is seen for the control group with no MOP construct (Group 1) in healthy liver tissue (normal liver), with expression reduced by 2-3 orders of magnitude using the MOP containing constructs (Group 2 and 3). Little expression is seen in other organs (data not shown).

### Example 6: In Vivo Biodistribution of DMP^{CTx} of Luciferase Expressing mRNAs with MOP Sequences Following Intramuscular (IM) Administration

This experiment is similar to the approach taken in Example 5. However, since most vaccine compositions are administered via intramuscular injection (IM), it was necessary to demonstrate biodistribution leading to differential expression of a FLuc ORF in vivo following IM administration. The mRNA was modified as in Example 5, but this time to include three different combinations of miRNA target sequences in the 3′ UTR of the mRNA construct. The first mRNA MOP sequence contains the target sequences for Let7b, miRNA-126 and miRNA-30a (Luc-MOP1). The second mRNA MOP sequence contains the target sequences for miRNA-122, miRNA-192, miRNA-30a (Luc-MOP2). The third mRNA MOP sequence contains the target sequences for miRNA-122, miRNA-1, miRNA-203a, miRNA-30a (Luc-MOP3). All MOP constructs contained perfect match target sequences to the corresponding cellular miRNAs. A control FLuc mRNA sequence was also used without MOP sequences in the construct (Luc). A control group without mRNA cargo was also included, in which mice received phosphate saline buffer (Vehicle).

Formulations were made as described above and had the following characteristics:

TABLE 11
Delivery formulations for in vivo biodistribution with IM administration

| | | Conc | Encapsulation | Zₐᵥₑ | |
|---|---|---|---|---|---|
| Group | mRNA | (mg/mL) | Efficiency (%) | (nm) | PDI |
| Luc | FLuc | 0.227 | 94.2 | 79.2 | 0.160 |
| Luc-MOP1 | FLuc-let7b-126-30a PM | 0.221 | 93.1 | 74.9 | 0.114 |
| Luc-MOP2 | FLuc-122-192-30a PM | 0.226 | 93.2 | 85.2 | 0.226 |
| Luc-MOP3 | FLuc-122-1-203a-30a | 0.232 | 95.2 | 59.6 | 0.090 |
| | PM | | | | |

Healthy, female balb/c mice 7-9 weeks old were injected with 10 ug of formulation (DMP^{CTx}-mRNA) encoding for firefly luciferase (FLuc) either with or without MOP sequences through an IM injection. 15 min prior to imaging, mice were injected (subcutaneous) with 150 mg/kg d-Luciferin, then anesthetized 10 min later and placed in an imaging chamber for luminescence detection (ventral and dorsal views). At the 4 h time point, the liver, kidneys, spleen, and the muscle and skin at the site of injection were removed and imaged ex vivo.

Results

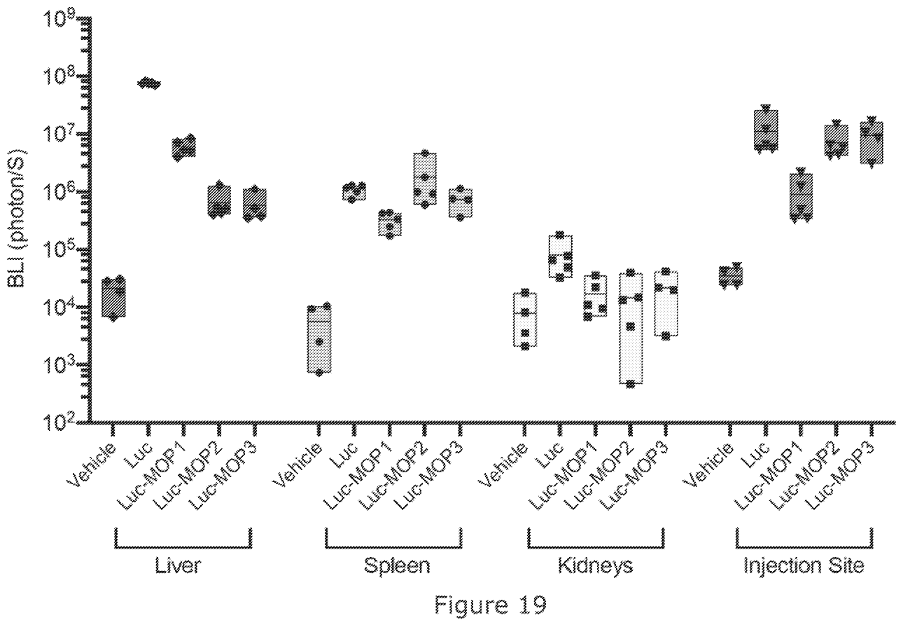

In FIG. 19, ex vivo imaging of the organs show decreased Luciferase expression in the liver (miRNA-122) for all groups. In the spleen (Let7b, miRNA-126), the Luc-MOP1 showed most effect. In kidney (miRNA-192, miRNA-30a), all MOP containing mRNAs showed a trend of reduced expression. At the injection site, Luc-MOP1 reduced production of Luciferase but the other MOPs did not. The results confirm that the different MOP constructs provide valuable multi organ protection from expression of the ORF that can be varied according to need.

### Example 7: In Vivo Biodistribution of DMP^{CTx} of Luciferase Expressing mRNA with MOP Sequences Following Intravenous (IV) Administration

This experiment is similar to the approach taken in Example 5. To assess multi organ protection, we administered the DMP^{CTx} formulations intravenously to ensure high delivery and signal in the organs. The mRNA was modified as in Example 6. The first mRNA MOP sequence contains the target sequences for Let7b, miRNA-126 and miRNA-30a (Luc-MOP1). The second mRNA MOP sequence contains the target sequences for miRNA-122, miRNA-192, miRNA-30a (Luc-MOP2). The third mRNA MOP sequence contains the target sequences for miRNA-122, miRNA-1, miRNA-203a, miRNA-30a (Luc-MOP3). All MOP constructs contained perfect match target sequences to the corresponding cellular miRNAs. A control FLuc mRNA sequence was also used without MOP sequences in the construct (Luc). A control group without mRNA cargo was also included, in which mice received phosphate-buffered saline (Vehicle).

Formulations were made as described above and had the following characteristics:

TABLE 12
Delivery formulations for in vivo biodistribution with IV administration

| | | Conc | Encapsulation | Zₐᵥₑ | |
|---|---|---|---|---|---|
| Group | mRNA | (mg/mL) | Efficiency (%) | (nm) | PDI |
| Luc | FLuc | 0.264 | 94.5 | 79.7 | 0.137 |
| Luc-MOP1 | FLuc-let7b-126-30a PM | 0.291 | 93.2 | 82.5 | 0.115 |
| Luc-MOP2 | FLuc-122-192-30a PM | 0.223 | 92.9 | 84.3 | 0.108 |
| Luc-MOP3 | FLuc-122-1-203a-30a PM | 0.260 | 95.0 | 59.7 | 0.131 |

Healthy, female balb/c mice 7-9 weeks old were injected with 1 mg/kg formulation (DMP^{CTx}-mRNA) encoding for firefly luciferase (FLuc) either with or without MOP sequences through a bolus tail vein injection. Whole body images were taken 6 hours post dose and the amount of luciferase signal was quantified using Living Image Software (Caliper LS, US). 15 min prior to imaging, mice were injected (subcutaneous) with 150 mg/kg d-Luciferin, then anesthetized 10 min later and placed in an imaging chamber for luminescence detection (ventral and dorsal views). At the 6 h time point, the liver, kidneys, spleen, heart, pancreas and lungs were removed and imaged ex vivo.

Results

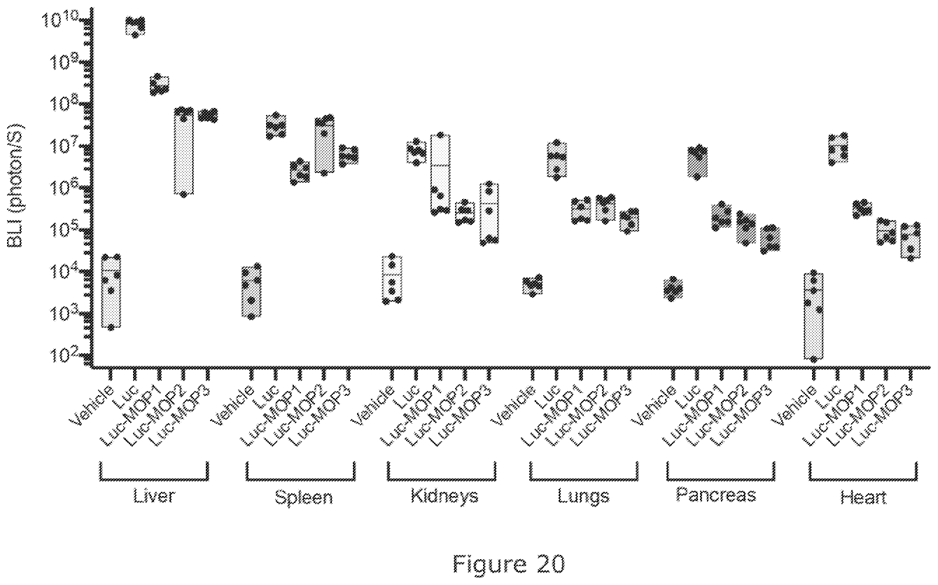

The presence of the MOP is again surprisingly effective in minimizing off-target expression in vivo in tissues of the liver, lungs, spleen, pancreas, heart, and kidneys. In FIG. 20, ex vivo imaging of the organs show decreased Luciferase expression in the liver (miRNA-miRNA-126, miR30a), spleen (Let7b, miRNA-126), pancreas (miRNA-30 family, Let7 family, miRNA-122), heart (miRNA-30 family, miRNA-126, Let7 family) and kidney (miRNA-192, miRNA-30a) for mice in group dosed with MOP containing constructs (Luc-MOP1, Luc-MOP2, and Luc-MOP3) as compared to group Luc (no MOP control), confirming that all MOP constructs provide valuable multiple-organ protection from expression of the ORF.

### Example 8: In Vivo Assessment of an Antigen-Specific Immune Response

To investigate the ability of compositions according to the invention to act as vaccines and to demonstrate adjuvant effects of a co-administered cytokine, a study was conducted in vivo to generate an antibody response against a particular exogenous antigen, in this case, ovalbumin (OVA), an egg white protein.

For this experiment, mRNA constructs were encapsulated in nanoparticle compositions, DMP^{CTx} as described previously. The compositions used were nanoparticle compositions comprising mRNA encoding ovalbumin protein (DMP^{CTx}-OVA), and nanoparticle compositions comprising mRNA encoding murine single-chain IL-12 protein with MOP sequences (DMP^{CTx}-mscIL-12-MOP). The MOP sequences, where used, comprised perfect matched binding sites for miRNA-122, miRNA-1, miRNA-203a, miRNA-30a (see SEQ ID NO: 68).

TABLE 13
ORF and 3′ UTR for IL-12
Murine single chain IL-12 MOP (mscIL12-MOP),
without codon optimisation, with 3′ UTR
perfect matching complementary sequence to
miRNA-122-5P, miRNA-1-3P, miRNA-203a-3P,
miRNA-30a-5P are underlined (MOPV)
[SEQ ID NO: 68]

AUGUGUCCUCAGAAGCUAACCAUCUCCUGGUUUGCCAUCGUUUUGCUGGU
GUCUCCACUCAUGGCCAUGUGGGAGCUGGAGAAAGACGUUUAUGUUGUAG
AGGUGGACUGGACUCCCGAUGCCCCUGGAGAAACAGUGAACCUCACCUGU
GACACGCCUGAAGAAGAUGACAUCACCUGGACCUCAGACCAGAGACAUGG
AGUCAUAGGCUCUGGAAAGACCCUGACCAUCACUGUCAAAGAGUUUCUAG
AUGCUGGCCAGUACACCUGCCACAAAGGAGGCGAGACUCUGAGCCACUCA
CAUCUGCUGCUCCACAAGAAGGAAAAUGGAAUUUGGUCCACUGAAAUUUU
AAAAAAUUUCAAAAACAAGACUUUCCUGAAGUGUGAAGCACCAAAUUACU
CCGGACGGUUCACGUGCUCAUGGCUGGUGCAAAGAAACAUGGACUUGAAG
UUCAACAUCAAGAGCAGUAGCAGUUCCCCUGACUCUCGGGCAGUGACAUG
UGGAAUGGCGUCUCUGUCUGCAGAGAAGGUCACACUGGACCAAAGGGACU
AUGAGAAGUAUUCAGUGUCCUGCCAGGAGGAUGUCACCUGCCCAACUGCC
GAGGAGACCCUGCCCAUUGAACUGGCGUUGGAAGCACGGCAGCAGAAUAA
AUAUGAGAACUACAGCACCAGCUUCUUCAUCAGGGACAUCAUCAAACCAG
ACCCGCCCAAGAACUUGCAGAUGAAGCCUUUGAAGAACUCACAGGUGGAG
GUCAGCUGGGAGUACCCUGACUCCUGGAGCACUCCCCAUUCCUACUUCUC
CCUCAAGUUCUUUGUUCGAAUCCAGCGCAAGAAAGAAAAGAUGAAGGAGA
CAGAGGAGGGGUGUAACCAGAAAGGUGCGUUCCUCGUAGAGAAGACAUCU
ACCGAAGUCCAAUGCAAAGGCGGGAAUGUCUGCGUGCAAGCUCAGGAUCG
CUAUUACAAUUCCUCAUGCAGCAAGUGGGCAUGUGUUCCCUGCAGGGUCC
GAUCCGGUGGCGGUGGCUCGGGCGGUGGUGGGUCGGGUGGCGGCGGAUCU
AGGGUCAUUCCAGUCUCUGGACCUGCCAGGUGUCUUAGCCAGUCCCGAAA
CCUGCUGAAGACCACAGAUGACAUGGUGAAGACGGCCAGAGAAAAACUGA
AACAUUAUUCCUGCACUGCUGAAGACAUCGAUCAUGAAGACAUCACACGG
GACCAAACCAGCACAUUGAAGACCUGUUUACCACUGGAACUACACAAGAA
CGAGAGUUGCCUGGCUACUAGAGAGACUUCUUCCACAACAAGAGGGAGCU
GCCUGCCCCCACAGAAGACGUCUUUGAUGAUGACCCUGUGCCUUGGUAGC
AUCUAUGAGGACUUGAAGAUGUACCAGACAGAGUUCCAGGCCAUCAACGC
AGCACUUCAGAAUCACAACCAUCAGCAGAUCAUUCUAGACAAGGGCAUGC
UGGUGGCCAUCGAUGAGCUGAUGCAGUCUCUGAAUCAUAAUGGCGAGACU
CUGCGCCAGAAACCUCCUGUGGGAGAAGCAGACCCUUACAGAGUGAAAAU
GAAGCUCUGCAUCCUGCUUCACGCCUUCAGCACCCGCGUCGUGACCAUCA
ACAGGGUGAUGGGCUAUCUGAGCUCCGCCUAACAAACACCAUUGUCACAC
UCCAUUUAAAAUACAUACUUCUUUACAUUCCAUUUAAACUAGUGGUCCUA
AACAUUUCACUUUAAACUUCCAGUCGAGGAUGUUUACA

Formulations were made as described above and had the following characteristics:

TABLE 14
Delivery formulations for in vivo OVA immunogenicity study

| | Conc | Encapsulation | Zₐᵥₑ | |
|---|---|---|---|---|
| mRNA | (mg/mL) | Efficiency (%) | (nm) | PDI |
| OVA | 0.447 | 96.6 | 59.6 | 0.108 |
| Murine IL-12-MOPV | 0.424 | 96.8 | 61.9 | 0.120 |

6-8 week old Balb/c female mice were randomised by body weight on study day −1 into 4 groups each. On Day 0, mice received intramuscular injections of 50 μl in the left thigh of:
3 μg of DMP^{CTx}-OVA (Group 2, LNP-OVA);
3 μg of DMP^{CTx}-OVA and DMP^{CTx}-mscIL-12 at a dose of 5 μg of the IL-12 construct (Group 3; LNP-OVA+IL12);
10 μg of DMP^{CTx}-OVA (Group 4; LNP-OVA)(
Control Group 1 received intramuscular injections of 50 μl in the left thigh of vehicle phosphate-buffered saline only on study day 0.

On study day 14, blood was collected via terminal cardiac puncture for serum isolated by centrifugation (90 sec, RT, 10.000×g), and aliquots frozen. Collected serum was processed for detection of mouse anti-OVA IgG according to the manufacturer's instructions (Chondrex).

Results

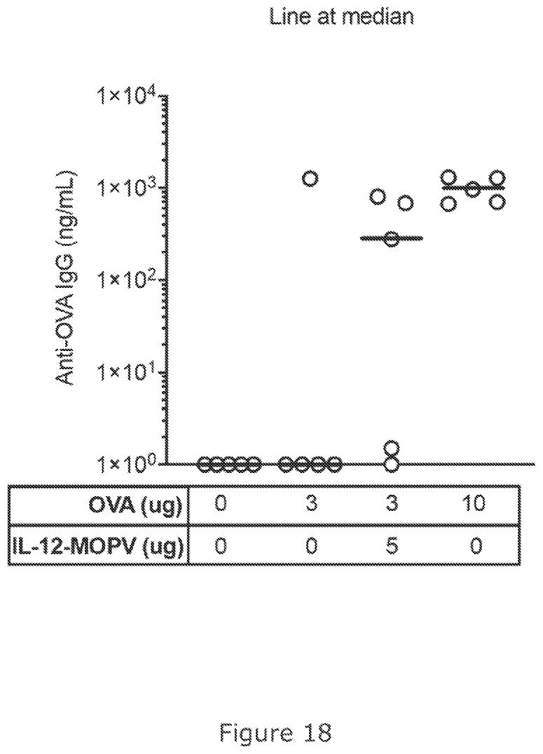

FIG. 18 shows the results of the experiment. At a low dose of 3 μg mRNA, DMP^{CTx}-OVA (ovalbumin) induced a weak response with only one mouse responder. This is increased substantially by increasing the dosage of mRNA more than three-fold to 10 μg. However, co-administration of the low dose of ovalbumin together with the pro-inflammatory cytokine IL12 as adjuvant resulted in a significant improvement in the number of responders. This indicates that low dose antigen response can be boosted in vivo by the presence of co-administered IL-12.

These results demonstrate that an IgG antibody response can be induced against a given target. Given that ovalbumin is not a pathogen-specific antigen, this also demonstrates that this outcome is a result of the intervention, and should be applicable to any provided exogenous polypeptide antigen. The results are further surprising because the in vivo administration of IL-12 was not systemic and was controlled by the MOP sequence thereby reducing off target expression of the proinflammatory molecule.

### Example 9: In Vivo Assessment of SARS-CoV-2 Spike Protein-Specific Immune Response

To further investigate the ability of compositions according to the invention to act as vaccines against a specific pathogenic target, that is, to provoke an in vivo immune response against viral antigen, mice were injected with nanoparticle compositions comprising mRNA encoding the SARS-CoV-2 spike protein, and nanoparticle compositions comprising mRNA encoding the immunomodulatory cytokine IL-12 (as described in previous Example 8). The humoral immune response of the mice to the compositions was tested.

For this experiment, mRNA constructs were encapsulated in nanoparticle compositions as described previously. The compositions used were nanoparticle compositions comprising mRNA encoding a SARS-CoV-2 Spike protein as set out in SEQ ID NO: 63 (DMP^{CTx}-Spike CoV) as the antigen, and nanoparticle compositions comprising mRNA encoding murine single-chain IL-12 protein as set out in SEQ ID NO: 68 (DMP^{CTx}-mscIL-12) as the adjuvant. Both comprise MOPV (miRNA-122, miRNA-1, miRNA-203a, miRNA-30a) sequences at the 3′ UTR.

Formulations were made as described above and had the following characteristics:

TABLE 15
Delivery formulations for in vivo immunogenicity study

| | | Conc | Encapsulation | Zₐᵥₑ | |
|---|---|---|---|---|---|
| Dose | mRNA | (mg/mL) | Efficiency (%) | (nm) | PDI |
| Prime | Spike-MOPV | 0.114 | 94.7 | 55.0 | 0.131 |
| Prime | Murine IL-12-MOPV | 0.121 | 94.4 | 58.9 | 0.065 |
| Boost | Spike-MOPV | 0.373 | 95.5 | 68.2 | 0.144 |
| Boost | Murine IL-12-MOPV | 0.299 | 94.7 | 60.2 | 0.092 |

Balb/c female mice were randomized on study day 0 by body weight into three groups each. On Day 0 and Day 14, mice received intramuscular injections of 50 μl in the left thigh of:

DMP^{CTx}-Spike CoV (LNP-spike dose 1 μg at day 0 and boosted 10 μg at day 14—a so called 1/10 dosage regime);
or of DMP^{CTx}-Spike CoV (LNP-spike dose 1 μg at day 0 and boosted 10 μg at day 14—a so called 1/10 dosage regime) and the IL-12 construct DMP^{CTx}-mscIL12 (1 ug at day 0 and day 14). A control group received intramuscular injections of 50 ul in the left thigh of vehicle phosphate-buffered saline on study day 0 and 14.

At day 42 of the study (42 days post-prime and 28 days post-boost), blood was collected by terminal cardiac puncture from all groups and serum isolated by centrifugation (90 sec, RT, 10.000×g), and aliquots frozen and stored at −80° C. To detect anti-Spike antibodies in the collected serum, Mouse Anti-SARS-CoV-2 IgG Antibody ELISA Kit (AcroBiosystems, Catalog #RAS-T023) was used according to the manufacturer's instructions.

Results

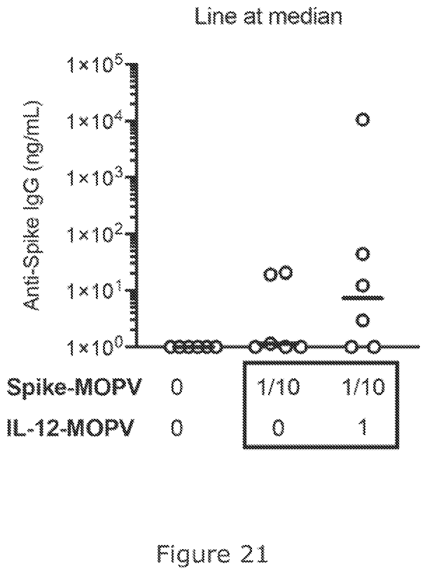

FIG. 21 shows that there is a definite increase in IgG production in response to the spike protein as IL12 is added.

These results demonstrate that in vivo the addition of an IL-12 immunomodulatory cytokine as an adjuvant is advantageous to generation of an immune response to a SARS-CoV-2 spike protein antigen. This response is maintained surprisingly well even when both the antigen and the adjuvant were administered as mRNA under the control of a MOP sequence that reduces systemic/off-target production of both the antigen and the adjuvant. In combination with the in vitro data shown in Example 4 (see FIG. 12(a)), which indicated that IL-12 could be produced in human PBMCs in as little as six hours from administration, this is a clear further indication that a protective immune response can be generated from vaccine and adjuvant compositions that comprise MOP sequences in a relatively short time from administration of the vaccine and adjuvant.

### Example 10: In Vitro Transfection of Human PBMCs with Human IL-12

To further investigate the ability of compositions according to the invention to act as a vaccine adjuvant, we transfected human peripheral blood mononuclear cells (PBMCs) with DMP^{CTx}-hscIL-12 with/without MOP and measured IL-12-mediated induction of interferon-gamma.

PBMC cells from four different donors were obtained from StemCell Technologies and cultured in suspension in AIM V medium (Gibco) at 37° C. in an atmosphere of 5% CO2. 300,000 PBMC cells were seeded per well in a round-bottom 96-well plate and transfected with DMP^{CTx}-hscIL-12 single either with or without MOP sequences. The first mRNA MOP sequence contains the target sequences for miRNA-122, miRNA-1, miRNA-203a, miRNA-30a (MOPV). The second mRNA MOP sequence contains the target sequences for miRNA-122, miRNA-192, miRNA-30a (MOPC). All MOP constructs contained perfect match target sequences to the corresponding cellular miRNAs. Three doses of DMP^{CTx}-hscIL-12 (with or without MOP) were used. Negative controls were performed in parallel, with PBMCs not transfected or PBMCs transfected with a human single chain IL-12 non-coding mRNA (hIL-12 NC—no ATG start codon). The plates were incubated at 37° C., 5% CO₂ for 4 hours.

Formulations were made as described above and had the following characteristics:

TABLE 16
Delivery formulations for in vitro transfection
of human PBMC with IL-12

| | Conc | Encapsulation | Zₐᵥₑ | |
|---|---|---|---|---|
| mRNA | (mg/mL) | Efficiency (%) | (nm) | PDI |
| hscIL-12 NC | 0.154 | 95.3 | 59.8 | 0.103 |
| hscIL-12 | 0.127 | 95.3 | 62.1 | 0.120 |
| hscIL-12-MOPV | 0.140 | 95.3 | 62.1 | 0.112 |
| hscIL-12-MOPC | 0.150 | 95.1 | 80.0 | 0.094 |

4 hours after transfection, human AB heat-inactivated serum (Valley Biomedical) was added to a final concentration of 5%. 24 hours after transfection, 100 μL of the supernatant of each well were transferred to a new 96-well plate, cells were removed by centrifugation and the supernatants were frozen at −80° C. for human IL-12 ELISA assays (Invitrogen, Catalog 88-7126). 72 hours after transfection, 100 μL of the supernatant of each well were transferred to a new 96-well plate, cells were removed by centrifugation and the supernatants were frozen at −80° C. for Interferon-gamma ELISA assays (Biolegend Catalog 430116). ELISA assays for human IL-12p70 and human interferon-gamma was following the manufacturer's instructions. The data were plotted into a bar graph using Graph Pad Prism.

Results

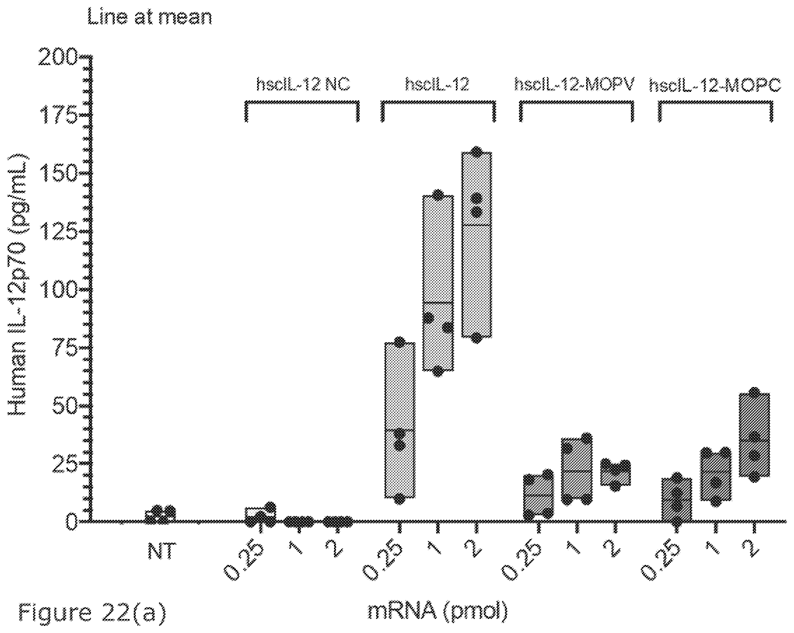

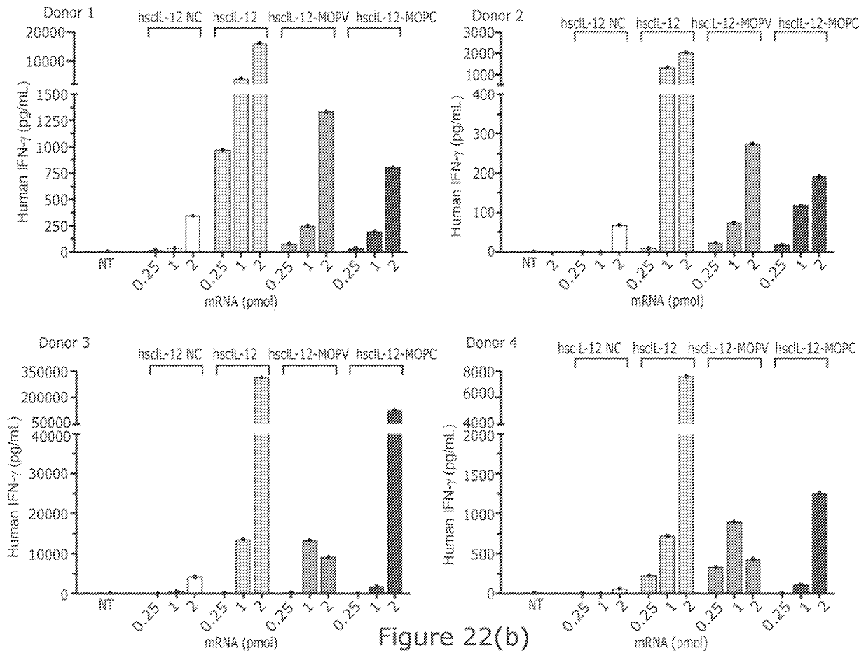

FIG. 22a shows a dose-dependent expression of human IL-12 in human PBMC transfected with DMP^{CTx}-hscIL-12 products. FIG. 22b (with each donor shown separately), shows an IL-12-mediated induction of IFN-γ, which is an immunostimulatory cytokine critical for both innate and adaptive immunity. While the amplitude of IL-12-mediated IFN-γ expression varies between the donors, there is an evident correlation between IL-12 presence and IFN-γ expression.

### Example 11: In Vitro Transfection of Human PBMCs with Human IL-12 and SARS-CoV-2 Spike

To further investigate the ability of compositions according to the invention to activate innate and adaptive immune response, that is, to induce interferon-gamma, peripheral blood mononuclear cells (PBMCs) were transfected using nanoparticle compositions comprising mRNA encoding the SARS-CoV-2 spike protein, and nanoparticle compositions comprising mRNA encoding the immunomodulatory agent IL-12.

For this experiment, mRNA constructs were encapsulated in nanoparticle compositions as described above. The compositions used were nanoparticle compositions comprising mRNA encoding the SARS-CoV-2 Spike protein with MOP sequences (DMP^{CTx}-SCoV-MOPV), and nanoparticle compositions comprising mRNA encoding human single-chain IL-12 protein (DMP^{CTx}-hscIL-12), with or without MOP sequences (DMP^{CTx}-hscIL-12-MOPV). The MOP sequences, where used, comprised the target sequences for miRNA-122, miRNA-1, miRNA-203a, miRNA-30a (MOPV).

Formulations were made as described above and had the following characteristics:

TABLE 17
Delivery formulations for in vitro transfection
of human PBMC wit IL-12 and SARS-CoV-2 Spike

| | Conc | Encapsulation | Zₐᵥₑ | |
|---|---|---|---|---|
| mRNA | (mg/mL) | Efficiency (%) | (nm) | PDI |
| Spike-MOPV | 0.384 | 95.3 | 63.4 | 0.125 |
| hscIL-12 | 0.382 | 94.7 | 60.7 | 0.077 |
| hscIL-12-MOPV | 0.343 | 93.7 | 61.7 | 0.123 |

Five healthy donor PBMCs were transfected with DMP^{CTx}-SCoV-MOP and with or without DMP^{CTx}-hscIL-12 and DMP^{CTx}-hscIL-12-MOPV. All PBMCs were seeded on anti-CD3 coated plates and treated with soluble anti-CD28 monoclonal antibody to induce general T cell activation. Five days after transfection, supernatants were harvested for IFN-γ quantification by ELISA.

On Day 0, PMBC (300 000 cells) were seeded and the nanoparticle compositions or sterile PBS was added. The plates were incubated at 37° C., 5% CO₂ for 4 hours. 10 μl of human AB serum (hAB) was added for a total volume of 200 μl and a total hAB serum concentration of 5%.

On day 5, supernatant was harvested as follows. The plates were spun down at 300×g, 5 minutes at RT. Supernatant was transferred into V-bottom 96 well plates and spun at 400×g for 5 minutes at RT. Supernatant was transferred into fresh V-bottom 96 well plates and stored at −80° C. Readouts for IFN-gamma expression by Meso-Scale Discovery (MSD Catalog K151TTK-2) were carried out according to the manufacturer's instructions.

Results

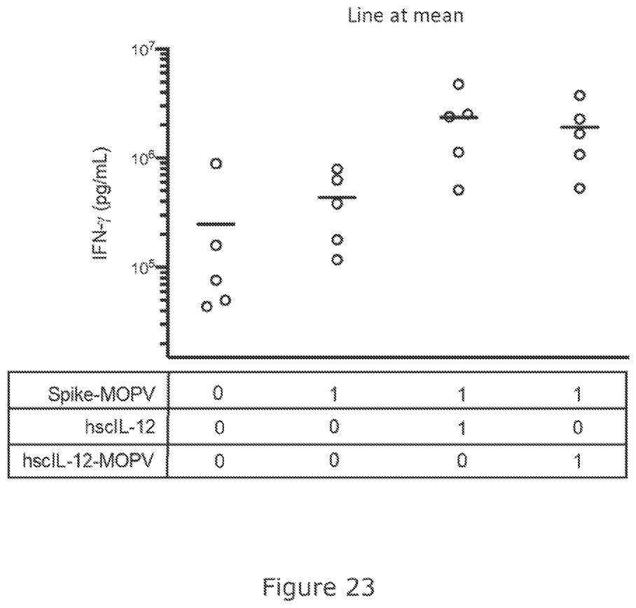

FIG. 23 shows that interferon-gamma (IFN-γ) expression increases similarly in the presence of mRNA expressing human IL-12 with and without MOP. Early synthesis of IFN-γ after immunization, which develops before the appearance of adaptive immune responses, is a sign of high-quality immune response against a vaccine, as its early release will help dendritic cell maturation and consequently the polarisation of CD4⁺ T cells to a T_{H}1 lineage.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims. Any non-human nucleic acid and/or polypeptide sequences that have been included in constructs and vectors according to embodiments of the invention have been obtained from sources within the UK, USA and European Union. To the inventor's knowledge, no genetic resources that would be subject to access and benefit sharing agreements, or associated traditional knowledge has been utilised in the creation of the present invention.

1. A composition comprising:
a first mRNA construct comprising a first open reading frame (ORF), wherein the first ORF encodes a first antigen, wherein the first antigen is selected from a pathogenic microbial antigen or a tumor-associated antigen, or an epitope containing fragment thereof;
wherein the first ORF is operatively linked to at least one untranslated region (UTR), wherein the UTR comprises at least a first organ protection sequence (OPS), and wherein the first OPS comprises at least three miRNA target (miRNA) sequences which are all different from each other, and wherein each of the at least three miRNA target sequences are optimised to hybridise with a corresponding miRNA sequence.

2. The composition of claim 1, wherein the first mRNA construct is comprised within or adsorbed to an in vivo delivery composition.

3. (canceled)

4. The composition of claim 1, wherein the pathogenic microbial antigen is selected from a viral protein; a bacterial protein; a fungal protein; a parasite protein; or a prion.

5. The composition of claim 1, further comprising a second mRNA construct comprising a second open reading frame (ORF), wherein the second ORF encodes a proinflammatory cytokine.

6. The composition of claim 5, wherein the proinflammatory cytokine is selected from the group consisting of: interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), interleukin 19 (IL-19), interleukin 20 (IL-20), interleukin 21 (IL-21), interleukin 22 (IL-22), interleukin 23 (IL-23), interleukin 24 (IL-24), interleukin 25 (IL-25), interleukin 26 (IL-26), interleukin 27 (IL-27), interleukin 28 (IL-28), interleukin 29 (IL-29), interleukin 30 (IL-30), interleukin 31 (IL-31), interleukin 32 (IL-32), interleukin 33 (IL-33), interleukin 35 (IL-35), interleukin 36 (IL-36); CXCL9: IFNγ; IFNα; IFNβ; TNFα; and GM-CSF.

7. The composition of claim 5, wherein the second mRNA construct is comprised within or adsorbed to a delivery composition.

8. The composition of claim 6, wherein the second ORF codes for an IL-12 protein, or a subunit, derivative, fragment, agonist or homologue thereof.

9. The composition of claim 8, wherein the second ORF comprises a sequence at least 90% identical to SEQ ID NO: 59.

10. The composition of claim 5, wherein the second ORF is operatively linked to a second untranslated region (UTR), wherein the UTR comprises a second organ protection sequence (OPS) and wherein the second OPS comprises at least three micro-RNA (miRNA) target sequences which are all different from each other.

11. The composition of claim 10, wherein the at least three miRNA target sequences are optimised to hybridise with a corresponding miRNA sequence.

12. The composition of claim 10, wherein the first OPS includes at least one different miRNA target sequence to the second OPS.

13. The composition of claim 10, wherein the first OPS and the second OPS include the same miRNA target sequences.

14. The composition of claim 1, wherein the composition includes a delivery composition that comprises a delivery vector selected from a particle, such as a polymeric particle; a lipid nanoparticle (LNP); a liposome; a lipidoid particle; or a viral vector.

15. The composition of claim 1, wherein the first OPS comprises at least three, at least four, or at least five miRNA target sequences.

16. (canceled)

17. The composition of claim 1, wherein the first OPS comprises miRNA sequences selected to protect one or more organs or tissues selected from the group consisting of muscle, liver, brain, breast, endothelium, pancreas, colon, kidney, lungs, spleen and skin, heart, gastrointestinal organs, reproductive organs, and esophagus.

18. The composition of claim 1, wherein the first OPS comprises at least three miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family; miRNA-877; miRNA-4300; miRNA-4720; and/or miRNA-6761.

19-20. (canceled)

21. The composition of claim 1, wherein the first OPS comprises sequences selected from one or more of SEQ ID NOs: 44-57.

22-28. (canceled)

29. The composition of claim 1, wherein the antigen comprises a viral protein or an epitope containing fragment thereof.

30. The composition of claim 29, wherein the antigen is selected from a coronavirus spike protein, an influenza protein, a respiratory syncytial virus (RSV) protein or a Human Immunodeficiency Virus (HIV) protein, or a variant thereof, or an epitope containing fragment thereof.

31-39. (canceled)

40. The composition of claim 1, wherein the antigen comprises a protein from the Mycobacterium tuberculosis bacterium or an epitope containing fragment thereof.

41. The composition of claim 40, wherein the protein from the Mycobacterium tuberculosis bacterium is selected from ESAT-6, Ag85B, TB10.4, Rv2626 and/or RpfD-B.

42. The composition of claim 1, wherein the antigen is a tumor-associated antigen selected from a colorectal tumor antigen; a breast tumor antigen; a lung tumor antigen; a liver tumor antigen; or a pancreas tumor antigen.

43. The composition of claim 1, wherein the antigen is a tumor-associated antigen which is MUC1.

44. The composition of claim 1, wherein the antigen is a tumor-associated antigen which is a neoantigen.

45-157. (canceled)

158. The composition of claim 1, wherein the first mRNA comprises a second ORF encoding a second antigen, wherein the second antigen is different to the first antigen.

159. The composition of claim 158, wherein the second antigen is selected from: a pathogenic microbial antigen or a tumor-associated antigen, or an epitope containing fragment thereof.

160. The composition of claim 1, further comprising at least a second mRNA construct comprising at least a second open reading frame (ORF), wherein the second ORF encodes a second antigen, wherein the second antigen is different to the first antigen.

161. The composition of claim 160, wherein the second antigen is selected from: a pathogenic microbial antigen or a tumor-associated antigen, or an epitope containing fragment thereof.
